(19) United States
(12) Patent Application Publication
(10) Pub. No.: US 2007/0049603 A1
(43) Pub. Date: Mar. 1, 2007

(54) Raf inhibitor compounds and methods of use thereof

(76) Inventors: Greg Miknis, Broomfield, CO (US); Joseph P. Lyssikatos, Superior, CO (US); Ellen Laird, Longmont, CO (US); Eugene Tarlton, Louisville, CO (US); Alexandre J. Buckmelter, Superior, CO (US); Li Ren, Superior, CO (US); Bryson Rast, Lafayette, CO (US); Stephen T. Schlacter, Boulder, CO (US); Steven Mark Wenglowsky, Boulder, CO (US)

Correspondence Address: VIKSNINS HARRIS & PADYS PLLP, P.O. BOX 111098, ST. PAUL, MN, 55111-1098, US

(21) Appl. No.: 11/513,625

(22) Filed: Aug. 31, 2006

Related U.S. Application Data
(60) Provisional application No. 60/713,630, filed on Sep. 1, 2005.

(51) Int. Cl. A61K 31/517; A61K 31/506; A61K 31/4743; A61K 31/4741; C07D 491/02; C07D 498/02

(52) U.S. Cl. 514/252.18; 514/252.19; 514/301; 544/295; 546/113; 546/114; 514/302; 514/266.21; 544/284

(57) ABSTRACT

Compounds of Formula I are useful for inhibiting Raf kinase and for treating disorders mediated thereby. Methods of using compounds of Formula I, and stereoisomers, geometric isomers, tautomers, solvates and pharmaceutically acceptable salts thereof, for in vitro, in situ, and in vivo diagnosis, prevention or treatment of such disorders in mammalian cells, or associated pathological conditions are disclosed.

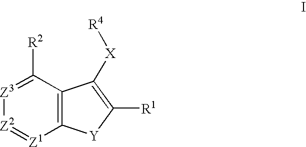

### CROSS REFERENCE TO RELATED APPLICATIONS

This application claims priority to U.S. Provisional Application Ser. No. 60/713,630 filed Sep. 1, 2005, which is incorporated herein by reference in its entirety.

### BACKGROUND OF THE INVENTION

1. Field of the Invention

Provided are compounds that are inhibitors of Raf kinase, as well as compositions containing these compounds and methods of use thereof. The compounds are useful for inhibiting Raf kinase and for treating disorders mediated thereby. Also provided are methods of using the compounds of the present invention for in vitro, in situ, and in vivo diagnosis or treatment of mammalian cells and/or associated pathological conditions.

2. Description of the State of the Art

The Raf/MEK/ERK (extracellular signal-regulated kinase) kinase cascade is pivotal in transmitting signals from membrane receptors to transcription factors that control gene expression culminating in the regulation of cell cycle progression (Robinson, M J and Cobb, M H (1997) Curr. Opin. Cell Biol., 9:180-186). This cascade can prevent cell death through ERK2 and p90(Rsk) activation and phosphorylation of apoptotic and cell cycle regulatory proteins (Shelton, J G, et al. (2003) Oncogene, 22(16):2478-92). The PI3K/Akt kinase cascade also controls apoptosis and can phosphorylate many apoptotic and cell cycle regulatory proteins. These pathways are interwoven as Akt can phosphorylate Raf and result in its inactivation, and Raf can be required for the anti-apoptotic effects of Akt. Raf is a key serine-threonine protein kinase which participates in the transmission of growth, anti-apoptotic and differentiation messages. These signals can be initiated after receptor ligation and are transmitted to members of the MAP kinase cascade that subsequently activate transcription factors controlling gene expression.

Raf is a multigene family which expresses oncoprotein kinases: A-Raf, B-Raf and C-Raf (also known as Raf-1), and isoformic variants that result from differential splicing of mRNA are known (McCubrey, J A, et al., (1998) Leukemia 12(12):1903-1929; Ikawa, et al., (1988) Mol. and Cell. Biol., 8(6):2651-2654; Sithanandam, et al., (1990) Oncogene, 5:1775-1780; Konishi, et al., (1995) Biochem. and Biophys. Res. Comm., 216(2):526-534). All three Raf kinases are functionally present in certain human hematopoietic cells, and their aberrant expression can result in abrogation of cytokine dependency. Their regulatory mechanisms differ because C-Raf and A-Raf require additional serine and tyrosine phosphorylation within the N region of the kinase domain for full activity (Mason et al., (1999) EMBO J., 18:2137-2148), and B-Raf has a much higher basal kinase activity than either A-Raf or C-Raf. The three Raf oncoproteins play critical roles in the transmission of mitogenic and anti-apoptotic signals. Recently, it has been shown that B-Raf is frequently mutated in various human cancers (Wan, et al., (2004) Cell, 116:855-867). Development of specific Raf inhibitors may prove efficacious in cancer therapy. The cytoplasmic serine/threonine kinase B-Raf and receptor tyrosine kinases of the platelet-derived growth factor receptor (PDGFR) family are frequently activated in cancer by mutations of an equivalent amino acid. Structural studies have provided important insights into why these very different kinases share similar oncogenic hot spots and why the PDGFR juxtamembrane region is also a frequent oncogenic target (Dibb, N J (2004) Nature Reviews Cancer, 4(9):718-27).

Transformation of normal melanocytes into melanoma cells is accomplished by the activation of growth stimulatory pathways, typically leading to cellular proliferation and the inactivation of apoptotic and tumor suppressor pathways. Small molecule inhibitors of proteins in the growth stimulatory pathways are under active investigation, and their application to melanoma patients would represent a new treatment strategy to inhibit cell proliferation or induce cell death (Polsky, D., (2003) Oncogene, 22(20):3087-3091; Konopleva, M., et al., (2003) Blood, 102(11):625a).

B-Raf encodes a RAS-regulated kinase that mediates cell growth and malignant transformation kinase pathway activation. Activating B-Raf mutations have been identified in 66% of melanomas and a smaller percentage of many other human cancers. B-Raf mutations also account for the MAP kinase pathway activation common in non-small cell lung carcinomas (NSCLCs), including V600E and other mutations identified as novel, altering residues important in AKT-mediated B-Raf phosphorylation, which suggest that disruption of AKT-induced B-Raf inhibition can play a role in malignant transformation. Although >90% of B-Raf mutations in melanoma involve codon 600 (57 of 60), 8 of 9 B-Raf mutations reported to date in NSCLC are non-V600 (89%; P<10⁻⁷), strongly suggesting that B-Raf mutations in NSCLC are qualitatively different from those in melanoma; thus, there may be therapeutic differences between lung cancer and melanoma in response to RAF inhibitors. Although uncommon, B-Raf mutations in human lung cancers may identify a subset of tumors sensitive to targeted therapy (Brose, M S, et al., (2002) Cancer Research, 62(23):6997-7000).

Raf protein kinases are key components of signal transduction pathways by which specific extracellular stimuli elicit precise cellular responses in mammalian cells. Activated cell surface receptors activate ras/rap proteins at the inner aspect of the plasma membrane, which in turn recruit and activate Raf proteins. Activated Raf proteins phosphorylate and activate the intracellular protein kinases MEK1 and MEK2. In turn, activated MEKs catalyze phosphorylation and activation of p42/p44 mitogen-activated protein kinase (MAPK). A variety of cytoplasmic and nuclear substrates of activated MAPK are known which directly or indirectly contribute to the cellular response to environmental change.

Small molecule inhibitors of the Raf/MEK/ERK pathway are being developed for anticancer therapy (Thompson et al., (2005) Current Opinion in Pharmacology 5:1-7). Inhibitors of Raf kinases have been suggested for use in disruption of tumor cell growth and hence in the treatment of cancers, e.g. histiocytic lymphoma, lung adenocarcinoma, small cell lung cancer and pancreatic and breast carcinoma; and also in the treatment and/or prophylaxis of disorders associated with neuronal degeneration resulting from ischemic events, including cerebral ischemia after cardiac arrest, stroke and multi-infarct dementia and also after cerebral ischemic events such as those resulting from head injury, surgery and/or during childbirth (neurotrauma). In particular, it has been suggested that B-Raf is the major Raf isoform activated by the neurotrophin, nerve growth factor (NGF), for NGF induced extracellular signaling by kinase activation (York et al., (2000) Mol. and Cell. Biol. 20(21):8069-8083).

### SUMMARY OF THE INVENTION

In one aspect, the invention relates to compounds that are inhibitors of Raf kinases, in particular inhibitors of B-Raf kinase. Certain hyperproliferative disorders are characterized by the overactivation of Raf kinase function, for example by mutations or overexpression of the protein. Accordingly, the compounds of the invention are useful in the treatment of hyperproliferative disorders such as cancer.

More specifically, one aspect of the invention provides compounds of Formula I

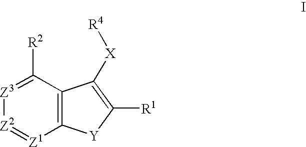

and stereoisomers, geometric isomers, tautomers, solvates, metabolites and pharmaceutically acceptable salts and prodrugs thereof, wherein X, Y, Z¹, Z², Z³, R¹, R² and R⁴ are as defined herein.

Also provided are compounds of Formula VI

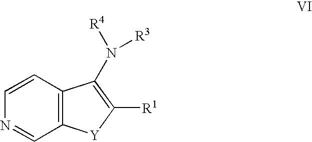

and stereoisomers, geometric isomers, tautomers, solvates, metabolites, salts (including pharmaceutically acceptable salts) and pharmaceutically acceptable prodrugs thereof, wherein Y, R¹, R³ and R⁴ are as defined herein.

Also provided are compounds of Formula II

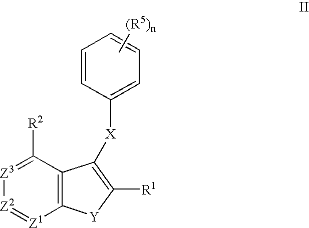

and stereoisomers, geometric isomers, tautomers, solvates, metabolites and pharmaceutically acceptable salts thereof, wherein X, Y, Z¹, Z², Z³, R¹, R², R⁵ and n are as defined herein.

Also provided are compounds of Formula VII

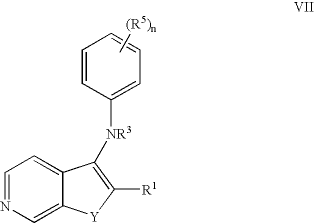

and stereoisomers, geometric isomers, tautomers, solvates, metabolites, salts (including pharmaceutically acceptable salts) and pharmaceutically acceptable prodrugs thereof, wherein Y, R¹, R³, R⁵ and n are as defined herein.

Another aspect of the invention provides methods of inhibiting Raf kinase activity, comprising contacting a Raf kinase with an effective inhibitory amount of a compound of this invention, or a stereoisomer, geometric isomer, tautomer, solvate, metabolite, or pharmaceutically acceptable salt or prodrug thereof.

Another aspect of the invention provides methods of preventing or treating a disease or disorder modulated by Raf kinases, comprising administering to a mammal in need of such treatment an effective amount of a compound of this invention or a stereoisomer, geometric isomer, tautomer, solvate, metabolite, or pharmaceutically acceptable salt or prodrug thereof. Examples of such diseases and disorders include, but are not limited to, hyperproliferative disorders (such as cancer, including melanoma and other cancers of the skin), neurodegeneration, cardiac hypertrophy, pain, migraine and neurotraumatic disease.

Another aspect of the invention provides methods of preventing or treating cancer, comprising administering to a mammal in need of such treatment an effective amount of a compound of this invention, or a stereoisomer, geometric isomer, tautomer, solvate, metabolite, or pharmaceutically acceptable salt or prodrug thereof, alone or in combination with one or more additional compounds having anti-cancer properties.

Another aspect of the invention includes kits comprising a compound of this invention, or a stereoisomer, geometric isomer, tautomer, solvate, metabolite, or pharmaceutically acceptable salt or prodrug thereof, a container, and optionally a package insert or label indicating a treatment.

Another aspect of the invention includes methods of preparing, methods of separation, and methods of purification of the compounds of this invention.

### DETAILED DESCRIPTION OF THE INVENTION

Raf Inhibitor Compounds

Reference will now be made in detail to certain embodiments of the invention, examples of which are illustrated in the accompanying structures and formulas. While the invention will be described in conjunction with the enumerated embodiments, it will be understood that they are not intended to limit the invention to those embodiments. On the contrary, the invention is intended to cover all alternatives, modifications, and equivalents, which may be included within the scope of the present invention as defined by the claims. One skilled in the art will recognize many methods and materials similar or equivalent to those described herein, which could be used in the practice of the present invention. The present invention is in no way limited to the methods and materials described. In the event that one or more of the incorporated literature and similar materials differs from or contradicts this application, including but not limited to defined terms, term usage, described techniques, or the like, this application controls.

Definitions

The term “alkyl” as used herein refers to a saturated linear or branched-chain monovalent hydrocarbon radical of one to twelve carbon atoms, wherein the alkyl radical may be optionally substituted independently with one or more substituents described below. Examples of alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—CH(CH₃)₂CH₂CH₂CH₃), 3-methyl-2-pentyl CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃, 1-heptyl, 1-octyl, cyclopropyl, cyclobutyl, cyclopentyl, cyclohexyl, cycloheptyl, and cyclooctyl.

The term “alkenyl” refers to linear or branched-chain monovalent hydrocarbon radical of two to twelve carbon atoms with at least one site of unsaturation, i.e., a carbon-carbon, sp² double bond, wherein the alkenyl radical may be optionally substituted independently with one or more substituents described herein, and includes radicals having “cis” and “trans” orientations, or alternatively, “E” and “Z” orientations. Examples include, but are not limited to, ethylenyl or vinyl (—CH═CH₂), allyl (—CH₂CH═CH₂), 1-cyclopent-1-enyl, 1-cyclopent-2-enyl, 1-cyclopent-3-enyl, 5-hexenyl, 1-cyclohex-1-enyl, 1-cyclohex-2-enyl, and 1-cyclohex-3-enyl.

The term “alkynyl” refers to a linear or branched monovalent hydrocarbon radical of two to twelve carbon atoms with at least one site of unsaturation, i.e., a carbon-carbon, sp triple bond, wherein the alkynyl radical may be optionally substituted independently with one or more substituents described herein. Examples include, but are not limited to, ethynyl (—C≡CH) and propynyl (propargyl, —CH₂C≡CH).

“Carbocycle” and “carbocyclyl” mean a monovalent non-aromatic, saturated or unsaturated ring having 3 to 12 carbon atoms as a monocyclic ring or 7 to 12 carbon atoms as a bicyclic ring. Bicyclic carbocycles having 7 to 12 atoms can be arranged, for example, as a bicyclo [4,5], [5,5], [5,6] or [6,6] system, and byciclic carbocycles having 9 or 10 ring atoms can be arranged as a bicyclo [5,6] or [6,6] system, or as bridged systems such as bicyclo[2.2.1]heptane, bicyclo[2.2.2]octane and bicyclo[3.2.2]nonane. Examples of monocyclic carbocycles include, but are not limited to, cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl and cyclododecyl.

“Aryl” means a monovalent aromatic hydrocarbon radical of 6-20 carbon atoms derived by the removal of one hydrogen atom from a single carbon atom of a parent aromatic ring system. Some aryl groups are represented in the exemplary structures as “Ar”. Aryl includes bicyclic radicals comprising an aromatic ring with a fused non-aromatic ring, a partially unsaturated ring, or an aromatic ring. Typical aryl groups include, but are not limited to, radicals derived from benzene, substituted benzenes, naphthalene, anthracene, biphenyl, indenyl, indanyl, 1,2-dihydronapthalene, 1,2,3,4-tetrahydronapthyl, and the like.

“Heteroaryl”, “heterocyclyl”, and “heterocycle” all refer to a ring system in which one or more ring atoms are a heteroatom, e.g., nitrogen, oxygen, and sulfur. The heterocyclyl radical comprises 1 to 20 carbon atoms and 1 to 6 heteroatoms selected from N, O, P, and S. The heterocyclyl radical may be saturated, partially unsaturated or fully unsaturated. The heterocyclyl radical may be aromatic or non-aromatic. A heterocycle may be a monocycle having 3 to 7 ring members (2 to 6 carbon atoms and 1 to 3 heteroatoms selected from N, O, P, and S) or a bicycle having 7 to 10 ring members (4 to 9 carbon atoms and 1 to 3 heteroatoms selected from N, O, P, and S), for example: a bicyclo [4,5], [5,5], [5,6], or [6,6] system. Heterocycles are described in Paquette, Leo A.; “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and J. Am. Chem. Soc. (1960) 82:5566.

Examples of heterocyclyl radicals include, but are not limited to, pyridyl, dihydroypyridyl, 4-dialkylaminopyridinium, tetrahydropyridyl (piperidyl), thiazolyl, tetrahydrothiophenyl, sulfur-oxidized tetrahydrothiophenyl, pyrimidinyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, piperidinyl, 4-piperidonyl, pyrrolidinyl, 2-pyrrolidonyl, pyrrolinyl, tetrahydrofuranyl, 3-oxo-tetrahydrofuranyl, 3-oximinio-tetrahydrofuranyl, bis-tetrahydrofuranyl, tetrahydropyranyl, 4-oxo-tetrahydropyranyl, 4-oximino-tetrahydropyranyl, bis-tetrahydropyranyl, tetrahydroquinolinyl, tetrahydroisoquinolinyl, decahydroquinolinyl, octahydroisoquinolinyl, azocinyl, triazinyl, 6H-1,2,5-thiadiazinyl, 2H,6H-1,5,2-dithiazinyl, thienyl, thianthrenyl, pyranyl, isobenzofuranyl, chromenyl, xanthenyl, phenoxathinyl, 2H-pyrrolyl, isothiazolyl, isoxazolyl, pyrazinyl, pyridazinyl, indolizinyl, isoindolyl, 3H-indolyl, 1H-indazolyl, purinyl, 4H-quinolizinyl, phthalazinyl, naphthyridinyl, quinoxalinyl, quinazolinyl, cinnolinyl, pteridinyl, 4H-carbazolyl, carbazolyl, β-carbolinyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, piperazinyl, indolinyl, isoindolinyl, quinuclidinyl, morpholinyl, oxazolidinyl, benzotriazolyl, benzisoxazolyl, oxindolyl, benzoxazolinyl, and isatinoyl.

The term “heteroaryl” also includes 1) monocyclic aromatic 5-, 6-, and 7-membered rings containing one or more heteroatoms independently selected from nitrogen, oxygen, and sulfur, and 2) fused ring systems of 8 to 20 atoms wherein at least one aromatic ring contains one or more heteroatoms independently selected from nitrogen, oxygen, and sulfur. Examples of heteroaryl groups are pyridinyl (including, for example, 2-hydroxypyridinyl), imidazolyl, imidazopyridinyl, pyrimidinyl (including, for example, 4-hydroxypyrimidinyl), pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, triazolyl, thiadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. Some heteroaryl groups are represented herein as “hetAr”. Heteroaryl groups are optionally substituted independently with one or more substituents described herein.

By way of example and not limitation, carbon bonded heterocycles are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline. Still more typically, carbon bonded heterocycles include 2-pyridyl, 3-pyridyl, 4-pyridyl, 5-pyridyl, 6-pyridyl, 3-pyridazinyl, 4-pyridazinyl, 5-pyridazinyl, 6-pyridazinyl, 2-pyrimidinyl, 4-pyrimidinyl, 5-pyrimidinyl, 6-pyrimidinyl, 2-pyrazinyl, 3-pyrazinyl, 5-pyrazinyl, 6-pyrazinyl, 2-thiazolyl, 4-thiazolyl, or 5-thiazolyl.

By way of example and not limitation, nitrogen bonded heterocycles are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or β-carboline. Still more typically, nitrogen bonded heterocycles include 1-aziridyl, 1-azetedyl, 1-pyrrolyl, 1-imidazolyl, 1-pyrazolyl, and 1-piperidinyl.

Substituents may also be combinations of alkyl, alkenyl, alkynyl, carbocycle, aryl, and heteroaryl radicals, such as cyclopropylmethyl, cyclohexylethyl, benzyl, and N-ethylmorpholino, and substituted forms thereof.

“Substituted alkyl”, “substituted aryl”, “substituted heterocyclyl” and “substituted cycloalkyl” mean alkyl, aryl, heterocyclyl and cycloalkyl respectively, in which one or more hydrogen atoms are each independently replaced with a substituent. Typical substituents include, but are not limited to, F, Cl, Br, I, CN, CF₃, OR, R, ═O, ═S, ═NR, ═N⁺(O)(R), ═N(OR), ═N⁺(O)(OR), ═N—NRR′, —C(═O)R, —C(═O)OR, —C(═O)NRR′, —NRR′, —N⁺RR′R″, —N(R)C(═O)R′, —N(R)C(═O)OR′, —N(R)C(═O)NR′R″, —SR, —OC(═O)R, —OC(═O)OR, —OC(═O)NRR′, —OS(O)₂(OR), —OP(═O)(OR)₂, —OP(OR)₂, —P(═O)(OR)₂, —P(═O)(OR)NR′R″, —S(O)R, —S(O)₂R, —S(O)₂NR, —S(O)(OR), —S(O)₂(OR), —SC(═O)R, —SC(═O)OR, ═O and —SC(═O)NRR′; wherein each R, R′ and R″ is independently selected from H, C₁-C₁₀ alkyl, C₁-C₁₀ alkenyl, C₁-C₁₀ alkynyl, C₆-C₂₀ aryl and C₂-C₂₀ heterocycle. Alkenyl and alkynyl groups as described above may also be similarly substituted.

The terms “treat” or “treatment” refer to both therapeutic treatment and prophylactic or preventative measures, wherein the object is to prevent or slow down (lessen) an undesired physiological change or disorder, such as the development or spread of cancer. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented. The terms “treating”, “treat”, or “treatment” embrace both preventative, i.e., prophylactic, and palliative treatment.

The phrase “therapeutically effective amount” means an amount of a compound of the present invention that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. For cancer therapy, efficacy can be measured, for example, by assessing the time to disease progression (TTP) and/or determining the response rate (RR).

The term “bioavailability” refers to the systemic availability (i.e., blood/plasma levels) of a given amount of drug administered to a patient. Bioavailability is an absolute term that indicates measurement of both the time (rate) and total amount (extent) of drug that reaches the general circulation from an administered dosage form.

The terms “cancer” and “cancerous” refer to or describe the physiological condition in mammals that is typically characterized by unregulated cell growth. A “tumor” comprises one or more cancerous cells. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), lung cancer including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

A “chemotherapeutic agent” is a chemical compound useful in the treatment of cancer. Examples of chemotherapeutic agents include Erlotinib (TARCEVA®, Genentech/OSI Pharm.), Bortezomib (VELCADE®, Millenium Pharm.), Fulvestrant (FASLODEX®, AstraZeneca), Sutent (SU11248, Pfizer), Letrozole (FEMARA®, Novartis), Imatinib mesylate (GLEEVEC®, Novartis), PTK787/ZK 222584 (Novartis), Oxaliplatin (Eloxatin®, Sanofi), 5-FU (5-fluorouracil), Leucovorin, Rapamycin (Sirolimus, RAPAMUNE®, Wyeth), Lapatinib (GSK572016, Glaxo Smith Kline), Lonafarnib (SCH 66336), Sorafenib (BAY43-9006, Bayer Labs), and Gefitinib (IRESSA®, AstraZeneca), AG1478, AG1571 (SU 5271; Sugen), alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (Angew Chem. Intl. Ed. Engl. (1994) 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumberg, Ill.), and TAXOTERE® (doxetaxel; Rhône-Poulenc Rorer, Antony, France); chloranbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; xeloda; ibandronate; CPT-11; topoisomerase inhibitor RFS 2000; difluorometlhylornithine (DMFO); retinoids such as retinoic acid; capecitabine; and pharmaceutically acceptable salts, acids and derivatives of any of the above.

Also included in the definition of “chemotherapeutic agent” are:

(i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide, and goserelin; as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN® rIL-2; a topoisomerase 1 inhibitor such as LURTOTECAN®; ABARELIX® rmRH; (ix) anti-angiogenic agents such as bevacizumab (AVASTIN®, Genentech); and (x) pharmaceutically acceptable salts, acids and derivatives of any of the above.

The term “prodrug” as used in this application refers to a precursor or derivative form of a compound of this invention that is less cytotoxic to cells compared to the parent compound or drug and is capable of being enzymatically or hydrolytically activated or converted into the more active parent form. See, e.g., Wilman, “Prodrugs in Cancer Chemotherapy” Biochemical Society Transactions, 14, pp. 375-382, 615th Meeting Belfast (1986) and Stella et al., “Prodrugs: A Chemical Approach to Targeted Drug Delivery,” Directed Drug Delivery, Borchardt et al., (ed.), pp. 247-267, Humana Press (1985). The prodrugs of this invention include, but are not limited to, phosphate-containing prodrugs, thiophosphate-containing prodrugs, sulfate-containing prodrugs, peptide-containing prodrugs, D-amino acid-modified prodrugs, glycosylated prodrugs, β-lactam-containing prodrugs, optionally substituted phenoxyacetamide-containing prodrugs, optionally substituted phenylacetamide-containing prodrugs, 5-fluorocytosine and other 5-fluorouridine prodrugs which can be converted into the more active cytotoxic free drug. Examples of cytotoxic drugs that can be derivatized into a prodrug form for use in this invention include, but are not limited to, compounds of this invention and chemotherapeutic agents such as described above.

A “metabolite” is a product produced through metabolism in the body of a specified compound or salt thereof. Metabolites of a compound may be identified using routine techniques known in the art and their activities determined using tests such as those described herein. Such products may result for example from the oxidation, reduction, hydrolysis, amidation, deamidation, esterification, deesterification, enzymatic cleavage, and the like, of the administered compound. Accordingly, the invention includes metabolites of compounds of this invention, including compounds produced by a process comprising contacting a compound of this invention with a mammal for a period of time sufficient to yield a metabolic product thereof.

A “liposome” is a small vesicle composed of various types of lipids, phospholipids and/or surfactant which is useful for delivery of a drug (such as the Raf inhibitors disclosed herein and, optionally, a chemotherapeutic agent) to a mammal. The components of the liposome are commonly arranged in a bilayer formation, similar to the lipid arrangement of biological membranes.

The term “package insert” is used to refer to instructions customarily included in commercial packages of therapeutic products, that contain information about the indications, usage, dosage, administration, contraindications and/or warnings concerning the use of such therapeutic products.

The term “chiral” refers to molecules which have the property of non-superimposability of the mirror image partner, while the term “achiral” refers to molecules which are superimposable on their mirror image partner.

The term “stereoisomers” refers to compounds which have identical chemical constitution, but differ with regard to the arrangement of the atoms or groups in space.

“Diastereomer” refers to a stereoisomer with two or more centers of chirality and whose molecules are not mirror images of one another. Diastereomers have different physical properties, e.g. melting points, boiling points, spectral properties, and reactivities. Mixtures of diastereomers may separate under high resolution analytical procedures such as electrophoresis and chromatography.

“Enantiomers” refer to two stereoisomers of a compound which are non-superimposable mirror images of one another.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the invention may contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof such as racemic mixtures, form part of the present invention. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and l or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity.

The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons.

The phrase “pharmaceutically acceptable salt,” as used herein, refers to pharmaceutically acceptable organic or inorganic salts of a compound of the invention. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

If the compound of the invention is a base, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like. If the compound of the invention is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include, but are not limited to, organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

The phrase “pharmaceutically acceptable” indicates that the substance or composition must be compatible chemically and/or toxicologically, with the other ingredients comprising a formulation, and/or the mammal being treated therewith.

The compounds of this invention also include other salts of such compounds which are not necessarily pharmaceutically acceptable salts, and which may be useful as intermediates for preparing and/or purifying compounds of this invention and/or for separating enantiomers of compounds of this invention.

A “solvate” refers to an association or complex of one or more solvent molecules and a compound of the invention. Examples of solvents that form solvates include, but are not limited to, water, isopropanol, ethanol, methanol, DMSO, ethyl acetate, acetic acid, and ethanolamine. The term “hydrate” refers to the complex where the solvent molecule is water.

The term “protecting group” or “Pg” refers to a substituent that is commonly employed to block or protect a particular functionality while reacting other functional groups on the compound. For example, an “amino-protecting group” is a substituent attached to an amino group that blocks or protects the amino functionality in the compound. Suitable amino-protecting groups include acetyl, trifluoroacetyl, t-butoxycarbonyl (BOC), benzyloxycarbonyl (CBz) and 9-fluorenylmethylenoxycarbonyl (Fmoc). Similarly, a “hydroxy-protecting group” refers to a substituent of a hydroxy group that blocks or protects the hydroxy functionality. Suitable protecting groups include acetyl and silyl. A “carboxy-protecting group” refers to a substituent of the carboxy group that blocks or protects the carboxy functionality. Common carboxy-protecting groups include —CH₂CH₂SO₂Ph, cyanoethyl, 2-(trimethylsilyl)ethyl, 2-(trimethylsilyl)ethoxymethyl, 2-(p-toluenesulfonyl)ethyl, 2-(p-nitrophenylsulfenyl)ethyl, 2-(diphenylphosphino)-ethyl, nitroethyl and the like. For a general description of protecting groups and their use, see T. W. Greene, Protective Groups in Organic Synthesis, John Wiley & Sons, New York, 1991.

The term “animal” refers to humans (male or female), companion animals (e.g., dogs, cats and horses), food-source animals, zoo animals, marine animals, birds and other similar animal species. “Edible animals” refers to food-source animals such as cows, pigs, sheep and poultry.

The phrase “pharmaceutically acceptable” indicates that the substance or composition must be compatible chemically and/or toxicologically, with the other ingredients comprising a formulation, and/or the mammal being treated therewith.

Raf Inhibitor Compounds

The present invention provides compounds, and pharmaceutical formulations thereof, that are potentially useful in the treatment of diseases, conditions and/or disorders modulated by Raf kinases.

One aspect of this invention provised compounds of Formula I

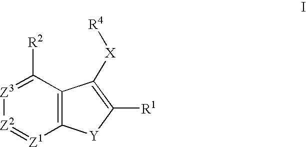

and stereoisomers, geometric isomers, tautomers, solvates, metabolites and pharmaceutically acceptable salts thereof, wherein:

X is selected from NR³, O, C(═O), and S;

Y is O or S;

Z¹, Z², and Z³ are independently selected from CR⁵ and N, and one or two of Z¹, Z² and Z³ is N;

R¹, R² and R⁵ are independently selected from H, F, Cl, Br, I, —C(═Y¹)R, —C(═Y¹)OR, —C(═Y¹)NR₂, —NR₂, —N⁺R₃, —N(R)C(═Y¹)R, —N(R)C(═Y¹)OR, —N(R)C(═Y¹)NR₂, —NR-alkylaryl, —NRSO₂NRR, —OR, —OC(═Y¹)R, —OC(═Y¹)OR, —OC(═Y¹)NR₂, —OS(O)₂(OR), —OP(═Y¹)(OR)₂, —OP(OR)₂, —P(═Y¹)(OR)₂, —P(═Y¹)(OR)NR₂, —SR, —S(O)R, —S(O)₂R, —S(O)₂NRR, —S(O)(OR), —S(O)₂(OR), —SC(═Y¹)R, —SC(═Y¹)OR, —SC(═Y¹)NR₂, C₁-C₈ alkylhalide, C₁-C₈ alkylsulfonate, C₁-C₈ alkylamino, C₁-C₈ alkylhydroxyl, C₁-C₈ alkylthiol, 5-7 membered ring lactam, 5-7 membered ring lactone, 5-7 membered ring sultam, C₁-C₈ alkyl, C₂-C₈ alkenyl, C₂-C₈ alkynyl, C₆-C₂₀ aryl, C₃-C₁₂ carbocycle, and C₁-C₂₀ heterocyclyl;

R³ is selected from H, C₁-C₈ alkyl, C₂-C₈ alkenyl, C₂-C₈ alkynyl, C₆-C₂₀ aryl, C₃-C₁₂ carbocycle, C₁-C₂₀ heterocyclyl, and a protecting group;

R⁴ is selected from phenyl,

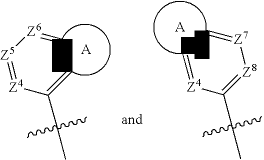

wherein the wavy line indicates the attachment to X;

Z⁴, Z⁵, Z⁶, Z⁷, and Z⁸ are independently selected from CR⁵ and N;

A is (i) an optionally substituted 5 or 6 membered fused heterocyclic ring having one or two heteroatoms independently selected from O, N, and S, (ii) an optionally substituted 5 or 6 membered fused carbocyclic ring, or (iii) an optionally substituted fused phenyl ring;

each R is independently H, C₁-C₈ alkyl, C₂-C₈ alkenyl, C₂-C₈ alkynyl, C₆-C₂₀ aryl, C₁-C₂₀ heterocyclyl, or a protecting group;

Y¹ is independently selected from O, S, NR, N⁺(O)(R), N(OR), N⁺(O)(OR), and N—NRR; and

each alkyl, alkenyl, alkynyl, aryl, phenyl, carbocyclyl, and heterocyclyl is optionally substituted with one or more substituents independently selected from F, Cl, Br, I, CN, CF₃, OR, SR, R, ═O, ═S, ═NR, ═N⁺(O)(R), ═N(OR), ═N⁺(O)(OR), ═N—NR₂, —C(═Y¹)R, —C(═Y¹)OR, —C(═Y¹)NR₂, —NR₂, —N⁺R₃, —N(R)C(═Y¹)R, —N(R)C(═Y¹)OR, —N(R)C(═Y¹)NR₂, —SR, —OC(═Y¹)R, —OC(═Y¹)OR, —OC(═Y¹)NR₂, —OS(O)₂(OR), —OP(═Y¹)(OR)₂, —OP(OR)₂, —P(═Y¹)(OR)₂, —P(═Y¹)(OR)NR₂, —S(O)R, —S(O)₂R, —S(O)₂NR, —S(O)(OR), —S(O)₂(OR), —SC(═Y¹)R, —SC(═Y¹)OR, and —SC(═Y¹)NR₂.

In certain embodiments, R is a protecting group selected from trialkylsilyl, dialkylphenylsilyl, benzoate, benzyl, benzyloxymethyl, methyl, methoxymethyl, triarylmethyl, phthalimido and tetrahydropyranyl.

In certain embodiments, (i) Z¹ and Z² are CR⁵, and Z³ is N; (ii) Z¹ and Z³ are CR⁵, and Z² is N; (iii) Z² and Z³ are CR⁵, and Z¹ is N; (iv) Z¹ and Z³ are N, and Z² is CR⁵; or (vi) Z¹ and Z² are N, and Z³ is CR⁵.

In certain embodiments, the compounds of Formula I include substituted forms of the following parent heterocycles:

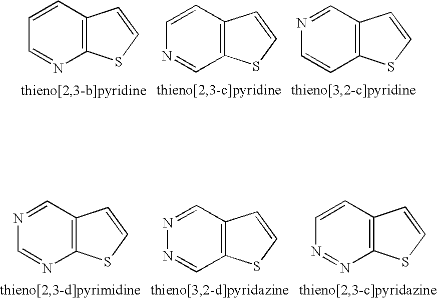

In certain embodiments, the compounds of Formula I include substituted forms of the following parent heterocycles:

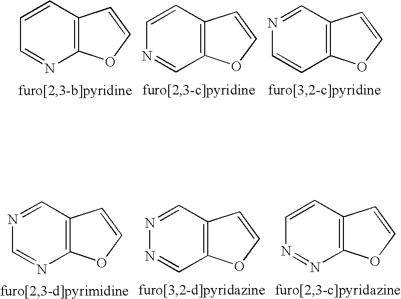

In certain embodiments, fused ring A is an optionally substituted 5 or 6 membered fused heterocyclic ring selected from tetrahydrofuranyl, tetrahydropyranyl, tetrahydropyridyl, piperazinyl, pyrrolidinyl, pyridyl, pyrimidinyl, dihydrothiophenyl, thiophenyl, imidazolyl, thiazolyl, oxazolyl, isoxazolyl, and pyrazolyl. In particular embodiments, the 5 or 6 membered fused heterocyclic ring may be substituted with ═O, ═S, ═NR, ═N⁺(O)(R), ═N(OR), ═N⁺(O)(OR), or ═N—NR₂. In particular embodiments, the 5 or 6 membered fused heterocyclic ring is substituted with ═NOR.

In certain embodiments, fused ring A is an optionally substituted 5 or 6 membered carbocyclic ring selected from cyclopentyl, cyclopentenyl, cyclohexyl, and cyclohexenyl. In particular embodiments, the 5 or 6 membered carbocyclic ring may be substituted with ═O, ═S, ═NR, ═N⁺(O)(R), ═N(OR), ═N⁺(O)(OR), or ═N—NR₂. In particular embodiments, the 5 or 6 membered carbocyclic ring is substituted with ═N(OR).

Exemplary embodiments of R⁴ include, but are not limited to, the following structures:

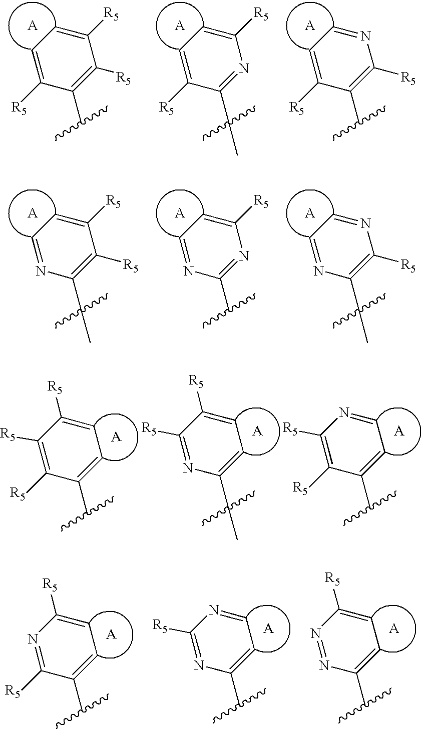

wherein the wavy line indicates the attachment to X, each R⁵ is independent of the other R⁵, and A is: (i) an optionally substituted 5 or 6 membered fused heterocyclic ring having one or two heteroatoms independently selected from O, N, and S, (ii) an optionally substituted 5 or 6 membered carbocyclic ring, or (iii) an optionally substituted phenyl ring.

In certain embodiment of compounds of Formula I, R¹ is C(═O)R, C(═O)OR, CH(OH)-aryl or C(═O)NRR. In particular embodiments, each R is independently selected from H, alkyl, aryl and heterocycle. In certain embodiments, said alkyl, aryl and heterocycle are optionally substituted with one or more groups independently selected from alkyl, O-alkyl and NRR.

In certain embodiments, R¹ is NRR, NR-alkylaryl or NRC(═O)R. In particular embodiments, each R is independently H or alkyl. In certain embodiments, said alkyl is optionally substituted with one or more groups independently selected from O-alkyl and NRR.

In certain embodiments, R¹ is phenyl, wherein said phenyl is optionally substituted with one or more substituents independently selected from F, Cl, Br, I, CN, CF₃, OR, R, —C(═Y¹)R, —C(═Y¹)OR, —C(═Y¹)NR₂, —NR₂, —N⁺(R)₃, —N(R)C(═Y¹)R, —N(R)C(═Y¹)OR, —N(R)C(═Y¹)NR₂, —SR, —OC(═Y¹)R, —OC(═Y¹)OR, —OC(═Y¹)NR₂, —OS(O)₂(OR), —OP(═Y¹)(OR)₂, —OP(OR)₂, —P(═Y¹)(OR)₂, —P(═Y¹)(OR)NR₂, —S(O)R, —S(O)₂R, —S(O)₂NRR, —S(O)(OR), —S(O)₂(OR), —SC(═Y¹)R, —SC(═Y¹)OR, and —SC(═Y¹)NR₂. In particular embodiments, R¹ is phenyl optionally substituted with one or more substituents selected from OR, C(═O)RR, alkyl, CN or CF₃.

In certain embodiments, R¹ is a heterocycle including, but not limited to, 2-pyridyl, 3-pyridyl, 4-pyridyl, 3-isoxazolyl, 4-isoxazolyl, 5-isoxazolyl, 2-imidazolyl, 4-imidazolyl, 3-pyrazolyl, 4-pyrazolyl, 2-pyrrolyl, 3-pyrrolyl, 2-thiazolyl, 4-thiazolyl, 5-thiazolyl, 3-pyridazinyl, 4-pyridazinyl, 5-pyridazinyl, 2-pyrimidinyl, 5-pyrimidinyl, 6-pyrimidinyl, 2-pyrazinyl, 2-oxazolyl, 4-oxazolyl, 5-oxazolyl, 2-furanyl, 3-furanyl, 2-thienyl, 3-thienyl, 3-indolyl, and substituted forms thereof, and shown as:

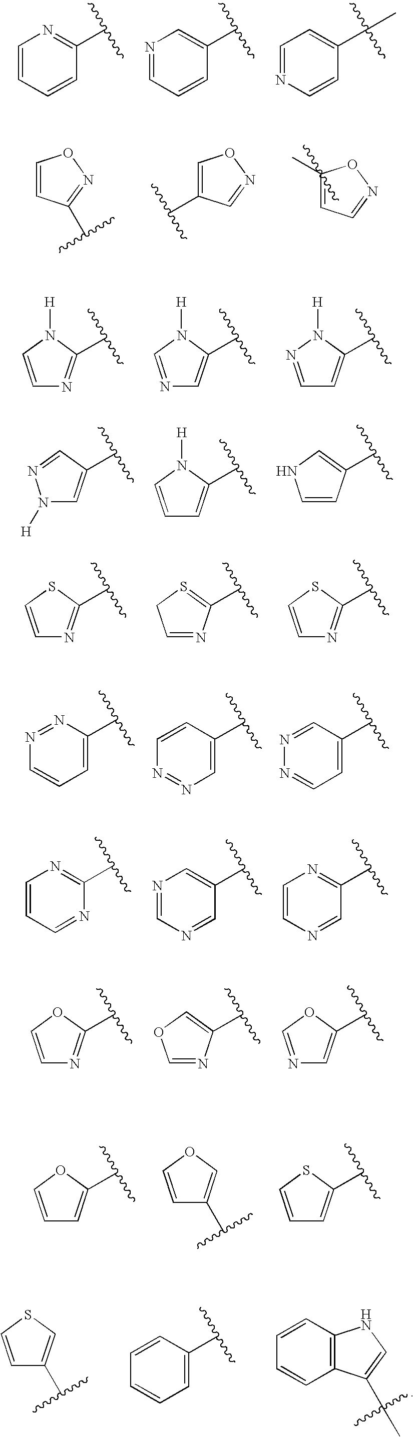

In particular embodiments, R¹ is selected from 2-pyridyl, 3-pyridyl, 4-pyridyl, 2-pyrimidinyl, 3-isoxazolyl, and substituted forms thereof. In certain embodiments, said 2-pyridyl, 3-pyridyl, 4-pyridyl, 2-pyrimidinyl, and 3-isoxazolyl are substituted with one or more alkyl groups.

In certain embodiments, R¹ is selected from C₁-C₈ alkyl, C₂-C₈ alkenyl, and C₂-C₈ alkynyl, wherein said alkyl, alkenyl and alkynyl are optionally substituted with one or more substituents selected from F, Cl, Br, I, OH, R, —C(═Y¹)R, —C(═Y¹)OR, —C(═Y¹)NR₂, —NR₂, —N⁺(R)₃, —N(R)C(═Y¹)R, —N(R)C(═Y¹)OR, —N(R)C(═Y¹)NR₂, —SR, —OC(═Y¹)R, —OC(═Y¹)OR, —OC(═Y¹)NR₂, —OS(O)₂(OR), —OP(═Y¹)(OR)₂, —OP(OR)₂, —P(═Y¹)(OR)₂, —P(═Y¹)(OR)NR₂, —S(O)R, —S(O)₂R, —S(O)₂NRR, —S(O)(OR), —S(O)₂(OR), —SC(═Y¹)R, —SC(═Y¹)OR, and —SC(═Y¹)NR₂.

Exemplary embodiments of compounds of Formula I include Formulas Ia-vv:

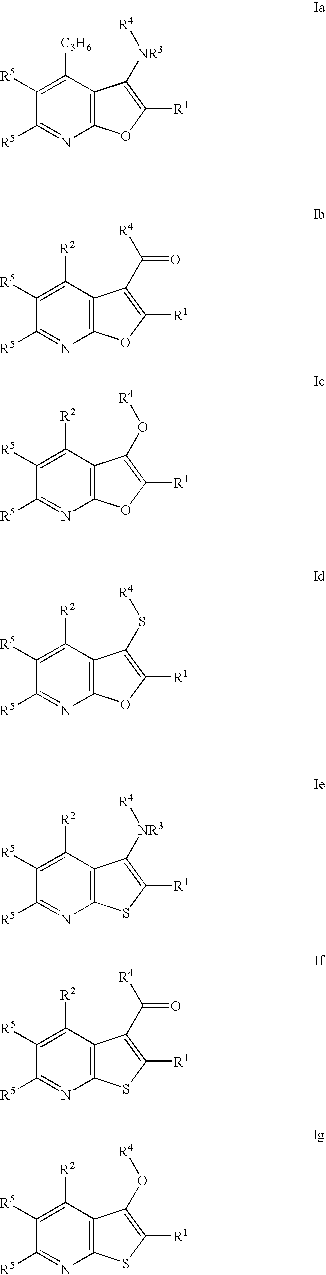

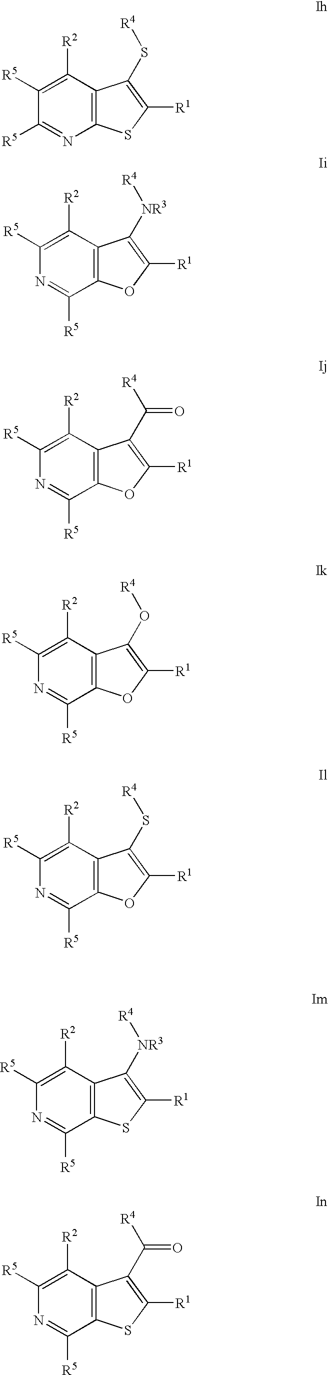

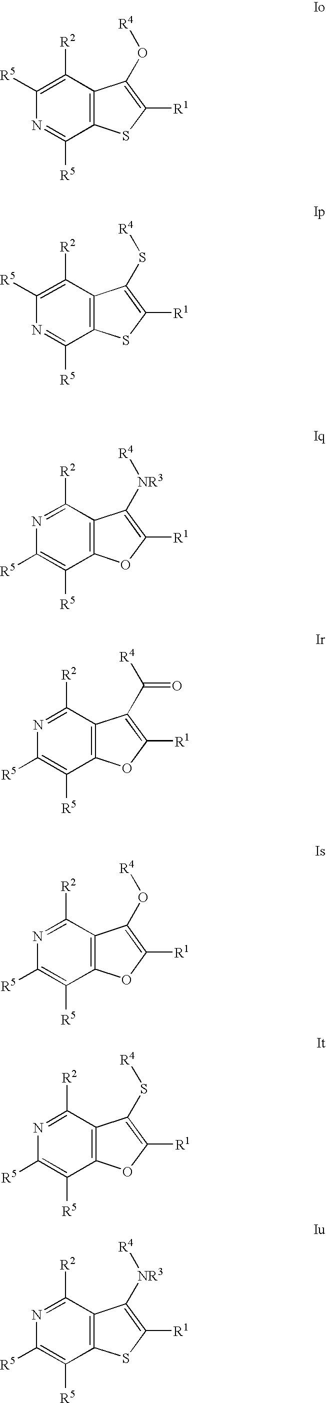

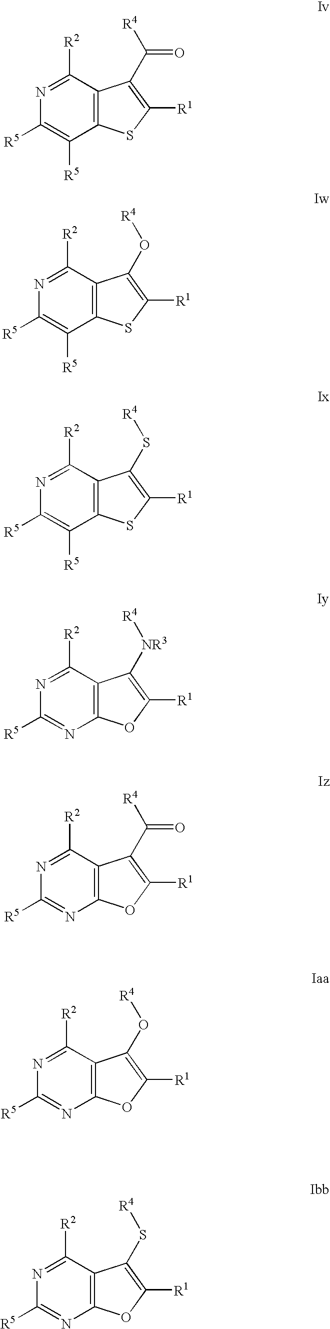

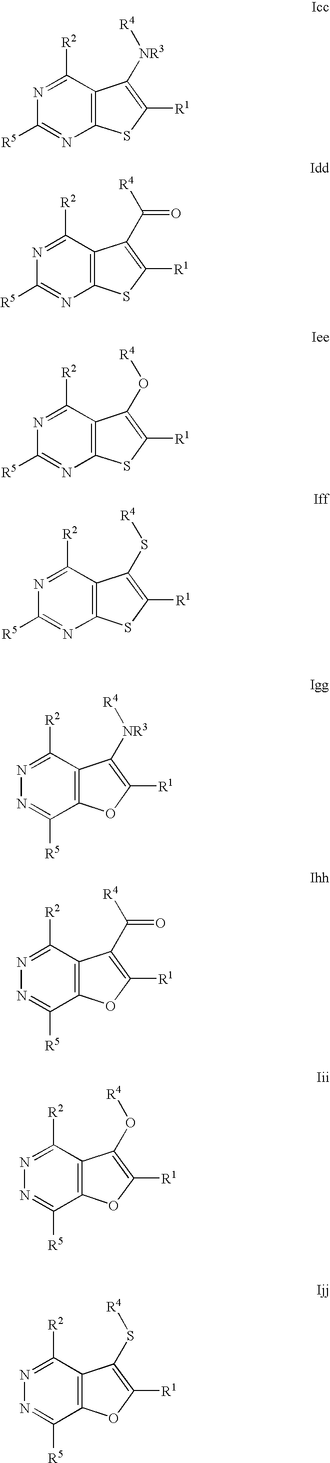

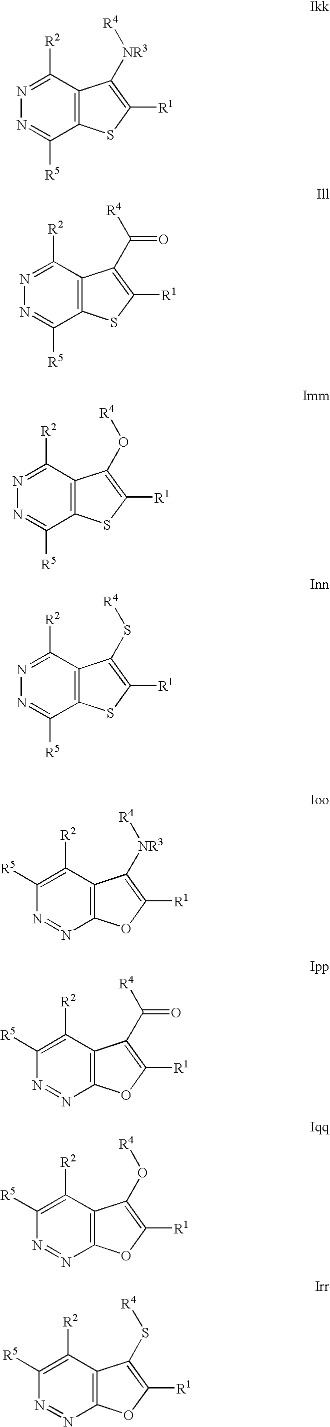

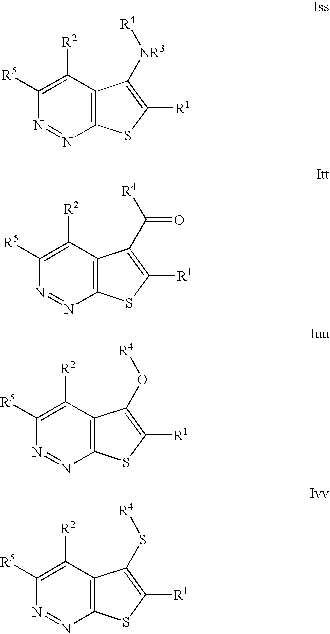

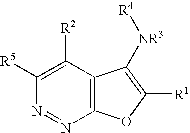

In certain embodiments of compounds of Formula I, fused ring A is an optionally substituted ring selected from phenyl, cyclopentyl, cyclopentenyl, cyclohexyl, cyclohexenyl, tetrahydrofuranyl, tetrahydropyranyl, tetrahydropyridyl, piperazinyl, pyrrolidinyl, pyridyl, pyrimidinyl, dihydrothiophenyl, thiophenyl, imidazolyl, thiazolyl, oxazolyl, isoxazolyl, and pyrazolyl, including, but not limited to the structures:

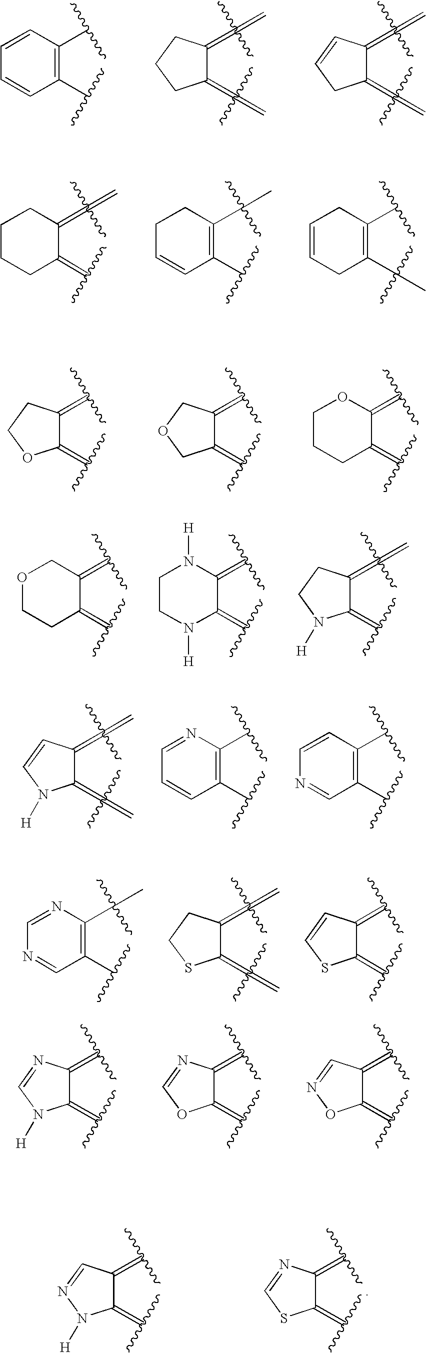

In certain embodiments of compounds of Formula I, R⁴ is selected from structures IIa-o:

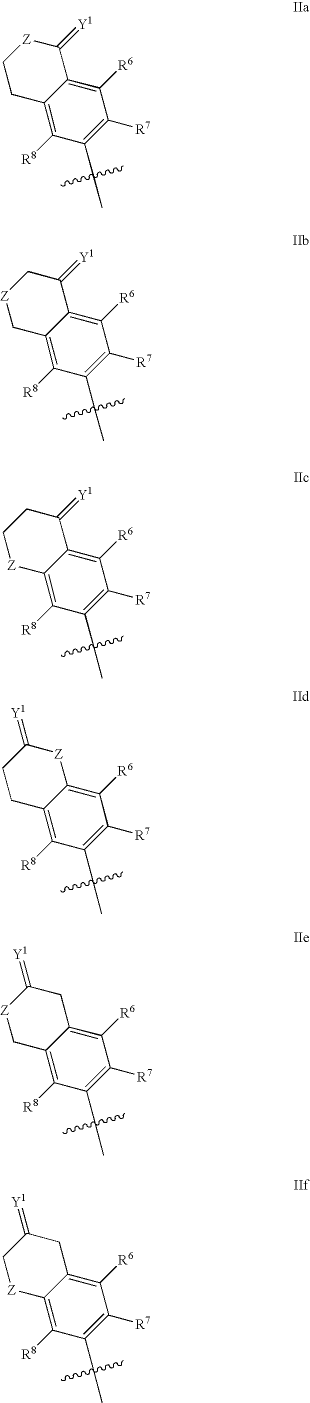

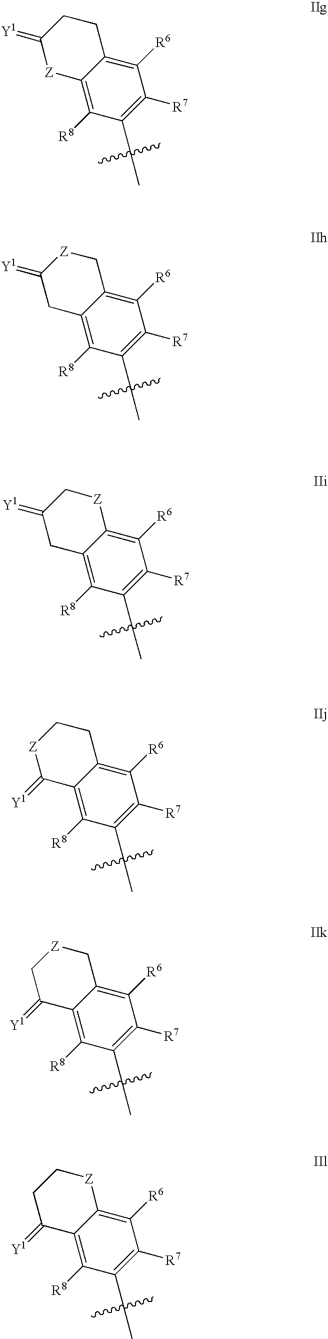

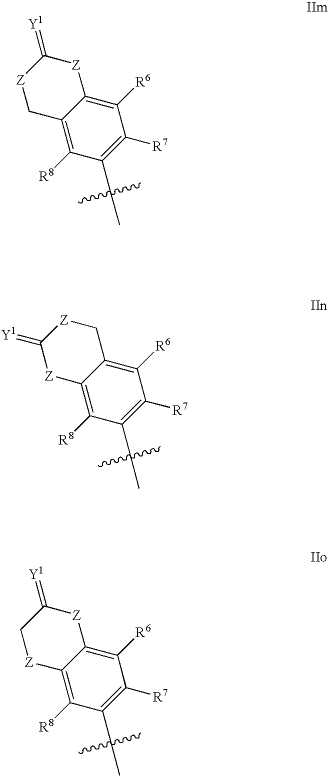

wherein the wavy line indicates the attachment to X;

Y¹ is independently selected from O, S, NR, N⁺(O)(R), N(OR), N⁺(O)(OR), and N—NRR;

each Z is independently selected from CR₂, C(═Y), NR, O, and S; and

R⁶, R⁷ and R⁸ are independently selected from H, F, Cl, Br, I, —C(═Y¹)R, —C(═Y¹)OR, —C(═Y¹)NR₂, —NR₂, —N⁺R³, —N(R)C(═Y¹)R, —N(R)C(═Y¹)OR, —N(R)C(═Y¹)NR₂, —OR, —OC(═Y¹)R, —OC(═Y¹)OR, —OC(═Y¹)NR₂, —OS(O)₂(OR), —OP(═Y¹)(OR)₂, —OP(OR)₂, —P(═Y¹)(OR)₂, —P(═Y¹)(OR)NR₂, —SR, —S(O)R, —S(O)₂R, —S(O)₂NRR, —S(O)(OR), —S(O)₂(OR), —SC(═Y¹)R, —SC(═Y¹)OR, —SC(═Y¹)NR₂, C₁-C₈ alkylhalide, C₁-C₈ alkylsulfonate, C₁-C₈ alkylamino, C₁-C₈ alkylhydroxyl, C₁-C₈ alkylthiol, 5-7 membered ring lactam, 5-7 membered ring lactone, 5-7 membered ring sultam, C₁-C₈ alkyl, C₂-C₈ alkenyl, C₂-C₈ alkynyl, C₆-C₂₀ aryl, C₃-C₁₂ carbocycle, and C₁-C₂₀ heterocyclyl.

In embodiments of structures IIa-o wherein ═Y¹ is ═N—OR, the oxime moiety can exist as either the E or Z isomer or as a mixture of both.

In certain embodiments of compounds of Formula I, R⁴ is selected from structures IIIa-o:

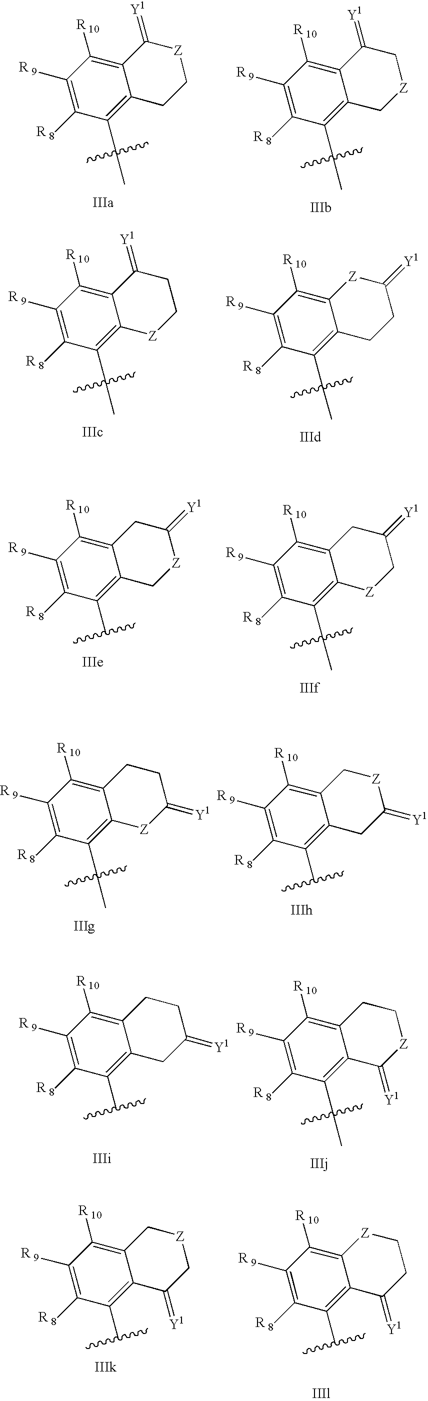

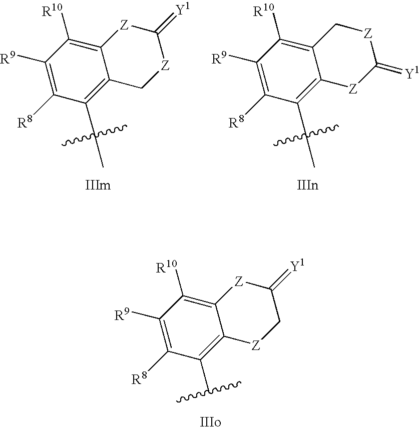

wherein the wavy line indicates the attachment to X;

Y¹ is independently selected from O, S, NR, N⁺(O)(R), N(OR), N⁺(O)(OR), and N—NRR;

each Z is independently selected from CR₂, C(═Y), NR, O, and S; and

R⁸, R⁹ and R¹⁰ are independently selected from H, F, Cl, Br, I, —C(═Y¹)R, —C(═Y¹)OR, —C(═Y¹)NR₂, —NR₂, —N⁺R₃, —N(R)C(═Y¹)R, —N(R)C(═Y¹)OR, —N(R)C(═Y¹)NR₂, —OR, —OC(═Y¹)R, —OC(═Y¹)OR, —OC(═Y¹)NR₂, —OS(O)₂(OR), —OP(═Y¹)(OR)₂, —OP(OR)₂, —P(═Y¹)(OR)₂, —P(═Y¹)(OR)NR₂, —SR, —S(O)R, —S(O)₂R, —S(O)₂NRR, —S(O)(OR), —S(O)₂(OR), —SC(═Y¹)R, —SC(═Y¹)OR, —SC(═Y¹)NR₂, C₁-C₈ alkylhalide, C₁-C₈ alkylsulfonate, C₁-C₈ alkylamino, C₁-C₈ alkylhydroxyl, C₁-C₈ alkylthiol, 5-7 membered ring lactam, 5-7 membered ring lactone, 5-7 membered ring sultam, C₁-C₈ alkyl, C₂-C₈ alkenyl, C₂-C₈ alkynyl, C₆-C₂₀ aryl, C₃-C₁₂ carbocycle, and C₁-C₂₀ heterocyclyl.

In embodiments of structures IIIa-o wherein ═Y¹ is ═N—OR, the oxime moiety can exist as either the E or Z isomer or as a mixture of both.

In certain embodiments of compounds of Formula I, R⁴ is selected from structures IVa-j:

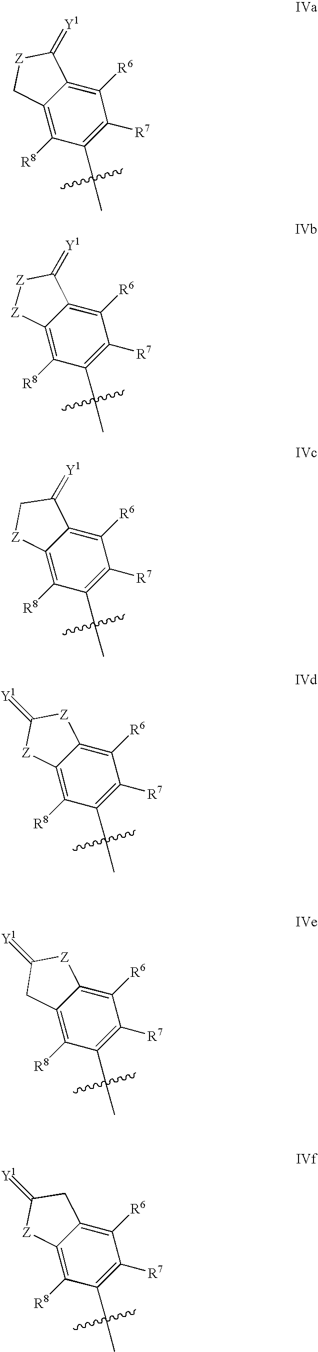

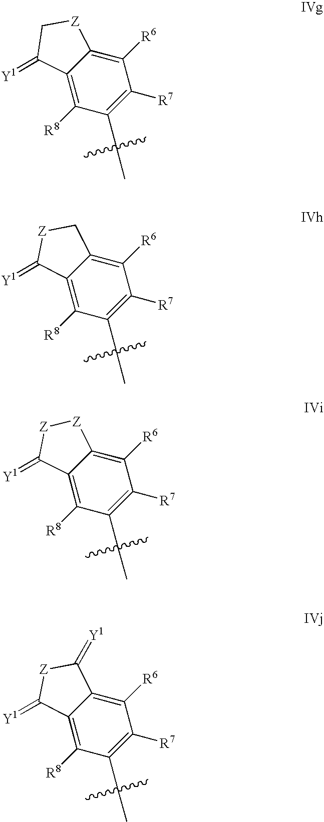

wherein Y¹, Z, R⁶, R⁷ and R⁸ are as defined above. In embodiments of structures IVa-j wherein ═Y¹ is ═N—OR, the oxime moiety can exist as either the E or Z isomer or as a mixture of both.

In certain embodiments of compounds of Formula I, R⁴ is selected from structures Va-j:

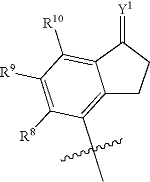

wherein Y¹, Z, R⁸, R⁹ and R¹⁰ are as defined above. In embodiments of structures Va-j wherein ═Y¹ is ═N—OR, the oxime moiety can exist as either the E or Z isomer or as a mixture of both.

Also provided are compounds of Formula VI

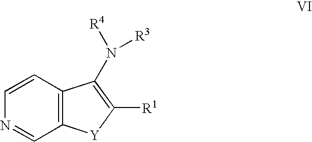

and stereoisomers, geometric isomers, tautomers, solvates, metabolites, salts (including pharmaceutically acceptable salts) and pharmaceutically acceptable prodrugs thereof, wherein:

Y is S or O;

R¹ is H, I, Br, CH═CH₂, C(═O)OR^{a}, C(═O)R^{b}, CH(OH)Ar, (C₁-C₆ alkyl)OH, C(═NNH₂)(C₁-C₃ alkyl)-O(C₁-C₃ alkyl), C(═O)NR^{c}R^{d}, NHR^{e}, NHC(═O)(C₁-C₆ alkyl), Ar, hetAr or a saturated or partially unsaturated heterocyclyl;

R³ is H, C₁-C₆ alkyl, or CH₂CH₂OH;

R⁴ is

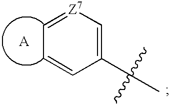

Z⁷ is N or CR⁵;

R⁵ is H or OH;

A is:

(i) a fused 6 membered heteroaryl ring having one or two ring nitrogen atoms and optionally substituted with one to three groups independently selected from C₁-C₃ alkyl, OH, OCH₃, NH₂, F, Cl, Br, I, oxo, and ═NOR^{f};

(ii) a fused 5 membered heteroaryl ring having a ring nitrogen atom and optionally having a second ring heteroatom selected from N and O, wherein said ring is optionally substituted with one or two groups independently selected from NH₂, OR^{f}, F, Cl, Br, I, C₁-C₃ alkyl, oxo and ═NOR^{f};

(iii) a fused 5-6 membered saturated or partially unsaturated heterocyclic ring having one or two ring heteroatoms independently selected from N and O and optionally substituted with one or two groups independently selected from C₁-C₆ alkyl, oxo, and ═NOR^{f};

(iv) a fused 5-6 membered carbocyclic ring optionally substituted with oxo, NH₂, and ═NOR^{f}; or

(v) a fused phenyl ring optionally substituted with one to three groups independently selected from F, Cl, Br, I, OR^{f}, and NH₂;

Ar is phenyl optionally substituted with one to three groups independently selected from OCH₃, CN, C(═O)NR^{f}R^{g}, CF₃, F, Cl, Br, I, NR^{f}R^{g}, C(═O)OR^{f}, and C₁-C₆ alkyl;

hetAr is a 5-6 membered heteroaryl having a ring nitrogen atom and optionally having one or two additional ring heteroatoms independently selected from N, O and S, wherein said heteroaryl is optionally substituted with one to three groups independently selected from (i) C₁-C₆ alkyl, (ii) (C₁-C₆ alkyl)OH, (iii) NR^{f}R^{g}, (iv) (CH₂)₀₋₁-heterocycle or C(═O)heterocycle, wherein said heterocycle is a 6 membered ring having 1 or 2 ring atoms independently selected from N and O and optionally substituted with C₁-C₆ alkyl, (v) C(═O)OR^{f}, (vi) (C₁-C₆ alkyl)NR^{f}R^{g}, (vii) C(═O)NH(C₁-C₆ alkyl)NR^{f}R^{g}, (viii) O—(C₁-C₆)NR^{f}R^{g}, (ix) SMe and (x) CF₃;

R^{a} is H, C₁-C₆ alkyl, or (C₁-C₆ alkyl)-NR^{f}R^{g};

R^{b} is H, Ar, C₁-C₆ alkyl, (C₁-C₆ alkyl)-O(C₁-C₆ alkyl), or a 6 membered heterocycle having 1-2 ring heteroatoms independently selected from N and O;

R^{c} is H or (C₁-C₆ alkyl);

R^{d} is H, C₁-C₆ alkyl, (C₁-C₆ alkyl)NR^{f}R^{g}, NH₂, Ar, (CH₂)₀₋₂-hetAr, (C₁-C₆ alkyl)-OR^{f}, (C₁-C₆ alkyl)-SO₂CH₃, (C₁-C₆ alkyl)CH(OH)(C₁-C₆ alkyl), (C₁-C₆ alkyl)CH(OH)(C₁-C₆ alkyl)OH, (C₁-C₆ alkyl)C(═O)NR^{f}R^{g}, or (CH₂)₀₋₂-heterocycle wherein said heterocycle is a 5-6 membered ring having 1-2 ring atoms independently selected from N and O and optionally substituted with C₁-C₆ alkyl,

or R^{c} and R^{d} together with the nitrogen atom to which they are attached form a 5-6 membered heterocyclic ring having a ring nitrogen atom and optionally having a second ring heteroatom selected from N and O, said ring being optionally substituted with one to three groups independently selected from C₁-C₆ alkyl;

R^{e} is H, C₁-C₆ alkyl, (C₁-C₆ alkyl)O(C₁-C₆ alkyl), or (C₁-C₆ alkyl)NR^{f}R^{g}; and

R^{f} and R^{g} are independently H or C₁-C₆ alkyl, or R^{g} is CH₂Ph.

Examples of hetAr groups in the above Formula VI include, but are not limited to, 2-pyridyl, 3-pyridyl, 4-pyridyl, 2-pyrimidyl, 4-pyrimidyl, 5-pyrimidyl, 6-pyrimidyl, 2-pyrazinyl, 3-pyrazinyl, 2-thiazolyl, 4-thiazolyl, 5-thiazolyl, 2-oxazolyl, 4-oxazolyl, 5-oxazolyl, 3-isoxazolyl, 4-isoxzaolyl, 5-isoxazolyl, 2-imidazolyl, 4-imidazolyl, 5-imidazolyl, and oxadiazolyl. In certain embodiments, the exemplary herAr groups are substituted with Me, Et, Pr, iPr, tBu, CO₂H, CO₂Me, NH₂, CH₂OH, CH₂NMe₂, C(═O)(4-methylpiperizin-1-yl), C(═O)NHCH₂CH₂NMe₂, morpholinyl, CH₂-piperazinyl, CH₂-(4-methylpiperazin-1-yl), CH₂-morpholin-4-yl, or 4-methylpiperazinyl.

Examples of heterocyclic groups in the above Formula VI include, but are not limited to, 1-pyrrolidinyl, 2-pyrrolidinyl, 3-pyrrolidinyl, 1-piperazinyl, 2-piperazinyl, 3-piperazinyl, 1-piperidinyl, 2-piperidinyl, 3-piperidinyl, 4-piperidinyl, 2-morpholinyl, 3-morpholinyl, and 4-morpholinyl.

Examples of Ar groups in the above Formula VI include, but are not limited to,

In one embodiment of Formula VI, R³ is H.

In one embodiment of Formula V¹, Z⁷ is CR⁵. In certain embodiments, R⁵ is H. In other embodiment, R⁵ is methyl. In other embodiments, R⁵ is CH₂CH₂OH.

In one embodiment of Formula VI, A is a fused 6 membered heteroaryl ring having one or two ring nitrogen atoms and optionally substituted with C₁-C₆ alkyl, NH₂, OH, or OCH₂OCH₂Ph.

In a particular embodiment, R⁴ is selected from the structures:

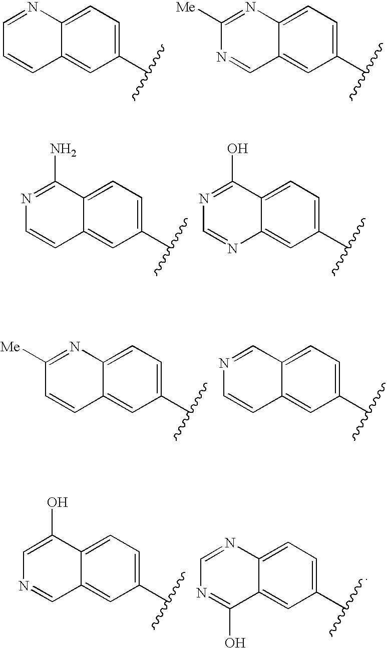

In one embodiment of Formula VI, A is a fused 5 membered heteroaryl ring having a ring nitrogen atom and optionally having a second ring heteroatom selected from N and O and optionally substituted with NH₂, or OH.

In a particular embodiment, R⁴ is selected from the structures:

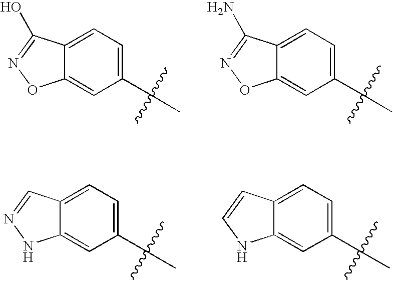

In one embodiment of Formula VI, A is a fused 5-6 membered saturated or partially unsaturated heterocyclic ring having one or two ring heteroatoms independently selected from N and O and optionally substituted with oxo, C₁-C₆ alkyl, or ═NOR^{f}. In a particular embodiment, A is a fused 5-6 membered saturated or partially unsaturated heterocyclic ring having one or two ring heteroatoms independently selected from N and O and with oxo, or ═NOH. In embodiments wherein the heterocyclic ring is substituted with ═NOR^{f}, the oxime moiety can exist in the E or Z configuration.

In a particular embodiment, R⁴ is selected from the structures:

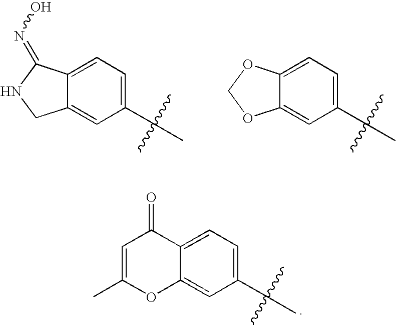

In one embodiment of Formula VI, A is a fused 5 membered carbocyclic ring optionally substituted with oxo, NH₂, or ═NOR^{f}. In a particular embodiment, A is a fused 5 membered carbocyclic ring substituted with oxo or ═NOR^{f}. In certain embodiments, R^{f} is H. In embodiments wherein the carbocyclic ring is substituted with ═NOR^{f}, the oxime moiety can exist in the E or Z configuration.

In a particular embodiment, R⁴ is selected from the structures:

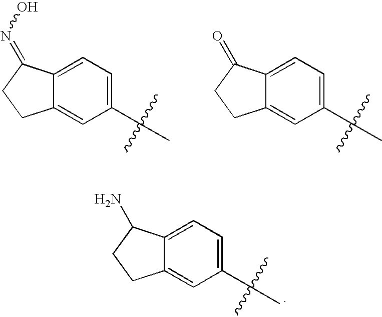

In one embodiment of Formula VI, A is a fused phenyl ring optionally substituted with one to three groups independently selected from F, OH, OMe or NH₂.

In a particular embodiment, R⁴ is selected from the structures:

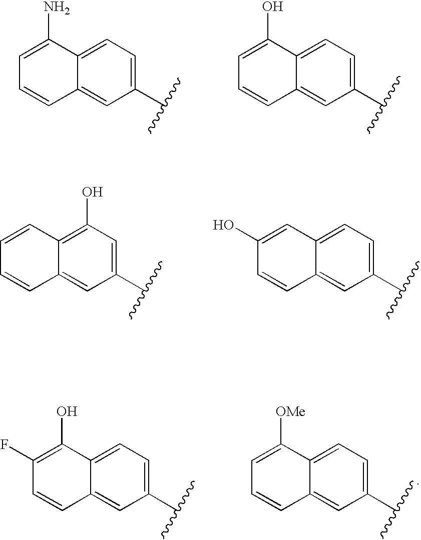

In one embodiment of Formula IV, Z⁷ is N. In a particular embodiment, R⁴ is selected from the structures:

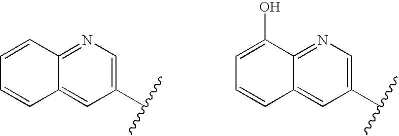

In one embodiment of Formula VI, R¹ is C(═O)OR^{a}. Exemplary embodiments include, but are not limited to, CO₂H, CO₂CH₃, CO₂CH₂CH₃, CO₂CH₂CH₂CH₃, CO₂CH(CH₃)₂ and CO₂CH₂CH₂N(CH₃)₂.

In one embodiment of Formula VI, R¹ is C(═O)R^{b}. Exemplary embodiments include, but are not limited to, C(═O)(4-methoxyphenyl), C(═O)(tetrahydro-2H-pyran-4-yl)C(═O)CH₂CH₂CH₃, C(═O)CH(CH₃)₂, and C(═O)CH₂CH₂CH₂OCH₃.

In one embodiment of Formula VI, R¹ is CH(OH)R^{b}. An exemplary embodiment includes, but is not limited to, CH(OH)(4-methoxyphenyl).

In one embodiment of Formula VI, R¹ is (C₁-C₆ alkyl)OH. Exemplary embodiments include, but are not limited to, CH₂OH and CH₂CH₂OH.

In one embodiment of Formula VI, R¹ is C(═O)NR^{c}R^{d}. In certain embodiments, R^{c} is H. In certain embodiments, R^{c} is H, R^{d} is (C₁-C₆ alkyl)NH₂, (C₁-C₆ alkyl)NH(C₁-C₆ alkyl), (C₁-C₆ alkyl)N(C₁-C₆ alkyl)₂, (C₁-C₆ alkyl)-heteorcyclyl, (C₁-C₆ alkyl)SO₂CH₃, or (C₁-C₆ alkyl)C(═O)NR^{f}R^{g} and A is other than a cycloalkyl or heterocyclic ring substituted with oxo or ═NOR^{f}.

Exemplary embodiments of R¹ include, but are not limited to, the following structures:

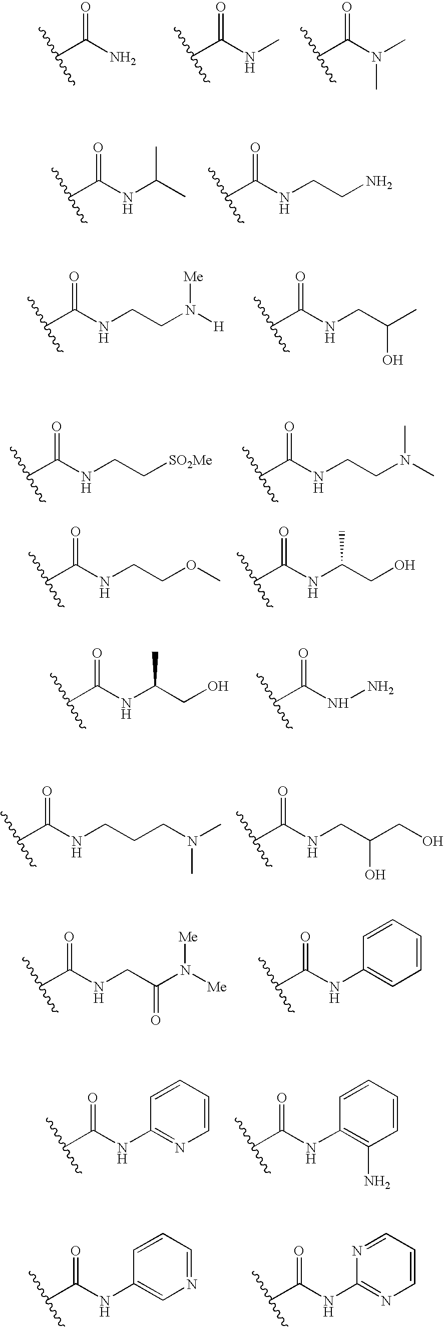

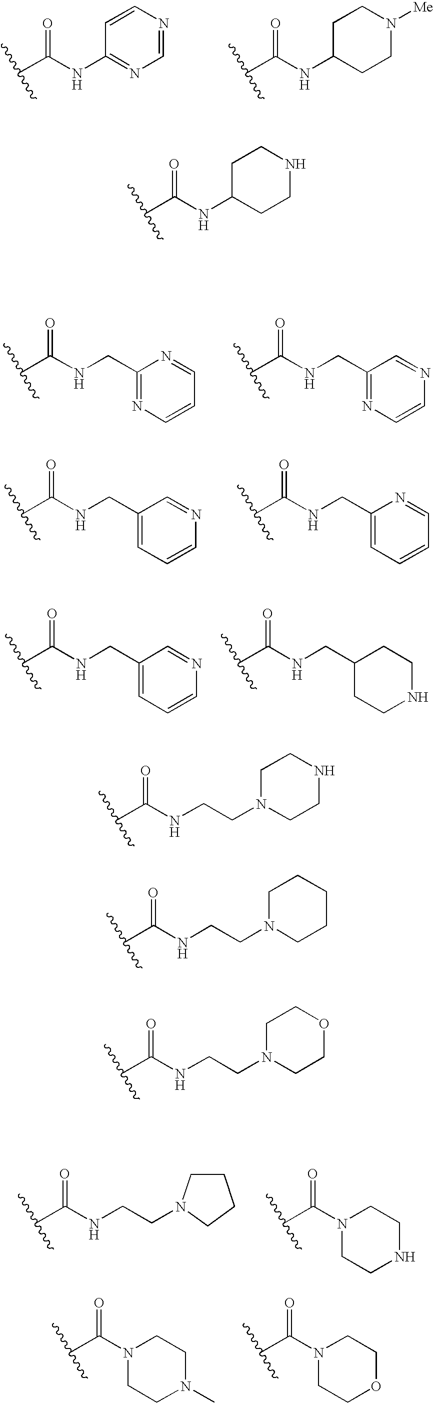

In one embodiment of Formula VI, R¹ is NHR^{e}. Exemplary embodiments include, but are not limited to, NHCH₂CH₃, NHCH₂CH₂CH₃, NHCH₂CH₂OCH₃, NHCH₂CH₂CH₂N(CH₂CH₃)₂, and NH(4-methoxyphenyl).

In one embodiment of Formula VI, R¹ is NHC(═O)(C₁-C₆ alkyl). An exemplary embodiment includes, but is not limited to, NHC(═O)CH₂CH₃.

In one embodiment of Formula VI, R¹ is Ar. Exemplary embodiments include, but are not limited to, the following structures:

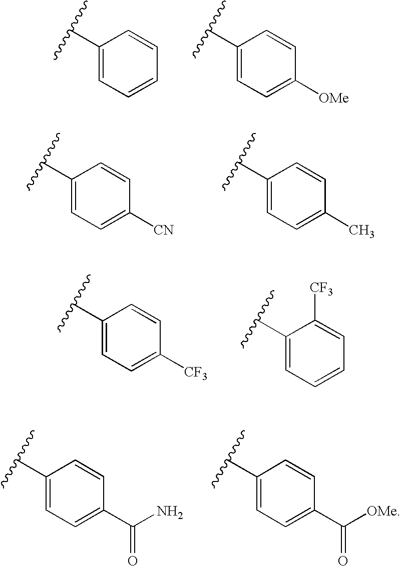

In one embodiment of Formula VI, R¹ is hetAr. Exemplary embodiments include, but are not limited to, the following structures

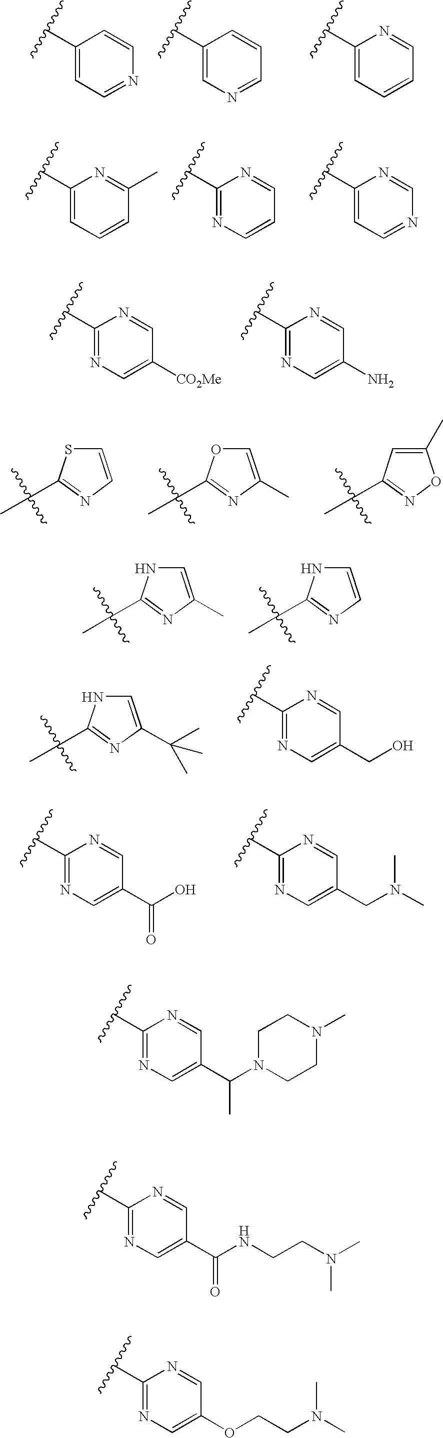

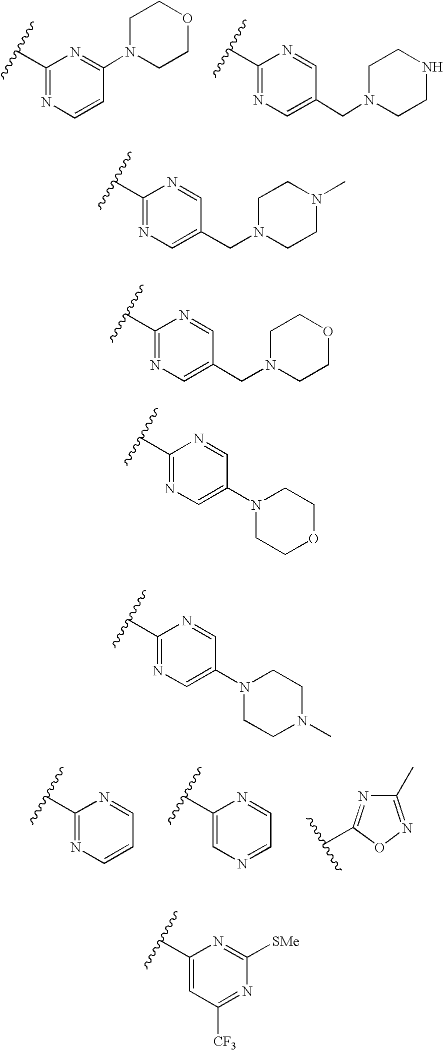

In other embodiments, R¹ is or a saturated or partially unsaturated heterocyclyl, such as, but not limited to, dihydroimidazolyl.

Also provided are compounds of Formula II

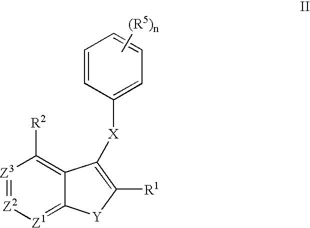

and stereoisomers, geometric isomers, tautomers, solvates, metabolites and pharmaceutically acceptable salts thereof, wherein:

X is selected from NR³, O, C(═O), and S;

Y is O or S;

Z¹, Z², and Z³ are independently selected from CR⁵ and N, and one or two of Z¹, Z², and Z³ is N;

R¹, R² and R⁵ are independently selected from H, F, Cl, Br, I, —C(═Y¹)R, —C(═Y¹)OR, —C(═Y¹)NR₂, —NR₂, —N⁺R₃, —N(R)C(═Y¹)R, —N(R)C(═Y¹)OR, —N(R)C(═Y¹)NR₂, —NR-alkylaryl, —NRSO₂NRR, —OR, —OC(═Y¹)R, —OC(═Y¹)OR, —OC(═Y¹)NR₂, —OS(O)₂(OR), —OP(═Y¹)(OR)₂, —OP(OR)₂, —P(═Y¹)(OR)₂, —P(═Y¹)(OR)NR₂, —SR, —S(O)R, —S(O)₂R, —S(O)₂NRR, —S(O)(OR), —S(O)₂(OR), —SC(═Y¹)R, —SC(═Y¹)OR, —SC(═Y¹)NR₂, C₁-C₈ alkylhalide, C₁-C₈ alkylsulfonate, C₁-C₈ alkylamino, C₁-C₈ alkylhydroxyl, C₁-C₈ alkylthiol, 5-7 membered ring lactam, 5-7 membered ring lactone, 5-7 membered ring sultam, C₁-C₈ alkyl, C₂-C₈ alkenyl, C₂-C₈ alkynyl, C₆-C₂₀ aryl, C₃-C₁₂ carbocycle, and C₁-C₂₀ heterocyclyl;

R³ is selected from H, C₁-C₈ alkyl, C₂-C₈ alkenyl, C₂-C₈ alkynyl, C₆-C₂₀ aryl, C₃-C₁₂ carbocycle, C₁-C₂₀ heterocyclyl, and a protecting group;

each R is independently H, C₁-C₈ alkyl, C₂-C₈ alkenyl, C₂-C₈ alkynyl, C₆-C₂₀ aryl, C₁-C₂₀ heterocyclyl, or a protecting group;

Y¹ is independently selected from O, S, NR, N⁺(O)(R), N(OR), N⁺(O)(OR), and N—NRR;

each alkyl, alkenyl, alkynyl, aryl, phenyl, carbocyclyl, and heterocyclyl is optionally substituted with one or more substituents independently selected from F, Cl, Br, I, CN, CF₃, OR, SR, R, ═O, ═S, ═NR, ═N⁺(O)(R), ═N(OR), ═N⁺(O)(OR), ═N—NR₂, —C(═Y¹)R, —C(═Y¹)OR, —C(═Y¹)NR₂, —NR₂, —N⁺R³, —N(R)C(═Y¹)R, —N(R)C(═Y¹)OR, —N(R)C(═Y¹)NR₂, —SR, —OC(═Y¹)R, —OC(═Y¹)OR, —OC(═Y¹)NR₂, —OS(O)₂(OR), —OP(═Y¹)(OR)₂, —OP(OR)₂, —P(═Y¹)(OR)₂, —P(═Y¹)(OR)NR₂, —S(O)R, —S(O)₂R, —S(O)₂NR, —S(O)(OR), —S(O)₂(OR), —SC(═Y¹)R, —SC(═Y¹)OR, and —SC(═Y¹)NR₂; and

n is 0, 1, 2, 3, 4 or 5.

In certain embodiments of compounds of Formula II, each R⁵ is independently selected from F, Cl, Br, I and OR.

In certain embodiments of compounds of Formula II, each R⁵ is independently selected from OH and Cl.

Also included herein are compounds of Formula VII

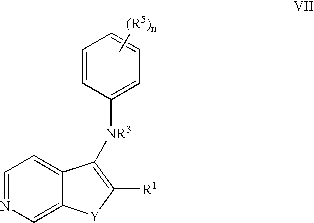

and stereoisomers, geometric isomers, tautomers, solvates, metabolites, salts (including pharmaceutically acceptable salts) and pharmaceutically acceptable prodrugs thereof, wherein:

Y is O or S;

R¹ is H, I, Br, CH═CH₂, C(═O)OR^{a}, C(═O)R^{b}, CH(OH)Ar, (C₁-C₆ alkyl)OH, C(═NNH₂)(C₁-C₃ alkyl)-O(C₁-C₃ alkyl), C(═O)NR^{c}R^{d}, NHR^{e}, NHC(═O)(C₁-C₆ alkyl), Ar, hetAr, or a saturated or partially unsaturated heterocyclyl;

R³ is H C₁-C₆ alkyl or CH₂CH₂OH;

each R⁵ is independently selected from F, Cl, Br, I, CN, CF₃, C₁-C₆ alkyl, phenyl, O-phenyl, OH, OMe, CH₂OH, C(═O)(C₁-C₆ alkyl), NHC(═O)(C₁-C₄ alkyl), and 4-methylpyrazol-3-yl;

n is 0, 1, 2 or 3;

Ar is phenyl optionally substituted with one to three groups independently selected from OCH₃, CN, C(═O)NR^{f}R^{g}, CF₃, F, Cl, Br, I, NR^{f}R^{g}, C(═O)OR^{f}, and C₁-C₆ alkyl;

hetAr is a 5-6 membered heteroaryl having a ring nitrogen atom and optionally having one or two additional ring heteroatoms independently selected from N, O and S, wherein said heteroaryl is optionally substituted with one to three groups independently selected from (i) C₁-C₆ alkyl, (ii) (C₁-C₆ alkyl)OH, (iii) NR^{f}R^{g}, (iv) (CH₂)₀₋₁-heterocycle or C(═O)heterocycle, wherein said heterocycle is a 6 membered ring having 1 or 2 ring atoms independently selected from N and O and optionally substituted with C₁-C₆ alkyl, (v) C(═O)OR^{f}, (vi) (C₁-C₆ alkyl)NR^{f}R^{g}, (vii) C(═O)NH(C₁-C₆ alkyl)NR^{f}R^{g}, (viii) O—(C₁-C₆)NR^{f}R^{g}, (ix) SMe and (x) CF₃.

R^{a} is H, C₁-C₆ alkyl, or (C₁-C₆ alkyl)-NR^{f}R^{g};

R^{b} is H, Ar, C₁-C₆ alkyl, (C₁-C₆ alkyl)-O(C₁-C₆ alkyl), or a 6 membered heterocycle having 1-2 ring heteroatoms independently selected from N and O;

R^{c} is H or (C₁-C₆ alkyl);

R^{d} is H, C₁-C₆ alkyl, (C₁-C₆ alkyl)NR^{f}R^{g}, NR^{f}R^{g}, Ar, (CH₂)₀₋₂-hetAr, (C₁-C₆ alkyl)-OR^{f}, (C₁-C₆ alkyl)-SO₂CH₃, (C₁-C₆ alkyl)CH(OH)(C₁-C₆ alkyl), (C₁-C₆ alkyl)CH(OH)(C₁-C₆ alkyl)OH, (C₁-C₆ alkyl)C(═O)NR^{f}R^{g}, or (CH₂)₀₋₁-heterocycle wherein said heterocycle is a 5-6 membered ring having 1-2 ring atoms independently selected from N and O and optionally substituted with C₁-C₆ alkyl,

or R^{c} and R^{d} together with the nitrogen atom to which they are attached form a 5-6 membered heterocyclic ring having a ring nitrogen atom and optionally having a second ring heteroatom selected from N and O and optionally substituted with C₁-C₆ alkyl;

R^{e} is H, C₁-C₆ alkyl, (C₁-C₆ alkyl)O(C₁-C₆ alkyl), or (C₁-C₆ alkyl)NR^{f}R^{g}; and

R^{f} and R^{g} are independently H or C₁-C₆ alkyl, or R^{g} is CH₂Ph.

In one embodiment of Formula VII, R³ is H.

In certain embodiments of compounds of Formula VII, each R⁵ is independently selected from F, Cl, Br, CN, OCH₃, OH, Me, Et, Pr, CF₃, NHC(═O)CH₃, CH₂OH, C(═O)CH₂CH₃, C(═O)CH₃, O-phenyl, phenyl, and 4-methylpyrazol-3-yl.

In one embodiment of Formula VII, the group

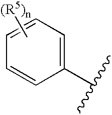

is selected from the structures:

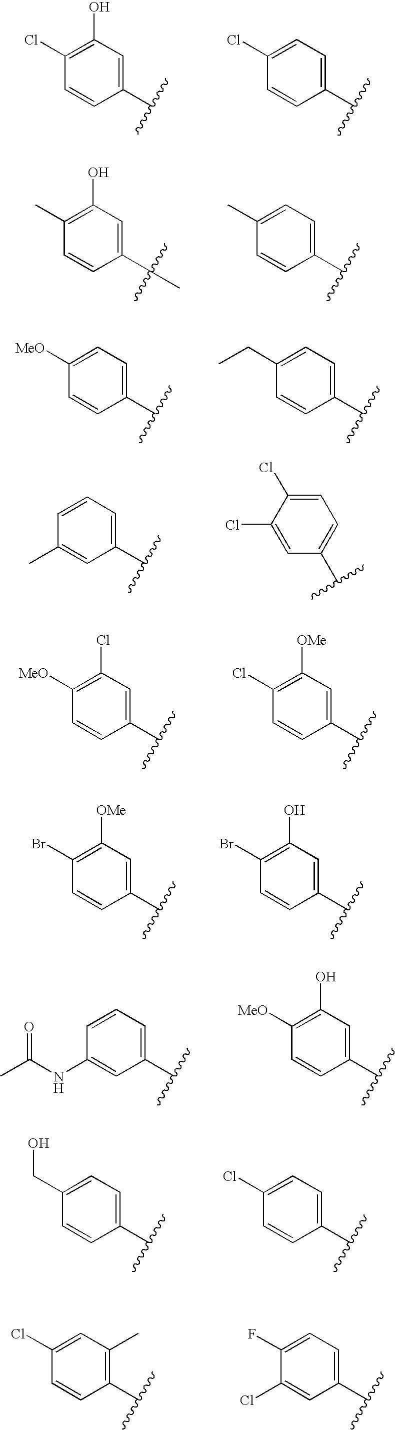

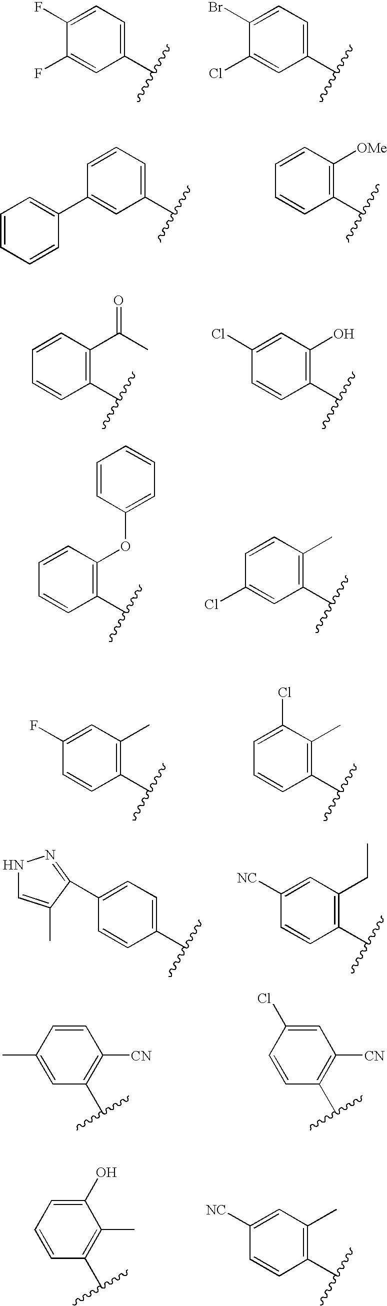

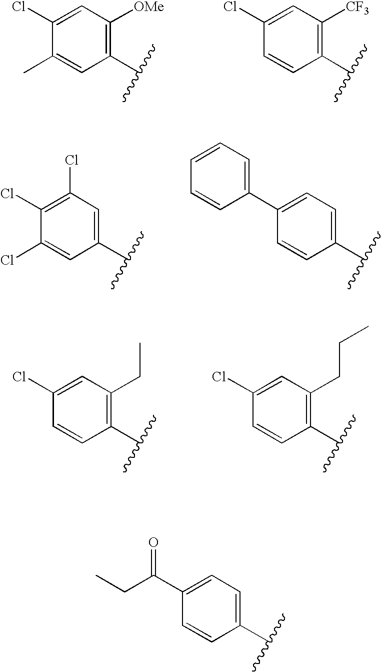

In a particular embodiment, the group is

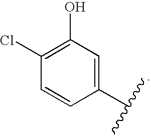

In one embodiment of Formula VII, R¹ is C(═O)OR^{a}. In a particular embodiment, R¹ is CO₂CH₂CH₃.

In one embodiment of Formula VII, R¹ is H.

In one embodiment of Formula VII, R¹ is hetAr. Exemplary embodiments include, but are not limited to, the following structures:

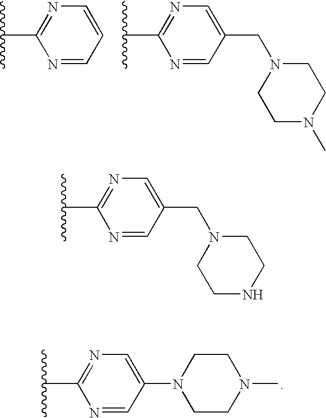

In one embodiment of Formula VII, R¹ is C(═O)NR^{c}R^{d}. In certain embodiments, R^{c} is H. Exemplary embodiments of R¹ include, but are not limited to, C(═O)NH(CH₂CH₂)N(CH₃)₂C(═O)NHCH(CH₃)₂, C(═O)NH(CH₂CH₂)OCH₃, C(═O)NHCH₂CH(OH)CH₃, and C(═O)NHCH₂(pyrid-3-yl).

The compounds of the invention may contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof such as racemic mixtures, form part of the present invention.

In addition, the present invention embraces all geometric and positional isomers. For example, if a compound of the present invention incorporates a double bond or a fused ring, the cis- and trans-forms, as well as mixtures thereof, are embraced within the scope of the invention. Both the single positional isomers and mixture of positional isomers, e.g., resulting from the N-oxidation of the pyrimidine and pyrazine rings, are also within the scope of the present invention.

In the structures shown herein, where the stereochemistry of any particular chiral atom is not specified, then all stereoisomers are contemplated and included as the compounds of the invention. Where stereochemistry is specified by a solid wedge or dashed line representing a particular configuration, then that stereoisomer is so specified and defined.

The compounds of the present invention may exist in unsolvated as well as solvated forms with pharmaceutically acceptable solvents such as water, ethanol, and the like, and it is intended that the invention embrace both solvated and unsolvated forms.

The compounds of the present invention may also exist in different tautomeric forms, and all such forms are embraced within the scope of the invention. The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons.

Hydroxyimino or alkoxyimino (oxime) moieties of the compounds of the invention can be positioned on any of carbon atoms of ring A. Although the oxime geometry may be depicted in a particular configuration, e.g., compounds of Examples 1-52, an oxime moiety of the compounds of the invention can exist as either the E or Z isomer, or as a mixture of both

The present invention also embraces isotopically-labeled compounds of the present invention which are identical to those recited herein, but for the fact that one or more atoms are replaced by an atom having an atomic mass or mass number different from the atomic mass or mass number usually found in nature. All isotopes of any particular atom or element as specified is contemplated within the scope of the compounds of the invention, and their uses. Exemplary isotopes that can be incorporated into compounds of the invention include isotopes of hydrogen, carbon, nitrogen, oxygen, phosphorus, sulfur, fluorine, chlorine and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I and ¹²⁵I. Certain isotopically-labeled compounds of the present invention (e.g., those labeled with ³H and ¹⁴C) are useful in compound and/or substrate tissue distribution assays. Tritiated (i.e., 3H) and carbon-14 (i.e., ¹⁴C) isotopes are useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Positron emitting isotopes such as ¹⁵O, ¹³N, ¹¹C and ¹⁸F are useful for positron emission tomography (PET) studies to examine substrate receptor occupancy. Isotopically labeled compounds of the present invention can generally be prepared by following procedures analogous to those disclosed in the Schemes and/or in the Examples herein below, by substituting an isotopically labeled reagent for a non-isotopically labeled reagent.

Synthesis of Raf Inhibitor Compounds

Compounds of the present invention may be synthesized by synthetic routes that include processes analogous to those well-known in the chemical arts, particularly in light of the description contained herein. The starting materials are generally available from commercial sources such as Aldrich Chemicals (Milwaukee, Wis.) or are readily prepared using methods well known to those skilled in the art (e.g., prepared by methods generally described in Louis F. Fieser and Mary Fieser, Reagents for Organic Synthesis, v. 1-19, Wiley, N.Y. (1967-1999 ed.), or Beilsteins Handbuch der organischen Chemie, 4, Aufl. ed. Springer-Verlag, Berlin, including supplements (also available via the Beilstein online database).

In certain embodiments, compounds of this invention may be readily prepared using procedures well-known to prepare other heterocycles, which are described in: Comprehensive Heterocyclic Chemistry, Editors Katrizky and Rees, Pergamon Press, 1984; Klemm et al (1970) J. Hetero. Chem. 7(2):373-379; Klemm et al (1974) J. Hetero. Chem. 11(3): 355-361; Klemm et al (1976) J. Hetero. Chem. 13:273-275; Klemm et al (1985) J. Hetero. Chem. 22(5):1395-1396; Bisagni et al (1974) Bull. Soc. Chim. Fr. (3-4, Pt. 2):515-518; Frehel et al (1984) Heterocycles 22(5):1235-1247; WO 93/13664; WO 2004/012671; WO 2005/061476; U.S. Application Publication Nos. 2003/0045540, US 2003/0105089, and 2004/0024210; and U.S. Pat. Nos. 5,252,581, 6,232,320, and 6,579,882.

Compounds of this invention may be prepared singly or as compound libraries comprising at least 2, for example 5 to 1,000 compounds, or 10 to 100 compounds. Libraries of compounds of this invention may be prepared by a combinatorial ‘split and mix’ approach or by multiple parallel synthesis using either solution phase or solid phase chemistry, by procedures known to those skilled in the art. Thus according to a further aspect of the invention there is provided a compound library comprising at least 2 compounds, or pharmaceutically acceptable salts thereof.

For illustrative purposes, Schemes 1-6 show general method for preparing the compounds of the present invention as well as key intermediates. For a more detailed description of the individual reaction steps, see the Examples section below. Those skilled in the art will appreciate that other synthetic routes may be used to synthesize the inventive compounds. Although specific starting materials and reagents are depicted in the Schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

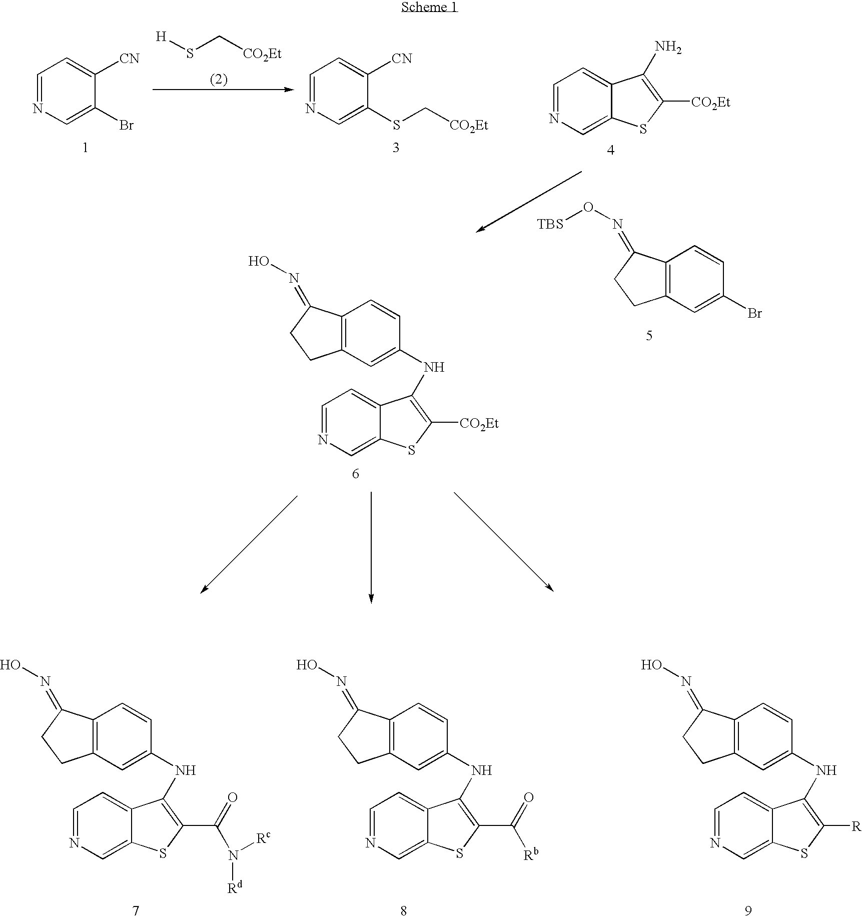

Scheme 1 shows a method of preparing compounds of Formula I wherein Y is S, X is NH, Z¹ and Z³ are CH, Z² is N, R^{b}, R^{c} and R^{d} are as defined herein, R² and R³ are H and R is alkyl or aryl. According to Scheme 1, condensation of (1) with ethyl thioglycolate under basic catalysis affords the thioether (3). Treatment of this compound with base results in cyclization to afford the thieno[2,3-c]pyridine ester (4). Palladium-catalyzed (Buchwald type) condensation in the presence of bromide (4) leads to the key intermediate (6). Subsequent manipulation of the ester group in (6) afford amide derivatives of general formula (7) or ketone derivatives of general formula (8) are performed according to Scheme 2. Alkyl or aryl derivatives of general formula (9, R=alkyl, aryl) are prepared via Scheme 1 using the appropriately substituted aryl mercaptan in place of ethyl thioglycolate.

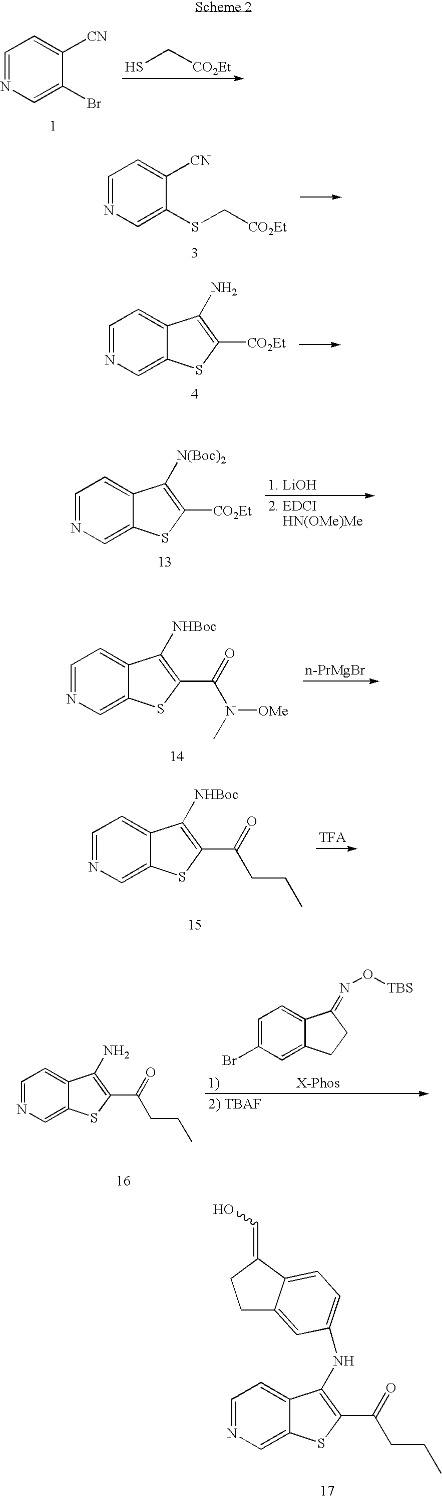

Scheme 2 shows an alternative method of preparing compounds of Formula I wherein Y is S, X is NH, Z¹ and Z³ are CH, Z² is N, R² and R³ are H, and R¹ is alkyl. According to Scheme 2, the amine (4) is protected as its bis-Boc derivative (13). Saponification under basic conditions followed by standard amide bond-forming conditions affords the key Weinreb amide intermediate (14). Treatment of amide (14) with various Grignard reagents affords the corresponding ketone derivatives (15). Treatment with TFA affords the amine (16), which can be elaborated in a similar fashion to Scheme 1 to afford the ketone derivative (17).

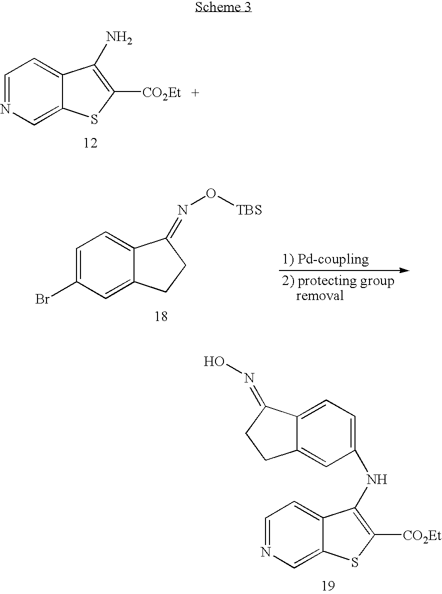

Scheme 3 shows an alternative method of preparing compounds of Formula I wherein Y is S, X is NH, Z¹ and Z³ are CH, Z² is N, R² and R³ are H, and R¹ is CO₂(alkyl). According to Scheme 3, palladium catalyzed coupling of amine (12) with compound (18) using standard Buchwald coupling conditions known to those skilled in the art, followed by removal of the tetrabutylsilyl protecting group using standard reagents known to those skilled in the art, affords compound (19). In one embodiment, the coupling takes place in the presence of Pd₂(dba)₃ and X-Phos and a base such as sodium t-butoxide at elevated temperatures, such as about 110° C. In certain embodiment, the reagent used to remove the silyl protecting group is tetrabutylammonium fluoride.

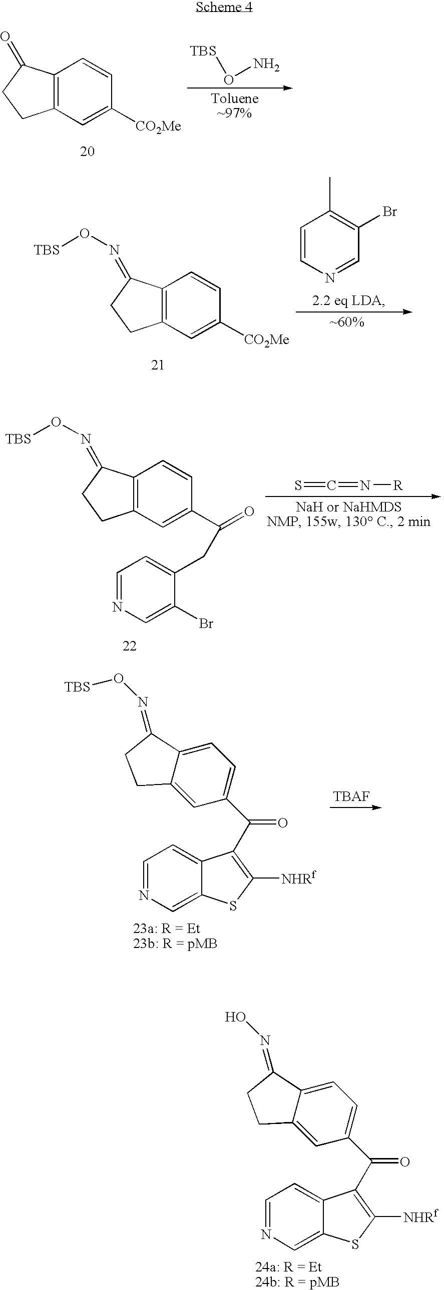

Scheme 4 shows a method of preparing compounds of Formula I wherein Y is S, X is C(═O), Z¹ and Z³ are CH, Z² is N. R² is H, and R^{f} is alkyl (e.g., ethyl) or pMB (4-methoxybenzyl). According to Scheme 4, preparation of the oxime ester (21) can be carried out by condensing the ketone (20) with a suitable oxime. Preparation of the thienopyridine is carried out as similarly described in the literature (see Bremner, D. H, et al., Synthesis, 1998, 1095 and Synthesis 1997, 949), or using microwave conditions as described herein. For example, preparation of the aryl ketone (22) is readily achieved by condensation of ester (21) with a carbon nucleophile. Cyclization to the bicyclic thienopyridine ring system (23) is carried out using microwave conditions in the presence of strong base and a thiocyanate. Deprotection of the oxime functionality under basic (e.g. TBAF) or acidic conditions (e.g. TFA) affords the final product.

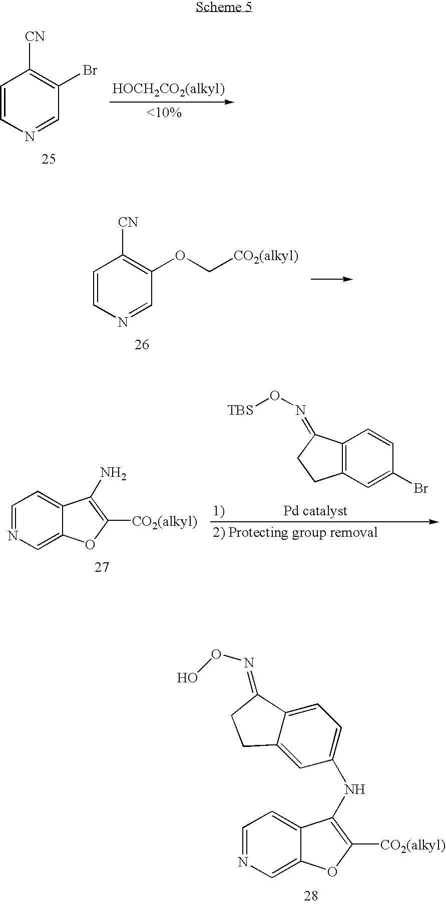

Scheme 5 shows a method of preparing furanopyridine compounds of Formula I wherein Y is O, X is NH, Z¹ and Z³ are CH, Z² is N, R² and R³ are H, R¹ is COOR^{a} and R^{a} is C₁-C₆ alkyl. Alklyation of compound (25) with ethyl glycolate under basic catalytic conditions affords hydroxyl ester (26). Treatment of compound (26) with a strong base promotes cyclization to give compound (27). Palladium catalyzed coupling with an aryl bromide using standard coupling conditions known in the art, followed by removal of the silyl protecting group, affords compound (28). In one embodiment, the coupling takes place in the presence of Pd₂(dba)₃ and X-Phos and a base such as sodium t-butoxide at elevated temperatures, such as about 110° C. In certain embodiment, the reagent used to remove the silyl protecting group is tetrabutylammonium fluoride.

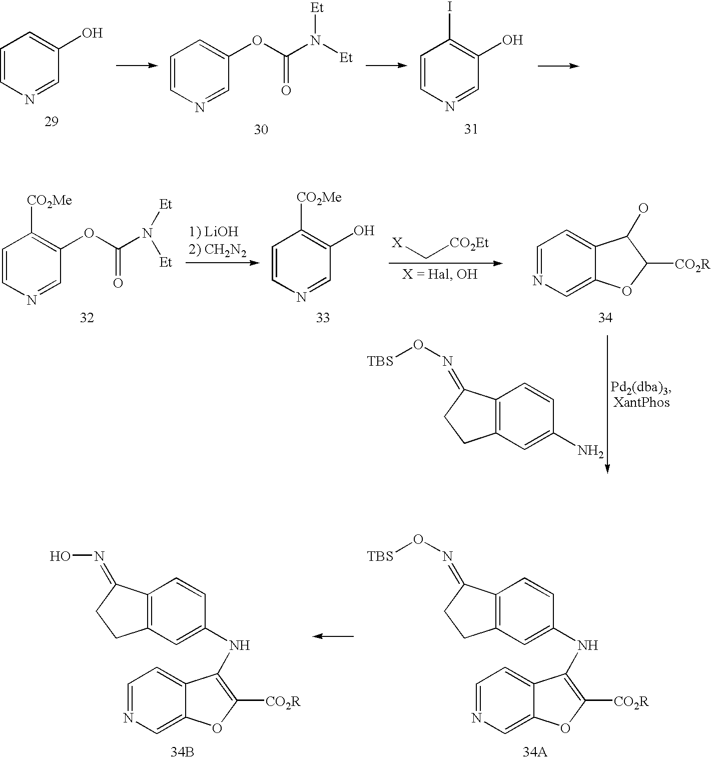

Scheme 6 shows an alternative method of preparing compounds of Formula I wherein Y is O, X is NH, Z¹ and Z³ are CH, and Z² is N. According to Scheme 6, regioselective halogenation can be carried out using known procedures to afford compound (31). Ester formation can be carried out using palladium-catalyzed coupling conditions in the presence of carbon monoxide and alcohol solvent to afford compound (33). Alkylation of the phenol with glycolate or an alpha-bromo ester in the presence of a strong base (eg n-BuLi, or NaH) affords the hydroxyl furanopyridine (34). Following conversion of the hydroxyl group of compound (34) to a triflate, palladium-catalyzed coupling with an aryl amine affords derivative (34A). Deprotection of compound (34A) affords the compound (34B).

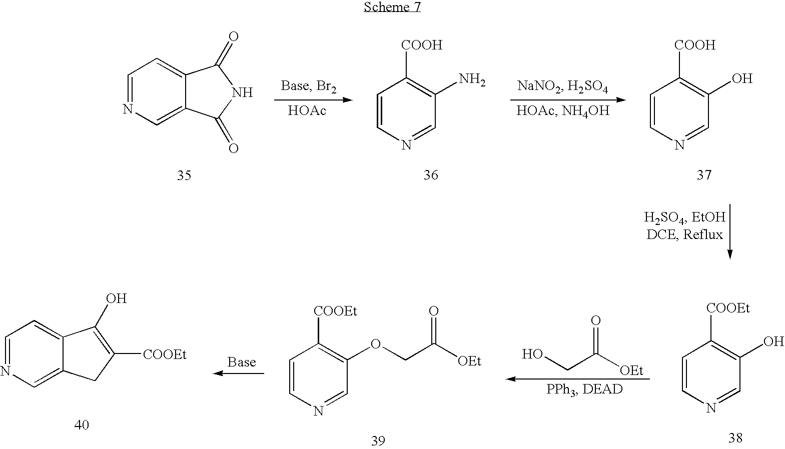

Scheme 7 shows an alternative method of preparing compounds of Formula I. According to Scheme 7, treatment of compound (35) with a base such as NaOH in the presence of bromine promotes formation of 3-amino isonicotinic acid (36). Compound (36) is converted to 3-hydroxyl isonicotinic acid (37) using sodium nitrite and concentrate sulfuric acid to provide compound (38). Compound (38) is obtained from compound (37) via a modified Fisher esterification procedure. Compound (38) is then condensed with ethyl glycolate under Mistunobu conditions to afford hydroxyl ester (39), which can be cyclized to compound (40) in the presence of a base such as NaH. Subsequent transformation of compound (40) to compound (41) is carried out as previously described in Scheme 6.

In preparing compounds of this invention, protection of remote functionality (e.g., primary or secondary amine) of intermediates may be necessary. The need for such protection will vary depending on the nature of the remote functionality and the conditions of the preparation methods. Suitable amino-protecting groups (NH-Pg) include acetyl, trifluoroacetyl, t-butoxycarbonyl (BOC), benzyloxycarbonyl (CBz) and 9-fluorenylmethyleneoxycarbonyl (Fmoc). The need for such protection is readily determined by one skilled in the art. For a general description of protecting groups and their use, see T. W. Greene, Protective Groups in Organic Synthesis, John Wiley & Sons, New York, 1991.

Methods of Separation

In each of the exemplary Schemes it may be advantageous to separate reaction products from one another and/or from starting materials. The desired products of each step or series of steps is separated and/or purified (hereinafter separated) to the desired degree of homogeneity by the techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium and low pressure liquid chromatography methods and apparatus; small scale analytical; simulated moving bed (SMB) and preparative thin or thick layer chromatography, as well as techniques of small scale thin layer and flash chromatography.

Another class of separation methods involves treatment of a mixture with a reagent selected to bind to or render otherwise separable a desired product, unreacted starting material, reaction by product, or the like. Such reagents include adsorbents or absorbents such as activated carbon, molecular sieves, ion exchange media, or the like. Alternatively, the reagents can be acids in the case of a basic material, bases in the case of an acidic material, binding reagents such as antibodies, binding proteins, selective chelators such as crown ethers, liquid/liquid ion extraction reagents (LIX), or the like.

Selection of appropriate methods of separation depends on the nature of the materials involved. For example, boiling point and molecular weight in distillation and sublimation, presence or absence of polar functional groups in chromatography, stability of materials in acidic and basic media in multiphase extraction, and the like. One skilled in the art will apply techniques most likely to achieve the desired separation.

Diastereomeric mixtures can be separated into their individual diastereomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereoisomers to the corresponding pure enantiomers. Also, some of the compounds of the present invention may be atropisomers (e.g., substituted biaryls) and are considered as part of this invention. Enantiomers can also be separated by use of a chiral HPLC column.

A single stereoisomer, e.g., an enantiomer, substantially free of its stereoisomer may be obtained by resolution of the racemic mixture using a method such as formation of diastereomers using optically active resolving agents (Eliel, E. and Wilen, S. “Stereochemistry of Organic Compounds,” John Wiley & Sons, Inc., New York, 1994; Lochmuller, C. H., (1975) J. Chromatogr., 113(3):283-302). Racemic mixtures of chiral compounds of the invention can be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. See: “Drug Stereochemistry, Analytical Methods and Pharmacology,” Irving W. Wainer, Ed., Marcel Dekker, Inc., New York (1993).

Under method (1), diastereomeric salts can be formed by reaction of enantiomerically pure chiral bases such as brucine, quinine, ephedrine, strychnine, α-methyl-β-phenylethylamine (amphetamine), and the like with asymmetric compounds bearing acidic functionality, such as carboxylic acid and sulfonic acid. The diastereomeric salts may be induced to separate by fractional crystallization or ionic chromatography. For separation of the optical isomers of amino compounds, addition of chiral carboxylic or sulfonic acids, such as camphorsulfonic acid, tartaric acid, mandelic acid, or lactic acid can result in formation of the diastereomeric salts.

Alternatively, by method (2), the substrate to be resolved is reacted with one enantiomer of a chiral compound to form a diastereomeric pair (E. and Wilen, S. “Stereochemistry of Organic Compounds”, John Wiley & Sons, Inc., 1994, p. 322). Diastereomeric compounds can be formed by reacting asymmetric compounds with enantiomerically pure chiral derivatizing reagents, such as menthyl derivatives, followed by separation of the diastereomers and hydrolysis to yield the pure or enriched enantiomer. A method of determining optical purity involves making chiral esters, such as a menthyl ester, e.g., (−) menthyl chloroformate in the presence of base, or Mosher ester, α-methoxy-α-(trifluoromethyl)phenyl acetate (Jacob III. J. Org. Chem., (1982) 47:4165), of the racemic mixture, and analyzing the ¹H NMR spectrum for the presence of the two atropisomeric enantiomers or diastereomers. Stable diastereomers of atropisomeric compounds can be separated and isolated by normal- and reverse-phase chromatography following methods for separation of atropisomeric naphthyl-isoquinolines (WO 96/15111). By method (3), a racemic mixture of two enantiomers can be separated by chromatography using a chiral stationary phase (“Chiral Liquid Chromatography” (1989) W. J. Lough, Ed., Chapman and Hall, New York; Okamoto, J. of Chromatogr., (1990) 513:375-378). Enriched or purified enantiomers can be distinguished by methods used to distinguish other chiral molecules with asymmetric carbon atoms, such as optical rotation and circular dichroism.

Biological Evaluation

B-Raf mutant protein 447-717 (V600E) was co-expressed with the chaperone protein Cdc37, complexed with Hsp90 (Roe, et al. Cell, (2004) 116:87-98; Stancato, et al. J. Biol. Chem., (1993) 268:21711-21716).

Determining the activity of Raf in the sample is possible by a number of direct and indirect detection methods (U.S. Patent Publication No. 2004/082014). Activity of human recombinant B-Raf protein may be assessed in vitro by assay of the incorporation of radiolabelled phosphate to recombinant MAP kinase (MEK), a known physiologic substrate of B-Raf, according to U.S. Publication No. 2004/127496 and WO 03/022840. The activity/inhibition of V600E full-length B-Raf was estimated by measuring the incorporation of radiolabeled phosphate from [γ-³³P]ATP into FSBA-modified wild-type MEK (Example 8).

Suitable methods of Raf activity depend on the nature of the sample. In cells, the activity of Raf is on the one hand determined by the amount of the Raf expressed in the cell, and on the other hand by the amount of the activated Raf. The activation of the transcription of the genes coding for Raf protein, in particular B-Raf protein, may be made, for example, by determining the amount of the Raf mRNA. Prior art standard methods comprise for instance the DNA chip hybridization, room temperature PCR, primer extension and RNA protection. Furthermore, the determination of the Raf activity based on the induction or repression of the transcription of the respective Raf gene(s), may also take place by the coupling of the Raf promoter to suitable reporter gene constructs. Examples for suitable reporter genes are the chloramphenicol transferase gene, the green fluorescent protein (GFP) and variants thereof, the luciferase gene and the Renilla gene. The detection of the increase of expression of Raf proteins may however also be made on the protein level, in this case the amount of protein being detected for instance by antibodies directed against Raf protein. The change of the activity of the Raf protein can however also be put down to increased or reduced phosphorylation or dephosphorylation of the protein. For instance, the B-Raf kinase is regulated by the phosphorylation of the 599Thr and 602Ser remainders (Zhang B. H. and Guan K. L. EMBO J., (2000) 19:5429). The change of the phosphorylation of B-Raf proteins may be detected, for example, by antibodies directed against phosphorylated threonine or serine.

Since Raf proteins are threonine/serine kinases, the activity of the Raf proteins can also be determined by their enzymatic activity. The protein MEK is for instance a substrate of B-Raf and the degree of the phosphorylation of MEK permits the determination of the B-Raf activity in the sample. In the same way, the phosphorylation of other substrates, as for instance MBP and peptides which are specifically phosphorylated by Raf (Salh, et al., Anticancer Res., (1999) 19:731-740; Bondzi, et al. Oncogene, (2000) 19:5030-5033), of the Raf proteins can be used for determining the respective activity. Since Raf is part of a signal cascade where a series of kinases are respectively phosphorylated and activated by a superordinated kinase, the activity of Raf can also be determined by evaluating the phosphorylation degree of each kinase subordinated to Raf. This so-called map kinase pathway also leads, among other features, to a specific activation of transcription factors and thus to a transcriptional activation of genes, such that the activity of Raf can indirectly be determined by measuring the activity of these target genes.

Administration of Compounds of the Invention

The compounds of the invention may be administered by any route appropriate to the condition to be treated. Suitable routes include oral, parenteral (including subcutaneous, intramuscular, intravenous, intraarterial, intradermal, intrathecal and epidural), transdermal, rectal, nasal, topical (including buccal and sublingual), vaginal, intraperitoneal, intrapulmonary and intranasal. For local immunosuppressive treatment, the compounds may be administered by intralesional administration, including perfusing or otherwise contacting the graft with the inhibitor before transplantation. It will be appreciated that the preferred route may vary with for example the condition of the recipient. Where the compound is administered orally, it may be formulated as a pill, capsule, tablet, etc. with a pharmaceutically acceptable carrier or excipient. Where the compound is administered parenterally, it may be formulated with a pharmaceutically acceptable parenteral vehicle and in a unit dosage injectable form, as detailed below.

Methods of Treatment with Compounds of the Invention

The invention includes methods of treating or preventing disease or condition by administering one or more compounds of this invention, or a stereoisomer, geometric isomer, tautomer, solvate, metabolite, or pharmaceutically acceptable salt or prodrug thereof. Disease and condition treatable according to the methods of this invention include, but are not limited to, cancer, stroke, diabetes, hepatomegaly, cardiovascular disease, Alzheimer's disease, cystic fibrosis, viral disease, autoimmune diseases, atherosclerosis, restenosis, psoriasis, allergic disorders, inflammation, neurological disorders, a hormone-related disease, conditions associated with organ transplantation, immunodeficiency disorders, destructive bone disorders, proliferative disorders, infectious diseases, conditions associated with cell death, thrombin-induced platelet aggregation, chronic myelogenous leukemia (CML), liver disease, pathologic immune conditions involving T cell activation, and CNS disorders in a patient. In one embodiment, a human patient is treated with a compound of this invention and a pharmaceutically acceptable carrier, adjuvant, or vehicle in an amount to detectably inhibit Raf kinase activity.

In another embodiment, a method of treating or preventing cancer in a mammal in need of such treatment, wherein the method comprises administering to said mammal a therapeutically effective amount of a compound of this invention or a stereoisomer, geometric isomer, tautomer, solvate, metabolite, or pharmaceutically acceptable salt or prodrug thereof. The cancer is selected from breast, ovary, cervix, prostate, testis, genitourinary tract, esophagus, larynx, glioblastoma, neuroblastoma, stomach, skin, keratoacanthoma, lung, epidermoid carcinoma, large cell carcinoma, non-small cell lung carcinoma (NSCLC), small cell carcinoma, lung adenocarcinoma, bone, colon, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, hairy cells, buccal cavity and pharynx (oral), lip, tongue, mouth, pharynx, small intestine, colon-rectum, large intestine, rectum, brain and central nervous system, Hodgkin's and leukemia.

In another embodiment, a method of treating or preventing cardiovascular disease selected from restenosis, cardiomegaly, atherosclerosis, myocardial infarction, or congestive heart failure in a mammal in need of such treatment, wherein the method comprises administering to a mammal a therapeutically effective amount of a pharmaceutical composition comprising a compound of this invention, or a stereoisomer, geometric isomer, tautomer, solvate, metabolite, or pharmaceutically acceptable salt or prodrug thereof.

In another embodiment, a method of treating or preventing neurodegenerative disease selected from Alzheimer's disease, Parkinson's disease, amyotrophic lateral sclerosis, Huntington's disease, cerebral ischemia or neurodegenerative disease caused by traumatic injury, glutamate neurotoxicity or hypoxia in a mammal in need of such treatment, wherein the method comprises administering to a mammal a therapeutically effective amount of a pharmaceutical composition comprising a compound of this invention, or a stereoisomer, geometric isomer, tautomer, solvate, metabolite, or pharmaceutically acceptable salt or prodrug thereof.

In another embodiment, a method of treating or preventing inflammatory diseases selected from rheumatoid arthritis, psoriasis, contact dermatitis, and delayed hypersensitivity reactions in a mammal in need of such treatment, wherein the method comprises administering to a mammal a therapeutically effective amount of a pharmaceutical composition comprising a compound of this invention, or a stereoisomer, geometric isomer, tautomer, solvate, metabolite, or pharmaceutically acceptable salt or prodrug thereof.

Pharmaceutical Formulations

Compounds of the present invention are useful for treating diseases, conditions and/or disorders, for example, but not limited to, those characterized by over expression of Raf kinases, e.g. B-Raf kinase. Therefore, another embodiment of the present invention is a pharmaceutical composition, i.e. formulation, comprising a therapeutically effective amount of a compound of the present invention and a pharmaceutically acceptable excipient, diluent or carrier. The pharmaceutical composition may be made by a process which comprises combining a compound of claim 1 with a pharmaceutically acceptable carrier. Compounds of the invention may be used in the manufacture of a medicament for the prophylactic or therapeutic treatment of cancer. Accordingly, another aspect of the invention provides methods of preventing or treating a hyperproliferative disorder, neurodegeneration, cardiac hypertrophy, pain, migraine or a neurotraumatic disease or event, by administering to a mammal in need of such treatment an effective amount of a compound of this invention, or a stereoisomer, geometric isomer, tautomer, solvate, metabolite, or pharmaceutically acceptable salt or prodrug thereof.

A typical formulation is prepared by mixing a compound of the present invention and a carrier, diluent or excipient. Suitable carriers, diluents and excipients are well known to those skilled in the art and include materials such as carbohydrates, waxes, water soluble and/or swellable polymers, hydrophilic or hydrophobic materials, gelatin, oils, solvents, water and the like. The particular carrier, diluent or excipient used will depend upon the means and purpose for which the compound of the present invention is being applied. Solvents are generally selected based on solvents recognized by persons skilled in the art as safe (GRAS) to be administered to a mammal. In general, safe solvents are non-toxic aqueous solvents such as water and other non-toxic solvents that are soluble or miscible in water. Suitable aqueous solvents include water, ethanol, propylene glycol, polyethylene glycols (e.g., PEG 400, PEG 300), etc. and mixtures thereof. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the drug (i.e., a compound of the present invention or pharmaceutical composition thereof) or aid in the manufacturing of the pharmaceutical product (i.e., medicament).

The formulations may be prepared using conventional dissolution and mixing procedures. For example, the bulk drug substance (i.e., compound of the present invention or stabilized form of the compound (e.g., complex with a cyclodextrin derivative or other known complexation agent) is dissolved in a suitable solvent in the presence of one or more of the excipients described above. The compound of the present invention is typically formulated into pharmaceutical dosage forms to provide an easily controllable dosage of the drug and to enable patient compliance with the prescribed regimen.

The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

Pharmaceutical formulations of the compounds of the present invention may be prepared for various routes and types of administration. For example, a compound of this invention, having the desired degree of purity may optionally be mixed with pharmaceutically acceptable diluents, carriers, excipients or stabilizers (Remington's Pharmaceutical Sciences (1980) 16th edition, Osol, A. Ed.), in the form of a lyophilized formulation, milled powder, or an aqueous solution. Formulation may be conducted by mixing at ambient temperature at the appropriate pH, and at the desired degree of purity, with physiologically acceptable carriers, i.e., carriers that are non-toxic to recipients at the dosages and concentrations employed. The pH of the formulation depends mainly on the particular use and the concentration of compound, but may range from about 3 to about 8. Formulation in an acetate buffer at pH 5 is a suitable embodiment.

The inhibitory compound for use herein is preferably sterile. In particular, formulations to be used for in vivo administration must be sterile. Such sterilization is readily accomplished by filtration through sterile filtration membranes.

The compound ordinarily can be stored as a solid composition, a lyophilized formulation or as an aqueous solution.

The pharmaceutical compositions of the invention will be formulated, dosed and administered in a fashion, i.e., amounts, concentrations, schedules, course, vehicles and route of administration, consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “therapeutically effective amount” of the compound to be administered will be governed by such considerations, and is the minimum amount necessary to prevent, ameliorate, or treat the coagulation factor mediated disorder. Such amount is preferably below the amount that is toxic to the host or renders the host significantly more susceptible to bleeding.

As a general proposition, the initial pharmaceutically effective amount of the inhibitor administered parenterally per dose will be in the range of about 0.01-100 mg/kg, namely about 0.1 to 20 mg/kg of patient body weight per day, with the typical initial range of compound used being 0.3 to 15 mg/kg/day.

Acceptable diluents, carriers, excipients and stabilizers are nontoxic to recipients at the dosages and concentrations employed, and include buffers such as phosphate, citrate and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). The active pharmaceutical ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980). A “liposome” is a small vesicle composed of various types of lipids, phospholipids and/or surfactant which is useful for delivery of a drug (such as the Raf inhibitors disclosed herein and, optionally, a chemotherapeutic agent) to a mammal. The components of the liposome are commonly arranged in a bilayer formation, similar to the lipid arrangement of biological membranes.

Sustained-release preparations of compounds of this invention may be prepared. Suitable examples of sustained-release preparations include semipermeable matrices of solid hydrophobic polymers containing a compound of this invention, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and gamma-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate) and poly-D-(−)-3-hydroxybutyric acid.

The formulations include those suitable for the administration routes detailed herein. The formulations may conveniently be presented in unit dosage form and may be prepared by any of the methods well known in the art of pharmacy. Techniques and formulations generally are found in Remington's Pharmaceutical Sciences (Mack Publishing Co., Easton, Pa.). Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Formulations of a compound of this invention suitable for oral administration may be prepared as discrete units such as pills, capsules, cachets or tablets each containing a predetermined amount of a compound of this invention.

Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered active ingredient moistened with an inert liquid diluent. The tablets may optionally be coated or scored and optionally are formulated so as to provide slow or controlled release of the active ingredient therefrom.

Tablets, troches, lozenges, aqueous or oil suspensions, dispersible powders or granules, emulsions, hard or soft capsules, e.g., gelatin capsules, syrups or elixirs may be prepared for oral use. Formulations of compounds of this invention intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

For treatment of the eye or other external tissues, e.g., mouth and skin, the formulations are preferably applied as a topical ointment or cream containing the active ingredient(s) in an amount of, for example, 0.075 to 20% w/w. When formulated in an ointment, the active ingredients may be employed with either a paraffinic or a water-miscible ointment base. Alternatively, the active ingredients may be formulated in a cream with an oil-in-water cream base.

If desired, the aqueous phase of the cream base may include a polyhydric alcohol, i.e., an alcohol having two or more hydroxyl groups such as propylene glycol, butane 1,3-diol, mannitol, sorbitol, glycerol and polyethylene glycol (including PEG 400) and mixtures thereof. The topical formulations may desirably include a compound which enhances absorption or penetration of the active ingredient through the skin or other affected areas. Examples of such dermal penetration enhancers include dimethyl sulfoxide and related analogs.

The oily phase of the emulsions of this invention may be constituted from known ingredients in a known manner. While the phase may comprise merely an emulsifier, it desirably comprises a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations. Emulsifiers and emulsion stabilizers suitable for use in the formulation of the invention include Tween® 60, Span® 80, cetostearyl alcohol, benzyl alcohol, myristyl alcohol, glyceryl mono-stearate and sodium lauryl sulfate.

Aqueous suspensions of the invention contain the active materials in admixture with excipients suitable for the manufacture of aqueous suspensions. Such excipients include a suspending agent, such as sodium carboxymethylcellulose, croscarmellose, povidone, methylcellulose, hydroxypropyl methylcelluose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxybenzoate, one or more coloring agents, one or more flavoring agents and one or more sweetening agents, such as sucrose or saccharin.

The pharmaceutical compositions of compounds of this invention may be in the form of a sterile injectable preparation, such as a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such as a solution in 1,3-butanediol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

The amount of active ingredient that may be combined with the carrier material to produce a single dosage form will vary depending upon the host treated and the particular mode of administration. For example, a time-release formulation intended for oral administration to humans may contain approximately 1 to 1000 mg of active material compounded with an appropriate and convenient amount of carrier material which may vary from about 5 to about 95% of the total compositions (weight:weight). The pharmaceutical composition can be prepared to provide easily measurable amounts for administration. For example, an aqueous solution intended for intravenous infusion may contain from about 3 to 500 μg of the active ingredient per milliliter of solution in order that infusion of a suitable volume at a rate of about 30 mL/hr can occur.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

Formulations suitable for topical administration to the eye also include eye drops wherein the active ingredient is dissolved or suspended in a suitable carrier, especially an aqueous solvent for the active ingredient. The active ingredient is preferably present in such formulations in a concentration of 0.5 to 20%, advantageously 0.5 to 10% particularly about 1.5% w/w.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate.

Formulations suitable for intrapulmonary or nasal administration have a particle size for example in the range of 0.1 to 500 microns (including particle sizes in a range between 0.1 and 500 microns in increments microns such as 0.5, 1, 30 microns, 35 microns, etc.), which is administered by rapid inhalation through the nasal passage or by inhalation through the mouth so as to reach the alveolar sacs. Suitable formulations include aqueous or oily solutions of the active ingredient. Formulations suitable for aerosol or dry powder administration may be prepared according to conventional methods and may be delivered with other therapeutic agents such as compounds heretofore used in the treatment or prophylaxis disorders as described below.

Formulations suitable for vaginal administration may be presented as pessaries, tampons, creams, gels, pastes, foams or spray formulations containing in addition to the active ingredient such carriers as are known in the art to be appropriate.

The formulations may be packaged in unit-dose or multi-dose containers, for example sealed ampoules and vials, and may be stored in a freeze-dried (lyophilized) condition requiring only the addition of the sterile liquid carrier, for example water, for injection immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

The invention further provides veterinary compositions comprising at least one active ingredient as above defined together with a veterinary carrier therefore. Veterinary carriers are materials useful for the purpose of administering the composition and may be solid, liquid or gaseous materials which are otherwise inert or acceptable in the veterinary art and are compatible with the active ingredient. These veterinary compositions may be administered parenterally, orally or by any other desired route.

Combination Therapy

The compounds of this invention and stereoisomers, geometric isomers, tautomers, solvates, metabolites, and pharmaceutically acceptable salts and prodrugs thereof may be employed alone or in combination with other therapeutic agents for the treatment of a hyperproliferative disorder (e.g., cancer). In certain embodiments, a compound of this invention is combined in a pharmaceutical combination formulation, or dosing regimen as combination therapy, with a second compound that has anti-hyperproliferative properties or that is useful for treating a hyperproliferative disorder (e.g., cancer). The second compound of the pharmaceutical combination formulation or dosing regimen preferably has complementary activities to the compound of this invention such that they do not adversely affect each other. Such molecules are suitably present in combination in amounts that are effective for the purpose intended. In one embodiment, a composition of this invention comprises a compound of this invention, or a stereoisomer, geometric isomer, tautomer, solvate, metabolite, or pharmaceutically acceptable salt or prodrug thereof, in combination with a chemotherapeutic agent such as described herein.

The combination therapy may be administered as a simultaneous or sequential regimen. When administered sequentially, the combination may be administered in two or more administrations. The combined administration includes coadministration, using separate formulations or a single pharmaceutical formulation, and consecutive administration in either order, wherein preferably there is a time period while both (or all) active agents simultaneously exert their biological activities.

Suitable dosages for any of the above coadministered agents are those presently used and may be lowered due to the combined action (synergy) of the newly identified agent and other chemotherapeutic agents or treatments.

The combination therapy may provide “synergy” and prove “synergistic”, i.e., the effect achieved when the active ingredients used together is greater than the sum of the effects that results from using the compounds separately. A synergistic effect may be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect may be attained when the compounds are administered or delivered sequentially, e.g., by different injections in separate syringes. In general, during alternation therapy, an effective dosage of each active ingredient is administered sequentially, i.e., serially, whereas in combination therapy, effective dosages of two or more active ingredients are administered together.

In a particular embodiment, in anti-cancer therapy, a compound of this invention, or a stereoisomer, geometric isomer, tautomer, solvate, metabolite, or pharmaceutically acceptable salt or prodrug thereof, may be combined with other chemotherapeutic, hormonal or antibody agents such as those described herein, as well as combined with surgical therapy and radiotherapy. Combination therapies according to the present invention thus comprise the administration of at least one compound of this invention, or a stereoisomer, geometric isomer, tautomer, solvate, metabolite, or pharmaceutically acceptable salt or prodrug thereof, and the use of at least one other cancer treatment method. In certain embodiments, combination therapies according to the present invention comprise the administration of at least one compound of this invention, or a stereoisomer, geometric isomer, tautomer, solvate, metabolite, or pharmaceutically acceptable salt or prodrug thereof, and at least one other pharmaceutically active chemotherapeutic agent. The compound(s) of this invention and the other pharmaceutically active chemotherapeutic agent(s) may be administered together in a unitary pharmaceutical composition or separately and, when administered separately this may occur simultaneously or sequentially in any order. Such sequential administration may be close in time or remote in time. The amounts of the compound(s) of this invention and the other pharmaceutically active chemotherapeutic agent(s) and the relative timings of administration will be selected in order to achieve the desired combined therapeutic effect.

Metabolites

Also falling within the scope of this invention are the in vivo metabolic products of compounds of this invention described herein. Such products may result for example from the oxidation, reduction, hydrolysis, amidation, deamidation, esterification, deesterification, enzymatic cleavage, and the like, of the administered compound. Accordingly, the invention includes metabolites of compounds of this invention, including compounds produced by a process comprising contacting a compound of this invention with a mammal for a period of time sufficient to yield a metabolic product thereof.

Metabolite products typically are identified by preparing a radiolabelled (e.g., ¹⁴C or ³H) isotope of a compound of the invention, administering it parenterally in a detectable dose (e.g., greater than about 0.5 mg/kg) to an animal such as rat, mouse, guinea pig, monkey, or to man, allowing sufficient time for metabolism to occur (typically about 30 seconds to 30 hours) and isolating its conversion products from the urine, blood or other biological samples. These products are easily isolated since they are labeled (others are isolated by the use of antibodies capable of binding epitopes surviving in the metabolite). The metabolite structures are determined in conventional fashion, e.g., by MS, LC/MS or NMR analysis. In general, analysis of metabolites is done in the same way as conventional drug metabolism studies well known to those skilled in the art. The metabolite products, so long as they are not otherwise found in vivo, are useful in diagnostic assays for therapeutic dosing of the compounds of the invention.

Articles of Manufacture

In another embodiment of the invention, an article of manufacture, or “kit”, containing materials useful for the treatment of the disorders described above is provided. In one embodiment, the kit comprises a container comprising a compound of this invention, or a stereoisomer, geometric isomer, tautomer, solvate, metabolite, or pharmaceutically acceptable salt or prodrug thereof. The kit may further comprise a label or package insert on or associated with the container. The term “package insert” is used to refer to instructions customarily included in commercial packages of therapeutic products, that contain information about the indications, usage, dosage, administration, contraindications and/or warnings concerning the use of such therapeutic products. Suitable containers include, for example, bottles, vials, syringes, blister pack, etc. The container may be formed from a variety of materials such as glass or plastic. The container may hold a compound of this invention or a formulation thereof which is effective for treating the condition and may have a sterile access port (for example, the container may be an intravenous solution bag or a vial having a stopper pierceable by a hypodermic injection needle). At least one active agent in the composition is a compound of this invention. The label or package insert indicates that the composition is used for treating the condition of choice, such as cancer. In addition, the label or package insert may indicate that the patient to be treated is one having a disorder such as a hyperproliferative disorder, neurodegeneration, cardiac hypertrophy, pain, migraine or a neurotraumatic disease or event. In one embodiment, the label or package inserts indicates that the composition comprising a compound of this invention can be used to treat a disorder resulting from abnormal cell growth. The label or package insert may also indicate that the composition can be used to treat other disorders. Alternatively, or additionally, the article of manufacture may further comprise a second container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kit may further comprise directions for the administration of the compound of this invention and, if present, the second pharmaceutical formulation. For example, if the kit comprises a first composition comprising a compound of this invention and a second pharmaceutical formulation, the kit may further comprise directions for the simultaneous, sequential or separate administration of the first and second pharmaceutical compositions to a patient in need thereof.

In another embodiment, the kits are suitable for the delivery of solid oral forms of a compound of this invention, such as tablets or capsules. Such a kit preferably includes a number of unit dosages. Such kits can include a card having the dosages oriented in the order of their intended use. An example of such a kit is a “blister pack”. Blister packs are well known in the packaging industry and are widely used for packaging pharmaceutical unit dosage forms. If desired, a memory aid can be provided, for example in the form of numbers, letters, or other markings or with a calendar insert, designating the days in the treatment schedule in which the dosages can be administered.

According to one embodiment, an article of manufacture may comprise (a) a first container with a compound of this invention contained therein; and optionally (b) a second container with a second pharmaceutical formulation contained therein, wherein the second pharmaceutical formulation comprises a second compound with anti-hyperproliferative activity. Alternatively, or additionally, the article of manufacture may further comprise a third container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

In certain other embodiments wherein the kit comprises a composition of this invention and a second therapeutic agent, the kit may comprise a container for containing the separate compositions such as a divided bottle or a divided foil packet, however, the separate compositions may also be contained within a single, undivided container. Typically, the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

### EXAMPLES

In order to illustrate the invention, the following examples are included. However, it is to be understood that these examples do not limit the invention and are only meant to suggest a method of practicing the invention. Persons skilled in the art will recognize that the chemical reactions described may be readily adapted to prepare a number of other Raf inhibitors of the invention, and alternative methods for preparing the compounds of this invention are deemed to be within the scope of this invention. For example, the synthesis of non-exemplified compounds according to the invention may be successfully performed by modifications apparent to those skilled in the art, e.g., by appropriately protecting interfering groups, by utilizing other suitable reagents known in the art other than those described, and/or by making routine modifications of reaction conditions. Alternatively, other reactions disclosed herein or known in the art will be recognized as having applicability for preparing other compounds of the invention.

In the examples described below, unless otherwise indicated all temperatures are set forth in degrees Celsius. Reagents were purchased from commercial suppliers such as Aldrich Chemical Company, Lancaster, TCI or Maybridge, and were used without further purification unless otherwise indicated.

The reactions set forth below were done generally under a positive pressure of nitrogen or argon or with a drying tube (unless otherwise stated) in anhydrous solvents, and the reaction flasks were typically fitted with rubber septa for the introduction of substrates and reagents via syringe. Glassware was oven dried and/or heat dried.

Column chromatography was done on a Biotage system (Manufacturer: Dyax Corporation) having a silica gel column or on a silica SepPak cartridge (Waters). ¹H NMR spectra were recorded on a Varian instrument operating at 400 MHz. ¹H-NMR spectra were obtained as CDCl₃, d₆-DMSO, CH₃OD or d₆-acetone solutions (reported in ppm), using chloroform as the reference standard (7.25 ppm). When peak multiplicities are reported, the following abbreviations are used: s (singlet), d (doublet), t (triplet), m (multiplet), br (broadened), dd (doublet of doublets), dt (doublet of triplets). Coupling constants, when given, are reported in Hertz (Hz).

In certain instances in the Examples, the oxime geometry shown is implied; however, the oxime moiety of the compounds of this invention can exist as either the E or Z isomer, or as a mixture of both.

### Example 1

### Preparation of Ethyl 3-aminothieno[2,3-c]pyridine-2-carboxylate

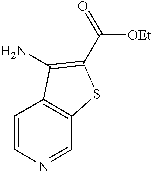

Step A: Preparation of (Z)-3-bromoisonicotinaldehyde oxime: 3-bromoisonicotinaldehyde (5073 mg, 27273 μmol) and sodium acetate (2797 mg, 34092 μmol) were suspended in 200 mL water and heated to 100° C. utilizing a condenser. H₂NOH—HCl (5686 mg, 40910 μmol) was added to the reaction mixture, resulting in immediate heavy precipitation. The reaction mixture was removed from heat and stirred 5 minutes while cooling to room temperature, then cooled further to 0° C. on ice and filtered, rinsing with ice-cold water. The desired product was isolated as white fibrous crystalline material (5.096 g, 93%). MS(+) m/z=202.3. Product was used directly in the next step without further purification.

Step B: Preparation of 3-bromoisonicotinonitrile: (Z)-3-bromoisonicotinaldehyde oxime (4975 mg, 24.75 mmol) was suspended in THF with triethylamine (13.80 mL, 98.99 mmol) and cooled to 0° C. in an ice bath. POCl₃ (2.379 mL, 25.99 mmol) was added via syringe and the reaction mixture was stirred for 2.5 hours. The reaction mixture was transferred to separatory funnel, diluted with EtOAc, washed with NaHCO₃ and extracted 3× with EtOAc. The combined organics were dried over Na₂SO₄ and concentrated to a pink solid. The solid was triturated with pentane and the crystalline material was isolated by filtration. A 2nd lot was prepared from the mother liquor. Yield=3.80 g (84%).

Step C: Preparation of ethyl 3-aminothieno[2,3-c]pyridine-2-carboxylate: 3-Bromoisonicotinonitrile (2000 mg, 10.93 mmol) was combined with ethyl 2-mercaptoacetate (1.205 mL, 10.93 mmol) in 50 mL DMF. Sodium ethanolate (4.080 mL, 10.93 mmol) was added, and the reaction mixture was stirred for 2 hours. The reaction mixture was transferred to separatory funnel, diluted with H₂O, brine, NaHCO₃ and extracted with EtOAc. The combined organic layers were combined, dried over Na₂SO₄, and concentrated to a yellow solid. LCMS confirmed desired product. The crude product was pre-adsorbed onto silica column and eluted with 1-5% MeOH/CH₂Cl₂ to provide 1.631 g (67%) of the desired product. MS(+) m/z=223.1.

### Example 2

### Preparation of (E)-ethyl 3-(1-(hydroxyimino)-2,3-dihydro-1H-inden-5-ylamino)thieno[2,3-c]pyridine-2-carboxylate

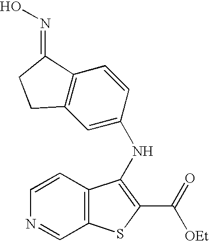

Step A: Preparation of 5-bromo-2,3-dihydroinden-1-one O-tert-butyldimethylsilyl oxime: 5-Bromo-2,3-dihydroinden-1-one (1.86 g, 8.8 mmol, 1.0 equiv), O-(tert-butyldimethylsily)hydroxylamine (1.84 g, 1.4 equiv), 4 Å molecular sieves (1.5 g), and TsOH.H₂O (0.18 g, 0.1 equiv) were refluxed in CHCl₃ (25 mL) under N₂ for 3 days, then cooled to room temperature and filtered through GF/F paper, rinsing with EtOAc. The solution was concentrated and purified by silica gel chromatography (5% ethyl acetate/hexanes) to afford the desired compound (2.98 g, 99%) as a colorless oil which solidified under high vacuum.

Step B: Preparation of ethyl 3-(1-(tert-butyldimethylsilyloxyimino)-2,3-dihydro-1H-inden-5-ylamino)thieno[2,3-c]pyridine-2-carboxylate: Ethyl 3-aminothieno[2,3-c]pyridine-2-carboxylate (prepared according to Example 1; 500 mg, 2.250 mmol), (E)-5-bromo-2,3-dihydroinden-1-one O-tert-butyldimethylsilyl oxime (765.6 mg, 2.250 mmol) and Cs₂CO₃ (1173 mg, 3.599 mmol) were combined in toluene (10 mL) and degassed 10 minutes with argon, and then X-Phos (32.17 mg, 0.06749 mmol) and Pd₂(dba)₃ (103.0 mg, 0.1125 mmol) were added to the reaction mixture. The reaction mixture was heated at reflux (110° C.) overnight under argon, then purified by column chromatography, eluting with 15-25% ethyl acetate/Hexanes. Both the E- and Z-oxime isomers were isolated and characterized by ¹H NMR. Yield=807 mg (75%). MS(+) m/z=482.3.

Step C: Preparation of (E)-ethyl 3-(1-(hydroxyimino)-2,3-dihydro-1H-inden-5-ylamino)thieno[2,3-c]pyridine-2-carboxylate: (E)-ethyl 3-(1-(tert-butyldimethylsilyloxyimino)-2,3-dihydro-1H-inden-5-ylamino)thieno[2,3-c]pyridine-2-carboxylate (15.0 mg, 0.0311 mmol) was dissolved in 2 mL CH₂Cl₂ and cooled to 0° C. TBAF (0.0311 mL, 0.0311 mmol) was added and the reaction mixture was stirred for 1 hour while warming to room temperature. The crude reaction mixture was purified by preparative TLC. The isolated top band provided 10.2 mg (89%) of the E isomer as determined by ¹H NMR and previous assignments. MS(+) m/z=368.2.

### Example 3

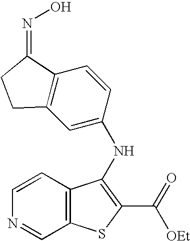

### Preparation of (Z)-ethyl 3-(1-(hydroxyimino)-2,3-dihydro-1H-inden-5-ylamino)thieno[2,3-c]pyridine-2-carboxylate

(Z)-Ethyl 3-(1-(tert-butyldimethylsilyloxyimino)-2,3-dihydro-1H-inden-5-ylamino)thieno[2,3-c]pyridine-2-carboxylate (prepared according to Example 2; 15.0 mg, 0.0311 mmol) was dissolved in 2 mL CH₂Cl₂ and cooled to 0° C. TBAF (0.0311 mL, 0.0311 mmol) was added and the reaction mixture was stirred for 1 hour while warming to room temperature. The crude reaction mixture was purified by preparative TLC to provide 5.2 mg (45%) of the desired product. MS(+) m/z=368.2.

### Example 4

### General Procedure for X-Phos/Pd₂(dba)₃-Catalyzed Coupling of Amines with 5-bromo-2,3-dihydroinden-1-one O-tert-butyldimethylsilyl oxime

The amine (1.0 equiv) and 5-bromo-2,3-dihydroinden-1-one O-tert-butyldimethylsilyl oxime (1.2 equiv) are taken up in toluene and degassed under argon for 15 minutes. 2-(Dicyclohexylphosphino)-2′,4′,6′-tri-isopropyl-1,1′-biphenyl (X-Phos) (0.1 equiv), NaO-t-Bu (1.6 equiv) and Pd₂(dba)₃ (0.05 equiv) are added, the reaction mixture is degassed an additional 10 minutes, and then heated to 110° C. until MS indicates formation of product. The reaction is cooled, filtered through GF/F paper, rinsed with CH₂Cl₂, and purified by silica gel chromatography to provide the product.

### Example 5

### General Procedure for TBAF Deprotection of O-tert-butyldimethylsilyl Oximes

The O-tert-butyldimethylsilyl oxime is taken up in THF (5 mL) and cooled in an ice bath. The solution is treated with a solution of tetrabutylammonium fluoride (TBAF) (1.0 M in THF, 1.3 equiv) and the reaction is stirred for 10 minutes at 0° C. The reaction is quenched with aqueous NH₄Cl, extracted with EtOAc, dried over MgSO₄, and purified by silica gel chromatography to afford the (E) and (Z) products.

### Example 6

### General Procedure for Grignard Addition to N,O-dimethylamides

The N,O-dimethylamide is taken up in THF and cooled to 0° C. An excess of the Grignard reagent is added dropwise (in portions) until no starting material remains as determined by MS. The reaction is quenched at 0° C. with aqueous NH₄Cl, extracted with EtOAc, and dried over MgSO₄. Purification by silica gel chromatography is used to separate the ketone from the N-methyl amide.

### Example 7

### General Procedure for the Removal of BOC Groups

The N—BOC amine is taken up in CH₂Cl₂ in an ice bath, and an equal volume of TFA is added at once. The reaction is warmed to room temperature for 2 hours, and the volatiles are removed by rotary evaporation. The reaction mixture is diluted with CH₂Cl₂ and NEt₃, and the residue is purified by silica gel chromatography to afford the desired amine.

### Example 8

### Preparation of (5-(2-phenylthieno[2,3-c]pyridin-3-ylamino)-2,3-dihydroinden-1-one oxime)

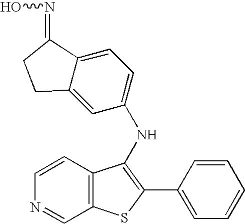

Step A: Preparation of 2-phenylthieno[2,3-c]pyridin-3-amine: A mixture of benzyl mercaptan (0.2719 g, 2.19 mmol, 1.0 equiv.) and DMF (3 mL) was stirred at room temperature, and NaOMe (250 mg, 2.1 equiv) was added. The solution was stirred for 5 minutes, and then 3-bromoisonicotinonitrile (400 mg, 1.0 equiv.) was added directly to the solution. The reaction mixture was stirred overnight at room temperature, then the volatiles were removed via rotary evaporation. Water was added, and the reaction mixture was extracted twice with ether. The combined organic layers were dried (Na₂SO₄) and purified by silica gel chromatography (eluting first with 100% Et₂O to remove non-polar impurities, then switching to a CHCl₃/MeOH gradient) to afford 221 mg (45%) of the desired product.

Step B: Preparation of 5-(2-phenylthieno[2,3-c]pyridin-3-ylamino)-2,3-dihydroinden-1-one O-tert-butyldimethylsilyl oxime: The general X-Phos coupling procedure according to Example 4 was followed to provide 5-(2-phenylthieno[2,3-c]pyridin-3-ylamino)-2,3-dihydroinden-1-one O-tert-butyldimethylsilyl oxime as a yellow solid. MS (APCI-pos) M+1=486.3.

Step C: Preparation of (5-(2-Phenylthieno[2,3-c]pyridin-3-ylamino)-2,3-dihydroinden-1-one oxime): (5-(2-Phenylthieno[2,3-c]pyridin-3-ylamino)-2,3-dihydroinden-1-one oxime) was prepared from 5-(2-phenylthieno[2,3-c]pyridin-3-ylamino)-2,3-dihydroinden-1-one O-tert-butyldimethylsilyl oxime following the general procedure of Example 5 for the TBAF deprotection of O-tert-butyldimethylsilyl oxime. MS (APCI-pos) M+1=372.3.

### Example 9

### Preparation of 3-(1-(hydroxyimino)-2,3-dihydro-1H-inden-5-ylamino)-N-methylthieno[2,3-c]pyridine-2-carboxamide

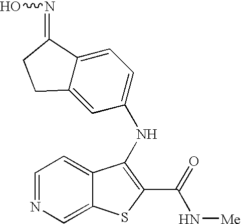

Step A: Preparation of ethyl 3-(di-tert-butoxycarbonyl)thieno[2,3-c]pyridine-2-carboxylate: Prepared according to Example 18, Steps A and B.

Step B: Preparation of tert-butyl 2-(methoxy(methyl)carbamoyl)thieno[2,3-c]pyridin-3-ylcarbamate: 3-(tert-Butoxycarbonyl)thieno[2,3-c]pyridine-2-carboxylic acid (506 mg, 1.0 equiv.), N,O-dimethylhydroxylamine hydrochloride (1.1 equiv.), HOBT-H₂O (0.05 equiv), DIEA (3.5 equiv), and EDCI-HCl (2.0 equiv) were taken up in CH₂Cl₂ (8 mL) and stirred overnight at room temperature. The reaction was diluted with water, extracted twice with CH₂Cl₂, dried (Na₂SO₄), and purified by silica gel chromatography (20% ethyl acetate/hexanes) to afford the desired product as a yellow solid (382 mg, 66%). MS (APCI-pos) M+1=338.0.

Step B: Preparation of tert-butyl 2-(methylcarbamoyl)thieno[2,3-c]pyridin-3-ylcarbamate: The general Grignard addition procedure of Example 6 was followed using 3-amino-N-methoxy-N-methylthieno[2,3-c]pyridine-2-carboxamide to provide the desired product as a white solid. MS (APCI-pos) M+1=308.0.

Step C: Preparation of 3-amino-N-methylthieno[2,3-c]pyridine-2-carboxamide: tert-Butyl 2-(methylcarbamoyl)thieno[2,3-c]pyridin-3-ylcarbamate was cleanly deprotected according to the general BOC deprotection procedure of Example 7 to provide 3-amino-N-methylthieno[2,3-c]pyridine-2-carboxamide. MS (APCI-pos) M+1=208.2.

Step D: Preparation of 3-(1-(tert-butyldimethylsilyloxyimino)-2,3-dihydro-1H-inden-5-ylamino)-N-methylthieno[2,3-c]pyridine-2-carboxamide: The general X-Phos coupling procedure of Example 4 was followed utilizing 5-bromo-2,3-dihydroinden-1-one O-tert-butyldimethylsilyl oxime and 3-amino-N-methylthieno[2,3-c]pyridine-2-carboxamide to provide 3-(1-(tert-butyldimethylsilyloxyimino)-2,3-dihydro-1H-inden-5-ylamino)-N-methylthieno[2,3-c]pyridine-2-carboxamide. MS (APCI-pos) M+1=467.3.

Step E: Preparation of 3-(1-(hydroxyimino)-2,3-dihydro-1H-inden-5-ylamino)-N-methylthieno[2,3-c]pyridine-2-carboxamide: 3-(1-(tert-Butyldimethylsilyloxyimino)-2,3-dihydro-1H-inden-5-ylamino)-N-methylthieno[2,3-c]pyridine-2-carboxamide was deprotected according to the general TBAF deprotection procedure of Example 5 to provide 3-(1-(hydroxyimino)-2,3-dihydro-1H-inden-5-ylamino)-N-methylthieno[2,3-c]pyridine-2-carboxamide. MS (APCI-pos) M+1=353.2.

### Example 10

### Preparation of the E- and Z-oximes of (3-(1-(hydroxyimino)-2,3-dihydro-1H-inden-5-ylamino)thieno[2,3-c]pyridin-2-yl)(4-methoxyphenyl)methanone

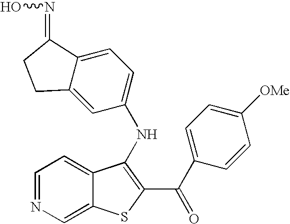

Step A: Preparation of tert-butyl 2-(4-methoxybenzoyl)thieno[2,3-c]pyridin-3-ylcarbamate: The general the Grignard addition procedure of Example 6 was followed utilizing (4-methoxyphenyl)magnesium bromide and tert-butyl 2-(methoxy(methyl)carbamoyl)thieno[2,3-c]pyridin-3-ylcarbamate (prepared according to Example 4) to provide tert-butyl 2-(4-methoxybenzoyl)thieno[2,3-c]pyridin-3-ylcarbamate in 31% yield. MS (APCI-pos) M+1=385.0.

Step B: Preparation of (3-aminothieno[2,3-c]pyridin-2-yl)(4-methoxyphenyl)methanone: tert-Butyl 2-(4-methoxybenzoyl)thieno[2,3-c]pyridin-3-ylcarbamate was deprotected according to the general procedure for BOC-deprotection of Example 7 to provide (3-aminothieno[2,3-c]pyridin-2-yl)(4-methoxyphenyl)methanone in 76% yield. MS (APCI-pos) M+1=285.3.

Step C: Preparation of (3-(1-(tert-butyldimethylsilyloxyimino)-2,3-dihydro-1H-inden-5-ylamino)thieno[2,3-c]pyridin-2-yl)(4-methoxyphenyl)methanone: 5-Bromo-2,3-dihydroinden-1-one O-tert-butyldimethylsilyl oxime and (3-aminothieno[2,3-c]pyridin-2-yl)(4-methoxyphenyl)methanone were coupled according to the general X-Phos coupling procedure of Example 4, heating at 110° C. for 20 hours, to provide the desired product in 70% yield. MS (APCI-pos) M+1=544.3.

Step D: Preparation of (3-(1-(hydroxyimino)-2,3-dihydro-1H-inden-5-ylamino)thieno[2,3-c]pyridin-2-yl)(4-methoxyphenyl methanone: (3-(1-(tert-butyldimethylsilyloxyimino)-2,3-dihydro-1H-inden-5-ylamino)thieno[2,3-c]pyridin-2-yl)(4-methoxyphenyl)methanone was deprotected according to the general procedure TBAF-promoted deprotection procedure of Example 5 to provide the desired product in 70% yield. The E- and Z-oxime isomers were easily separable by silica gel chromatography. MS for each isolated oxime isomer shows (APCI-pos) M+1=430.2.

### Example 11

### Preparation of (E)-5-(2-(hydroxy(4-methoxyphenyl)methyl)thieno[2,3-c]pyridin-3-ylamino)-2,3-dihydroinden-1-one oxime

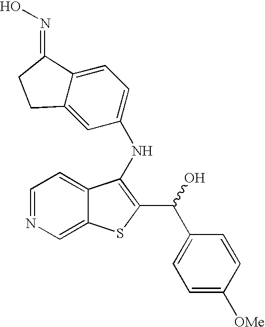

(E)-(3-(1-(hydroxyimino)-2,3-dihydro-1H-inden-5-ylamino)thieno[2,3-c]pyridin-2-yl)(4-methoxyphenyl)methanone (14 mg, 32.6 mmol, 1.0 equiv; prepared according to Example 10) was slurried in EtOH (3 mL) at 0° C. and NaBH₄ was added. The reaction was warmed to room temperature for 6 hours, and then quenched with saturated NH₄Cl, extracted with EtOAc, and dried over MgSO₄ to provide the desired product as a yellow solid (7 mg, 50% yield) after silica gel chromatography.

### Example 12

### Preparation of (E)-3-(1-(hydroxyimino)-2,3-dihydro-1H-inden-5-ylamino)thieno[2,3-c]pyridine-2-carboxylic acid

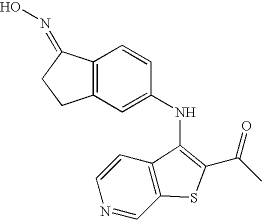

(E)-Ethyl 3-(1-(tert-butyldimethylsilyloxyimino)-2,3-dihydro-1H-inden-5-ylamino)thieno[2,3-c]pyridine-2-carboxylate (57 mg, 120 mmol, 1.0 equiv.; prepared according to Example 2, Steps A and B) and LiOH.H₂O (24 mg, 4.8 equiv.) were stirred overnight at room temperature in EtOH (2 mL). The reaction was quenched by the addition of aqueous NH₄Cl, and the resulting solids were collected by vacuum filtration, rinsing with water, and dried under high vacuum, to provide 34 mg (85%) of the desired compound as a yellow solid. MS (APCI-pos) M+1=340.2.

### Example 13

### Preparation of (E)-1-(3-(1-(hydroxyimino)-2,3-dihydro-1H-inden-5-ylamino)thieno[2,3-c]pyridin-2-yl)butan-1-one

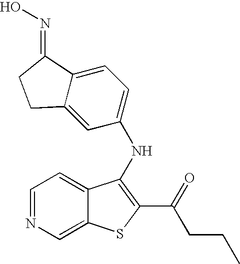

Step A: Preparation of tert-butyl 2-butyrylthieno[2,3-c]pyridin-3-ylcarbamate: The general the Grignard addition procedure of Example 6 was followed, utilizing n-propylmagnesium bromide and tert-butyl 2-(methoxy(methyl)carbamoyl)thieno[2,3-c]pyridin-3-ylcarbamate, to provide the desired product in 31% yield. MS (APCI-pos) M+1=320.9.

Step B: Preparation of 1-(3-aminothieno[2,3-c]pyridin-2-yl)butan-1-one: tert-butyl 2-butyrylthieno[2,3-c]pyridin-3-ylcarbamate was deprotected according to the general BOC-deprotection procedure of Example 7 to provide the desired product in quantitative yield. MS (APCI-pos) M+1=221.3.

Step C: Preparation of 1-(3-(1-(tert-butyldimethylsilyloxyimino)-2,3-dihydro-1H-inden-5-ylamino)thieno[2,3-c]pyridin-2-yl)butan-1-one: The general X-Phos coupling procedure of Example 4 was followed, using 5-bromo-2,3-dihydroinden-1-one O-tert-butyldimethylsilyl oxime and 1-(3-aminothieno[2,3-c]pyridin-2-yl)butan-1-one and heating at 110° C. for 17 hours, to provide the desired product in 54% yield. The E- and Z-oxime isomers were easily separated at this stage. MS (APCI-pos) M+1=480.3.

Step D: Preparation of (E)-1-(3-(1-(hydroxyimino)-2,3-dihydro-1H-inden-5-ylamino)thieno[2,3-c]pyridin-2-yl)butan-1-one: (E)-1-(3-(1-(tert-butyldimethylsilyloxyimino)-2,3-dihydro-1H-inden-5-ylamino)thieno[2,3-c]pyridin-2-yl)butan-1-one was deprotected according to the general TBAF-promoted deprotection procedure of Example 5 to provide the desired product in 79% yield. MS (APCI-pos) M+1=366.3.

### Example 14

### Preparation of (E)-isopropyl 3-(1-(hydroxyimino)-2,3-dihydro-1H-inden-5-ylamino)thieno[2,3-c]pyridine-2-carboxylate

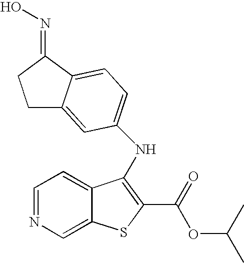

Step A: Preparation of isopropyl 3-aminothieno[2,3-c]pyridine-2-carboxylate: To a solution of ethyl 3-aminothieno[2,3-c]pyridine-2-carboxylate in i-PrOH is added 1.0 equiv. of Ti(Oi-Pr)₄ and the reaction mixture was stirred at 70° C. for 7 days. The i-PrOH was removed, and the residue was taken up in EtOAc (50 mL) and water (50 mL). The aqueous layer was extracted twice with EtOAc and the combined organics were dried, filtered and concentrated to afford the desired product as a yellow solid. MS (APCI-pos) M+1=237.0.

Step B: Preparation of isopropyl 3-(1-(tert-butyldimethylsilyloxyimino)-2,3-dihydro-1H-inden-5-ylamino)thieno[2,3-c]pyridine-2-carboxylate: The general X-Phos coupling procedure of Example 4 was followed, using 5-bromo-2,3-dihydroinden-1-one O-tert-butyldimethylsilyl oxime and isopropyl 3-aminothieno[2,3-c]pyridine-2-carboxylate and substituting Cs₂CO₃ for NaOtBu, to provide the desired product in 28% yield. The E- and Z-oxime isomers were easily separated at this stage. MS shows (APCI-pos) M+1=496.1.

Step C: Preparation of (E)-isopropyl 3-(1-(hydroxyimino)-2,3-dihydro-1H-inden-5-ylamino)thieno[2,3-c]pyridine-2-carboxylate: Isopropyl 3-(1-(tert-butyldimethylsilyloxyimino)-2,3-dihydro-1H-inden-5-ylamino)thieno[2,3-c]pyridine-2-carboxylate was deprotected according to the general procedure for TBAF-promoted deprotection of Example 5 to provide the desired product in 83% yield. MS (APCI-pos) M+1=382.1.

### Example 15

### Preparation of 5-(2-(pyridin-3-yl)thieno[2,3-c]pyridin-3-ylamino)-2,3-dihydroinden-1-one oxime

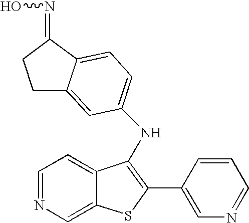

Step A: Preparation of tert-butyl 2-(pyridin-3-yl)thieno[2,3-c]pyridin-3-ylcarbamate: A solution of tert-butyl 2-iodothieno[2,3-c]pyridin-3-ylcarbamate (1.0 equiv.; prepared according to Example 18), pyridin-3-ylboronic acid (1.5 equiv), K₂CO₃ (3.0 equiv) in MeCN:water (4:1) was degassed for 15 minutes, and then Pd(PPh₃)₄ (0.1 equiv) was added. The reaction mixture was heated to 80° C. overnight. The solution was cooled to room temperature, diluted with water and extracted with EtOAc. After drying the organic layer over MgSO₄ and purification by silica gel chromatography, tert-butyl 2-(pyridin-3-yl)thieno[2,3-c]pyridin-3-ylcarbamate was obtained in 83% yield. MS (APCI-pos) M+1=328.0.

Step B: Preparation of 2-(pyridin-3-yl)thieno[2,3-c]pyridin-3-amine: tert-Butyl 2-(pyridin-3-yl)thieno[2,3-c]pyridin-3-ylcarbamate was deprotected according to the general BOC deprotection procedure of Example 7 to provide 2-(pyridin-3-yl)thieno[2,3-c]pyridin-3-amine in 62% yield. MS (APCI-pos) M+1=228.4.

Step C: Preparation of 5-(2-(pyridin-3-yl)thieno[2,3-c]pyridin-3-ylamino)-2,3-dihydroinden-1-one O-tert-butyldimethylsilyl oxime: The general X-Phos coupling procedure of Example 4 was followed, using 5-bromo-2,3-dihydroinden-1-one O-tert-butyldimethylsilyl oxime and 2-(pyridin-3-yl)thieno[2,3-c]pyridin-3-amine, to provide the desired product in 50% yield. MS shows (APCI-pos) M+1=487.3.

Step D: Preparation of 5-(2-(pyridin-3-yl)thieno[2,3-c]pyridin-3-ylamino)-2,3-dihydroinden-1-one oxime: 5-(2-(Pyridin-3-yl)thieno[2,3-c]pyridin-3-ylamino)-2,3-dihydroinden-1-one O-tert-butyldimethylsilyl oxime was deprotected according to the general TBAF-promoted deprotection procedure of Example 5 to provide 5-(2-(pyridin-3-yl)thieno[2,3-c]pyridin-3-ylamino)-2,3-dihydroinden-1-one oxime in 33% yield. MS (APCI-pos) M+1=373.2.

### Example 16

### Preparation of 3(1-(hydroxyimino)-2,3-dihydro-1H-inden-5-ylamino)-N,N-dimethylthieno[2,3-c]pyridine-2-carboxamide

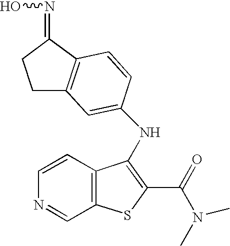

Step A: Preparation of 3-(1-(tert-butyldimethylsilyloxyimino)-2,3-dihydro-1H-inden-5-ylamino)-N,N-dimethylthieno[2,3-c]pyridine-2-carboxamide: A solution of N-methoxymethanamine (25.4 mg, 0.415 mmol) in CH₂Cl₂ was prepared, and trimethylaluminum (0.415 mL, 0.830 mmol) was added. The reaction mixture was stirred at room temperature for 15 minutes. A solution of ethyl 3-(1-(tert-butyldimethylsilyloxyimino)-2,3-dihydro-1H-inden-5-ylamino)thieno[2,3-c]pyridine-2-carboxylate (100 mg, 0.208 mmol; prepared according to Example 2, Steps A and B) in dichloroethane and heated at reflux for 30 hours. The crude reaction was purified by silica gel chromoatography (3:1 ethyl acetate/hexane) to provide 34 mg (34%) of the desired product.

Step B: Preparation of 3-(1-(hydroxyimino)-2,3-dihydro-1H-inden-5-ylamino)-N N-dimethylthieno[2,3-c]pyridine-2-carboxamide: 3-(1-(tert-butyldimethylsilyloxyimino)-2,3-dihydro-1H-inden-5-ylamino)-N,N-dimethylthieno[2,3-c]pyridine-2-carboxamide (34 mg, 0.0707 mmol) was dissolved in CH₂Cl₂ and cooled to 0° C. on ice. TBAF (0.0743 mL, 0.0743 mmol) was added and the reaction mixture was stirred while warming to room temperature over 1 hour. The reaction mixture was transferred to a separatory funnel, diluted with CH₂Cl₂ and water, and the aqueous layer was extracted twice with CH₂Cl₂. The combined organic layers were dried over Na₂SO₄, filtered, concentrated, and purified by preparative TLC to provide 9.9 mg (38%) of the desired product as a yellow film. MS(+) m/z=367.2.

### Example 17

### Preparation of 3-(1-(hydroxyimino)-2,3-dihydro-1H-inden-5-ylamino)-N-phenylthieno[2,3-c]pyridine-2-carboxamide

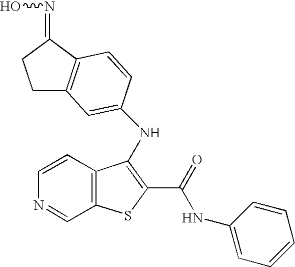

Step A: Preparation of 3-(1-(tert-butyldimethylsilyloxyimino)-2,3-dihydro-1H-inden-5-ylamino)-N-phenylthieno[2,3-c]pyridine-2-carboxamide: Aniline (0.01040 mL, 0.1142 mmol) and trimethylaluminum (0.1038 mL, 0.2076 mmol) were combined in CH₂Cl₂ at room temperature under Argon. Evolution of gas was observed and a dark brown colored solution was produced. This mixture was added to a solution of ethyl 3-(1-(tert-butyldimethylsilyloxyimino)-2,3-dihydro-1H-inden-5-ylamino)thieno[2,3-c]pyridine-2-carboxylate (50.0 mg, 0.1038 mmol; prepared according to Example 2, Steps A and B) in CH₂Cl₂, and the reaction vessel was flushed with Argon. The reaction mixture was heated at 50° C. for 30 hours, and then diluted with water (150 mL) and EtOAc (3×50 mL). The combined organic layers were dried over Na₂SO₄ and concentrated to provide the product (26 mg) as a yellow film. The crude product was used in the next step without purification.

Step B: Preparation of 3-(1-(hydroxyimino)-2,3-dihydro-1H-inden-5-ylamino)-N-phenylthieno[2,3-c]pyridine-2-carboxamide: 3-(1-(tert-butyldimethylsilyloxyimino)-2,3-dihydro-1H-inden-5-ylamino)-N-phenylthieno[2,3-c]pyridine-2-carboxamide (25.9 mg, 0.0490 mmol) was dissolved in CH₂Cl₂ and cooled to 0° C. on ice. TBAF (0.0514 mL, 0.0514 mmol) was added with stirring, and the reaction mixture was warmed to room temperature over 1 hour. The reaction mixture was diluted with CH₂Cl₂ and water and extracted twice. The combined organic layers were dried over Na₂SO₄, filtered, concentrated, and purified by preparative TLC to provide 13.3 mg (65%) of the desired product as a yellow film. ¹H NMR is consistent with desired structure.

### Example 18

### Preparation of tert-butyl 2-iodothieno[2,3-c]pyridin-3-ylcarbamate

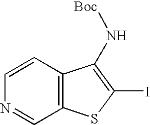

Step A: Preparation of ethyl 3(di-tert-butoxycarbonyl)thieno[2,3-c]pyridine-2-carboxylate: Ethyl 3-aminothieno[2,3-c]pyridine-2-carboxylate (273 mg, 1.228 mmol; prepared according to Example 1) was dissolved in CH₂Cl₂ (10 mL). Dimethylaminopyridine (75.03 mg, 0.6141 mmol) and triethylamine (0.1883 mL, 1.351 mmol) were added, followed by Boc₂O (536.1 mg, 2.457 mmol). The reaction mixture was stirred at room temperature 2 hours, then an additional 1.0 equiv. of Boc₂O was added and the reaction mixture was stirred for 1 hour at room temperature to provide 361 mg of the di-Boc protected amine.

Step B: Preparation of 3-(tert-butoxycarbonyl)thieno[2,3-c]pyridine-2-carboxylic acid: Ethyl 3(di-tert-butoxycarbonyl)thieno[2,3-c]pyridine-2-carboxylate (650.1 mg, 1.539 mmol) was dissolved in EtOH (12 mL), lithium hydroxide (92.12 mg, 3.847 mmol) was added, and the reaction mixture was heated at reflux for 2 hours. The reaction mixture was then purified by an aqueous wash, adjusting the pH to ˜6 with 1N HCl. The aqueous layer was extracted 2× with EtOAc, dried, filtered and concentrated to provide 337 mg (75%) of the desired product as a yellow solid.

Step C: Preparation of tert-butyl 2-iodothieno[2,3-c]pyridin-3-ylcarbamate: 3-(tert-Butoxycarbonyl)thieno[2,3-c]pyridine-2-carboxylic acid (150.7 mg, 0.5120 mmol) was dissolved in 5 mL DMF, and 12 was added. The reaction mixture was heated at 80° C. for 2 hours, then extracted with Na₂SO₃, dried, filtered and concentrated to yellow solid. The solid was purified by column chromatography to provide 86 mg (45%) of the desired product.

### Example 19

### Preparation of 5-(2-(4-methoxyphenyl)thieno[2,3-c]pyridin-3-ylamino)-2,3-dihydroinden-1-one oxime

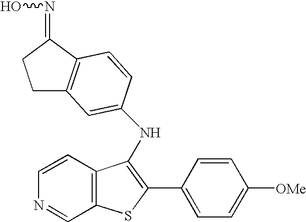

Step A: Preparation of tert-butyl 2-(4-methoxyphenyl)thieno[2,3-c]pyridin-3-ylcarbamate: tert-Butyl 2-iodothieno[2,3-c]pyridin-3-ylcarbamate (73.5 mg, 0.195 mmol; prepared according to Example 18) and 4-methoxyphenylboronic acid (44.5 mg, 0.293 mmol) were dissolved in 4 mL acetonitrile. An aqueous solution of K₂CO₃ (81.0 mg, 0.586 mmol, in 1 mL H₂O) was added to the reaction mixture with stirring, and the reaction mixture was degassed with Argon for 5 minutes. Pd(PPh₃)₄ (11.3 mg, 0.00977 mmol) was added, and the reaction mixture was heated to 80° C. The reaction mixture was then diluted with water, extracted 3× with ethyl acetate, dried, filtered and concentrated to yellow film. The film was purified by preparative TLC to provide the desired product in quantitative yield. MS (+) m/z=357.0.

Step B: Preparation of 2-(4-methoxyphenyl)thieno[2,3-c]pyridin-3-amine: tert-Butyl 2-(4-methoxyphenyl)thieno[2,3-c]pyridin-3-ylcarbamate (69 mg, 0.194 mmol) was dissolved in 2 mL CH₂Cl₂, then TFA (0.0149 mL, 0.194 mmol) was added and the reaction mixture was stirred at room temperature for 1 hour. The reaction mixture was then diluted with water, extracted 3 times with CH₂Cl₂, dried, filtered and concentrated to provide the crude product as a yellow solid in approximately 80% yield. The crude material was used in the next step without further purification.

Step C: Preparation of 5-(2-(4-methoxyphenyl)thieno[2,3-c]pyridin-3-ylamino)-2,3-dihydroinden-1-one O-tert-butyldimethylsilyl oxime: 2-(4-methoxyphenyl)thieno[2,3-c]pyridin-3-amine (40 mg, 0.156 mmol), 5-bromo-2,3-dihydroinden-1-one O-tert-butyldimethylsilyl oxime (53.1 mg, 0.156 mmol) and Cs₂CO₃ (81.4 mg, 0.250 mmol) were combined in toluene and the solution was degassed with Argon for 10 minutes. Pd₂(dba)₃ (5.72 mg, 0.00624 mmol) and X-Phos (2.98 mg, 0.00624 mmol) were added, and the reaction mixture was heated at 110° C. overnight. The reaction mixture was diluted with water and extracted with EtOAc. The EtOAc layers were dried, filtered and concentrated to a brown film. The film was purified by preparative TLC to provide 37 mg (46%) of the desired product. MS (+) m/z=516.3.

Step D: Preparation of 5-(2-(4-methoxyphenyl)thieno[2,3-c]pyridin-3-ylamino)-2,3-dihydroinden-1-one oxime: 5-(2-(4-methoxyphenyl)thieno[2,3-c]pyridin-3-ylamino)-2,3-dihydroinden-1-one O-tert-butyldimethylsilyl oxime (35 mg, 0.0679 mmol) was dissolved in 2 mL CH₂Cl₂ and the reaction mixture was cooled to 0° C. TBAF (0.0713 mL, 0.0713 mmol) was added, and the reaction mixture was stirred for 1 hour. The reaction mixture was concentrated under reduced pressure, and the residue was dissolved in CH₂Cl₂ and washed with NH₄Cl and brine. The aqueous layer was extracted 3 times with CH₂Cl₂, and the combined organic layers were dried, filtered and concentrated to a film. The film was purified by preparative TLC to provide 15.3 mg (56%) of the desired product.

### Example 20

### Preparation of 5-(2-iodothieno[2,3-c]pyridin-3-ylamino)-2,3-dihydroinden-1-one oxime

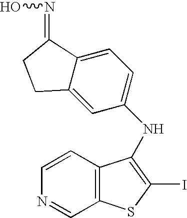

Step A: Preparation of 3-(1-(hydroxyimino)-2,3-dihydro-1H-inden-5-ylamino)thieno[2,3-cpyridine-2-carboxylic acid: ethyl 3-(1-(tert-butyldimethylsilyloxyimino)-2,3-dihydro-1H-inden-5-ylamino)thieno[2,3-c]pyridine-2-carboxylate (63 mg, 0.13 mmol; prepared according to Example 2, Steps A and B), was dissolved in 10 mL ethanol and heated to 50° C. LiOH (7.8 mg, 0.33 mmol) was added, and the reaction mixture was stirred for 1 hour. Water was added, the pH was adjusted to ˜6 with 1N HCl, and the reaction mixture was extracted 3× with EtOAc. The combined organic layers were dried over Na₂SO₄, filtered and concentrated to provide the desired product as a yellow film. The crude product was used in the next step without purification.

Step B: Preparation of 5-(2-iodothieno[2,3-c]pyridin-3-ylamino)-2,3-dihydroinden-1-one oxime: 3-(1-(hydroxyimino)-2,3-dihydro-1H-inden-5-ylamino)thieno[2,3-cpyridine-2-carboxylic acid (59 mg, 0.1301 mmol) was combined with iodine in DMF and the reaction mixture was stirred for 2 hours. The reaction mixture was diluted with EtOAc and washed with aqueous Na₂SO₃. The combined organic layers were dried, filtered and concentrated to provide 44 mg (80%) of the crude product.

### Example 21

### Preparation of ethyl 3-(4-chloro-3-hydroxyphenylamino)thieno[2,3-c]pyridine-2-carboxylate

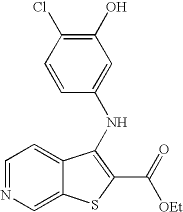

Step A: Preparation of ethyl 3-(4-chloro-3-methoxyphenylamino)thieno[2,3-c]pyridine-2-carboxylate: Ethyl 3-aminothieno[2,3-c]pyridine-2-carboxylate (273 mg, 1.228 mmol; prepared according to Example 1) (103 mg, 0.4634 mmol) and 4-bromo-1-chloro-2-methoxybenzene (0.06293 mL, 0.4634 mmol) were combined in 10 mL toluene with Cs₂CO₃ (241.6 mg, 0.7415 mmol) and degassed 10 minutes with Argon. Pd₂(dba)₃ (16.97 mg, 0.01854 mmol) and X-Phos (6.628 mg, 0.01390 mmol) were added, and the reaction mixture at was heated at 110° C. for 1 hour. The reaction mixture was partitioned between EtOAc and water, and the organic layer was dried, filtered and concentrated to brown residue. The residue was purified by preparative TLC to provide 95 mg (56%) of the desired product. MS (+) shows m/z=363.2.

Step B: Preparation of ethyl 3-(4-chloro-3-hydroxyphenylamino)thieno[2,3-c]pyridine-2-carboxylate: A solution of ethyl 3-(4-chloro-3-methoxyphenylamino)thieno[2,3-c]pyridine-2-carboxylate (95 mg, 0.2618 mmol) was cooled to −78° C. in dry ice/acetone bath. Tribromoborane (0.7855 mL, 0.7855 mmol) was added via syringe, and the reaction mixture was stirred for 30 minutes, then stirred at 0° C. in ice bath over 2 hours and then at room temperature for 4 hours. The reaction mixture was carefully diluted with H₂O. A small amount of NaHCO₃ was added to adjust to ˜pH 7. The reaction mixture was extracted 3× with CH₂Cl₂, then dried, filtered and concentrated to provide a brown film. The residue was purified by preparative TLC eluting with 5% MeOH/CH₂Cl₂ providing separation of all 4 major components. MS (+) m/z=349.2. Yield=11 mg (12%).

### Example 22

### Preparation of (E,Z)-(3-(1-(hydroxyimino)-2,3-dihydro-1H-inden-5-ylamino)thieno[2,3-c]pyridin-2-yl)(morpholino)methanone

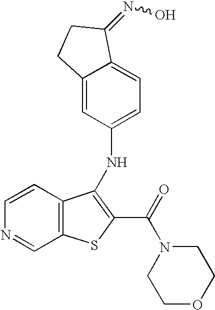

Step A: Preparation of ethyl 3-(di-tert-butoxycarbonyl)thieno[2,3-c]pyridine-2-carboxylate: Prepared according to Example 18, Step A.

Step B: Preparation of 3-(tert-butoxycarbonyl)thieno[2,3-c]pyridine-2-carboxylic acid: To a 50 mL round bottom flask was added ethyl 3-(di-tert-butoxycarbonyl)thieno[2,3-c]pyridine-2-carboxylate (550 mg, 1.30 mmol), 12 mL ethanol and lithium hydroxide (77.9 mg, 3.25 mmol) The resulting reaction mixture was allowed to reflux for 2 hours. The pH was adjusted to 6.0 with 1 M HCl. The reaction mixture was diluted with 25 mL brine and the organic layer was extracted with 3×50 mL ethyl acetate. The top organic layers were combined, dried over MgSO₄, and concentrated to an oil which crystallized. Isolated 350 mg (91% yield) of desired material. MS (M+1)=294.9.

Step C: Preparation of tert-butyl 2-(morpholine-4-carbonyl)thieno[2,3-c]pyridin-3-ylcarbamate: To a 0° C. solution of 3-(tert-butoxycarbonyl)thieno[2,3-c]pyridine-2-carboxylic acid (350 mg, 1.189 mmol), HOBT (95 mg 0.594 mmol) and triethylamine (0.8287 mL, 5.946 mmol) in 10 mL THF was added EDCI (285.0 mg, 1.486 mmol) followed by morpholine (0.1556 mL, 1.784 mmol). The reaction mixture was allowed to stir overnight while warming to room temperature. Silica gel (5 g) was added to the reaction mixture, which was then concentrated to a solid. The solids were placed on a 10 g silica gel column and eluted with 100% ethyl acetate to yield 146 mg (33.81% yield) of desired amide product. MS M+1=364.0.

Step D: Preparation of (3-aminothieno[2,3-c]pyridin-2-yl)(morpholino)methanone: A mixture of tert-Butyl 2-(morpholine-4-carbonyl)thieno[2,3-c]pyridin-3-ylcarbamate (100 mg, 0.275 mmol) and TFA (0.454 mL, 5.5 mmol) was allowed to stir overnight at room temperature. The reaction mixture was concentrated to an oil and the residue was diluted with 20 mL CH₂Cl₂. The organic layer was washed with NaHCO₃ (2×20 mL) and brine (2×20 mL), then dried over MgSO₄, filtered, and concentrated to oil. The oil was placed on a 10 gm silica gel column and eluted with 10% MeOH/ethyl acetate to yield 51 mg (70.3%) of desired product. MS M+1=264.2.

Step E: Preparation of (E,Z)-(3-(1-(tert-butyldimethylsilyloxyimino)-2,3-dihydro-1H-inden-5-ylamino)thieno[2,3-c]pyridin-2-yl)(morpholino)methanone: To a 10 mL conical reacti-vial was added (3-aminothieno[2,3-c]pyridin-2-yl)(morpholino)methanone (50 mg, 0.19 mmol), (E,Z)-5-bromo-2,3-dihydroinden-1-one, O-tert-butyldimethylsilyl oxime (80.8 mg, 0.237 mmol), X-Phos (2.72 mg, 0.005 mmol), Pd₂(dba)₃ (4.37 mg, 0.007 mmol), Cs₂CO₃ (124 mg, 0.38 mmol) and 5 mL toluene. The vial was sealed and the reaction mixture was heated at 70° C. for 18 hours. Three grams of silica gel were added to the reaction mixture and the resulting slurry was concentrated to a solid. The solid was placed on a 10 g silica gel column and eluted with 25% CH₂Cl₂/ethyl acetate to yield 20 mg (20.1% yield) of the desired product. MS M+1=523.4.

Step F: Preparation of (E,Z)-(3-(1-(hydroxyimino)-2,3-dihydro-1H-inden-5-ylamino)thieno[2,3-c]pyridin-2-yl)(morpholino)methanone: To a solution of (E,Z)-(3-(1-(tert-butyldimethylsilyloxyimino)-2,3-dihydro-1H-inden-5-ylamino)thieno[2,3-c]pyridin-2-yl)(morpholino)methanone (20 mg, 0.038 mmol) in THF was added TBAF (0.38 mL, 0.38 mmol) and the reaction mixture was stirred at room temperature overnight. The reaction mixture was concentrated, and the crude product was purified on a sep-pak (10 g, dry loading), eluting with CH₂Cl₂/MeOH (50:1), CH₂Cl₂/MeOH (20:1) to give 16 mg (100% yield) of the desired product. MS M+1=409.2.

### Example 23

### Preparation of 5-(2-(hydroxymethyl)thieno[2,3-c]pyridin-3-ylamino)-2,3-dihydroinden-1-one oxime

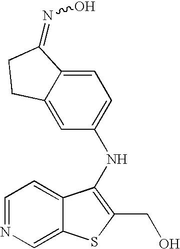

Step A: Preparation of (3-aminothieno[2,3-c]pyridin-2-yl)methanol: To a 0° C. solution of ethyl 3-aminothieno[2,3-c]pyridine-2-carboxylate (314.4 mg, 1.415 mmol; prepared according to Example 1) in 5 mL THF was added 1M LAH (2.829 mL, 2.829 mmol) in one portion. The reaction mixture was stirred for 3 hours while warming to room temperature. Na₂SO₄.6H₂O was added along with 20 mL MeOH. The reaction mixture was stirred overnight and then filtered, and the solids were washed with 2×50 mL ethyl acetate. The combined filtrates were concentrated to provide the desired product in quantitative yield as a solid. MS M+1=181.2.

Step B: Preparation of 2-((tert-butyldimethylsilyloxy)methyl)thieno[2,3-c]pyridin-3-amine: To a room temperature solution/slurry of (3-aminothieno[2,3-c]pyridin-2-yl)methanol (41 mg, 0.227 mmol) and imidazole (46.5 mg, 0.682 mmol) in 5 mL CH₂Cl₂ was added TBDMS-Cl (51.4 mg, 0.341 mmol) in one portion. The reaction mixture was allowed to stir overnight. Silica gel was added and concentrated to a solid. The solid was placed on a 5 g silica column and eluted with CH₂Cl₂ and then ethyl acetate to provide 22.4 mg (33.4%) of the desired product. MS M+1=295.2.

Step C: Preparation of 5-(2-((tert-butyldimethylsilyloxy)methyl)thieno[2,3-c]pyridin-3-ylamino)-2,3-dihydroinden-1-one O-tert-butyldimethylsilyl oxime: To a 10 mL reacti-vial was added 2-((tert-butyldimethylsilyloxy)methyl)thieno[2,3-c]pyridin-3-amine (22.4 mg, 0.0761 mmol), (E,Z)-5-bromo-2,3-dihydroinden-1-one O-tert-butyldimethylsilyl oxime (32.4 mg, 0.0951 mmol), X-Phos (2.90 mg, 0.00609 mmol), Pd₂(dba)₃ (4.37 mg, 0.00761 mmol), and Cs₂CO₃ (49.6 mg, 0.152 mmol) in 5 mL toluene and the reaction mixture was heated to 70° C. for 18 hours. Silica gel (3 g) was added, and the reaction mixture concentrated to a solid. The solid was placed on a 10 g silica column and eluted with CH₂Cl₂/25% ethyl acetate to provide 20 mg (47.5% yield) of the desired product. MS M+1=554.3.

Step D: Preparation of (E,Z)-5-(2-(hydroxymethyl)thieno[2,3-c]pyridin-3-ylamino)-2,3-dihydroinden-1-one oxime: To a solution of (E,Z)-5-(2-((tert-butyldimethylsilyloxy)methyl)thieno[2,3-c]pyridin-3-ylamino)-2,3-dihydroinden-1-one O-tert-butyldimethylsilyl oxime (20 mg, 0.036 mmol) in THF (1 mL) was added TBAF (0.11 mL, 0.11 mmol). The reaction mixture was stirred at room temperature overnight, then concentrated to an oil. The crude oil was purified on a silica gel column (10 g, dry loading) to give 7 mg (60% yield) of the desired product. MS M+1=326.2.

### Example 24

### Preparation of Ethyl 3-(1-(hydroxyimino)-2,3-dihydro-1H-inden-5-ylamino)thieno[3,2-c]pyridine-2-carboxylate

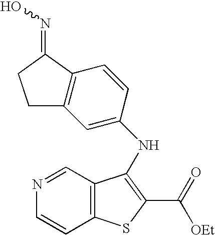

Step A: Preparation of 4-chloronicotinamide: To 4-chloronicotinic acid (10.00 g, 63.47 mmol) was added SOCl₂ (46.30 mL, 634.7 mmol), and resulting mixture was heated at reflux for 4 hours. SOCl₂ was removed via rotary evaporation and toluene (25 mL) was added to the reaction mixture. Toluene was removed and the resulting oil was slowly poured into 25 mL concentrated NH₄OH at 0° C. Solids formed and were filtered to yield 2.7 g (27.6% yield) of the desired product.

Step B: Preparation of 4-chloronicotinonitrile: 4-chloronicotinamide (2.7 g, 17.24 mmol) was suspended in cold (0° C.) THF (100 mL) and triethylamine (19.23 mL, 138.0 mmol). To this was slowly added phosphoryl trichloride (1.607 mL, 17.24 mmol). The reaction mixture was allowed to stir for 3 hours while warming to room temperature. Silica gel was added and the reaction mixture was concentrated (keeping the bath temperature ˜35° C.). The residue was dry loaded onto a Biotage 40M column and eluted with CH₂Cl₂ (100%) to give 2.1 g (87.89% yield) of the desired product.

Step C: Preparation of ethyl 3-aminothieno[3,2-c]pyridine-2-carboxylate: To a room temperature solution of 4-chloronicotinonitrile (1.0 g, 7.2 mmol) and ethyl 2-mercaptoacetate (0.8673 g, 7.2 mmol) in 50 mL dry DMF was added sodium methoxide in ethanol (5.5 mL, 14.4 mmol). After 12 hours, 25 mL of water and HOAc (1.0 mL, 18 mmol) were added, and the reaction mixture was extracted with 2×100 mL ethyl acetate. The organic layers were combined, dried over MgSO₄ and concentrated to a solid. The solid was placed on a Biotage column and eluted with CH₂Cl₂, followed by ethyl acetate (100%) to provide 1.2 g (74.8% yield) of the desired product. MS M+1=223.1.

Step D: Preparation of ethyl 3-(1-(tert-butyldimethylsilyloxyimino)-2,3-dihydro-1H-inden-5-ylamino)thieno[3,2-c]pyridine-2-carboxylate: To a 10 mL reacti-vial was added ethyl 3-aminothieno[3,2-c]pyridine-2-carboxylate (214.0 mg, 0.963 mmol), (E,Z)-5-bromo-2,3-dihydroinden-1-one O-tert-butyldimethylsilyl oxime (328.0 mg, 0.963 mmol; prepared according to Example 2, Step A), X-Phos (13.8 mg, 0.039 mmol), Pd₂(dba)₃ (35.0 mg, 0.04 mmol), and Cs₂CO₃ (502.0 mg, 1.54 mmol) along with 5 mL toluene and contents were heated to 70° C. for 18 hours. Added 3 g of silica gel and concentrated to a solid. The solid was loaded onto a 10 g column and eluted with CH₂Cl₂/25% ethyl acetate to provide 300 mg (64.7% yield) of the desired product. MS M+1=482.2.

Step E: Preparation of ethyl 3-(1-(hydroxyimino)-2,3-dihydro-1H-inden-5-ylamino)thieno[3,2-c]pyridine-2-carboxylate: To a solution of ethyl 3-(1-(tert-butyldimethylsilyloxyimino)-2,3-dihydro-1H-inden-5-ylamino)thieno[3,2-c]pyridine-2-carboxylate (300 mg, 0.623 mmol) in THF (10 mL) was added TBAF (1.2 mL, 1.2 mmol). The reaction mixture was stirred at room temperature overnight. The reaction was concentrated and the resulting oil was purified on a silica gel column (10 g, dry loading) to give 174 mg (76% yield) of the desired product. MS M+1=368.2.

### Example 25

### Preparation of 2-(dimethylamino)ethyl-3-(1-(hydroxyimino)-2,3-dihydro-1H-inden-5-ylamino)thieno[2,3-c]pyridine-2-carboxylate

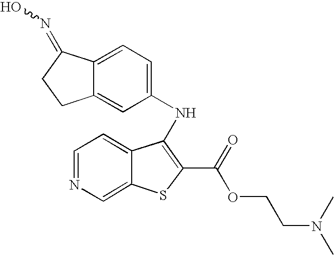

Step A: Preparation of 2-(dimethylamino)ethyl-3-(tert-butoxycarbonyl)thieno[2,3-c]pyridine-2-carboxylate: To a suspension of 3-(tert-butoxycarbonyl)thieno[2,3-c]pyridine-2-carboxylic acid (0.093 g, 0.316 mmol; prepared according to Example 9, Steps A and B) in CH₂Cl₂ (5.0 mL) was added 2-dimethylamino ethanol (0.062 g, 0.696 mmol), EDCI (0.122 g, 0.622 mmol) and a catalytic amount of DMAP (˜5 mg). The reaction mixture was left at room temperature overnight, then diluted with water. The aqueous layer was extracted with CH₂Cl₂ (3×50 mL), and the combined organic layers were dried, filtered and concentrated. The crude product was purified by flash column chromatography, eluting with CH₂Cl₂/MeOH (50:1) to give 0.034 g of the desired product as a yellow oil. MS (APCI) m/z 366.0 (M+1).

Step B: Preparation of 2-(dimethylamino)ethyl-3-aminothieno[2,3-c]pyridine-2-carboxylate: To a cold (0° C.) solution of 2-(dimethylamino)ethyl 3-(tert-butoxycarbonyl)thieno[2,3-c]pyridine-2-carboxylate (0.034 g, 0.093 mmol) in CH₂Cl₂ (2.0 mL) was added TFA (2.0 mL) dropwise. The reaction mixture was allowed to warm up to room temperature overnight, then concentrated. The residue was taken up in triethylamine (2.0 mL) and reconcentrated. The crude product was purified by flash column chromatography, eluting with CH₂Cl₂/MeOH (50:1), CH₂Cl₂/MeOH (20:1) to give the desired product as a yellow oil. MS (APCI) m/z 266.1 (M+1).

Step C: Preparation of (2-(dimethylamino)ethyl 3-(1-(tert-butyldimethylsilyloxyimino)-2,3-dihydro-1H-inden-5-ylamino)thieno[2,3-c]pyridine-2-carboxylate: 5-bromo-2,3-dihydroinden-1-one O-tert-butyldimethylsilyl oxime (0.092 g, 0.269 mmol) and 2-(dimethylamino)ethyl 3-aminothieno[2,3-c]pyridine-2-carboxylate (0.065 g, 0.245 mmol) was suspended in toluene (4.0 mL) and degassed with N₂ for 15 minutes. To this was added X-Phos (0.024 g, 0.0490 mmol), Pd₂(dba)₃ (0.022 g, 0.0245 mmol) and Cs₂CO₃ (0.128 g, 0.392 mmol). The reaction mixture was degassed for another 15 minutes and then heated to 110° C. for 3 hours. After cooling to room temperature, the reaction mixture was filtered through GF/F paper, rinsed with CH₂Cl₂ and concentrated. The crude product was purified by flash column chromatography, eluting with hexane, hexane/ethyl acetate (20:1), hexane/ethyl acetate (10:1), ethyl acetate, and CH₂Cl₂/MeOH (20:1) to give 2-(dimethylamino)ethyl-3-(1-(tert-butyldimethylsilyloxyimino)-2,3-dihydro-1H-inden-5-ylamino)thieno[2,3-c]pyridine-2-carboxylate as a yellow oil. MS (APCI) m/z 525.1 (M+1).

Step D: Preparation of 2-(dimethylamino)ethyl-3-(1-(hydroxyimino)-2,3-dihydro-1H-inden-5-ylamino)thieno[2,3-c]pyridine-2-carboxylate: 2-(dimethylamino)ethyl 3-(1-(tert-butyldimethylsilyloxyimino)-2,3-dihydro-1H-inden-5-ylamino)thieno[2,3-c]pyridine-2-carboxylate was dissolved in THF (2.0 mL) and treated with TBAF (1.0 equiv.) for 20 minutes. The solvent was removed and the residue was purified by flash column chromatography, eluting with CH₂Cl₂, CH₂Cl₂/MeOH (50:1), CH₂Cl₂/MeOH (25:1) to give 2-(dimethylamino)ethyl-3-(1-(hydroxyimino)-2,3-dihydro-1H-inden-5-ylamino)thieno[2,3-c]pyridine-2-carboxylate as a yellow oil. MS (APCI) m/z 411.0 (M+1).

### Example 26

### Preparation of Ethyl 3-(1-(hydroxyimino)-2,3-dihydro-1H-inden-5-ylamino)furo[2,3-c]pyridine-2-carboxylate

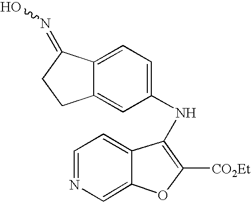

Step A: Preparation of ethyl 3-aminofuro[2,3-c]pyridine-2-carboxylate: To a cold solution (0° C.) of 3-bromoisonicotinoitrile (5.0 g, 27.3 mmol) in DMF (50 mL) was added NaH (60% dispersion in mineral oil, 1.15 g, 28.7 mmol). To this was slowly added a solution of ethyl glycolate (3.13 g, 2.84 mL, 30.1 mmol) in DMF (6.0 mL) via an addition funnel. The reaction mixture was allowed to warm up to room temperature and stirred for 1 hour, then diluted with water (50 mL) and ethyl acetate (100 mL). The aqueous layer was extracted with ethyl acetate (2×100 mL). The combined organic layers were dried, filtered and concentrated. The crude product was purified by flash column chromatography, eluting with hexanes/ethyl acetate (4:1), hexanes/ethyl acetate (5:2) to give the desired product as a yellow solid (0.270 g). MS (APCI) m/z 407.1 (M+1).

Step B: Preparation of ethyl 3-(1-(tert-butyldimethylsilyloxyimino)-2,3-dihydro-1H-inden-5-yl)furo[2,3-c]pyridine-2-carboxylate: 3-aminofuro[2,3-c]pyridine-2-carboxylate (0.27 g, 1.31 mmol) and 5-bromo-2,3-dihydroinden-1-one O-tert-butyldimethylsilyl oxime (0.49 g, 1.44 mmol) were suspended in toluene (10.0 mL) and degassed with N₂ for 15 minutes. To this was added X-Phos (0.124 g, 0.262 mmol), Pd₂(dba)₃ (0.120 g, 0.132 mmol) and Cs₂CO₃ (0.683 g, 2.10 mmol). The reaction mixture was degassed for another 15 minutes and then heated to 110° C. for 72 hours. After cooling to room temperature, the reaction mixture was filtered through GF/F paper, rinse with CH₂Cl₂ and concentrated. The crude material was purified by flash column chromatography, eluting with hexanes/ethyl acetate (9:1) and hexanes/ethyl acetate (7:3) to give 0.326 g the desired product as a yellow oil. MS (APCI) m/z 466.3 (M+1).

Step C: Preparation of (E)-ethyl 3-(1-(hydroxyimino)-2,3-dihydro-1H-inden-5-ylamino)furo[2,3-c]pyridine-2-carboxylate: (E)-ethyl 3-(1-(tert-butyldimethylsilyloxyimino)-2,3-dihydro-1H-inden-5-yl)furo[2,3-c]pyridine-2-carboxylate was dissolved in THF (2.0 mL) and treated with TBAF (1.0 equiv.) for 20 minutes. The solvent was removed and the residue was purified by flash column chromatography, eluting with ethyl acetate/hexanes (1:1), ethyl acetate, and ethyl acetate/EtOH (200:3) to give the desired product as a yellow solid. MS (APCI) m/z 352.2 (M+1).

### Example 27

### Preparation of Methyl 3-(1-(hydroxyimino)-2,3-dihydro-1H-inden-5-ylamino)furo[2,3-c]pyridine-2-carboxylate

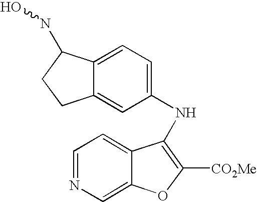

Ethyl 3-(1-(hydroxyimino)-2,3-dihydro-1H-inden-5-ylamino)furo[2,3-c]pyridine-2-carboxylate (0.07 g, 0.21 mmol; prepared according to Example 26) was suspended in MeOH (10.0 mL) and to this was added catalytic amount of Ti(OEt)₄ (˜10 mg). The reaction mixture was refluxed overnight, then cooled to room temperature and concentrated. The crude product was purified by flash column chromatography, eluting with ethyl acetate/hexanes (1:1), ethyl acetate, and ethyl acetate/EtOH (200:3) to give 0.020 g of (E)-methyl 3-(1-(hydroxyimino)-2,3-dihydro-1H-inden-5-ylamino)furo[2,3-c]pyridine-2-carboxylate as a yellow solid. MS (APCI) m/z 338.2 (M+1).

### Example 28

### Preparation of Isopropyl 3-(1-(hydroxyimino)-2,3-dihydro-1H-inden-5-ylamino)furo[2,3-c]pyridine-2-carboxylate

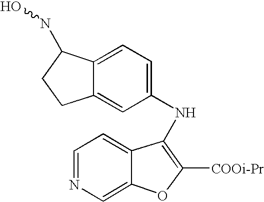

(E)-Ethyl 3-(1-(hydroxyimino)-2,3-dihydro-1H-inden-5-ylamino)furo[2,3-c]pyridine-2-carboxylate (0.010 g, 0.028 mmol; prepared according to Example 26) was suspended in i-PrOH (10.0 mL) and to this was added catalytic amount of Ti(OEt)₄ (˜3 mg). The reaction mixture was refluxed for 3 hours, then cooled to room temperature and concentrated. The crude product was purified by flash column chromatography, eluting with ethyl acetate/CH₂Cl₂ (1:1) and ethyl acetate/CH₂Cl₂ (7:3) to give 0.004 g of the desired product as a yellow solid. MS (APCI) m/z 366.1 (M+1).

### Example 29

### Preparation of 5-(2-(pyridin-4-yl)furo[2,3-c]pyridin-3-ylamino)-2,3-dihydroinden-1-one oxime

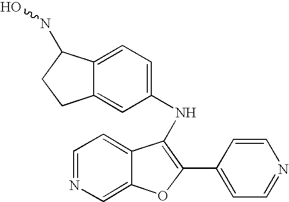

Step A: Preparation of 2-(pyridin-4-yl)furo[2,3-c]pyridin-3-amine: To a cold (0° C.) suspension of NaH (60% dispersion in mineral oil, 0.053 g, 1.31 mmol) in DMF (2.0 mL) was added a solution of pyridin-4-ylmethanol (0.131, 1.20 mmol) in DMF (2.0 mL) dropwise. The reaction mixture was stirred for 10 minutes, and then a solution of 3-bromoisonicotinoitrile (0.200 g, 1.09 mmol) in DMF (5.0 mL) was added dropwise. The reaction mixture was stirred for 1 hour at room temperature before quenching with water (20 mL). The aqueous layer was extracted with ethyl acetate (3×50 mL). The combined organic layers were dried, filtered and concentrated. The crude product was purified by flash column chromatography, eluting with CH₂Cl₂/MeOH (20:1) and CH₂Cl₂/MeOH (100:7) to give 0.070 g of the desired product as a yellow solid. MS (APCI) m/z 212.5 (M+1):

Step B: Preparation of 5-(2-(pyridine-4-yl)furo[2,3-c]pyridine-3-ylamino)-2,3-dihydroinden-1-one O-tert-butyldimethylsilyl oxime: (E)-5-bromo-2,3-dihydroinden-1-one O-tert-butyldimethylsilyl oxime (0.12 g, 0.36 mmol) and 2-(pyridin-4-yl)furo[2,3-c]pyridin-3-amine (0.070 g, 0.33 mmol) were suspended in toluene (4.0 mL) and the reaction mixture was degassed with N₂ for 15 minutes. To this was added X-Phos (0.016 g, 0.033 mmol), Pd₂(dba)₃ (0.015 g, 0.017 mmol) and Cs₂CO₃ (0.17 g, 0.63 mmol). The reaction mixture was degassed for another 15 minutes and then heated to 110° C. overnight. After cooling to room temperature, the reaction mixture was filtered through GF/F paper, rinse with CH₂Cl₂ and concentrated. The crude material was purified by flash column chromatography, eluting with Hexane/ethyl acetate (9:1) and hexanes/ethyl acetate (7:3) to give the desired product as a yellow oil. MS (APCI) m/z 471.2 (M+1).

Step C: Preparation of 5-(2-(pyridin-4-yl)furo[2,3-c]pyridin-3-ylamino)-2,3-dihydroinden-1-one oxime: (5-(2-(pyridine-4-yl)furo[2,3-c]pyridine-3-ylamino)-2,3-dihydroinden-1-one O-tert-butyldimethylsilyl oxime (0.16 g, 0.34 mmol) was dissolved in THF (2.0 mL) and treated with TBAF (1.0 equiv.; 1.0 M in THF) for 20 minutes. The solvent was removed by rotary evaporation and the residue was purified by flash column chromatography, eluting with ethyl acetate/hexane (9:1) and ethyl acetate to give 0.050 g of the desired product as a yellow solid. MS (APCI) m/z 357.3 (M+1).

### Example 30

### Preparation of 5-(2-(pyridin-2-yl)furo[2,3-c]pyridin-3-ylamino)-2,3-dihydroinden-1-one oxime

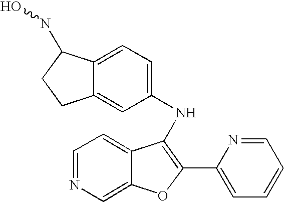

Step A: Preparation of 2-(pyridin-2-yl)furo[2,3-c]pyridin-3-amine: Prepared using the general procedure described in Example 29, Step A. MS (APCI) m/z 212.3 (M+1).

Step B: Preparation of (E)-5-(2-(pyridin-2-yl)furo[2,3-c]pyridin-3-ylamino)-2,3-dihydroinden-1-one oxime: Prepared using the general procedure described in Example 29, Steps B and C. MS (APCI) m/z 357.3 (M+1).

### Example 31

### Preparation of 4-(3-(1-(hydroxyimino)-2,3-dihydro-1H-inden-5-ylamino)furo[2,3-c]pyridin-2-yl)benzonitrile

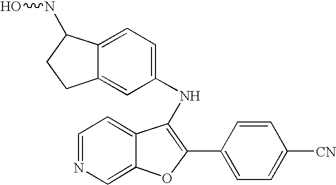

Step A: Preparation of 4-(3-aminofuro[2,3-c]pyridin-2-yl)benzonitrile: Prepared using the general procedure described in Example 29, Step A. MS (APCI) m/z 236.3 (M+1).

Step B: Preparation of 4-(3-(1-(hydroxyimino)-2,3-dihydro-1H-inden-5-ylamino)furo[2,3-c]pyridin-2-yl)benzonitrile: Prepared using the general procedure described in Example 29, Steps B and C. MS (APCI) m/z 381.3 (M+1).

### Example 32

### Preparation of 4-(3-(1-(hydroxyimino)-2,3-dihydro-1H-inden-5-ylamino)furo[2,3-c]pyridin-2-yl)benzamide

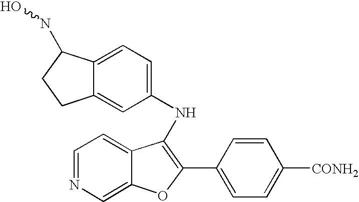

4-(3-(1-(tert-butyldimethylsilyloxyimino)-2,3-dihydro-1H-inden-5-ylamino)furo[2,3-c]pyridin-2-yl)benzonitrile (0.047 g, 0.095 mmol; prepared according to Example 31) was refluxed in EtOH (5.0 mL) with KOH (0.008 g, 0.143 mmol) for 2 hours. The crude reaction mixture was concentrated and was purified by flash column chromatography, eluting with ethyl acetate/MeOH (20:1) to give 0.016 g of the desired product as a yellow solid. MS (APCI) m/z 399.2 (M+1).

### Example 33

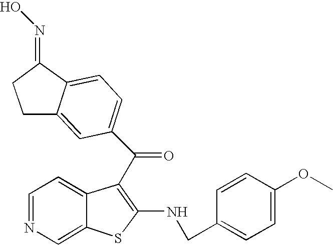

### Preparation of (1-(hydroxyimino)-2,3-dihydro-1H-inden-5-yl)(2-(4-methoxybenzylamino)-thieno[2,3-c]pyridin-3-yl)methanone

Step A: Preparation of methyl 1-oxo-2,3-dihydro-1H-indene-5-carboxylate: 5-Bromo-2,3-dihydroinden-1-one (10 g, 48 mmol, 1 equiv.) and triethylamine (20 mL, 3 equiv.) were slurried in MeOH (100 mL) in a reaction bomb and the solution was degassed with Argon for 5 minutes. Pd(OAc)₂ (0.54 g, 0.05 equiv.) and PPh₃ (2.5 g, 0.2 equiv.) were added and the bomb was pressurized with CO to 85 psi and heated to 70° C. overnight. The reaction was cooled to room temperature and the volatiles were removed by rotary evaporation. The residue was taken up in EtOAc and 1N HCl, and the solids were filtered off solids through GF/F paper. The layers were separated and the organic layer was washed with saturated NaHCO₃ and brine. The combined aqueous extracts were extracted with EtOAc and the combined organic extracts were dried over MgSO₄, filtered, and concentrated to afford 10 g of the desires product as an off-white solid, which was carried on crude to the next step.

Step B: Preparation of methyl 1-(tert-butyldimethylsilyloxyimino)-2,3-dihydro-1H-indene-5-carboxylate: A 50 mL toluene solution containing methyl 1-oxo-2,3-dihydro-1H-indene-5-carboxylate (0.500 g, 2.63 mmol), TBS-ONH₂ (0.387 g, 2.63 mmol) and catalytic toluenesulfonic acid (0.0500 g, 0.263 mmol) in a round bottom flask equipped with a Dean-Stark trap was heated to 150° C. for 4 hours and the solution azeotroped. The reaction mixture was concentrated, and the residue was taken up in ether, washed with water, dried over sodium sulfate, filtered and concentrated to an off-white solid. The solid was dissolved in CH₂Cl₂ and purified by column chromatography eluting with 100% hexanes-15% EtOAc/Hexanes. The desired product was isolated in 91% yield as a white solid.

Step C: Preparation of 2-(3-bromopyridin-4-yl)-1-(1-(tert-butyldimethylsilyloxyimino)-2,3-dihydro-1H-inden-5-yl)ethanone: An LDA solution was prepared in 30 mL dry THF at room temperature by adding n-butyl lithium (0.574 mL, 1.43 mmol) dropwise to diisopropylamine (0.201 mL, 1.43 mmol) in THF. The LDA solution was stirred at room temperature for 20 minutes. A 3 mL solution of 3-bromo-4-methylpyridine (0.112 g, 0.652 mmol) in dry THF was added and the solution was stirred for 10 minutes before cooling to 0° C. After 15 minutes a 2 mL solution of methyl 1-(tert-butyldimethylsilyloxyimino)-2,3-dihydro-1H-indene-5-carboxylate (0.250 g, 0.783 mmol) in THF was added dropwise. The reaction mixture was stirred for 30 minutes. Saturated ammonium chloride (3 mL) was added and the reaction mixture was poured into ethyl acetate, washed with ammonium chloride, saturated brine, dried over sodium sulfate, filtered and concentrated. The residue was purified by column chromatography using 30% EtOAc/Hexanes. The desired product was isolated in 61% yield as a yellow glass.

Step D: Preparation of (2-(4-methoxybenzylamino)thieno[2,3-c]pyridine-3-yl)(1-(tert-butyldimethylsilyloxyimino)-2,3-dihydro-1H-inden-5-y)methanone: To a microwave vessel charged with sodium hydride (0.00322 g, 0.0805 mmol) under N₂ was added a 1.5 mL solution of 2-(3-bromopyridin-4-yl)-1-(1-(tert-butyldimethylsilyloxyimino)-2,3-dihydro-1H-inden-5-yl)ethanone (0.037 g, 0.0805 mmol). Immediately the resulting solution turned orange and then brown, and gas evolution occurred. After 10-15 minutes the gas evolution had ceased. 1-(Isothiocyanatomethyl)-4-methoxybenzene (0.0126 mL, 0.0805 mmol) was added and the solution was stirred at room temperature for 5 minutes before being subjected to microwave conditions (155 watts, 130° C., 2 minutes). The solution was cooled, poured into excess ammonium chloride solution, extracted with ethyl acetate, dried over sodium sulfate, filtered and purified by column chromatography using 1% MeOH/CHCl₃ to provide the desired product in 89% yield.

Step E: Preparation of (1-(hydroxyimino)-2,3-dihydro-1H-inden-5-yl)(2-(4-methoxybenzylamino)-thieno[2,3-c]pyridin-3-yl)methanone: (2-(4-methoxybenzylamino)thieno[2,3-c]pyridin-3-yl)(1-(tert-butyldimethylsilyloxyimino)-2,3-dihydro-1H-inden-5-yl)methanone (0.040 g, 0.0717 mmol) was dissolved in 1 mL THF at room temperature. TBAF (0.0789 mL, 0.0789 mmol) was added, and the resulting solution was stirred at room temperature for 30 minutes. The reaction mixture was poured into brine, extracted with ethyl acetate, dried over sodium sulfate, filtered and concentrated to a brown solid. The compound was purified by column chromatography using 1% MeOH/EtOAc (or alternatively 1-5% MeOH/CHCl₃+1% NH₄OH) to provide the desired product in 33% yield. MS (APCI) m/z=444.2 (M+1).

### Example 34

### Preparation of (2-(ethylamino)thieno[2,3-c]pyridin-3-yl)(1-(hydroxyimino)-2,3-dihydro-1H-inden-5-yl)methanone

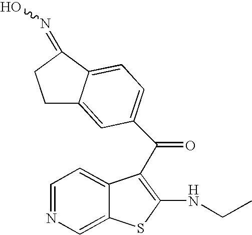

Step A: Preparation of (1-(tert-butyldimethylsilyloxyimino)-2,3-dihydro-1H-inden-5-yl)(2-(ethylamino)thieno[2,3-c]pyridin-3-yl)methanone: 2-(3-bromopyridin-4-yl)-1-(1-(tert-butyldimethylsilyloxyimino)-2,3-dihydro-1H-inden-5-yl)ethanone (0.050 g, 0.109 mmol; prepared according to Example 33, Steps A-C) was dissolved in 1.5 mL dry NMP, and a solution of NaHMDS (0.200 mL, 0.120 mmol) in toluene was added. The resulting solution was stirred for 5 minutes before adding isothiocyanatoethane (0.0104 mL, 0.120 mmol). The reaction mixture was stirred at room temperature for 5 minutes, microwaved (150 watts, 130, 2 minutes). The reaction mixture was cooled to room temperature, diluted with ethyl acetate, washed with saturated ammonium chloride, brine, dried over sodium sulfate, filtered, concentrated to a black oil, and purified by column using 1% MeOH/EtOAc to provide the desired product in 23% yield.

Step B: Preparation of (2-(ethylamino)thieno[2,3-c]pyridin-3-yl)(1-(hydroxyimino)-2,3-dihydro-1H-inden-5-yl)methanone: (1-(tert-Butyldimethylsilyloxyimino)-2,3-dihydro-1H-inden-5-yl)(2-(ethylamino)thieno[2,3-c]pyridin-3-yl)methanone (0.023 g, 0.0494 mmol) was dissolved in 1 mL THF at room temperature. TBAF (0.0543 mL, 0.0543 mmol) was added and the reaction mixture was stirred at room temperature for 15 minutes. Purification by column chromatography using 5% MeOH/CHCl₃+1% NH₄OH provided the desired product as a light yellow solid. MS (APCI) m/z=352.2 (M+1).

### Example 35

### Preparation of (1-(hydroxyimino)-2,3-dihydro-1H-inden-5-yl)(2-(propylamino)thieno[2,3-c]pyridin-3-yl)methanone

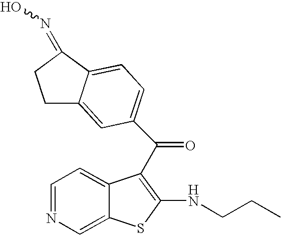

2-(3-Bromopyridin-4-yl)-1-(1-(tert-butyldimethylsilyloxyimino)-2,3-dihydro-1H-inden-5-yl)ethanone (0.125 g, 0.272 mmol; prepared according to Example 33, Steps A-C) was dissolved in 2 mL dry NMP under N₂. NaHMDS (0.453 mL, 0.272 mmol) was added and the reaction mixture was stirred at room temperature for 15 minutes before adding 1-isothiocyanatopropane (0.0281 mL, 0.272 mmol). The reaction mixture was stirred for 15 minutes at room temperature before subjecting the reaction to microwave conditions (130 degrees, for 2 minutes, 30 seconds). The reaction mixture was then cooled, diluted with ethyl acetate, washed with saturated ammonium chloride, water, dried over sodium sulfate, filtered and concentrated to a brown film. The residue was taken up in 2 mL THF and TBAF (0.544 mL, 0.544 mmol) was added. The reaction mixture was stirred for 10 minutes and passed thru a silica plug, eluting with THF. The filtrate was concentrated and purified by column chromatography using 1-4% MeOH/CHCl₃+1% NH₄OH. The desired product was isolated as a light yellow solid in 30% yield. MS (APCI) m/z=366.3 (M+1).

### Example 36

### Preparation of (1-(hydroxyimino)-2,3-dihydro-1H-inden-5-yl)(2-(2-methoxyethylamino)thieno[2,3-c]pyridin-3-yl)methanone

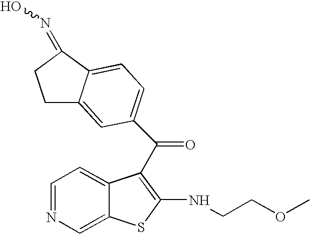

Prepared according to Example 33. MS (APCI) m/z=382.3 (M+1).

### Example 37

### (2-(3-(diethylamino)propylamino)thieno[2,3-c]pyridin-3-yl)(1-(hydroxyimino)-2,3-dihydro-1H-inden-5-yl)methanone

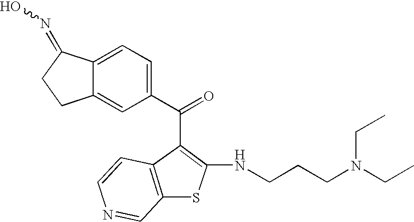

Prepared according to Example 33. MS (APCI) m/z=437.2 (M+1).

### Example 38

### Preparation of N-(3-(1-(hydroxyimino)-2,3-dihydro-1H-indene-5-carbonyl)thieno[2,3-c]pyridin-2-yl)propionamide

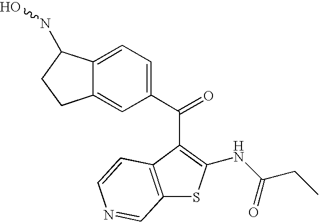

Step A: Preparation of N-(4-methoxybenzyl)-N-(3-(1-(hydroxyimino)-2,3-dihydro-1H-indene-5-carbonyl)thieno[2,3-c]pyridin-2-yl)propionamide: (2-(4-methoxybenzylamino)thieno[2,3-c]pyridin-3-yl)(1-(tert-butyldimethylsilyloxyimino)-2,3-dihydro-1H-inden-5-yl)methanone (0.015 g, 0.02689 mmol; prepared according to Example 32, Steps A-D) was dissolved in 1 mL dry THF at room temperature under N₂. NaHMDS in toluene (0.04930 mL, 0.02958 mmol) was added and the reaction mixture was stirred for 5 minutes before adding propionyl chloride (0.002804 mL, 0.03227 mmol). The reaction mixture was stirred at room temperature, then poured into water, extracted with ethyl acetate, filtered and purified by preparative TLC (50/40/10 EtOAc/Hex/MeOH) to provide the desired product in 71% yield.

Step B: Preparation of N-(3-(1-(hydroxyimino)-2,3-dihydro-1H-indene-5-carbonyl)thieno[2,3-c]pyridin-2-yl)propionamide: N-(4-methoxybenzyl)-N-(3-(1-(hydroxyimino)-2,3-dihydro-1H-indene-5-carbonyl)thieno[2,3-c]pyridin-2-yl)propionamide (0.024 g, 0.048 mmol) was dissolved in 700 μL MeCN at room temperature. Water (50 μL) was added, followed by addition of ceric ammonium nitrate (0.066 g, 0.12 mmol) in a single portion. The reaction mixture was stirred at room temperature, then concentrated. The residue was loaded onto a preparative silica TLC plate, and the desired compound was isolated in 53% yield after 2 plates using 10% MeOH/CH₂Cl₂, followed by 50/40/10 EtOAc/Hexanes/MeOH. MS (APCI) m/z=380.1 (M+1).

### Example 39

### Preparation of 5-(2-(pyrimidin-2-yl)furo[2,3-c]pyridin-3-ylamino)-2,3-dihydroinden-1-one oxime

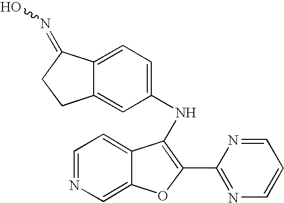

Step A: Preparation of methylpyrimidine-2-carboxlate: To a cold (0° C.) solution of saturated HCl in MeOH (60 mL) was added a solution of pyrimidine-2-carbonitrile (1.4 g, 13 mmol) in MeOH (10 mL). The reaction mixture was stirred at room temperature overnight. Methanol was removed and the resulting white solids were triturated with ether (200 mL). The solids were dissolved in water (20 mL) and the pH was adjusted to 4 with saturated NaHCO₃ The aqueous layer was extracted with CH₂Cl₂ (3×100 mL). The combined organics were dried, filtered and concentrated to give a white solid (0.8 g), which was used in the next step without purification.

Step B: Preparation of pyrimidin-2-ylmethanol: NaBH₄ (0.22 g, 5.79 mmol) was added in one portion to a solution of methylpyrimidine-2-carboxlate (0.80, 5.79 mmol) in EtOH (30 mL). The reaction mixture was left at room temperature overnight before carefully quenching with water (5.0 mL) and concentrating to dryness. The residue was taken up in MeOH and filtered. The filtrate was concentrated and was purified by flash column chromatography, eluting with CH₂Cl₂/MeOH (100:1) to give 0.18 g (28%) of the desired product as a yellow oil.

Step C: Preparation of 2-(pyrimidin-2-yl)furo[2,3-c]pyridin-3-amine: Prepared using the general procedure described in Example 29, Step A. MS (APCI) m/z 213.3 (M+1).

Step D: Preparation of 5-(2-(pyrimidin-2-yl)furo[2,3-c]pyridin-3-ylamino)-2,3-dihydroinden-1-one oxime: Prepared using the general procedure described in Example 29, Steps B and C. MS (APCI) m/z 358.3 (M+1).

### Example 40

### Preparation of 5-(2-(5-methylisoxazole-3-yl)furo[2,3-c]pyridin-3-ylamino)-2,3-dihydroinden-1-one oxime

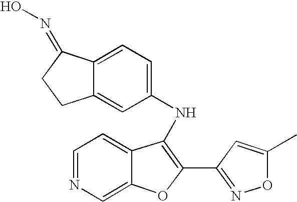

Step A: Preparation of 2-(5-methylisoxazole-3-yl)furo[2,3-c]pyridin-3-amine: To a cold (0° C.) suspension of NaH (60% dispersion in mineral oil, 0.053 g, 1.31 mmol) in DMF (2.0 mL) was added a solution of (5-methylisoxazole-3-yl)methanol (0.136, 1.20 mmol) in DMF (2.0 mL) dropwise. The reaction mixture was stirred for 10 minutes, and then a solution of 3-bromoisonicotinoitrile (0.200 g, 1.09 mmol) in DMF (5.0 mL) was added in dropwise. The reaction mixture was stirred at room temperature for 1 hour, then 80° C. for 1 hour before quenching with water (20 mL). The aqueous layer was extracted with ethyl acetate (3×50 mL). The combined organics were dried, filtered and concentrated. The crude product was purified by flash column chromatography, eluting with hexanes/ethyl acetate (2:1) to give 0.040 g (28% yield) of the desire product as a yellow solid. MS (APCI) m/z 216.2 (M+1).

Step B: Preparation of 5-(2-(5-methylisoxazole-3-yl)furo[2,3-c]pyridin-3-ylamino)-2,3-dihydroinden-1-one oxime: Prepared using the general procedure described in Example 29, Steps B and C. MS (APCI) m/z 361.2 (M+1).

### Example 41

### Preparation of 5-(2-(2-(trifluoromethyl)phenyl)furo[2,3-c]pyridin-3-ylamino)-2,3-dihydroinden-1-one oxime

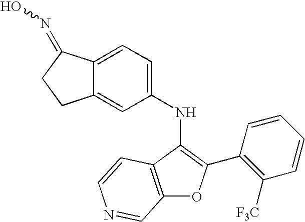

Step A: Preparation of 2-(2-(trifluoromethyl)phenyl)furo[2,3-c]pyridin-3-amine: To an ice cooled (0° C.) suspension of NaH (61.93 mg, 1.548 mmol) in DMF (5 mL) was added (2-(trifluoromethyl)phenyl)methanol (0.2262 mL 1.703 mmol) and the reaction mixture was stirred 10 minutes. 3-Bromoisonicotinonitrile (283.4 mg, 1.548 mmol) in 5 mL DMF was added, and the reaction mixture was stirred overnight while warming to 60° C., then cooled to room temperature, diluted with water and EtOAc, and the layers were separated. Purification by column chromatography (10% MeOH/CH₂Cl₂) provided 67 mg (15%) of the desired product. MS (APCI-pos) M+1=279.5.

Step B: Preparation of 5-(2-(2-(trifluoromethyl)phenyl)furo[2,3-c]pyridin-3-ylamino)-2,3-dihydroinden-1-one O-tert-butyldimethylsilyl oxime: 5-Bromo-2,3-dihydroinden-1-one O-tert-butyldimethylsilyl oxime (81.95 mg, 0.2408 mmol) and 2-(2-(trifluoromethyl)phenyl)furo[2,3-c]pyridin-3-amine (67.0 mg, 0.2408 mmol) were combined in toluene (8 mL) and Cs₂CO₃ (125.5 mg, 0.3853 mmol) was added. The reaction mixture was degassed with Ar for 10 minutes, then X-Phos (3.444 mg, 0.007224 mmol) and Pd₂(dba)₃ (11.03 mg, 0.01204 mmol) were added. The reaction mixture was heated under N₂ with a condenser at 110° C. for 4 hours. The reaction mixture was filtered (GF/F paper) and the filtrate was purified by silica gel chromatography to afford 72 mg (55%) of the desired product. MS (APCI-pos) M+1=538.3

Step C: Preparation of 5-(2-(2-(trifluoromethyl)phenyl)furo[2,3-c]pyridin-3-ylamino)-2,3-dihydroinden-1-one oxime: To a cooled (0° C.) solution of (5-(2-(2-(trifluoromethyl)phenyl)furo[2,3-c]pyridin-3-ylamino)-2,3-dihydroinden-1-one O-tert-butyldimethylsilyl oxime (71.9 mg, 0.134 mmol) in CH₂Cl₂ was added TBAF (0.140 mL, 0.140 mmol). After stirring 1 hour, the solution was quenched with aqueous NH₄Cl. The organic layer was separated, concentrated and purified via silica gel chromatography (5% MeOH/EtOAc) to provide 5.2 mg (9%) of the desired product. MS (APCI-pos) M+1=424.2.

### Example 42

### Preparation of 5-(2-(6-methylpyridin-2-yl)furo[2,3-c]pyridin-3-ylamino)-2,3-dihydroinden-1-one oxime

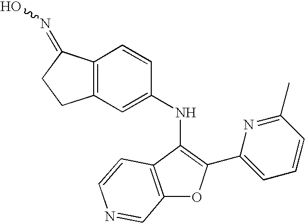

Step A: Preparation of 2-(6-methylpyridin-2-yl)furo[2,3-c]pyridin-3-amine: To a 0° C. solution of NaH (70.86 mg, 1.772 mmol) in DMF was added (6-methylpyridin-2-yl)methanol (200 mg, 1.624 mmol). After 10 minutes, 3-bromoisonicotinonitrile (270.2 mg, 1.476 mmol) in 5 mL DMF was added and the solution was warmed to 60° C. overnight. The reaction mixture was cooled to room temperature, diluted with H₂O and EtOAc, and the layers were separated. The organic layer was dried (MgSO₄), filtered, and concentrated to afford crude. 3-((6-methylpyridin-2-yl)methoxy)isonicotinonitrile as the intermediate. This material was dissolved in DMF and combined with NaH (59.66 mg, 1.492 mmol). The mixture was heated at 60° C. overnight, then cooled to room temperature, diluted with H₂O and EtOAc, and the layers were separated. The organic layer was concentrated and purified by silica gel chromatography (8% MeOH/CH₂Cl₂) provided 89 mg (32%) of the desired product. MS (APCI-pos) M+1=226.3.

Step B: Preparation of 5-(2-(6-methylpyridin-2-yl)furo[2,3-c]pyridin-3-ylamino)-2,3-dihydroinden-1-one O-tert-butyldimethylsilyl oxime: 5-Bromo-2,3-dihydroinden-1-one O-tert-butyldimethylsilyl oxime (134.0 mg, 0.3938 mmol) and 2-(2-(trifluoromethyl)phenyl)furo[2,3-c]pyridin-3-amine (88.7 mg, 0.3938 mmol) were combined in toluene (10 mL) and Cs₂CO₃ (205.3 mg, 0.6301 mmol) was added. The reaction mixture was degassed with Ar for 10 minutes, then X-Phos (5.632 mg, 0.01181 mmol) and Pd₂(dba)₃ (18.03 mg, 0.01969 mmol) were added. The reaction mixture was heated 110° C. for 4 hours under N₂ with a condenser. The reaction mixture was diluted with water and EtOAc, and the layers were separated. The organic layers were dried (MgSO₄) and purified by silica gel chromatography to afford 97 mg (51%) of the desired product. MS (APCI-pos) M+1=485.3.

Step C: Preparation of 5-(2-(6-methylpyridin-2-yl)furo[2,3-c]pyridin-3-ylamino)-2,3-dihydroinden-1-one oxime: To a cooled (0° C.) solution of 5-(2-(6-methylpyridin-2-yl)furo[2,3-c]pyridin-3-ylamino)-2,3-dihydroinden-1-one O-tert-butyldimethylsilyl oxime (96.6 mg, 0.199 mmol) in CH₂Cl₂ (2 mL) was added TBAF (0.199 mL, 0.199 mmol). After stirring 1 hour, the solution was quenched with aqueous NH₄Cl and the organic layer was isolated. Purification by silica gel chromatography (75% EtOAc/hexanes) afforded 32.3 mg (44%) of the desired product. MS (APCI-pos) M+1=371.3.

### Example 43

### Preparation of N-(2-(dimethylamino)ethyl)-3-(1-(hydroxyimino)-2,3-dihydro-1H-inden-5-ylamino)furo[2,3-c]pyridine-2-carboxamide

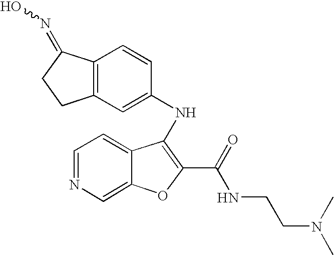

N1,N1-dimethylethane-1,2-diamine (0.0284 mL, 0.258 mmol) was dissolved in 3 mL dry toluene at 0° C. Trimethylaluminum (0.129 mL, 0.258 mmol) was added and the solution was stirred for 15 minutes before adding ethyl 3-(1-(tert-butyldimethylsilyloxyimino)-2,3-dihydro-1H-inden-5-ylamino)furo[2,3-c]pyridine-2-carboxylate (0.040 g, 0.0859 mmol) in a single portion. The reaction mixture was stirred for 5 minutes, heated to 80° C. for 90 minutes, and then cooled. Ice was added and the reaction mixture was extracted with ethyl acetate, filtered through celite, dried over sodium sulfate, filtered and concentrated to a yellow film. The film was taken up in 5 mL THF at room temperature, and TBAF (0.172 mL, 0.172 mmol) was added. The reaction mixture was stirred for 30 minutes, and then concentrated and purified by column chromatography using 14% MeOH/CHCl₃+1% NH₄OH to provide the desired product (79% yield) as a yellow solid. MS (APCI) m/z 394.1 (M+1).

### Example 44

### Preparation of 3-(1-(hydroxyimino)-2,3-dihydro-1H-inden-5-ylamino)-N-isopropylfuro[2,3-c]pyridine-2-carboxamide

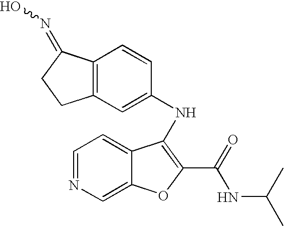

Propan-2-amine (0.0366 mL, 0.430 mmol) was dissolve in 3 mL dry toluene at 0° C. Trimethylaluminum (0.215 mL, 0.430 mmol) was added and the solution was stirred for 15 minutes before adding ethyl 3-(1-(tert-butyldimethylsilyloxyimino)-2,3-dihydro-1H-inden-5-ylamino)furo[2,3-c]pyridine-2-carboxylate (0.040 g, 0.0859 mmol) in a single portion. The reaction mixture was stirred for 5 minutes and then heated to 80° C. for 3 hours. The reaction mixture was cooled, and ice was added followed by sodium bicarbonate. The reaction mixture was extracted with ethyl acetate, dried over sodium sulfate, filtered and concentrated to a yellow film. The film was taken up in 5 mL THF, and TBAF (0.172 mL, 0.172 mmol) was added. The reaction mixture was stirred for 30 minutes, concentrated and purified by column chromatography using 1-4% MeOH/DCM+1% NH₄OH to provide the desired product (57% yield) as a yellow solid. MS (APCI) m/z 365.2 (M+1).

### Example 45

### 3-(1-(hydroxyimino)-2,3-dihydro-1H-inden-5-ylamino)-N-(pyridin-2-yl)furo[2,3-c]pyridine-2-carboxamide

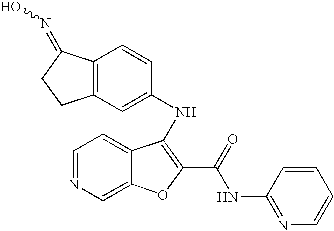

Prepared according to Example 44, substituting 2-aminopyridine for propan-2-amine. MS (APCI) m/z 400.2 (M+1).

### Example 46

### 3-(1-(hydroxyimino)-2,3-dihydro-1H-inden-5-ylamino)-N-(pyridin-2-ylmethyl)furo[2,3-c]pyridine-2-carboxamide

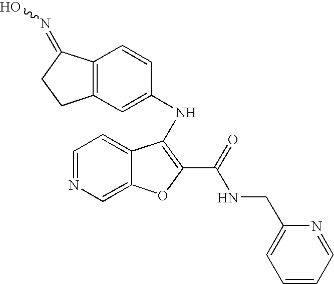

Prepared according to Example 44, substituting 2-(aminomethyl)pyridine for propan-2-amine. MS (APCI) m/z 414.3 (M+1).

### Example 47

### 3-(1-(hydroxyimino)-2,3-dihydro-1H-inden-5-ylamino)-N-(pyridin-3-ylmethyl)furo[2,3-c]pyridine-2-carboxamide

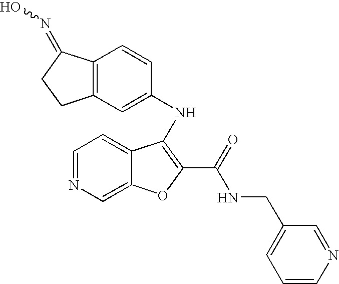

Prepared according to Example 44, substituting 3-(aminomethyl)pyridine for propan-2-amine. MS (APCI) m/z 414.3 (M+1).

### Example 48

### Ethyl 3-(trifluoromethylsulfonyloxy)furo[2,3-c]pyridine-2-carboxylate

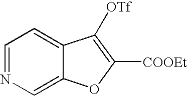

Step A: Ethyl 3-(2-ethoxy-2-oxoethoxy)isonicotinate: Triphenylphosphine (150.6 g, 574 mmol) was dissolved in THF (1 L) and cooled to −10° C. To this was added DIAD dropwise via an addition funnel over 30 minutes. The resulting white suspension was kept at −10° C. for another 30 minutes. Ethyl glycolate (50.84 mL, 526.4 mmol) was added as a solution in THF (500 mL) via the addition funnel at a rate to maintain the internal temperature below −10° C. Upon completion of addition, the reaction mixture was kept at −10° C. for additional 30 minutes before a suspension of ethyl 3-hydroxyisonicotinate (80 g, 478.6 mmol) in THF (500 mL) was added. The reaction was allowed to warm up slowly to ambient temperature overnight. The reaction mixture was concentrated, and the residue was taken up in ethyl acetate (1 L) and extracted with 1N HCl. The aqueous layer was treated with NaHCO₃ to pH ˜8 and then extracted with ethyl acetate. The combined organic layers were dried, filtered and concentrated to give the desired product (92.0 g, 76%). MS (APCI) m/z 254.3 (M+1).

Step B: Ethyl 3-hydroxyfuro[2,3-c]pyridine-2-carboxylate: Ethyl 3-(2-ethoxy-2-oxoethoxy)isonicotinate (92.0 g, 363 mmol) was added dropwise via an addition funnel as a solution in THF (300 mL) to a suspension of NaH (17.4 g, 436 mmol, 60% suspension in mineral oil) in 200 mL of cold THF (0° C.). Upon complete addition, the reaction mixture was allowed to warm up to ambient temperature overnight. The reaction mixture was cooled to 0° C., carefully quenched with ice and then concentrated to remove most of the THF. The remaining yellow slurry was diluted with saturated NaHCO3 (1 L) and stirred for 30 minutes. The solids were collected by filtration, washed with water and ethyl acetate. The filtrate was washed with ethyl acetate. The aqueous layer was pooled with the solids and carefully acidified to pH-5 with AcOH (100 ml). The resulting yellow solids were collected by filtration and dried under vacuum overnight to give the desired product (63.4 g, 84%). ¹HNMR (400 MHz, CDCl₃) δ 8.9 (s, 1H), 8.5 (d, J=4.8 Hz, 1H), 7.7 (d, J=5.2 Hz, 1H), 4.5 (q, J=7.0 Hz, 2H), 1.5 (t, J=7.0 Hz, 3H) ppm. MS (APCI) m/z 208.2 (M+1).

Step C: Ethyl 3-(trifluoromethylsulfonyloxy)furo[2,3-c]pyridine-2-carboxylate: To a cold (0° C.) solution of 3-hydroxyfuro[2,3-c]pyridine-2-carboxylate (4.6 g, 22.2 mmol), pyridine (2.33 mL, 28.9 mmol) in dichloromethane (50 mL) was added Tf2O (4.50 mL, 26.6 mmol) dropwise. After 2 hours, the reaction mixture was quenched with water and the aqueous layer was extracted with DCM. The combined organic layers were dried, filter and concentrated. The crude product was purified by flash column chromatography, eluting with hexanes/ethyl acetate (4:1) to give the desired product (6.74 g, 90%). MS (APCI) m/z 340.0 (M+1).

### Example 49

### General Procedure for the Removal of BOM Protecting Groups

Dissolve the benzyloxymethoxy protected hydroxide in 10 mL EtOH. Added 2 mL 6M HCl and heat the mixture to 60° C. for 2 hours. The reaction mixture is transferred to a separatory funnel, diluting with water and brine, and the pH of the mixture is adjusted to ˜5 using saturated aqueous NaHCO₃. Extract with ethyl acetate, separate the organic layer and dry over sodium sulfate, filter and concentrate under vacuum. Purify the residue by silica gel chromatography to isolate the desired compound.

### Example 50

### General Procedure for the Boron Tribromide Deprotection of Methyl Ethers

The starting methyl ether is dissolved in dichloromethane and cooled to −78° C. using acetone/dry ice. BBr3 (3.00 equiv) is added the reaction mixture is stirred while warming to ambient temperature. The mixture is transferred to a separatory funnel, diluting with dichloromethane and water. The pH of the mixture is adjusted to 4-5 and extracted with CH₂Cl₂. The organic layers are combined and dried over sodium sulfate, then filtered and concentrated under vacuum. The crude product is purified by silica gel chromatography.

### Example 51

### General Procedure for TFA Deprotection of O-tert-butyldimethylsilyl Oximes

The starting tert-butyldimethylsilyl-protected oxime is dissolved in organic solvent (dichloromethane or THF) and TFA is added via pipette. The mixture is stirred at ambient temperature for 2 hours. The reaction mixture is transferred to a separatory funnel and diluted with CH₂Cl₂ and water. The pH is adjusted to ˜4-5 using saturated aqueous NaHCO₃ and the mixture is extracted with dichloromethane. The organic layers are combined, dried over sodium sulfate, filtered and concentrated under vacuum. The crude product is purified by silica gel chromatography.

### Example 52

### General Procedure for TBAF Deprotection of O-tert-butyldimethylsilyl Protected Hydroxides

The O-tert-butyldimethylsilyl hydroxide is dissolved in THF (5 mL) and cooled in an ice bath. The solution is treated with a solution of tetrabutylammonium fluoride (1.0 M in THF, 1.3 equiv) and the reaction is stirred 10 minutes at 0° C. The reaction is quenched with aqueous NH₄Cl and extracted with EtOAc. The combined organic layers are dried (sodium sulfate), filtered and concentrated, and the crude product purified by silica gel chromatography.

### Example 53

### General Procedure for Xantphos/Pd₂(dba)₃-Catalyzed Coupling of Amines with Aryl Triflates

The amine (1.1 equiv), ethyl 3-(trifluoromethylsulfonyloxy)furo[2,3-c]pyridine-2-carboxylate (1.0 equiv), potassium phosphate (1.2 equiv), Xantphos (0.15 equiv) and Pd₂(dba)₃ (0.15 equiv) are dissolved in toluene and the mixture is degassed with Ar bubbling for about 15 minutes. The mixture is heated to 110° C. for 20 hours, then cooled to ambient temperature, diluted with CH₂Cl₂, and filtered through GF/F paper, and rinsed with CH₂Cl₂. The crude product purified by silica gel chromatography. (eluting with either ethyl acetate/hexanes or ethanol/chloroform gradients).

### Example 54

### General Procedure for the Condensation of furo[2,3-c]pyridin-3(2H)-one Hydrochloride with Anilines

A solution of [2,3-c]pyridin-3(2H)-one hydrochloride and the aniline is heated at reflux for 20 hours in methanol. Aqueous NaHCO₃ is added and the mixture is extracted with ethyl acetate. The combined organic layers are dried (sodium sulfate), filtered and concentrated, and the crude product purified by silica gel chromatography (eluting with either ethyl acetate/hexanes or ethanol/chloroform gradients).

### Example 55

### General Procedure for the Triflation of furo[2,3-c]pyridin-3-ols

The furo[2,3-c]pyridin-3-ol (1.0 equiv) is stirred in CH₂Cl₂ with pyridine (1.5 equiv) at 0° C., and Tf₂O (1.2 equiv) was added. If reaction is not complete by TLC analysis after 1 hour, additional pyridine and Tf₂O may be added. Once reaction is complete, water is added and the layers are separated. The aqueous layer is extracted once with CHCl₃, and the combined organics are dried (sodium sulfate). After filtration, the crude material is purified by silica gel chromatography (eluting with EtOAc/hexanes) to afford the desired triflate.

### Example 56

### General Procedure for the EDCI-Mediated Transformation of Carboxylic Acids to Amides

The carboxylic acid (1.0 equiv) is dissolved in CH₂Cl₂ and the appropriate amine hydrochloride (1.5 equiv), DIEA (4 equiv), EDCI•HCl (2 equiv) and HOBT•H₂O (0.1 equiv) are added successively. The reaction mixture is stirred 15 hours at ambient temperature, then diluted with saturated NaHCO₃ and extract with CH₂Cl₂. The organic layers are washed with brine, dried over sodium sulfate, and concentrated, and the crude product is purified by silica gel chromatography.

### Example 57

### 5-(2-(4-(Trifluoromethyl)phenyl)furo[2,3-c]pyridin-3-ylamino)-2,3-dihydro-1H-inden-1-one oxime

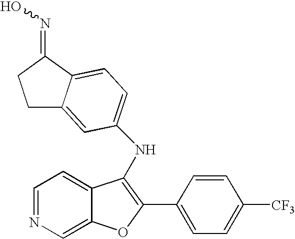

Prepared according to the method of Example 41. MS (APCI-pos) M+1=424.3. ¹H NMR (400 MHz, d₄-MeOD) δ 8.92 (s, 1H), 8.34-8.32 (m, 1H), 8.25-8.23 (m, 3H), 7.78-7.76 (m, 3H), 7.49-7.46 (m, 2H), 6.74-6.64 (m, 2H), 2.93-2.67 (m, 4H).

### Example 58

### 5-(2-(Pyridin-2-yl)furo[2,3-c]pyridin-3-ylamino)-2,3-dihydro-1H-inden-1-one

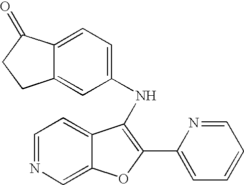

Prepared according to the method of Example 41. MS (APCI-neg) M−1=340.5 ¹H NMR (400 MHz, CDCl₃) δ 9.21 (s, 1H), 8.96 (s, 1H), 8.67-8.66 (d, J=4.8 Hz, 1H), 8.43-8.41 (d, J=5.4 Hz, 1H), 7.95-7.93 (d, J=7.6 Hz, 1H), 7.88-7.84 (m, 1H), 7.72-7.70 (d, J=8.1 Hz, 1H), 7.40-7.39 (d, J=5.7 Hz, 1H), 7.05-7.02 (m, 2H), 3.08-3.05 (t, J=5.9 Hz, 2H), 2.70-2.67 (m, 2H).

### Example 59

### 5-(2-p-Tolylfuro[2,3-c]pyridin-3-ylamino)-2,3-dihydro-1H-inden-1-one oxime

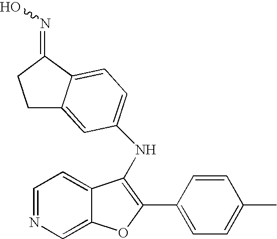

Step A: 2-p-Tolylfuro[2,3-c]pyridin-3-ol: A flame-dried 25 mL round-bottom flask was charged with NaOt-Bu (199 mg, 2.4 equiv), 1-bromo-4-methylbenzene (149 mg, 1.0 equiv), and toluene (2.5 mL). The mixture was degassed under Ar for 10 minutes, then Pd(OAc)₂ (7 mg, 0.03 equiv), X-Phos (29 mg, 0.07 equiv), and furo[2,3-c]pyridin-3(2H)-one hydrochloride (178 mg, 1.2 equiv) were sequentially added. The mixture was heated at 70° C. under Ar for 17 hours. The reaction was cooled, filtered through GF/F paper, and the contents were partitioned between EtOAc and water. The aqueous layer was separated and extracted 3× with additional EtOAc. The combined organics were dried (MgSO₄), filtered, concentrated, and purified by silica gel chromatography (100% EtOAc) to afford the product as a pale yellow solid (16 mg, 8%). MS (APCI-pos) M+1=226.3.

Step B: 2-p-tolylfuro[2,3-c]pyridin-3-yl trifluoromethanesulfonate: Following the procedure of Example 55, the desired product was prepared from the product of Step A as a white solid in 43% yield.

Step C: 5-(2-p-tolylfuro[2,3-c]pyridin-3-ylamino)-2,3-dihydro-1H-inden-1-one: Following the procedure of Example 53, the product was obtained from the product of Step B in 46% yield. MS (APCI-pos) M+1=355.4.

Step D: 5-(2-p-tolylfuro[2,3-c]pyridin-3-ylamino)-2,3-dihydro-1H-inden-1-one oxime: 5-(2-p-Tolylfuro[2,3-c]pyridin-3-ylamino)-2,3-dihydro-1H-inden-1-one (5 mg) in ethanol (5 mL). was treated with 50% NH₂OH in water (0.1 mL), and the mixture heated at reflux overnight. The volatiles were removed and the crude material was purified by silica gel chromatography (75% EtOAc/hexanes) to afford the product as a yellow solid (3 mg, 58%). MS (APCI-pos) M+1=370.3. ¹H NMR (400 MHz, CDCl₃) δ 8.92-8.90 (m, 1H), 8.39-8.28 (m, 1H), 7.93-7.89 (m, 2H), 7.55-7.51 (m, 1H), 7.30-7.26 (m, 4H), 6.73-6.68 (m, 1H), 6.62-6.60 (m, 1H), 5.54-5.45 (m, 1H), 2.95-2.61 (m, 4H), 2.40 (s, 3H).

### Example 60

### 6-(2-(4-ethyl-1H-imidazol-2-yl)furo[2,3-c]pyridin-3-ylamino)naphthalen-1-ol

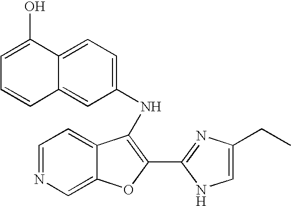

Step A: N-(5-(tert-butyldimethylsilyloxy)naphthalen-2-yl)-2-(4-ethyl-1H-imidazol-2-yl)furo[2,3-c]pyridin-3-amine: 3-(5-(tert-Butyldimethylsilyloxy)naphthalen-2-ylamino)furo[2,3-c]pyridine-2-carboxamidine (0.0086 g, 0.01988 mmol) was treated with KHCO₃ (0.01990 g, 0.1988 mmol) then a solution of 1-bromobutan-2-one (0.002030 mL, 0.01988 mmol) dissolved in THF/water (4:1, 0.1 mL) was added. The reaction was stirred at ambient temperature for 1 hour then heated to reflux for 3 h and stirred at ambient temperature for 12 hours. The reaction mixture was filtered and the filtrate was concentrated with N₂ (g). The residue was applied to samplet cartridge with methylene chloride then chromatographed on SiO₂ (Biotage 12S) eluting with 3% MeOH/methylene chloride then with 3% MeOH/1% NH₄OH/methylene chloride. The desired product was recovered as a yellow solid (4.3 mg, 45%).

Step B: 6-(2-(4-ethyl-1H-imidazol-2-yl)furo[2,3-c]pyridin-3-ylamino)naphthalen-1-ol: The product of Step A was deprotected with tetrabutylammonium fluoride as described is Example 52 to provide the desired product as a solid (1.6 mg, 50%). MS (ESI+) m/z 371.5. ¹H NMR (CDCl₃, 400 MHz) δ 8.79 (s, 1H), 8.26-8.22 (m, 1H), 8.20-8.14 (m, 1H), 7.36-7.29 (m, 2H), 7.28-7.18 (m, 2H), 7.16-7.09 (m, 2H), 6.70-6.66 (m, 1H), 2.77-2.67 (m, 2H), 1.26 (t, 3H).

### Example 61

### 6-(2-(4-tert-butyl-1H-imidazol-2-yl)furo[2,3-c]pyridin-3-ylamino)naphthalen-1-ol

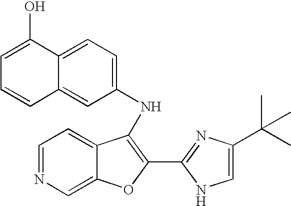

Prepared as described in Example 60, Step A, substituting 1-bromo-3,3-dimethylbutan-2-one. MS (ESI+) m/z 399.3. ¹H NMR (CDCl₃, 400 MHz) δ 9.47 (brd s, 1H), 9.37 (brd s, 1H), 8.87 (s, 1H), 8.37-8.29 (m, 1H), 8.16-8.10 (m, 1H), 8.17-8.00 (m, 1H), 7.40-7.28 (m, 1H), 7.25-7.10 (m, 4H), 6.70-6.61 (m, 1H), 1.40 (s, 9H).

### Example 62

### Ethyl 3-(4-chloro-3-methoxyphenylamino)furo[2,3-c}pyridine-2-carboxylate

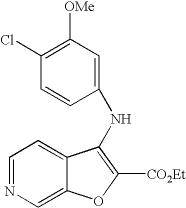

Prepared as in Example 26, step B, substituting biphenyl-2-yldi-tert-butylphosphine as the catalyst. ¹HNMR (400 MHz, CDCl₃) δ 9.0 (s, 1H), 8.3 (d, J=5.4 Hz, 1H), 7.8 (bs, 1H), 7.3 (d, J=7.7 Hz, 1H), 7.2 (d, J=5.5 Hz, 1H), 6.7 (m, 2H), 4.5 (q, J=7.0 Hz, 2H), 3.8 (s, 3H), 1.5 (t, J=7.1 Hz, 3H). MS (APCI) m/z 347.2 (M+1).

### Example 63

### Ethyl 3-(5-aminonaphthalen-2-ylamino)furo[2,3-c]pyridine-2-carboxylate

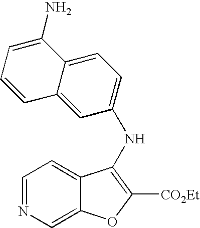

Step A: 2-(5-hydroxynaphthalen-2-yl)isoindoline-1,3-dione: 6-Aminonaphthalen-1-ol (1.04 g, 6.53 mmol) and isobenzofuran-1,3-dione (0.974 g, 6.58 mmol) were dissolved in toluene (10 mL) and the mixture was heated to 125° C. (employing Dean-Stark trap) for 20 hours. The residual toluene was removed under reduced pressure to provide the product as solid (1.6 g, 86%).

Step B: 6-(1,3-dioxoisoindolin-2-yl)naphthalen-1-trifluoromethanesulfonate: 2-(5-hydroxynaphthalen-2-yl)isoindoline-1,3-dione (614 mg, 2.12 mmol) and pyridine (0.45 mL, 2.6 equiv) were slurried in CH₂Cl₂ and Tf₂O (0.45 mL, 1.3 equiv) was added. After stirring 1.5 hours, the reaction was diluted with water, and the layers separated. The organic layer was dried (sodium sulfate), filtered, concentrated, and purified by silica gel chromatography (eluting with 50% EtOAc/hexanes) to afford the product as a solid (900 mg, quant.).

Step C: N1,N1-bis-(tert-butoxycarbonyl)-naphthalene-1,6-diamine: Following the general procedure for XantPhos coupling (53) using BocNH₂ (3.0 equiv) as the amine, Cs₂CO₃ (1.6 equiv) as the base, and THF as the solvent, the intermediate product was treated with Boc2O and DMAP, but no reaction appears to take place, suggesting that the product from the XantPhos coupling was the bis-Boc material. This material was dissolved in MeCN and hydrazine (1.3 equiv) was added and was stirred for 16 hours at ambient temperature. Water and EtOAc were added, and the layers were separated. The organics were dried (MgSO₄) and purified by silica gel chromatography (20% EtOAc/hexanes) to afford N1,N1-di-Boc-naphthalene-1,6-diamine.

Step D: Ethyl 3-(5-aminonaphthalen-2-ylamino)furo[2,3-c]pyridine-2-carboxylate: Following the procedure of Example 53, the product was obtained as a solid (74% yield). MS (APCI-pos) M+1=548.0. Subsequent deprotection using TFA afforded the desired product. MS (APCI-pos) M+1=348.1. ¹H NMR (400 MHz, CDCl₃) δ 8.97 (s, 1H), 8.28-8.26 (m, 1H), 7.89 (s, 1H), 7.86-7.83 (m, 1H), 7.48 (m, 1H), 7.31-7.27 (m, 2H), 7.19-7.17 (m, 2H), 6.76-6.74 (m, 1H), 4.54-4.49 (m, 2H), 4.18 (br s, 2H), 1.51-1.47 (m, 3H).

### Example 64

### Ethyl 3-(2-methylquinazolin-6-ylamino)furo[2,3-c]pyridine-2-carboxylate

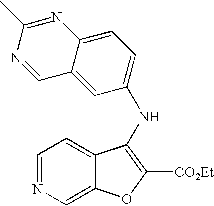

Step A: 2-Methyl-6-nitroquinazoline (prepared according to D. V. Dar'in, S. I. Selivanov, P. S. Lobanov, A. A. Potekhin, Chemistry of Heterocyclic Compounds, 2004, 40 (7), 888-894) was dissolved in methanol and Pd/C, and stirred for 3 hours under an atmosphere of H₂. The mixture was filtered though GF/F paper (rinsing with methanol) and purified by silica gel chromatography (eluting with 5% MeOH/CHCl₃) to afford 2-methylquinazolin-6-amine.

Step B: Following the procedure of Example 53, ethyl 3-(2-methylquinazolin-6-ylamino)furo[2,3-c]pyridine-2-carboxylate was obtained from the product of step A as a solid. MS (APCI-pos) M+1=349.2. ¹H NMR (400 MHz, CDCl₃) δ 9.19 (s, 1H), 9.02 (s, 1H), 8.37-8.35 (m, 1H), 7.98-7.94 (m, 2H), 7.71-7.69 (m, 1H), 7.46 (m, 1H), 7.19-7.18 (m, 1H), 4.55-4.50 (m, 2H), 2.90 (s, 3H), 1.60-1.48 (m, 3H).

### Example 65

### Ethyl 3-aminofuro[2,3-c]pyridine-2-carboxylate

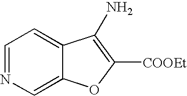

Step A: ethyl 3-(tert-butoxycarbonyl)furo[2,3-c]pyridine-2-carboxylate: Ethyl 3-(trifluoromethylsulfonyloxy)furo[2,3-c]pyridine-2-carboxylate and tert-butyl carbamate were reacted as described in Example 53 using XPhos as catalyst and cesium carbonate as the base to provide the desired compound.

Step B: Ethyl 3-aminofuro[2,3-c]pyridine-2-carboxylate: The crude product from step A was dissolved in cold (0° C.) dichloromethane (40 mL) and to this was added TFA (40 mL) dropwise via an addition funnel. The cold bath was removed and the reaction mixture was left at ambient temperature overnight. The reaction mixture was concentrated and the residue was dissolved in 2N HCl (200 mL). The aqueous layer was washed with ethyl acetate. The acidic aqueous layer was transferred to a 2 L Erlenmeyer flask containing 200 mL 2N NaOH and 400 mL ethyl acetate. Solid NaHCO₃ was carefully added in small portions until pH˜7. The aqueous layer was extracted with ethyl acetate. The combined organics were dried, filtered and concentrated to give the product as a solid. (3.0 g, 66%). ¹HNMR (400 MHz, CDCl₃) δ 8.9 (s, 1H), 8.5 (d, J=5.4 Hz, 1H), 7.5 (d, J=5.5 Hz, 1H), 5.0 (bs, 2H), 4.5 (q, J=7.0 Hz, 2H), 1.5 (t, J=7.1 Hz, 3H). MS (APCI) m/z 307.2 (M+1).

### Example 66

### Ethyl 3-(6-fluoro-5-hydroxynaphthalen-2-ylamino)furo[2,3-c]pyridine-2-carboxylate

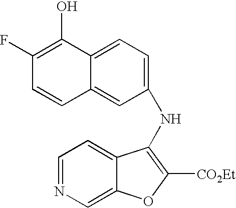

Step A: 2,2,2-trifluoro-N-(5-hydroxynaphthalen-2-yl)acetamide: To a cold (0° C.) solution of 5-(tert-butyldimethylsilyloxy)naphthalen-2-amine (12.3 g, 44.5 mmol) in dichloromethane (100 mL) was added DIPEA (10.2 mL, 58.5 mmol), followed by TFAA (7.0 mL, 49.5 mmol). The reaction was stirred at ambient temperature for 2 hours before quenching with water (100 mL). The aqueous layer was extracted with dichloromethane (200 mL×2). The combined organic extracts were dried, filtered and concentrated. The resulting brown oil was dissolved in THF (100 mL) and treated with TBAF (1.0 M in THF, 45.0 mL, 45.0 mmol). The reaction was stirred at ambient temperature for 30 minutes before quenching with water (50 mL). The aqueous layer was extracted with dichloromethane. The combined organics were dried, filtered and concentrated. The crude product was purified by flash column chromatography, eluting with dichloromethane/ethyl acetate (8:1) to give the desired product (10.0 g, 87%).

Step B: 2,2,2-Trifluoro-N-(6-fluoro-5-hydroxynaphthalen-2-yl)acetamide: To a solution of 2,2,2-trifluoro-N-(5-hydroxynaphthalen-2-yl)acetamide (1.50 g, 5.9 mmol) in dichloromethane (100 mL) was added 1-fluoro-4,6-bis(trifluoromethyl)pyridium-2-sulfonate (1.84 g, 5.9 mmol). The reaction was stirred at ambient temperature for 16 hours before quenching with 2N HCl (100 mL). The dark solids were removed by filtration. The aqueous layer was extracted with dichloromethane (100 mL×3). The combined organics were dried, filtered and concentrated. The crude product was purified by flash column chromatography eluting with dichloromethane to give the desired product (0.65 g, 40%).

Step C: 5-(tert-butyldimethylsilyloxy)-6-fluoronaphthalen-2-amine: To a solution of 2,2,2-trifluoro-N-(6-fluoro-5-hydroxynaphthalen-2-yl)acetamide (0.5 g, 1.28 mol) in MeOH (4.0 mL) was added 2N NaOH (4.0 mL). The reaction mixture was heated at reflux for 2 hours and then concentrated. The residue was diluted with water (10 mL) and ethyl acetate (50 mL). The pH was adjusted to ˜7 with HOAc. The aqueous layer was extracted with ethyl acetate (50 mL×2). The combined organics were dried, filtered and concentrated. The residue was suspended in dichloromethane (20 mL) and treated with imidazole (0.13 g, 1.92 mmol), followed by tert-butylchlorodimethylsilane (0.29 g, 1.92 mmol). The reaction mixture was stirred for 1 hour and quenched with water (20 mL). The aqueous layer was extracted with dichloromethane (50 mL×2). The combined organics were dried, filtered and concentrated. The crude product was purified by flash column chromatography, eluting with hexanes/ethyl acetate (10:1) to give the desired product (268 mg, 72%).

Step D: Ethyl 3-(6-fluoro-5-hydroxynaphthalen-2-ylamino)furo[2,3-c]pyridine-2-carboxylate: The product of step C was reacted according to the method of Example 53, followed by TBAF deprotection according to the method of Example 52, to provide the desired product. ¹HNMR (400 MHz, DMSO-d₆) δ 10.1 (bs, 1H), 9.1 (s, 1H), 8.6 (s, 1H), 8.3 (d, J=5.5 Hz, 1H), 8.1 (d, J=9.0 Hz, 1H), 7.4-7.2 (m, 5H), 4.4 (q, J=7.0 Hz, 2H), 1.3 (t, J=7.0 Hz, 3H). MS (APCI) m/z 367.2 (M+1).

### Example 67

### Ethyl 3-((5-hydroxynaphthalen-2-yl)(methyl)amino)furo[2,3-c]pyridine-2-carboxylate

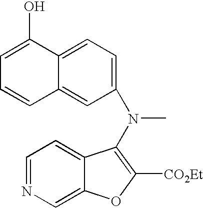

Step A: 5-(tert-butyldimethylsilyloxy)naphthalen-2-amine: To a cold (0° C.) suspension of 6-aminonaphthalen-1-ol (0.51 g, 3.2 mmol) in dichloromethane (20 mL) was added imidazole (0.327 g, 4.8 mmol), followed by tert-butylchlorodimethylsilane (0.745 g, 4.8 mmol). The reaction was stirred for 1 hour and then quenched with water (20 mL). The aqueous layer was extracted with dichloromethane. The combined organics were dried, filtered and concentrated. The crude product was purified by flash column chromatography, eluting with dichloromethane to give the desired product (824 mg, 94%). MS (APCI) m/z 274.2 (M+1).

Step B: tert-butyl 5-(tert-butyldimethylsilyloxy)naphthalen-2-ylcarbamate: To a solution of 5-(tert-butyldimethylsilyloxy)naphthalen-2-amine (0.30 g, 1.1 mmol) in dichloromethane (4.0 mL) was added catalytic amount of DMAP (˜10 mg) followed by (Boc)₂O (0.287 g, 1.32 mmol). Reaction mixture was stirred at ambient temperature overnight before quenching with water (4.0 mL). The aqueous layer was extracted with dichloromethane. The combined organics were dried, filtered and concentrated. The crude material was purified by flash column chromatography, eluting with hexanes/ethyl acetate (20:1) to give the desired product (383 mg, 94%).

Step C: 5-(tert-butyldimethylsilyloxy)-N-methylnaphthalen-2-amine: To a cold (0° C.) solution of 5-(tert-butyldimethylsilyloxy)naphthalen-2-ylcarbamate (0.383 g, 1.03 mmol) in THF (10 mL) was added LAH (0.156 g, 4.1 mmol). The cold bath was removed and the reaction mixture was heated at reflux under N₂ overnight, then cooled to 0° C. and carefully quenched with sNa₂SO₄.12H₂O. The solids were removed by filtration and the filtrate was concentrated and re-suspended in cold (0° C.) dichloromethane (4.0 mL). To this was added imidazole (0.105 g, 1.54 mmol), followed by tert-butylchlorodimethylsilane (0.232 g, 1.54 mmol). The reaction mixture was stirred for 2 hours and quenched with water (20 mL). The aqueous layer was extracted with dichloromethane. The combined organics were dried, filtered and concentrated. The crude product was purified by flash column chromatography, eluting with hexanes/ethyl acetate (50:1) to give the desired product (100 mg, 33%).

Step D: Ethyl 3-((5-hydroxynaphthalen-2-yl)(methyl)amino)furo[2,3-c]pyridine-2-carboxylate: The compound was prepared from the product of Step C using the procedure of Example 53, followed by the procedure of Example 52, in 30% yield. ¹HNMR (400 MHz, CDCl₃) δ 9.0 (s, 1H), 8.3 (d, J=5.5 Hz, 1H), 8.0 (d, J=9.2 Hz, 1H), 7.3-7.2 (m, 3H), 7.0 (m, 2H), 6.7 (d, J=6.0 Hz, 1H), 4.4 (q, J=7.0 Hz, 2H), 3.6 (s, 3H), 1.3 (t, J=7.0 Hz, 3H). MS (APCI) m/z 363.2 (M+1).

### Example 68

### Ethyl 3-(3-aminobenzo[d]isoxazol-6-ylamino)furo[2,3-c]pyridine-2-carboxylate

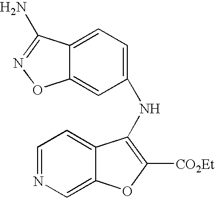

Step A: 6-Bromobenzo[d]isoxazol-3-amine: N-hydroxyacetamide (1.13 g, 15.0 mmol) was dissolved in DMF (20 mL). To this was added KOt-Bu (1.68 g, 15.0 mmol) and the reaction was stirred for 30 minutes before addition of 4-bromo-2-fluorobenzonitrile (2.0 g, 10.0 mmol). The reaction mixture was left at ambient temperature for 2 hours, then diluted with ethyl acetate (50 mL) and water (50 mL). The aqueous layer was extracted with ethyl acetate The combined organics were washed with water, dried, filtered and concentrated. The crude product was purified by flash column chromatography, eluting with ethyl acetate/hexanes (1:4), ethyl acetate/hexanes (1:3) to give the desired product (1.35 g, 63%). MS (APCI) m/z 215.2, 217.1 (M+1).

Step B: di-tert-butyl 6-bromobenzo[d]isoxazol-3-ylcarbamate: To a suspension of 6-bromobenzo[d]isoxazol-3-amine (0.5 g, 2.35 mmol) in dichloromethane (20 mL) was added (Boc)₂O (1.3 g, 5.7 mmol) and catalytic amount of DMAP (˜10 mg). The reaction mixture was stirred for 3 hours and quenched with water (20 mL). The aqueous layer was extracted with dichloromethane. The combined organics were dried, filtered and concentrated. The crude material was purified by flash column chromatography, eluting with dichloromethane to give the desired product (0.96 g, 99%).

Step C: Ethyl 3-(3-di-tert-butoxycarbonylaminobenzo[d]isoxazol-6-ylamino)furo[2,3-c]pyridine-2-carboxylate: Ethyl 3-aminofuro[2,3-c]pyridine-2-carboxylate and di-tert-butyl 6-bromobenzo[d]isoxazol-3-ylcarbamate were coupled according to the procedure of Example 4 using cesium carbonate as base (69% yield). MS (APCI) m/z 539.0 (M+1). TFA deprotection carried out as in Example 51. (80% yield). ¹HNMR (400 MHz, CDCl₃) δ 9.0 (s, 1H), 8.4 (d, J=4.5 Hz, 1H), 7.9 (s, 1H), 7.5 (d, J=7.8 Hz, 1H), 7.3 (d, J=4.8 Hz, 1H), 7.1 (s, 1H), 7.0 (d, J=8.9 Hz, 1H), 4.5 (q, J=7.0 Hz, 2H), 1.5 (t, J=7.0 Hz, 3H). MS (APCI) m/z 339.1 (M+1).

### Example 69

### 3-(3-Hydroxybenzo[d]isoxazol-6-ylamino)furo[2,3-c]pyridine-2-carboxylate

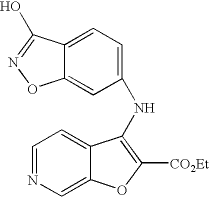

Step A: 6-bromobenzo[d]isoxazol-3-ol: N-hydroxyacetamide (0.99 g, 12.9 mmol) was dissolved in DMF (20 mL). To this was added KOt-Bu (1.44 g, 12.9 mmol) and the reaction was stirred for 30 minutes before addition of methyl 4-bromo-2-fluorobenzoate (2.0 g, 8.58 mmol). The reaction mixture was stirred at ambient temperature for 10 days, then diluted with ethyl acetate (50 mL) and 1N NaOH (50 mL). The aqueous layer was washed with ethyl acetate, then acidified with 2N HCl (30 mL). The desired product was collected by filtration (570 mg, 31%).

Step B: tert-butyl 6-bromo-3-oxobenzo[d]isoxazole-2(3H)-carboxylate: To a suspension of 6-bromobenzo[d]isoxazol-3-ol (0.183 g, 0.86 mmol) in THF (8.0 mL) was added 1N NaOH (4.28 mL, 4.28 mmol), followed by (Boc)₂ (0.93 g, 4.28 mmol). The reaction material was stirred at ambient temperature for 72 hours before quenching with water (10 mL). The aqueous layer was separated and extracted with ethyl acetate. The combined organics were dried, filtered and concentrated. The crude material was purified by flash column chromatography, eluting with hexanes/ethyl acetate (20:1) to give the desired product (265 mg, 99%).

Step C: 3-(3-hydroxybenzo[d]isoxazol-6-ylamino)furo[2,3-c]pyridine-2-carboxylate: Prepared using the general procedure described in Example 26 using cesium carbonate as the base. ¹HNMR (400 MHz, CDCl₃) δ 9.0 (s, 1H), 8.4 (d, J=5.4 Hz, 1H), 7.9 (s, 1H), 7.7 (d, J=8.7 Hz, 1H), 7.3 (d, J=5.4 Hz, 1H), 7.0 (m, 3H), 4.5 (q, J=7.2 Hz, 2H), 1.5 (t, J=7.2 Hz, 3H). MS (APCI) m/z 340.1 (M+1).

### Example 70

### Ethyl 3-(4-hydroxyisoquinolin-7-ylamino)furo[2,3-c]pyridine-2-carboxylate

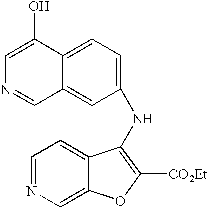

Step A: Methyl 2-(bromomethyl)-4-nitrobenzoate: Methyl 2-methyl-4-nitrobenzoate (4.2 g, 21.5 mmol) was dissolved in 100 mL CCl₄ under nitrogen. N-bromosuccinimide (6.13 g, 34.4 mmol) was added, followed by benzoyl peroxide (0.104 g, 0.430 mmol). The reaction mixture was heated overnight at 85° C. Added 1 g NBS followed by 100 mg benzoyl peroxide and continued heating the reaction for 6 hours. The reaction mixture was cooled to ambient temperature, poured into 1M HCl, extracted with dichloromethane, dried over magnesium sulfate, filtered and concentrated to an oil. Purification was carried out using column chromatography (5-10% EtOAc/hexanes).

Step B: Methyl 2-((N-(2-methoxy-2-oxoethyl)-4-methylphenylsulfonamido)methyl)-4-nitrobenzoate: Methyl 2-(4-methylphenylsulfonamido)acetate (4.314 g, 17.73 mmol) was dissolved in 50 mL DMF at ambient temperature under N₂. Sodium hydride (0.8669 g, 21.67 mmol) was added and the mixture stirred for 2 hours. To the solution was added a 50 mL DMF solution containing methyl 2-(bromomethyl)-4-nitrobenzoate (5.4 g, 19.70 mmol). The solution was stirred at ambient temperature for 12 hours. The reaction was quenched by adding 10% HCl, and the mixture diluted with copious amounts of water. The aqueous layer was extracted with ether, and the combined organic layers were dried over sodium sulfate, filtered and concentrated. The residue was purified by column chromatography using 10-30% EtOAc/hexanes to provide the desired product.

Step C: Methyl 4-hydroxy-7-nitroisoquinoline-3-carboxylate: A solution of methyl 2-((N-(2-methoxy-2-oxoethyl)-4-methylphenylsulfonamido)methyl)-4-nitrobenzoate (1.20 g, 2.75 mmol) in 100 mL dry methanol was heated to 50° C. under N₂. A freshly prepared solution (20 mL) of NaOMe (prepared by adding Na (0.190 g, 8.25 mmol) to methanol) was added. The reaction was heated to 75° C. for 4 hours. The reaction was concentrated to ¼ the original volume and neutralized with 10% HCl. The resulting solids were collected, washed with water and dried under vacuum to provide the desired product.

Step D: 7-Nitroisoquinolin-4-ol: The product of Step C (0.6 g, 2.42 mmol) was suspended in 20 mL dioxane. HCl (3.02 mL, 12.1 mmol) was added and the mixture heated to 120° C. for 18 hours. The reaction was cooled to ambient temperature and neutralized with sodium bicarbonate. The aqueous layer was extracted with ethyl acetate. The combined organic layers were dried over sodium sulfate, filtered and concentrated to a solid.

Step E: 4-(Benzyloxy)-7-nitroisoquinoline: 7-Nitroisoquinolin-4-ol was dissolved in 15 mL of 1:1 mixture of THF/acetone. Added K₂CO₃ (0.230 g, 1.66 mmol) followed by the addition of benzyl bromide (0.149 mL, 1.25 mmol) after 15 minutes. The solution was heated to 60° C. for 2 hours. The reaction was cooled and concentrated to a solid. The solid was suspended in dichloromethane and purified by column chromatography using 1-5% MeOH/dichloromethane to provide the desired compound.

Step F: 4-(Benzyloxy)isoquinolin-7-amine: 4-(Benzyloxy)-7-nitroisoquinoline (0.050 g, 0.18 mmol) was dissolved in 1 mL THF. Saturated ammonium chloride (2 mL) was added and the mixture was stirred rapidly. Zn dust (0.012 g, 0.18 mmol) was added and the solution was stirred for 20 minutes. The reaction was diluted with ethyl acetate and the organic layer was separated, dried over sodium sulfate, filtered and concentrated to a film. The film was dissolved in 2% MeOH/dichloromethane and purified by column chromatography, affording the product as a solid. MS (APCI) m/z=251.2 (M+H).

Step G: Ethyl 3-(4-(benzyloxy)isoquinolin-7-ylamino)furo[2,3-c]pyridine-2-carboxylate: 4-(Benzyloxy)isoquinolin-7-amine and ethyl 3-(trifluoromethylsulfonyloxy)furo[2,3-c]pyridine-2-carboxylate were coupled according to the method of Example 53 to provide the desired product.

Step H: Ethyl 3-(4-hydroxyisoquinolin-7-ylamino)furo[2,3-c]pyridine-2-carboxylate: Ethyl 3-(4-(benzyloxy)isoquinolin-7-ylamino)furo[2,3-c]pyridine-2-carboxylate was dissolved in ethyl acetate, purged with nitrogen, and then Pd/C was added. The reaction was hydrogenated under 1 atm of H₂ for 6 hours, then concentrated to a yellow film and purified by column chromatography using dichloromethane-10% MeOH/dichloromethane. ¹H NMR (400 MHz, MeOD-D₄) δ 8.96 (1H, s), 8.55 (1H, bs), 8.29 (1H, d, J=5.4 Hz), 8.20 (1H, d, J=8.6 Hz), 7.81 (1H, bs), 7.59 (1H, m), 7.50 (1H, m), 7.33 (1H, d, J=5.4 Hz), 4.45 (2H, qt, J=7.0 Hz), 1.38 (3H, t, J=7.0 Hz). MS (APCI) m/z=350.2 (M+1).

Additional compounds, shown in Table 1, were prepared according to the method of Example 53.

TABLE 1

| Example | Structure | Name | MS (m/z) |
|---|---|---|---|
| 71 | 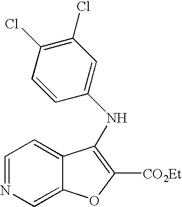 | ethyl 3-(3,4- dichlorophenylamino)furo[2,3- c}pyridine-2-carcoxylate | 351.1 (M + 1) |
| 72 | 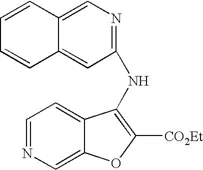 | ethyl 3-(isoquinolin-3- ylamino)furo[2,3-c]pyridine-2- carboxylate | 334.1 (M + 1) |
| 73 | 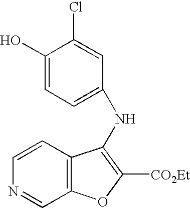 | Ethyl 3-(3-chloro-4- hydroxyphenylamino)furo[2,3- c]pyridine-2-carboxylate | 331.2 (M − 1) |
| 74 | 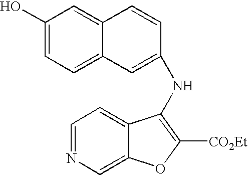 | Ethyl 3-(6-hydroxynaphthalen- 2-ylamino)furo[2,3-c]pyridine- 2-carboxylate | 347.2 (M − 1) |
| 75 | 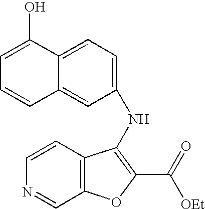 | Ethyl 3-(5-hydroxynaphthalen- 2-ylamino)furo[2,3-c]pyridine- 2-carboxylate | 349.2 (M + 1) |
| 76 | 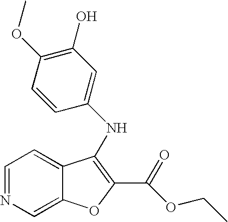 | Ethyl 3-(3-hydroxy-4- methoxyphenylamino)furo[2,3- c]pyridine-2-carboxylate | 327.2 (M − 1) |
| 77 | 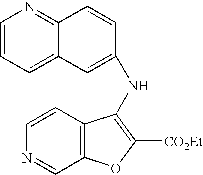 | Ethyl 3-(quinolin-6- ylamino)furo[2,3-c]pyridine-2- carboxylate | 332.2 (M − 1) |
| 78 | 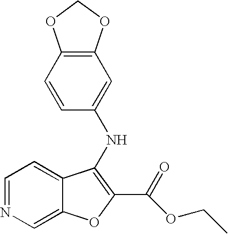 | Ethyl 3-(benzo[d][1,3]dioxol- 5-ylamino)furo[2,3-c]pyridine- 2-carboxylate | 326.2 (M − 1) |
| 79 | 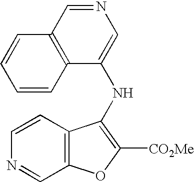 | Methyl 3-(isoquinolin-4- ylamino)furo[2,3-c]pyridine-2- carboxylate | 318.3 (M − 1) |
| 80 | 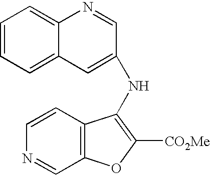 | Methyl 3-(quinolin-3- ylamino)furo[2,3-c]pyridine-2- carboxylate | 326.3 (M − 1) |
| 81 | 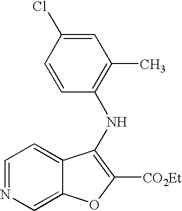 | ethyl 3-(4-chloro-2- methylphenylamino)furo[2,3- c]pyridine-2-carboxylate | 331.1 (M + 1) |
| 82 | 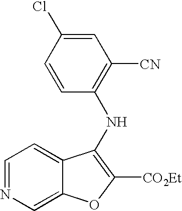 | 3-(4-chloro-2- cyanophenylamino)furo[2,3- c]pyridine-2-carboxylate | 342.1 (M + 1) |

### Example 83

### N-(2-(Dimethylamino)ethyl)-3-(8-hydroxyquinolin-3-ylamino)furo[2,3-c]pyridine-2-carboxamide

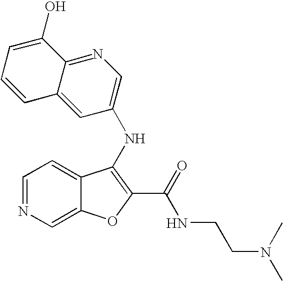

Step A: Diethyl 2-((2-methoxyphenylamino)methylene)malonate: 2-methoxybenzenamine (20.0 g, 162.4 mmol) and diethyl 2-(ethoxymethylene)malonate (35.1 g, 162.4 mmol) were mixed and heated to 130° C. overnight. The reaction was cooled to ambient temperature and concentrated to give the desired product as a solid (47.6 g, 99%).). MS (APCI) m/z 293.9 (M+1).

Step B: Ethyl 4-hydroxy-8-methoxyquinoline-3-carboxylate: 2-((2-methoxyphenylamino)methylene)malonate (47.6 g, 162 mmol) was suspended in Dowtherm (100 mL) and heated to 250° C. in a sand bath overnight, then cooled to ambient temperature and diluted with pentane (750 mL). The solid was collected by filtration and washed with hexanes (25.7 g, 64%). MS (APCI) m/z 248.0 (M+1).

Step C: Ethyl 4-chloro-8-methoxyquinoline-3-carboxylate: A mixture of 4-hydroxy-8-methoxyquinoline-3-carboxylate (5.5 g, 22.2 mmol) and POCl₃ (6.82 g, 44.5 mmol) was heated at reflux for 2 hours, then cooled to ambient temperature and carefully added to a cold solution of NH₄OH (20 mL). The aqueous layer was extracted with dichloromethane. The combined organics were dried, filtered and concentrated, and the resulting crude solid was purified by flash column chromatography, eluting with hexanes/ethyl acetate (5:1) then hexanes/ethyl acetate (1:1) to give the desired product as a solid (5.5 g, 93%).

Step D: Ethyl 8-methoxyquinoline-3-carboxylate: Ethyl 4-chloro-8-methoxyquinoline-3-carboxylate (5.5 g, 21 mmol), 10% wt. Pd/C (2.2 g) and HOAc (30 mL) was hydrogenated in a Parr shaker at 30 psi for 2 hours. The Pd was removed by filtration and the filtrate was concentrated. The residue was diluted with dichloromethane (100 mL), water (50 mL) and the pH was adjust to ˜7 with TEA. The aqueous layer was extracted with dichloromethane. The combined organic layer was dried, filtered and concentrated to give the desired product (4.8 g, 99%).

Step E: 8-methoxyquinolin-3-amine: To a solution of ethyl 8-methoxyquinoline-3-carboxylate (0.8 g, 4.0 mmol) and triethylamine (0.82 mL, 6.0 mmol) in DMF (15 mL) was added diphenylphosphoryl azide (1.27 mL, 6.0 mmol) in one portion at ambient temperature with stirring. After 1.5 hours, water (3 mL) was added and the reaction was heated to 100° C. for 1 hour. After cooling, the residue was treated with 1% NH4OH in 1 N NaOH (80 mL) and ethyl acetate (100 mL). The aqueous layer was extracted with ethyl acetate (100 mL×2). The combined organics were dried, filtered and concentrated. The crude product was purified by flash column chromatography, eluting with ethyl acetate/hexanes (7:3) and then ethyl acetate to give the desired product (0.32 g, 48%).

Step F: Preparation N-(2-(dimethylamino)ethyl)-3-(8-hydroxyquinolin-3-ylamino)furo[2,3-c]pyridine-2-carboxamide: Prepared from the product of Step E and ethyl 3-(trifluoromethylsulfonyloxy)furo[2,3-c]pyridine-2-carboxylate according to Example 53, followed by amide formation as described in Example 43 and deprotection of the methyl ether carried out as described in Example 50. ¹H NMR (400 MHz, DMSO-d₆) δ 10.1 (bs, 1H), 9.0 (s, 1H), 8.7 (d, J=3.4 Hz, 1H), 8.4 (m, 2H), 7.6 (d, J=3.4 Hz, 1H), 7.4 (m, 1H), 7.2 (m, 3H), 7.1 (d, J=7.8 Hz, 1H), 3.6 (m, 2H), 2.6 (m, 2H), 2.4 (s, 6H). MS (APCI) m/z 392.1 (M+1).

### Example 84

### N-(2-(Dimethylamino)ethyl)-3-(5-hydroxyquinolin-2-ylamino)furo[2,3-c]pyridine-2-carboxamide

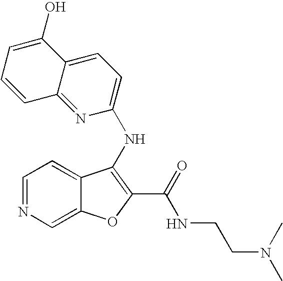

Step A: Ethyl 3-(5-methoxyquinolin-2-ylamino)furo[2,3-c]pyridine-2-carboxylate: 2-chloro-5-methoxyquinoline (0.123 g, 0.635 mmol) and ethyl 3-aminofuro[2,3-c]pyridine-2-carboxylate (0.144 g, 0.70 mmol) were suspended in p-dioxane (6.0 mL) and degassed with Ar for 15 minutes. To this was added 1,3-bis(2,6-diisopropylphenyl)-1H-imidazole-3-ium chloride (0.054 g, 0.127 mmol), Pd₂(dba)₃ (0.058 g, 0.0635 mmol), and KOt-Bu (0.107 g, 0.953 mmol). The reaction was degassed with Ar for another 15 minutes and then reflux under Ar overnight, then cooled to ambient temperature, filtered through GF/F paper, rinsed with dichloromethane and concentrated. The crude product was purified by flash column chromatography, eluting with hexanes/ethyl acetate (4:1) to give the desired product (265 mg, 99%). MS (APCI) m/z 364.1 (M+1).

Step B: N-(2-(dimethylamino)ethyl)-3-(5-methoxyquinolin-2-ylamino)furo[2,3-c]pyridine-2-carboxamide: Amide formation of the product of Step A was carried out as described in Example 43. MS (APCI) m/z 406.1 (M+1).

Step C: N-(2-(dimethylamino)ethyl)-3-(5-hydroxyquinolin-2-ylamino)furo[2,3-c]pyridine-2-carboxamide: Prepared from the product of Step B according to Example 50. ¹H NMR (400 MHz, DMSO-d₆) δ 10.2 (bs, 1H), 9.5 (bs, 1H), 9.0 (s, 1H), 8.8 (bs, 1H), 8.4 (d, J=5.5 Hz, 1H), 8.3 (d, J=9.5 Hz, 1H), 8.0 (d, J=5.5 Hz, 1H), 7.4 (m, 1H), 7.2 (d, J=9.5 Hz, 1H), 7.0 (d, J=8.4 Hz, 1H), 6.7 (d, J=8.0 Hz, 1H), 3.6 (m, 2H), 2.6 (m, 2H), 2.3 (s, 6H). MS (APCI) m/z 392.1 (M+1).

### Example 85

### N-(2-(dimethylamino)ethyl)-3-(1-oxo-2,3-dihydro-1H-inden-5-ylamino)furo[2,3-c]pyridine-2-carboxamide

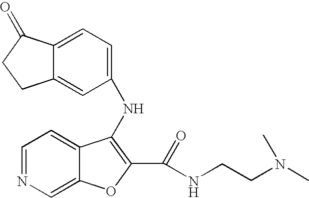

(E)-N-(2-(dimethylamino)ethyl)-3-(1-(hydroxyimino)-2,3-dihydro-1H-inden-5-ylamino)furo[2,3-c]pyridine-2-carboxamide (prepared according to Example 43; about 150 mg) was dissolved in 2 mL THF and 1M HCl (10 mL) was added. The solution was stirred for 18 hours. The reaction was quenched with saturated NaHCO₃ and the aqueous layer was extracted with ethyl acetate. The combined organic layers were dried over sodium sulfate, filtered and concentrated. The residue was purified by column using 1-3% MeOH/CHCl₃+0.1% NH₄OH to afford the product in 61% yield. MS (APCI) m/z=379.1 (M+1). ¹H NMR (400 MHz, MeOD-d₄) δ 8.95 (1H, bs), 8.37 (1H, d, J=5.4 Hz), 7.62 (1H, d, J=8.6 Hz), 7.49 (1H, m), 7.0 (2H, m), 3.56 (2H, dd, J=7.0, 6.2 Hz), 3.06 (2H, m), 2.65 (2H, m), 2.58 (2H, dd, J=7.0, 6.2 Hz), 2.31 (6H, s).

### Example 86

### N-(2,3-dihydroxypropyl)-3-(5-hydroxynaphthalen-2-ylamino)furo[2,3-c]pyridine-2-carboxamide

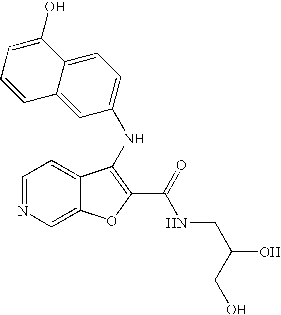

Step A: N-(2,3-bis(tert-butyldimethylsilyloxy)propyl)-3-(5-(tert-butyldimethylsilyloxy)naphthalen-2-ylamino)furo[2,3-c]pyridine-2-carboxamide: Prepared following Example 43 using 2,3-bis(tert-butyldimethylsilyloxy)propan-1-amine (0.207 g, 0.648 mmol) (prepared following procedures described in WO 89/07109.

Step B: N-(2,3-dihydroxypropyl)-3-(5-hydroxynaphthalen-2-ylamino)furo[2,3-c]pyridine-2-carboxamide: The product from step A (about 100 mg) was dissolved in 10 mL of 3:1:1 acetic acid/THF/water. The solution was heated to 50° C. for 12 hours. A few drops of 4N HCl were added and the solution was heated an additional 3 hours. The reaction was cooled, neutralized with saturated sodium bicarbonate solution, extracted several times with ethyl acetate, dried over sodium sulfate, filtered and concentrated. The residue was dissolved in dichloromethane/methanol and purified by column using 2-10% methanol/dichloromethane. The desired product was isolated as a foam (37 mg 69%). MS (APCI), m/z=394.1 (M+H). ¹H NMR (400 MHz, DMSO-d₆), δ 10.03 (1H, s), 9.04 (1H, s), 8.53 (1H, s), 8.49-8.46 (1H, m), 8.34 (1H, d, J=5.4 Hz), 8.05 (1H, d, J=8.6 Hz), 7.29 (1H, m), 7.23, (2H, m), 7.18 (1H, d, J=8.6 Hz), 6.70 (1H, d, J=7.0 Hz), 4.89 (1H, d, J=4.7 Hz), 4.36 (1H, m), 3.69-3.63 (1H, m), 3.48-3.41 (1H, m), 3.38-3.35 (1H, m), 3.27-3.21 (1H, m).

The following compounds shown in Table 2 were prepared as described for Example 43 by substituting the appropriate ester and amine.

TABLE 2

| Ex. | Structure | Name | MS, m/z |
|---|---|---|---|
| 87 | 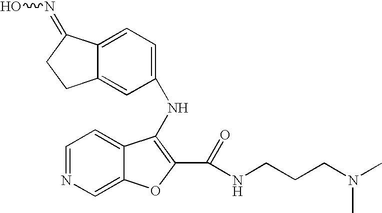 | N-(3-(dimethylamino)propyl)-3- (1-(hydroxyimino)-2,3-dihydro- 1H-inden-5-ylamino)furo[2,3- c]pyridine-2-carboxamide. | 408 (M + 1) |
| 88 | 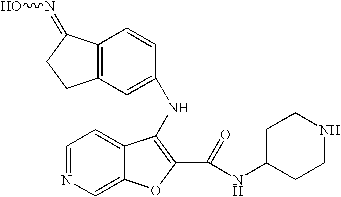 | 3-(1-(hydroxyimino)-2,3- dihydro-1H-inden-5-ylamino)- N-(piperidin-4-yl)furo[2,3- c]pyridine-2-carboxamide | 406.2 (M + 1) |
| 89 | 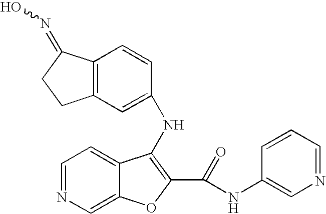 | 3-(1-(hydroxyimino)-2,3- dihydro-1H-inden-5-ylamino)- N-(pyridin-3-yl)furo[2,3- c]pyridine-2-carboxamide | 400.2 (M + 1) |
| 90 | 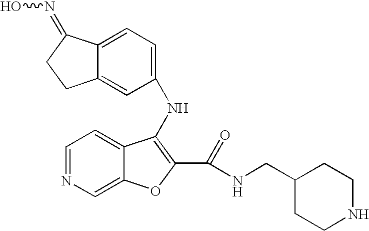 | 3-(1-(hydroxyimino)-2,3- dihydro-1H-inden-5-ylamino)- N-(piperidin-4- ylmethyl)furo[2,3-c]pyridine-2- carboxamide | 420.2 (M + 1) |
| 91 | 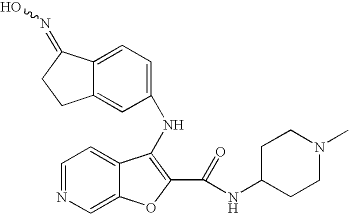 | 3-(1-(hydroxyimino)-2,3- dihydro-1H-inden-5-ylamino)- N-(1-methylpiperidin-4- yl)furo[2,3-c]pyridine-2- carboxamide | 420.2 (M + 1) |
| 92 | 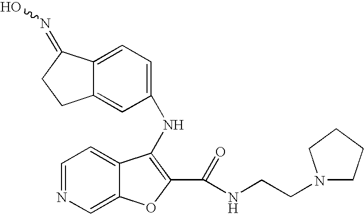 | 3-(1-(hydroxyimino)-2,3- dihydro-1H-inden-5-ylamino)- N-(2-(pyrrolidin-1- yl)ethyl)furo[2,3-c]pyridine-2- carboxamide | 420.2 (M + 1) |
| 93 | 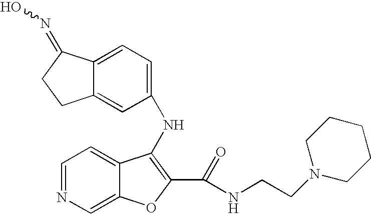 | 3-(1-(hydroxyimino)-2,3- dihydro-1H-inden-5-ylamino)- N-(2-(piperidin-1- yl)ethyl)furo[2,3-c]pyridine-2- carboxamide | 434.2 (M + 1) |
| 94 | 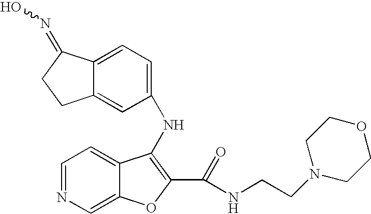 | 3-(1-(hydroxyimino)-2,3- dihydro-1H-inden-5-ylamino)- N-(2-morpholinoethyl)furo[2,3- c]pyridine-2-carboxamide | 436.2 (M + 1 |
| 95 | 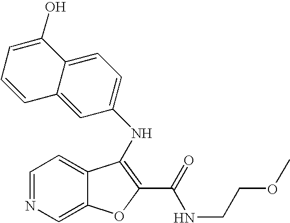 | 3-(5-hydroxynaphthalen-2- ylamino)-N-(2- methoxyethyl)furo[2,3- c]pyridine-2-carboxamide | 378.3 (M + 1) |
| 96 | 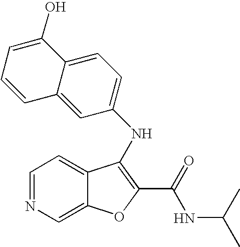 | 3-(5-hydroxynaphthalen-2- ylamino)-N-isopropylfuro[2,3- c]pyridine-2-carboxamide | 362.2 (M + 1) |
| 97 | 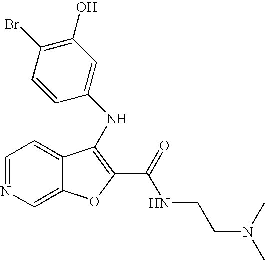 | 3-(4-bromo-3- hydroxyphenylamino)-N-(2- (dimethylamino)ethyl)furo[2,3- c]pyridine-2-carboxamide | 419.1, 421.0 (M + 1) |
| 98 | 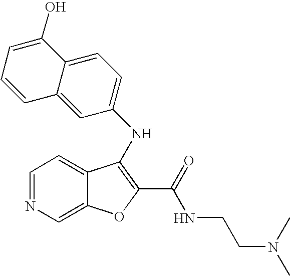 | N-(2-(dimethylamino)ethyl)-3- (5-hydroxynaphthalen-2- ylamino)furo[2,3-c]pyridine-2- carboxamide | 391.1 (M + 1) |
| 99 | 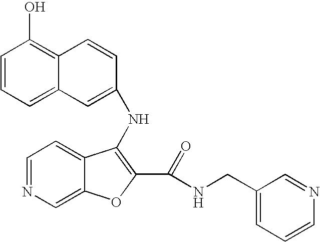 | 3-(5-hydroxynaphthalen-2- ylamino)-N-(pyridin-3- ylmethyl)furo[2,3-c]pyridine-2- carboxamide | 411.2 (M + 1) |
| 100 | 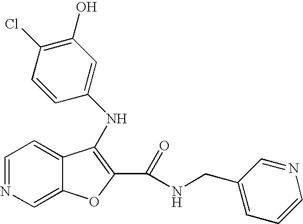 | 3-(4-chloro-3- hydroxyphenylamino)-N- (pyridin-3-ylmethyl)furo[2,3- c]pyridine-2-carboxamide | 395.2 (M + 1) |
| 101 | 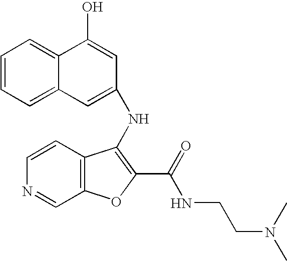 | N-(2-(dimethylamino)ethyl)-3- (4-hydroxynaphthalen-2- ylamino)furo[2,3-c]pyridine-2- carboxamide | 391.2 (M + 1) |
| 102 | 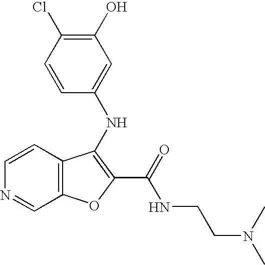 | 3-(4-chloro-3- hydroxyphenylamino)-N-(2- (dimethylamino)ethyl)furo[2,3- c]pyridine-2-carboxamide | 375.1 (M + 1) |
| 103 | 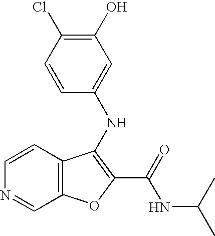 | 3-(4-chloro-3- hydroxyphenylamino)-N- isopropylfuro[2,3-c]pyridine-2- carboxamide: | 344.2 (M − 1) |
| 104 | 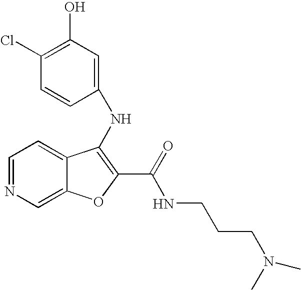 | 3-(4-chloro-3- hydroxyphenylamino)-N-(3- (dimethylamino)propyl)furo[2,3- c]pyridine-2-carboxamide | 389.1 (M + 1) |
| 105 | 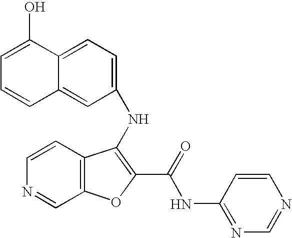 | 3-(5-hydroxynaphthalen-2- ylamino)-N-(pyrimidin-4- yl)furo[2,3-c]pyridine-2- carboxamide | 396.2 (M − 1) |
| 106 | 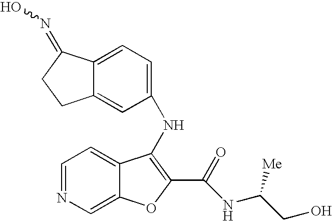 | (R)-3-(1-(hydroxyimino)-2,3- dihydro-1H-inden-5-ylamino)- N-(1-hydroxypropan-2- yl)furo[2,3-c]pyridine-2- carboxamide | 381.2 (M + 1) |
| 107 | 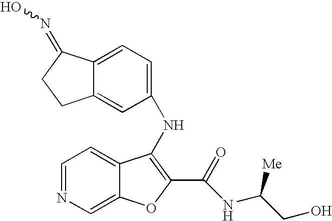 | (S)-3-(1-(hydroxyimino)-2,3- dihydro-1H-inden-5-ylamino)- N-(1-hydroxypropan-2- yl)furo[2,3-c]pyridine-2- carboxamide | 381.2 (M + 1) |
| 108 | 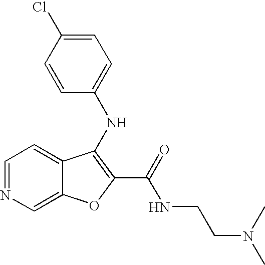 | 3-(4-chlorophenylamino)-N-(2- (dimethylamino)ethyl)furo[2,3- c]pyridine-2-carboxamide | 359.1 (M + 1) |
| 109 | 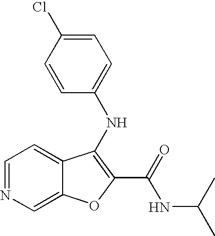 | 3-(4-chlorophenylamino)-N- isopropylfuro[2,3-c]pyridine-2- carboxamide | 330.2 (M + 1) |
| 110 | 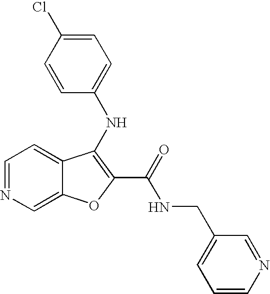 | 3-(4-chlorophenylamino)-N-(2- (dimethylamino)ethyl)furo[2,3- c]pyridine-2-carboxamide | 359.1 (M + 1) |
| 111 | 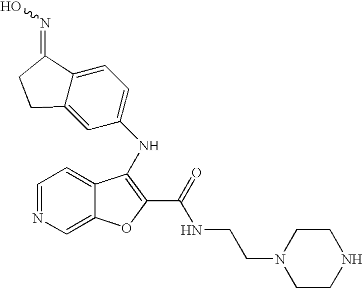 | 3-(1-(hydroxyimino)-2,3- dihydro-1H-inden-5-ylamino)- N-(2-(piperazin-1- yl)ethyl)furo[2,3-c]pyridine-2- carboxamide | 435.2 (M + 1) |
| 112 | 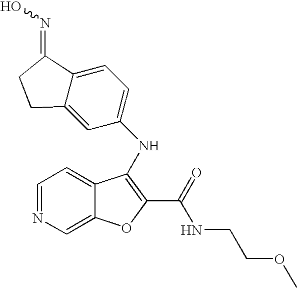 | 3-(1-(hydroxyimino)-2,3- dihydro-1H-inden-5-ylamino)- N-(2-methoxyethyl)furo[2,3- c]pyridine-2-carboxamide | 381.3 (M + 1) |
| 113 | 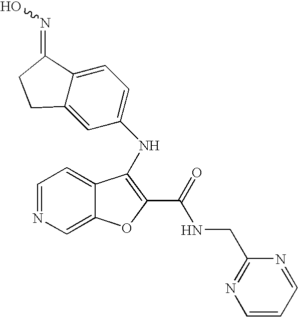 | 3-(1-(hydroxyimino)-2,3- dihydro-1H-inden-5-ylamino)- N-(pyrimidin-2- ylmethyl)furo[2,3-c]pyridine-2- carboxamide | 415.2 (M + 1) |
| 114 | 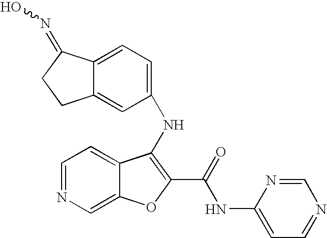 | 3-(1-(hydroxyimino)-2,3- dihydro-1H-inden-5-ylamino)- N-(pyrimidin-4-yl)furo[2,3- c]pyridine-2-carboxamide | 401.1 (M + 1) |
| 115 | 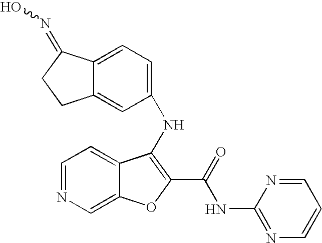 | 3-(1-(hydroxyimino)-2,3- dihydro-1H-inden-5-ylamino)- N-(pyrimidin-2-yl)furo[2,3- c]pyridine-2-carboxamide | 401.1 (M + 1) |
| 116 | 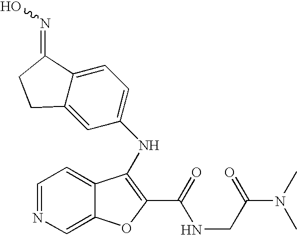 | N-(2-(dimethylamino)-2- oxoethyl)-3-(1-(hydroxyimino)- 2,3-dihydro-1H-inden-5- ylamino)furo[2,3-c]pyridine-2- carboxamide | 408.1 (M + 1) |
| 117 | 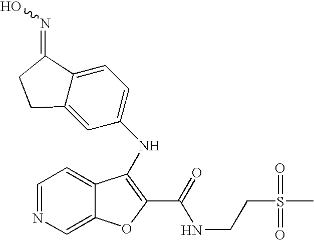 | 3-(1-(hydroxyimino)-2,3- dihydro-1H-inden-5-ylamino)- (methylsulfonyl)ethyl)furo[2,3- c]pyridine-2-carboxamide | 429.2 (M + 1) |
| 118 | 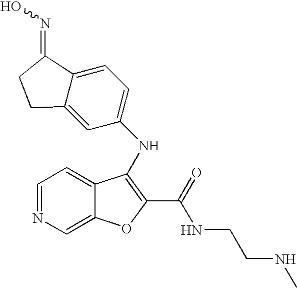 | 3-(1-(hydroxyimino)-2,3- dihydro-1H-inden-5-ylamino)- (methylamino)ethyl)furo[2,3- c]pyridine-2-carboxamide | 380.1 (M + 1) |
| 119 | 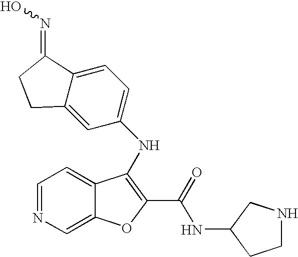 | 3-(1-(hydroxyimino)-2,3- dihydro-1H-inden-5-ylamino)- N-(pyrrolidin-3-yl)furo[2,3- c]pyridine-2-carboxamide | 392.2 (M + 1) |
| 120 | 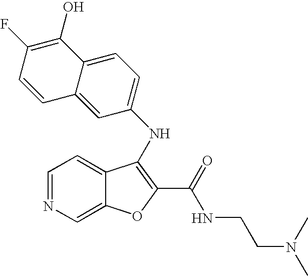 | N-(2-(dimethylamino)ethyl)-3- (6-fluoro-5-hydroxynaphthalen- 2-ylamino)furo[2,3-c]pyridine- 2-carboxamide | 409.1 (M + 1) |
| 121 | 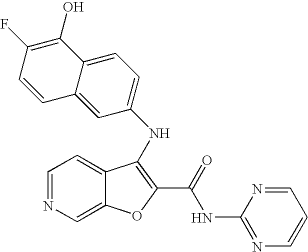 | 3-(6-fluoro-5- hydroxynaphthalen-2-ylamino)- N-(pyrimidin-2-yl)furo[2,3- c]pyridine-2-carboxamide | 416.2 (M + 1) |
| 122 | 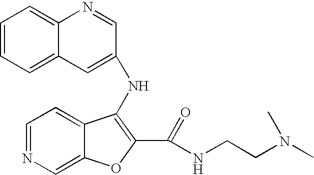 | N-(2-(dimethylamino)ethyl)-3- (quinolin-3-ylamino)furo[2,3- c]pyridine-2-carboxamide | 376 (M + 1) |
| 123 | 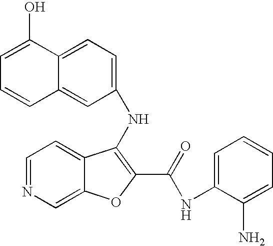 | N-(2-aminophenyl)-3-(5- hydroxynaphthalen-2- ylamino)furo[2,3-c]pyridine-2- carboxamide | 411.2 (M + 1) |

### Example 124

### 3-(4-chlorophenylamino)-N-(2-hydroxypropyl)furo[2,3-c]pyridine-2-carboxamide

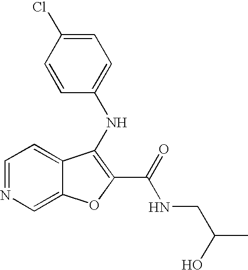

Step A: 3-(4-chlorophenylamino)furo[2,3-c]pyridine-2-carboxylic acid: To a slurry of ethyl 3-(4-chlorophenylamino)furo[2,3-c]pyridine-2-carboxylate (0.046 g, 0.145 mmol) in 1.5 mL MeOH and 1.5 mL THF was added a solution of LiOH (0.009 g, 0.218 mmol) in 1 mL of water and the solution was stirred for 2 hours. The solution was brought to pH 2 with 1.0 N HCl and extracted with EtOAc. The combined organics were dried over sodium sulfate and concentrated to provide the product as an oil. M+1=289.1.

Step B: 3-(4-chlorophenylamino)-N-(2-hydroxypropyl)furo[2,3-c]pyridine-2-carboxamide: A solution of 3-(4-chlorophenylamino)furo[2,3-c]pyridine-2-carboxylic acid (0.0153 g, 0.0530 mmol), HOBT (0.00143 g, 0.0106 mmol), HBTU (0.0201 g, 0.0530 mmol), and diisopropylethylamine (0.0384 mL, 0.265 mmol) in 1.0 mL DMF at 0° C. was stirred for 10 minutes. 1-Aminopropan-2-ol (0.00450 mL, 0.0583 mmol) was added and stirred at ambient temperature for 1 hour. The reaction was quenched with water and extracted with EtOAc. The combined organics were dried over sodium sulfate and concentrated to an oil. Purification by silica gel chromatography provided the title compound (0.013 g, 14% for two steps) as a solid. MS (APCI-pos) M+1=346.2. ¹H NMR (400 MHz, CDCl₃) δ 8.88 (s, 1H), 8.34 (d, 1H), 8.04 (s, 1H), 7.27-7.30 (m, 2H), 7.19 (d, 1H), 7.02 (d, 2H), 6.85-6.88 (m, 1H), 4.05-4.13 (m, 1H), 3.67-3.78 (m, 1H), 3.33-3.38 (m, 1H).

### Example 125

### 5-Amino-2,3-dihydro-1H-inden-1-one O-tert-butyldimethylsilyl oxime

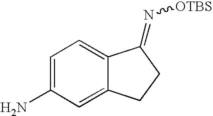

5-Amino-2,3-dihydro-1H-inden-1-one (8.0 g, 54.4 mmol, 1.0 equiv) was suspended in CHCl₃ (70 ml). O-(tert-butyldimethylsilyl)hydroxylamine (11.2 g, 76 mmol, 1.40 equiv), TsOH—H₂O (1.0 g, 5.26 mmol, 0.096 equiv) and oven-dried 4A MS (14 g) were added and the mixture was heated to reflux over the weekend. Upon cooling, the reaction mixture was filtered through GF/F filter paper and concentrated to residue under vacuum. The residue was purified by Biotage column chromatography to give the product as a solid (12.7 g, 85%) after drying under high vacuum. MS (APCI-pos) M+1=277.2. ¹H NMR (400 MHz, CDCl₃) δ 7.38-7.36 (m, J=8.5 Hz, 1H), 6.43-6.39 (m, 2H), 3.79-3.70 (m, 2H), 2.82-2.76 (m, 2H), 0.87-0.78 (m, 9H), 0.11-0.05 (m, 6H).

### Example 126

### N-(2-Aminoethyl)-3-(5-hydroxynaphthalen-2-ylamino)furo[2,3-c]pyridine-2-carboxamide

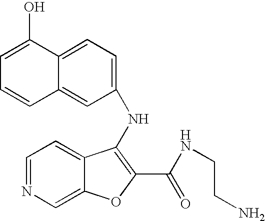

Step A: N-(2-aminoethyl)-3-(5-(tert-butyldimethylsilyloxy)naphthalen-2-ylamino)furo[2,3-c]pyridine-2-carboxamide: Ethyl 3-(5-(tert-butyldimethylsilyloxy)naphthalen-2-ylamino)furo[2,3-c]pyridine-2-carboxylate (0.316 g, 0.68308 mmol) was treated with ethane-1,2-diamine (0.45664 mL, 6.8308 mmol) and the mixture was heated to 100° C. for 2 hours while slowly passing a stream of N₂ (g) over the open vessel. The reaction mixture was cooled to ambient temperature. The yellow residue was dissolved in methylene chloride (ca. 3 mL) and chromatographed on SiO₂ (Biotage 25M, loaded with methylene chloride) eluting with 20% MeOH/ethyl acetate then switching to 20% MeOH/ethyl acetate containing 1% NH₄OH. The desired product was recovered as a yellow film (250 mg, 77%).

Step B: N-(2-Aminoethyl)-3-(5-hydroxynaphthalen-2-ylamino)furo[2,3-c]pyridine-2-carboxamide: Prepared from the product of Step A according to Example 52. The desired product was recovered a yellow solid (24.1 mg, 52%). MS (ESI+) m/z 363.1. ¹H NMR (CDCl₃, 400 MHz) δ 8.86 (s, 1H), 8.25 (d, 1H, J=5.5 Hz), 8.20 (d, 1H, J=9.1 Hz), 7.37-7.34 (m, 1H), 7.29-7.15 (m, 5H), 6.75 (d, 1H, J=7.4 Hz), 3.57 (t, 2H), 2.99 (t, 2H).

### Example 127

### 6-(2-(4,5-Dihydro-1H-imidazol-2-yl)furo[2,3-c]pyridin-3-ylamino)naphthalen-1-ol

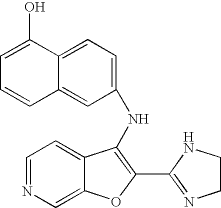

Step A: N-(5-(tert-butyldimethylsilyloxy)naphthalen-2-yl)-2-(4,5-dihydro-1H-imidazol-2-yl)furo[2,3-c]pyridin-3-amine: N-(2-Aminoethyl)-3-(5-(tert-butyldimethylsilyloxy)naphthalen-2-ylamino)furo[2,3-c]pyridine-2-carboxamide (0.250 g, 0.5245 mmol) was dissolved in toluene (1.0 mL) and cooled to 0° C. Trimethylaluminum (1.311 mL, 2.623 mmol, 2.0 M in toluene) was added slowly and the mixture was stirred for 30 minutes at 0° C., then heated to reflux for 60 hours. The reaction was cooled to ambient temperature, quenched with ice, and then diluted with saturated NaHCO₃ and ethyl acetate. The layers were separated, and the aqueous layer was washed with ethyl acetate. The combined organic layers were then washed successively with saturated NaHCO₃ and saturated NaCl. The organic layers were combined, dried over sodium sulfate and concentrated in vacuo to an orange oil (118 mg). This residue was chromatographed on SiO₂ (Biotage 12M, loaded with methylene chloride) eluting with methylene chloride then with 2% MeOH/1% NH₄OH/methylene chloride. The desired product was recovered as a yellow solid (28 mg, 11%).

Step B: 6-(2-(4,5-Dihydro-1H-imidazol-2-yl)furo[2,3-c]pyridin-3-ylamino)naphthalen-1-ol: Prepared from the product of Step A following Example 52. The product was recovered as a yellow solid (94%). MS (ESI+) m/z 345.3 ¹H NMR (CDCl₃, 400 MHz): 8.89 (s, 1H), 8.26 (d, 1H, J=5.4 Hz), 7.77 (d, 1H, J=8.6 Hz), 7.28-7.24 (m, 1H), 7.13 (t, 1H, J=7.8 Hz), 7.03-6.99 (m, 2H), 6.87-6.83 (m, 1H), 6.62 (d, 1H, J=7.3 Hz), 4.26-4.15 (brd s, 2H), 3.75-3.62 (brd s, 2H).

### Example 128

### Ethyl 3-hydroxyisonicotinate

Step A: 3-Aminoisonicotinic acid: 2H-Pyrrolo[3,4-c]pyridine-1,3-dione (204.16 g, 1378.4 mmol) was dissolved in 10% NaOH (3.3 L) and the solution was cooled to an internal temperature of 7° C. (ice/salt bath). Bromine (73.424 mL, 1433.5 mmol) was added dropwise while maintaining the internal temperature below 10° C. After completion of the addition, the reaction was heated to an internal temperature of 80-85° C. for 90 minutes. The reaction mixture was cooled to 20-30° C. in an ice bath then acetic acid (323.21 mL, 5651.2 mmol) was added dropwise. The reaction was stirred and cooled to 5° C. The solids were collected by vacuum filtration, washed with cold water then air-dried to provide the product (108.86 g, 57%).

Step B: 3-Hydroxyisonicotinic acid: 3-Aminoisonicotinic acid (108.86 g, 788.13 mmol) was dissolved in water (1740 mL) then treated with sulfuric acid (84.020 mL, 1576.3 mmol). The yellow slurry was cooled to <10° C. and a solution of sodium nitrite (60.359 g, 874.83 mmol) in water (510 mL) was added dropwise while maintaining the temperature at <10° C. The solution was heated to 80° C., which caused a thick precipitate to form. The suspension was cooled to 65° C. and treated with glacial acetic acid (88 mL, in a continuous pour) followed by concentrated ammonium hydroxide (190 mL) to a final pH of approximately 4.5. The solids were collected by vacuum filtration and washed with cold water. After air-drying 16 hours, a free-flowing granular solid was obtained (99.37 g, 91%).

Step C: Ethyl 3-hydroxyisonicotinate: 3-Hydroxyisonicotinic acid (99.37 g, 714.3 mmol) was combined with absolute EtOH (300 mL) and 1,2-dichloroethane (400 mL). Sulfuric acid (59.78 mL, 1122 mmol) was added and the mixture was heated to reflux for 5 days. The solution was cooled to ambient temperature and allowed to stand overnight. The solution was concentrated in vacuo and treated with water (500 mL). Solid NaHCO₃ was added slowly to bring the suspension to pH 8. The resultant solid was collected by vacuum filtration, washed with cold water, and air-dried to provide the desired product as a powder (93.6 g, 78%). ¹H NMR (DMSO-d₆, 400 MHz) δ 10.38 (brd s, 1H), 8.39 (s, 1H), 8.16 (d, 1H, J=5.0 Hz), 7.55 (d, 1H, J=4.6 Hz), 4.34 (q, 2H), 1.32 (t, 3H).

### Example 129

### 2-(Pyrimidin-2-yl)furo[2,3-c]pyridin-3-yl trifluoromethanesulfonate

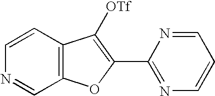

Step A: Methyl pyrimidine-2-carboxylate: HCl gas was bubbled through 700 ml MeOH as 0° C. to give a saturated solution. Pyrimidine-2-carbonitrile (21.585 g, 205.38 mmol)was added and the reaction was stirred at ambient temperature for 16 hours at, then heated at 40-50° C. for 3 hours. The solvent was evaporated under vacuum, leaving an off-white semi-solid, which was dissolved water and the pH adjusted 7.0 using NaHCO₃. The mixture was extracted with 20% iPrOH/CH₂Cl₂, dried over sodium sulfate and concentrated under vacuum to white residue (23.0 g, 81%).

Step B: Pyrimidin-2-ylmethanol: A solution of methyl pyrimidine-2-carboxylate (659 mg, 4.77 mmol, 1.00 equiv) in 25 mL EtOH was cooled to 0° C. in an ice bath, and sodium borohydride (181 mg, 4.77 mmol, 1.00 equiv) was added. The reaction mixture was warmed to ambient temperature, and stirred 2 hours, and then 5 ml water was added. The reaction was concentrated under reduced pressure, and the residue was purified using silica gel chromatography to give the desired product as a white solid (154 mg, 30%).

Step C: Ethyl 3-(pyrimidin-2-ylmethoxy)isonicotinate: A solution of triphenyl phosphine (14.29 g, 54.49 mmol, 1.20 equiv) in 150 ml THF was cooled to −15° C. DIAD was added via syringe (10.70 ml, 54.49 mmol, 1.20 equiv). The reaction mixture was and stirred 10 minutes at −15° C., then a solution of pyrimidin-2-ylmethanol (5.00 g, 45.41 mmol, 1.00 equiv) in 30 ml THF was added. After 10 minutes, a solution of ethyl 3-hydroxyisonicotinate (7.590 g, 45.41 mmol, 1.00 equiv) in 75 ml THF was added to the reaction mixture and the reaction mixture was allowed to warm to ambient temperature over 16 hours. The reaction was concentrated under reduced pressure and the residue was purified by silica gel chromatography to give the desired product as an oil (7.238 g, 61%). MS (APCI-pos) M+1=260.1.

Step D: 2-(Pyrimidin-2-yl)furo[2,3-c]pyridin-3-yl trifluoromethanesulfonate: A solution of ethyl 3-(pyrimidin-2-ylmethoxy)isonicotinate (7.238 g, 27.92 mmol, 1.00 equiv) in 100 ml DMF was cooled to 0° C., and a suspension of NaH (4.466 g, 111.7 mmol, 4.00 equiv) in 20 ml DMF was added to the reaction mixture. The reaction mixture was warmed to ambient temperature and stirred 1 hour. The crude mixture was treated with aqueous NH₄Cl and 1M citric acid and extracted twice with ethyl acetate. After drying over MgSO₄, the crude material was purified by silica gel chromatography to afford the product. MS (APCI-pos) M+1=214.3. Addition of the triflate group was carried out according to Example 55 to provide the desired product (4.20 g, 96%). MS (APCI-pos) M+1=214.3 ¹H NMR (400 MHz, CDCl₃) δ 9.11 (s, 1H), 8.97-8.96 (d, J=4.5 Hz, 2H), 8.62-8.61 (d, J=5.7 Hz, 1H), 7.64-7.62 (d, J=4.7 Hz, 1H), 7.41-7.39 (t, J=4.7 Hz, 1H).

### Example 130

### 5-(2-(Pyrazin-2-yl)furo[2,3-c]pyridin-3-ylamino)-2,3-dihydro-1H-inden-1-one oxime

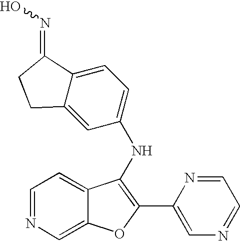

2-(Pyrazin-2-yl)furo[2,3-c]pyridin-3-yl trifluoromethanesulfonate and 5-amino-2,3-dihydro-1H-inden-1-one O-tert-butyldimethylsilyl oxime (Example 125) were coupled according to the method of Example 53. Subsequent deprotection according to Example 52 afforded the desired product in 34% yield. MS (APCI-pos) M+1=358.2. ¹H NMR (400 MHz, d₆-DMSO) δ 10.54 (s, 1H), 9.16 (s, 1H), 9.09 (s, 1H), 8.79-8.77 (m, 1H), 8.72 (s, 1H), 8.64-8.62 (m, 1H), 8.41-8.38 (m, 1H), 7.44-7.41 (m, 1H), 7.37-7.34 (m, 1H), 6.90-6.86 (m, 2H), 2.91-2.73 (m, 4H).

### Example 131

### 5-(2-(Pyrimidine-4-yl)furo[2,3-c]pyridin-3-ylamino)-2,3-dihydro-1H-inden-1-one oxime

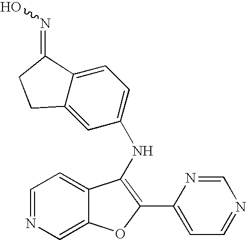

Prepared according to the method of Example 130. ¹H NMR (400 MHz, DMSO-d₆) δ 10.6 (bs, 1H), 9.3 (s, 1H), 9.1 (s, 1H), 8.9 (d, J=5.5 Hz, 1H), 8.4 (d, J=5.5 Hz, 1H), 7.9 (d, J=5.5 Hz, 1H), 7.5 (d, J=8.4 Hz, 1H), 7.3 (d, J=5.5 Hz, 1H), 7.0 (m, 3H), 2.9 (m, 2H), 2.8 (m, 2H). MS (APCI) m/z 358.3 (M+1).

### Example 132

### 2-Chloro-5-(2-(pyrimidin-2-yl)furo[2,3-c]pyridin-3-ylamino)phenol

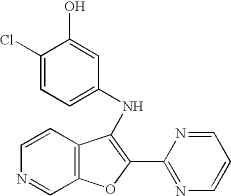

Step A: 2-(Benzyloxymethoxy)-1-chloro-4-nitrobenzene: Sodium hydride (0.4878 g, 12.20 mmol, 1.10 equiv) was suspended in 10 mL DMF and cooled to 0° C. A solution of 2-chloro-5-nitrophenol (1.008 g, 5.808 mmol, 1.05 equiv) in 5 mL DMF was added dropwise, and the mixture was warmed to 25° C. for 15 minutes while stirring. Benzyl chloromethyl ether (2.693 mL, 11.62 mmol) was added dropwise and the mixture was stirred for 30 minutes at ambient temperature. The reaction mixture was transferred to a separatory funnel, and diluted with water, brine and ethyl acetate. The layers were separated and the combined organics layers were washed with brine. The organics were separated, dried and concentrated to provide the product as a brown oil (1.7 g, 100%).

Step B: 3-(benzyloxymethoxy)-4-chloroaniline: A mixture of 2-(Benzyloxymethoxy)-1-chloro-4-nitrobenzene (1.365 g, 4.648 mmol), FeCl₃-6(H₂O) (82 mg), and activated carbon (200 mg) was heated to reflux in MeOH (70 deg) for 20 minutes. Added N₂H₄—H₂O (1.5 mL) and heated at reflux the mixture at 70° C. for 8 hours. Transferred the mixture to a separatory funnel, diluted with water, brine and ethyl acetate. Extracted with EtOAc, and dried and concentrated the organic layer to provide the desired compound.

Step C: 2-chloro-5-(2-(pyrimidin-2-yl)furo[2,3-c]pyridin-3-ylamino)phenol: The product of Step B and 2-(pyrimidin-2-yl)furo[2,3-c]pyridin-3-yl trifluoromethanesulfonate (Example 129) were coupled according to the method of Example 53, followed by removal of the protecting group using 6N HCl to provide the desired compound. MS (APCI-neg) M−1=337.3 ¹H NMR (400 MHz, d6-DMSO) δ 10.11 (s, 1H), 9.08 (s, 1H), 8.96-8.95 (d, J=4.7 Hz, 2H), 8.72 (s, 1H), 8.39-8.38 (d, J=4.6 Hz, 1H), 7.45-7.43 (t, J=5.3 Hz, 1H), 7.31-7.30 (d, J=5.4 Hz, 1H), 6.69-6.68 (d, J=2.4 Hz, 1H), 6.60-6.58 (dd, J=6.0, 2.4 Hz, 1H).

The following examples shown in Table 3 were prepared according the procedure described in Example 130.

TABLE 3

| Example | Structure | Name | MS m/z |
|---|---|---|---|
| 133 | 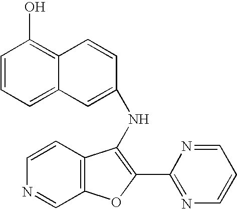 | 6-(2-(pyrimidin-2- yl)furo[2,3-c]pyridin- 3- ylamino)naphthalen-1-ol | 353.4 (M − 1) |
| 134 | 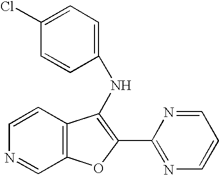 | N-(4-chlorophenyl)- 2-(pyrimidin-2- yl)furo[2,3-c]pyridin- 3-amine | 323.4 (M + 1) |
| 135 | 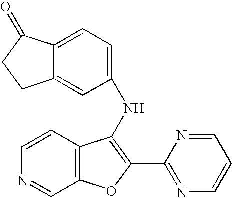 | 5-(2-(pyrimidin-2- yl)furo[2,3-c]pyridin- 3-ylamino)-2,3- dihydro-1H-inden-1- one | 341.5 (M − 1) |
| 136 | 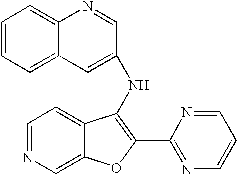 | 2-(pyrimidin-2-yl)- N-(quinolin-3- yl)furo[2,3-c]pyridin- 3-amine | 340.4 (M + 1) |
| 137 | 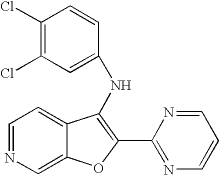 | N-(3,4- dichlorophenyl)-2- (pyrimidin-2- yl)furo[2,3-c]pyridin- 3-amine | 357.3 (M + 1) |

### Example 138

### Methyl 2-(3-(trifluoromethylsulfonyloxy)furo[2,3-c]pyridin-2-yl)pyrimidine-5-carboxylate

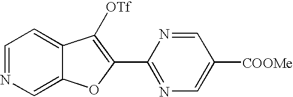

Step A: Sodium (Z)-2-(dimethoxymethyl)-3-methoxy-3-oxoprop-1-en-1-olate: A 1 L flask was charged with methyl 3,3-dimethoxypropanoate (50.1 g, 328 mmol), 1,2-dimethoxyethane (200 mL) and methyl formate (47.8 g, 787 mmol). The reaction mixture was cooled to 0° C. and NaH (60% suspension in mineral oil, 17.1 g, 426 mmol) was added portionwise. The reaction mixture was stirred at 0° C. for 30 minutes and then heated to 35° C. to initiate reaction. After stirring at ambianet temperature for 16 hours, the reaction mixture was diluted with ether (125 mL), the solids were collected by filtration and washed with ether (50 mL). The white solids were dried in vacuo to give the desired product (58.4 g, 90%).

Step B: 3-Hydroxyfuro[2,3-c]pyridine-2-carboxamidine hydrochloride: To a cold (0° C.) suspension of NH₄Cl (6.45 g, 121 mmol) in toluene (150 mL) was added AlMe₃ (2.0 M in toluene, 60.3 mL, 121 mmol) dropwise over 30 minutes. The cold bath was removed and the reaction mixture was stirred at ambient temperature for 30 minutes. Ethyl 3-hydroxyfuro[2,3-c]pyridine-2-carboxylate (5.0 g, 24.1 mmol) was added and the reaction mixture was heated at reflux overnight. The reaction mixture was cooled to 0° C. and carefully quenched with MeOH. The resulting suspension was stirred at ambient temperature for 1 hour and then concentrated to give the desired product as a solid. MS (APCI) m/z 178.1 (M+1).

Step C: Methyl 2-(3-hydroxyfuro[2,3-c]pyridin-2-yl)pyrimidine-5-carboxylate: The crude product from step B was suspended in DMF (100 mL), cooled to 0° C. and treated with solid NaOMe (5.22 g, 96.6 mmol) for 20 minutes then sodium (Z)-2-(dimethoxymethyl)-3-methoxy-3-oxoprop-1-en-1-olate (Step A, 15.7 g, 79.8 mmol) was added. The reaction mixture was heated to 100° C. under N₂ for 2 hours, cooled to 0° C., carefully quenched with water (1 L) and stirred at ambient temperature for 16 hours. The aqueous layer was washed with ethyl acetate and then acidified with HOAc (20 mL) to pH-5. The aqueous layer was extracted with ethyl acetate. The organic layers were combined, dried, filtered and concentrated. The crude product was purified by flash column chromatography, eluting with dichloromethane/MeOH (40:1) to give the desired product (2.85 g, 44%). MS (APCI) m/z 272.3 (M+1)

Step D: Methyl 2-(3-(trifluoromethylsulfonyloxy)furo[2,3-c]pyridin-2-yl)pyrimidine-5-carboxylate: To a cold (0° C.) solution of methyl 2-(3-hydroxyfuro[2,3-c]pyridin-2-yl)pyrimidine-5-carboxylate (5.7 g, 21.0 mmol) and pyridine (2.21 mL, 27.3 mmol) in dichloromethane (50 mL) was added Tf₂O (4.26 mL, 25.2 mmol) dropwise. The reaction mixture was stirred at 0° C. for 2 hours before quenching with water (50 mL). The aqueous layer was extracted with dichloromethane. The combined organics were dried, filtered and concentrated. The crude product was purified by flash column chromatography, eluting with hexane/ethyl acetate (4:1), hexane/ethyl acetate (1:1) to give the desired product (5.9 g, 70%). MS (APCI) m/z 403.9 (M+1). ¹H NMR (400 MHz, CDCl₃) δ 9.5 (s, 2H), 9.2 (s, 1H), 8.7 (d, 4.4 Hz, 1H), 7.7 (d, J=4.4 Hz, 1H), 4.0 (s, 3H).

### Example 139

### (Z,E)-methyl 2-(3-(1-(hydroxyimino)-2,3-dihydro-1H-inden-5-ylamino)furo[2,3-c]pyridin-2-yl)pyrimidine-5-carboxylate

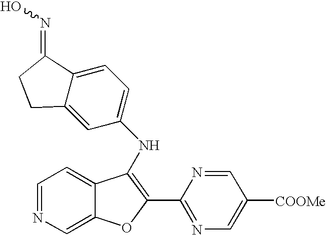

Prepared from 5-amino-2,3-dihydro-1H-inden-1-one O-tert-butyldimethylsilyl oxime (Example 125) and methyl 2-(3-(trifluoromethylsulfonyloxy)furo[2,3-c]pyridin-2-yl)pyrimidine-5-carboxylate (Example 138) using the methods described in Examples 53 and 51. MS (APCI) m/z 530.3 (M+1). MS (APCI) m/z 416.3 (M+1) ¹HNMR (400 MHz, DMSO-d₆) δ 10.7 (bs, 1H), 9.3 (s, 2H), 9.2-9.1 (m, 2H), 8.4 (m, 1H), 7.5 (m, 1H), 7.3 (m, 1H), 7.2-7.1 (m, 2H), 3.9 (s, 3H), 2.9 (m, 2H), 2.8 (m, 2H).

### Example 140

### (Z,E)-(2-(3-(1-(hydroxyimino)-2,3-dihydro-1H-inden-5-ylamino)furo[2,3-c]pyridin-2-yl)pyrimidin-5-yl)(4-methylpiperazin-1-yl)methanone

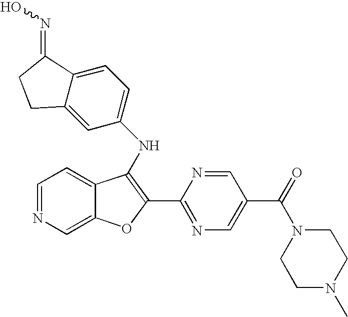

Prepared from (Z,E)-methyl 2-(3-(1-(tert-butyldimethylsilyloxyimino)-2,3-dihydro-1H-inden-5-ylamino)furo[2,3-c]pyridin-2-yl)pyrimidine-5-carboxylate and 1-methylpiperazine according to the method of Example 43 followed by TFA deprotection as described in Example 51. MS (APCI) m/z 484.2 (M+1). ¹H NMR (400 MHz, DMSO-d₆) δ 10.6 (bs, 1H), 9.1 (s, 1H), 9.0 (m, 3H), 8.4 (m, 1H), 7.5 (m, 1H), 7.3 (m, 1H), 7.1-7.0 (m, 2H), 3.8-3.4 (m, 4H), 2.9 (m, 2H), 2.8 (m, 2H), 2.6-2.4 (m, 4H), 2.3 (s, 3H).

### Example 141

### (Z,E)-N-(2-(dimethylamino)ethyl)-2-(3-(1-(hydroxyimino)-2,3-dihydro-1H-inden-5-ylamino)furo[2,3]pyridin-2-yl)pyrimidine-5-carboxamide

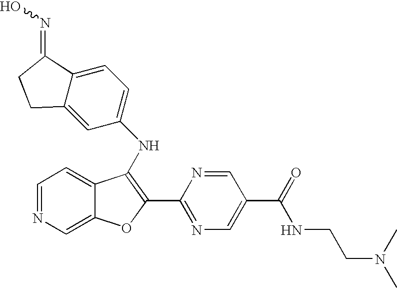

Prepared according to the method of Example 140. MS (APCI) m/z 472.3 (M+1). ¹H NMR (400 MHz, CDCl₃) δ 9.1 (m, 2H), 9.0 (s, 1H), 8.6-8.5 (m, 1H), 8.4-8.3 (m, 2H), 7.3 (m, 2H), 7.0-6.9 (m, 1H), 6.6 (m, 1H), 3.7 (m, 2H), 2.8-2.6 (m, 6H), 2.5 (s, 6H).

### Example 142

### 5-(2-(5-(hydroxymethyl)pyrimidin-2-yl)furo[2,3-c]pyridin-3-ylamino)-2,3-dihydroinden-1-one oxime

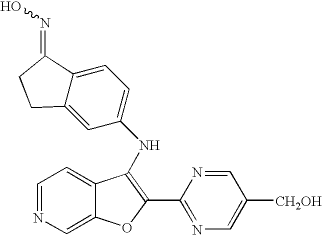

Step A: 5-(2-(5-(hydroxymethyl)pyrimidin-2-yl)furo[2,3-c]pyridin-3-ylamino)-2,3-dihydroinden-1-one O-tert-butyldimethylsilyl oxime: To a cold (−78° C.) solution of (Z,E)-methyl 2-(3-(1-(tert-butyldimethylsilyloxyimino)-2,3-dihydro-1H-inden-5-ylamino)furo[2,3-c]pyridin-2-yl)pyrimidine-5-carboxylate (1.40 g, 2.64 mmol) in dichloromethane (20 mL) was added a solution of DIBAL (1.5 M in toluene, 4.05 mL, 6.03 mmol). The reaction was stirred at −78° C. for 2 hours before quenching with MeOH. The reaction mixture was concentrated and the crude product was purified by flash column chromatography, eluting with ethyl acetate/hexanes (4:1), dichloromethane/MeOH (20:1) to give the desired product (900 mg, 68%). MS (APCI) m/z 502.3 (M+1).

Step B: 5-(2-(5-(hydroxymethyl)pyrimidin-2-yl)furo[2,3-c]pyridin-3-ylamino)-2,3-dihydroinden-1-one oxime: Prepared from the product of Step A using the procedure described in Example 51. MS (APCI) m/z 388.2 (M+1) ¹H NMR (400 MHz, DMSO-d6) δ 10.6 (bs, 1H), 9.1 (s, 1H), 8.9 (m, 3H), 8.4 (m, 1H), 7.5 (m, 1H), 7.3 (m, 1H), 7.0 (m, 2H), 5.5 (bs, 1H), 4.6 (s, 2H), 2.9 (m, 2H), 2.8 (m, 2H).

### Example 143

### (Z,E)-2-(3-(1-(hydroxyimino)-2,3-dihydro-1H-inden-5-ylamino)furo[2,3-c]pyridin-2-yl)pyrimidine-5-carboxylic acid

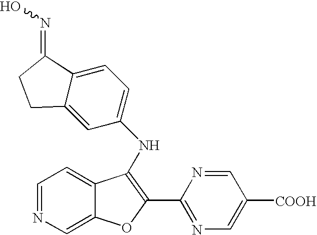

(Z,E)-Methyl 2-(3-(1-(tert-butyldimethylsilyloxyimino)-2,3-dihydro-1H-inden-5-ylamino)furo[2,3-c]pyridin-2-yl)pyrimidine-5-carboxylate (47 mg, 0.089 mmol) was suspended in MeOH (5.0 mL). To this was added 1N NaOH (1.0 M, 0.21 mL, 0.21 mmol) and the reaction mixture was heated at reflux for 1 hour and then concentrated under reduced pressure. The residue was suspended in water (5.0 mL) and the pH of the solution adjusted to ˜pH 5 with HOAc (0.1 mL). The resulting solid was collected by filtration, washed with dichloromethane and dried in vacuo to give the desired product (28 mg, 79%). MS (APCI) m/z 402.1 (M+1) ¹H NMR (400 MHz, DMSO-d₆) δ 10.7 (bs, 1H), 9.2 (s, 2H), 9.1 (m, 2H), 8.4 (m, 1H), 7.5 (m, 1H), 7.3 (m, 1H), 7.2-7.0 (m, 2H), 2.9 (m, 2H), 2.8 (m, 2H).

### Example 144

### 5-(2-(5-((4-Methylpiperazin-1-yl)methyl)pyrimidin-2-yl)furo[2,3-c]pyridin-3-ylamino)-2,3-dihydroinden-1-one oxime

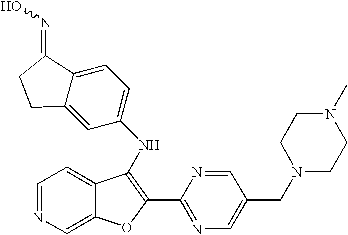

Step A: 2-(3-(1-(tert-butyldimethylsilyloxyimino)-2,3-dihydro-1H-inden-5-ylamino)furo[2,3-c]pyridin-2-yl)pyrimidine-5-carbaldehyde: To a solution of 5-(2-(5-(hydroxymethyl)pyrimidin-2-yl)furo[2,3-c]pyridin-3-ylamino)-2,3-dihydroinden-1-one O-tert-butyldimethylsilyl oxime (0.060 g, 0.12 mmol) in dichloromethane (5.0 mL) was added Dess-Martin periodinane (0.091 g, 0.215 mmol). The reaction mixture was stirred at ambient temperature overnight and then concentrated. The crude product was purified by flash column chromatography eluting with ethyl acetate/hexanes (1:1) to give the desired product (35 mg, 59%). MS (APCI) m/z 500.3 (M+1).

Step B: 5-(2-(5-((4-methylpiperazin-1-yl)methyl)pyrimidin-2-yl)furo[2,3-c]pyridin-3-ylamino)-2,3-dihydroinden-1-one O-tert-butyldimethylsilyl oxime: 2-(3-(1-(tert-butyldimethylsilyloxyimino)-2,3-dihydro-1H-inden-5-ylamino)furo[2,3-c]pyridin-2-yl)pyrimidine-5-carbaldehyde (35 mg, 0.070 mmol) and N-methyl piperazine (0.035 g, 0.35 mmol) were suspended in dichloromethane (5 mL) and NaBH(OAc)₃ (0.053 g, 0.25 mmol) was added and the reaction mixture was stirred at ambient temperature for 2 hours. The reaction mixture was quenched with MeOH (2.0 mL) and concentrated. The crude product was purified by flash column chromatography, eluting with dichloromethane/MeOH (50:1) and dichloromethane/MeOH (10:1) to give the desired product (40 mg, 98%). MS (APCI) m/z 584.1 (M+1).

Step C: 5-(2-(5-((4-methylpiperazin-1-yl)methyl)pyrimidin-2-yl)furo[2,3-c]pyridin-3-ylamino)-2,3-dihydroinden-1-one oxime: Deprotection of the product of Step B was carried out using the general procedure described in Example 51 to provide the desired product. MS (APCI) m/z 470.0 (M+1). ¹H NMR (400 MHz, DMSO-d6) δ 10.6 (bs, 1H), 9.1 (s, 1H), 8.9 (m, 3H), 8.4 (m, 1H), 7.5 (m, 1H), 7.3 (m, 1H), 7.0 (m, 2H), 3.6 (s, 2H), 2.9 (m, 2H), 2.8 (m, 2H), 2.7-2.4 (m, 8H), 2.3 (s, 3H).

Additional compounds prepared as described in Example 144 are shown in Table 4.

TABLE 4

| Ex. | Structure | Name | MS (m/z) |
|---|---|---|---|
| 145 | 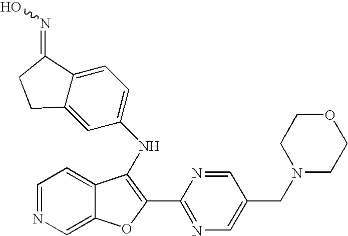 | 5-(2-(5- (morpholinomethyl)pyrimidin- 2-yl)furo[2,3-c]pyridin-3- ylamino)-2,3-dihydro-1H- inden-1-one oxime | 457.2 (M + 1) |
| 146 | 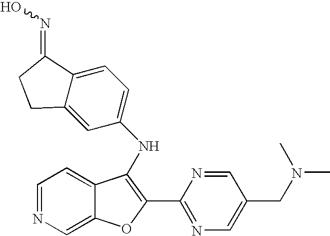 | 5-(2-(5-((dimethylamino) methyl)pyrimidin-2-yl)furo [2,3-c]pyridin-3-ylamino)-2,3- dihydro-1H-inden-1-one oxime | 415.2 (M + 1) |
| 147 | 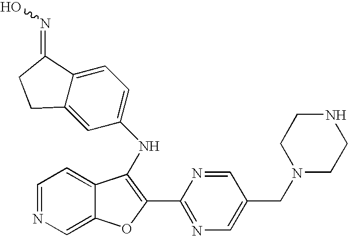 | 5-(2-(5-(piperazin-1- ylmethyl)pyrimidin-2-yl)furo [2,3-c]pyridin-3-ylamino)-2,3- dihydro-1H-inden-1-one oxime | 456.0 (M + 1) |
| 148 | 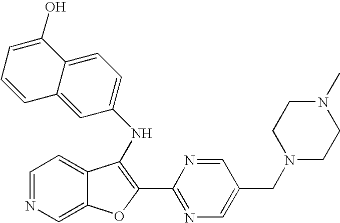 | 6-(2-(5-((4-methylpiperazin-1- yl)methyl)pyrimidin-2-yl)furo [2,3-c]pyridin-3- ylamino)naphthalen-1-ol | 467.1 (M + 1) |
| 149 | 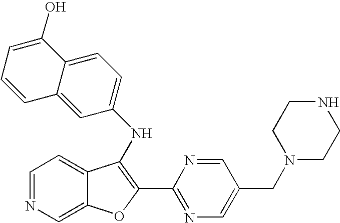 | 6-(2-(5-(piperazin-1- ylmethyl)pyrimidin-2-yl)furo [2,3-c]pyridin-3- ylamino)naphthalen-1-ol | 453.2 (M + 1) |
| 150 | 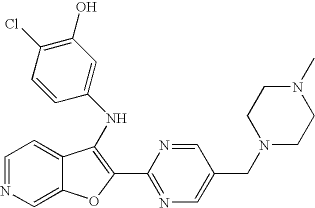 | 2-chloro-5-(2-(5-((4- methylpiperazin-1- yl)methyl)pyrimidin-2-yl)furo [2,3-c]pyridin-3- ylamino)phenol | 451.2, 453.2 (M + 1) |
| 151 | 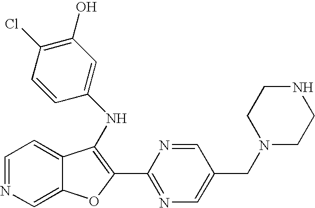 | 2-chloro-5-(2-(5-(piperazin-1- ylmethyl)pyrimidin-2-yl)furo [2,3-c]pyridin-3- ylamino)phenol | 437.2, 439.3 (M + 1) |

### Example 152

### Methyl 2-(3-(5-hydroxynaphthalen-2-ylamino)furo[2,3-c]pyridin-2-yl)pyrimidine-5-carboxylate

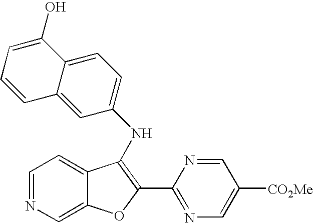

Step A: Methyl 2-(3-(5-(benzyloxymethoxy)naphthalen-2-ylamino)furo[2,3-c]pyridin-2-yl)pyrimidine-5-carboxylate: Prepared from 5-(benzyloxymethoxy)naphthalen-2-amine and methyl 2-(3-(trifluoromethylsulfonyloxy)furo[2,3-c]pyridin-2-yl)pyrimidine-5-carboxylate (Example 138) according to the method of Example 53 in 70% yield.

Step B: Methyl 2-(3-(5-hydroxynaphthalen-2-ylamino)furo[2,3-c]pyridin-2-yl)pyrimidine-5-carboxylate: Methyl-2-(3-(5-(benzyloxymethoxy)naphthalen-2-ylamino)furo[2,3-c]pyridin-2-yl)pyrimidine-5-carboxylate (69 mg) was dissolved in methanol (5 mL) and 6N HCl (0.5 mL) was added. The reaction was heated at 50° C. for 10 hours, and was then cooled to ambient temperature, and the solvent evaporated. Ethyl acetate and saturated NaHCO₃ were added, and the layers were separated, dried (MgSO₄), and concentrated. Silica gel chromatography (eluting with 75% EtOAc/hexanes) afforded the product as a solid (35 mg, 66%). MS (APCI-pos) M+1=413.4. ¹H NMR (400 MHz, d6-DMSO) δ 10.12 (s, 1H), 9.34 (s, 2H), 9.25 (s, 1H), 9.12 (s, 1H), 8.33-8.30 (m, 1H), 8.15-8.12 (m, 1H), 7.54-7.51 (m, 1H), 7.43-7.39 (m, 1H), 7.28-7.23 (m, 1H), 7.21-7.14 (m, 2H), 6.79-6.76 (m, 1H), 3.93 (s, 3H).

### Example 153

### 2-(3-(5-(tert-Butyldimethylsilyloxy)naphthalen-2-ylamino)furo[2,3-c]pyridin-2-yl)pyrimidine-5-carbaldehyde

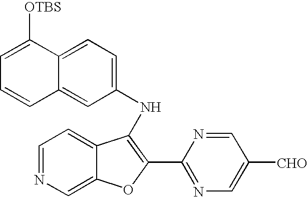

Prepared according to the method of Example 144, step A. MS (APCI) m/z=497.4 (M+1).

### Example 154

### 2-(3-(3-(tert-Butyldimethylsilyloxy)-4-chlorophenylamino)furo[2,3-c]pyridin-2-yl)pyrimidine-5-carbaldehyde

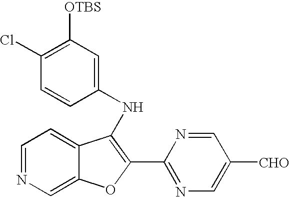

Prepared according to the method of Example 144, step A. MS (APCI) m/z 481.4, 483.4 (M+1).

### Example 155

### 6-(2-(5-Aminopyrimidin-2-yl)furo[2,3-c]pyridin-3-ylamino)naphthalen-1-ol

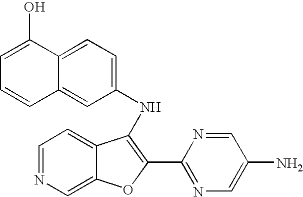

Step A: Methyl 2-(3-(5-methoxynaphthalen-2-ylamino)furo[2,3-c]pyridin-2-yl)pyrimidine-5-carboxylate: Prepared from methyl 2-(3-(trifluoromethylsulfonyloxy)furo[2,3-c]pyridin-2-yl)pyrimidine-5-carboxylate and 5-methoxynaphthalen-2-amine according to the method of Example 53, followed by basic hydrolysis MS (APCI) m/z 413.4 (M+1).

Step B: 2-(5-Aminopyrimidin-2-yl)-N-(5-methoxynaphthalen-2-yl)furo[2,3-c]pyridin-3-amine: Prepared from the product of Step A according to the method of Example 83, step E. MS (APCI) m/z 384.4 (M+1).

Step C: 6-(2-(5-Aminopyrimidin-2-yl)furo[2,3-c]pyridin-3-ylamino)naphthalen-1-ol: Prepared from the product of Step C according to the method o Example 50. ¹H NMR (400 MHz, MeOH-d⁴) δ 8.9 (bs, 1H), 8.3 (s 2H), 8.2 (bs, 1H), 8.1 (m, 1H), 7.3 (bs, 1H), 7.2 (m, 3H), 7.0 (m, 1H), 6.6 (m, 1H). MS (APCI) m/z 370.5 (M+1).

### Example 156

### 2-(5-Bromopyrimidin-2-yl)-3-(tert-butyldiphenylsilyloxy)furo[2,3-c]pyridine

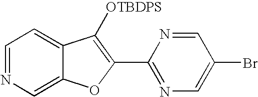

Step A: 3-(tert-Butyldiphenylsilyloxy)furo[2,3-c]pyridine: To a suspension of furo[2,3-c]pyridin-3(2H)-one hydrochloride (5.1 g, 29.7 mmol) in dichloromethane (100 mL) was added sequentially imidazole (6.07 g, 89.2 mmol) and tert-butylchlorodiphenylsilane (10.65 mL, 41.6 mmol). The reaction was stirred at ambient temperature for 1 hour before quenching with water (50 mL). The aqueous layer was extracted with dichloromethane. The combined organic layers were dried over Na₂SO₄, filtered and concentrated. The crude product was purified by flash column chromatography, eluting with hexanes/ethyl acetate (9:1) to give the desired product (6.8 g, 61%). ¹H NMR (400 MHz, CDCl₃) δ 8.7 (s, 1H), 8.4 (d, J=5.6 Hz, 1H), 7.7 (m, 4H), 7.5 (m, 3H), 7.4 (m, 4H), 6.8 (s, 1H), 1.2 (s, 9H). MS (APCI) m/z 374.3 (M+1).

Step B: 2-Bromo-3-(tert-butyldiphenylsilyloxy)furo[2,3-c]pyridine: To a solution of 3-(tert-butyldiphenylsilyloxy)furo[2,3-c]pyridine (1.30 g, 3.48 mmol) in CHCl₃ (20 mL) was added Br₂ (1.67 g, 10.4 mmol) as a solution in CHCl₃ (5.0 mL). The reaction was stirred at ambient temperature for 1 hour before quenching with saturated Na₂S₂O₃ and saturated NaHCO₃. The aqueous layer was extracted with dichloromethane. The combined organic layers were dried over Na₂SO₄, filtered and concentrated. The crude product was purified by flash column chromatography, eluting with dichloromethane and dichloromethane/ethyl acetate (9:1) to give the desired product (1.42 g, 90%). ¹H NMR (400 MHz, CDCl₃) δ 8.6 (s, 1H), 8.0 (d, J=5.6 Hz, 1H), 7.7 (m, 4H), 7.4 (m, 6H), 6.7 (d, J=5.6 Hz, 1H), 1.2 (s, 9H). MS (APCI) m/z 452.3, 454.2 (M+1).

Step C: 2-(5-bromopyrimidin-2-yl)-3-(tert-butyldiphenylsilyloxy)furo[2,3-c]pyridine: To a flame dried flask containing 2-bromo-3-(tert-butyldiphenylsilyloxy)furo[2,3-c]pyridine (0.674 g, 1.49 mmol) in cold (−10° C.) THF (20 mL) was added i-PrMgCl (2.0 M in THF, 1.12 mL, 2.23 mmol) slowly via a syringe. The reaction was stirred at −10° C. for 1 hour, and then ZnCl₂ (0.5 M solution in THY, 4.47 mL, 2.23 mmol) was added and the reaction mixture was stirred at ambient temperature for 15 minutes. In another flame dried flask under Ar was charged Pd(PPh₃)₄ (0.172 g, 0.149 mmol), 5.0 mL anhydrous THF and 5-bromo-2-iodopyrimidine (0.637 g, 2.23 mmol). To this was added the aryl zinc solution via a cannula. The reaction mixture was stirred at ambient temperature under Ar overnight. The reaction mixture was concentrated and the residue was diluted with water (20 mL) and ethyl acetate (50 mL). The aqueous layer was extracted with ethyl acetate. The combined organic layers were dried over Na₂SO₄, filtered and concentrated. The crude product was purified by flash column chromatography, eluting with hexanes/ethyl acetate (4:1) and hexanes/ethyl acetate (2:1) to give the desired product (0.62 g, 79%). ¹H NMR (400 MHz, CDCl₃) δ 8.9 (s, 1H), 8.7 (s, 2H), 8.1 (d, J=5.6 Hz, 1H), 7.8 (m, 4H), 7.4-7.3 (m, 6H), 6.9 (d, J=5.6 Hz, 1H), 1.2 (s, 9H). MS (APCI) m/z 530.3, 532.3 (M+1).

### Example 157

### 2-(5-(4-Methylpiperazin-1-yl)pyrimidin-2-yl)furo[2,3-c]pyridin-3-yl trifluoromethanesulfonate

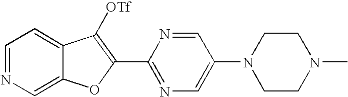

Step A: 2-(5-(4-Methylpiperazin-1-yl)pyrimidin-2-yl)furo[2,3-c]pyridin-3-ol: 2-(5-bromopyrimidin-2-yl)-3-(tert-butyldiphenylsilyloxy)furo[2,3-c]pyridine (0.500 g, 0.943 mmol), 1-methylpiperazine (0.142 g, 1.41 mmol) was suspended in toluene (15.0 mL) and argon gas was bubbled through the solution for 15 minutes. To this was added Pd₂(dba)₃ (0.0863 g, 0.0943 mmol), Xphos (0.180 mmol, 0.377 mmol) and NaOt-Bu (0.163 g, 1.70 mmol). Argon gas was bubbled through the solution another 15 minutes and the reaction was heated at reflux overnight. The reaction was diluted with water (10 mL), 1N NaOH (10 mL) and filtered through GF/F filter paper. The aqueous layer was washed with ethyl acetate. The pH of the aqueous layer was adjusted to ˜7 with 1 N HCl. The aqueous layer was then extracted with dichloromethane. The combined organic layers were dried over Na₂SO₄, filtered and concentrated to give the desired product (0.22 g, 75%). ¹H NMR (400 MHz, CDCl₃) δ 8.9 (bs, 1H), 8.5 (m, 3H), 7.6 (m, 1H), 3.4 (m, 4H), 2.6 (m, 4H), 2.4 (s, 3H). MS (APCI) m/z 312.5 (M+1).

Step B: 2-(5-(4-Methylpiperazin-1-yl)pyrimidin-2-yl)furo[2,3-c]pyridin-3-yl trifluoromethanesulfonate: Formation of the triflate of the product of step A was carried out using the procedure described in Example 55. MS (APCI) m/z 444.0 (M+1).

### Example 158

### (Z,E)-5-(2-(5-(4-methylpiperazin-1-yl)pyrimidin-2-yl)furo[2,3-c]pyridin-3-ylamino)-2,3-dihydroinden-1-one oxime

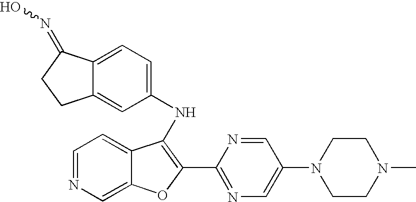

Prepared from 2-(5-(4-methylpiperazin-1-yl)pyrimidin-2-yl)furo[2,3-c]pyridin-3-yl trifluoromethanesulfonate (Example 157) and 5-amino-2,3-dihydro-1H-inden-1-one O-tert-butyldimethylsilyl oxime using the method of Example 53, followed by the method of Example 52. ¹H NMR (400 MHz, CDCl₃) δ 9.0 (s, 1H), 8.5 (s, 2H), 8.4 (m, 1H), 7.6 (m, 1H), 7.3 (m, 2H), 7.0 (m, 2H), 3.4 (m, 4H), 3.0 (m, 4H), 2.6 (m, 4H), 2.4 (s, 3H). MS (APCI) m/z 456.3 (M+1).

### Example 159

### 5-(2-(5-(4-methylpiperazin-1-yl)pyrimidin-2-yl)furo[2,3-c]pyridin-3-ylamino)-2,3-dihydroinden-1-one

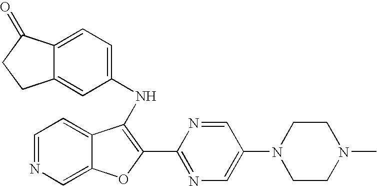

Prepared from 2-(5-(4-methylpiperazin-1-yl)pyrimidin-2-yl)furo[2,3-c]pyridin-3-yl trifluoromethanesulfonate (Example 157) and 5-Amino-2,3-dihydro-1H-inden-1-one O-tert-butyldimethylsilyl oxime (Example 125) according to the method of Example 53. MS (APCI) m/z 441.4 (M+1).

### Example 160

### N5-(2-(5-(4-Methylpiperazin-1-yl)pyrimidin-2-yl)furo[2,3-c]pyridin-3-yl)-2,3-dihydro-1H-indene-1,5-diamine

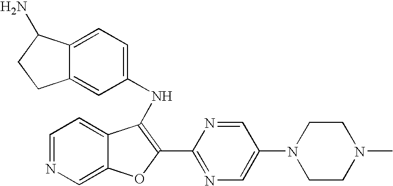

Step A: (Z)-2-((dimethylamino)methylene)-5-(2-(5-(4-methylpiperazin-1-yl)pyrimidin-2-yl)furo[2,3-c]pyridin-3-ylamino)-2,3-dihydroinden-1-one: To a suspension of 5-(2-(5-(4-methylpiperazin-1-yl)pyrimidin-2-yl)furo[2,3-c]pyridin-3-ylamino)-2,3-dihydroinden-1-one (Example 159; 0.020 g, 0.0454 mmol) in toluene (4.0 mL) was added Ti(OEt)₄ (0.038 mL, 0.182 mmol), followed by 2-methylpropane-2-sulfinamide (0.011 g, 0.0908 mmol). The reaction mixture was heated at reflux under N₂ overnight, cooled to ambient temperature, concentrated, and used directly in step B. MS (APCI) m/z 544.0 (M+1).

Step B: 2-Methyl-N-(5-(2-(5-(4-methylpiperazin-1-yl)pyrimidin-2-yl)furo[2,3-c]pyridin-3-ylamino)-2,3-dihydro-1H-inden-1-yl)propane-2-sulfinamide: The crude product from step A was cooled to −50° C. and to this was added NaBH₄ (8.7 mg, 0.23 mmol). The reaction mixture was allowed to warm to ambient temperature overnight, then quenched with MeOH (1.0 mL) and concentrated. The residue was used directly in step C. MS (APCI) m/z 546.1 (M+1).

Step C: N5-(2-(5-(4-methylpiperazin-1-yl)pyrimidin-2-yl)furo[2,3-c]pyridin-3-yl)-2,3-dihydro-1H-indene-1,5-diamine: The crude product from step B was suspended in MeOH (5.0 mL) and to this was added 0.5 mL of 4N HCl. The reaction mixture was left at ambient temperature overnight. The reaction mixture was concentrated and the residue was dissolved in dichloromethane (50 mL) and saturated NaHCO₃ (20 mL). The aqueous layer was extracted with dichloromethane. The combined organics were dried, filtered and concentrated. The crude product was purified by flash column chromatography, eluting with dichloromethane/MeOH (5:1), dichloromethane/MeOH/TEA (20:1:0.1) to give the desired product (3.0 mg, 15%). ¹H NMR (400 MHz, CDCl₃) δ 9.0 (s, 1H), 8.5 (s, 2H), 8.4 (s, 1H), 8.3 (m, 1H), 7.3 (m, 1H), 6.9 (m, 2H), 4.4 (m, 1H), 3.3 (m, 4H), 3.0 (m, 1H), 2.8 (m, 1H), 2.6 (m, 4H), 2.5 (m, 1H), 2.4 (s, 3H), 1.8 (m, 1H). MS (APCI) m/z 442.5 (M+1).

### Example 161

### N-(2-(5-(4-Methylpiperazin-1-yl)pyrimidin-2-yl)furo[2,3-c]pyridin-3-yl)quinolin-3-amine

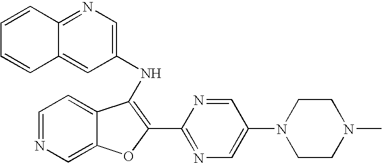

Prepared from 2-(5-(4-methylpiperazin-1-yl)pyrimidin-2-yl)furo[2,3-c]pyridin-3-yl trifluoromethanesulfonate (Example 157) and 3-aminoquinoline according to the method of Example 53. ¹H NMR (400 MHz, CDCl₃) δ 9.0 (s, 1H), 8.9 (s, 1H), 8.7 (s, 1H), 8.5 (s, 2H), 8.3 (m, 1H), 8.1 (m, 1H), 7.6-7.5 (m, 4H), 3.4 (m, 4H), 2.6 (m, 4H), 2.3 (s, 3H). MS (APCI) m/z 438.5 (M+1).

### Example 162

### N-(2-(2-(2-(Dimethylamino)ethoxy)pyrimidin-5-yl)furo[2,3-c]pyridin-3-yl)quinolin-3-amine

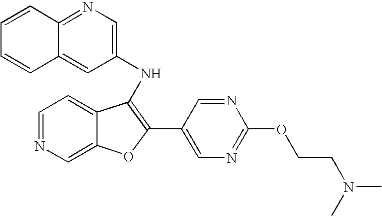

Step A: 3-(tert-Butyldiphenylsilyloxy)-2-(2-chloropyrimidin-5-yl)furo[2,3-c]pyridine: To a flame dried flask containing 2-bromo-3-(tert-butyldiphenylsilyloxy)furo[2,3-c]pyridine (2.00 g, 4.42 mmol) in cold (−10° C.) anhydrous THF (20 mL) was added i-PrMgCl (2.0 M in THF, 3.32 mL, 6.63 mmol) slowly via a syringe. Stirred at −10° C. for 1 hour and to this was added ZnCl₂ (0.5 M solution in THF, 13.3 mL, 6.63 mmol). The cold bath was removed and the reaction mixture was stirred at ambient temperature for 15 minutes. In another 100 mL flame dried flask under Ar was charged Pd(PPh₃)₄ (1.02 g, 0.884 mmol), 20 mL anhydrous THF and 5-bromo-2-chloropyrimidine (1.28 g, 6.63 mmol). To this was added the aryl zinc solution via a cannula. The reaction mixture was left at ambient temperature under Ar overnight. The reaction mixture was concentrated and the residue was diluted with water (20 mL) and ethyl acetate (50 mL). The aqueous layer was extracted with ethyl acetate. The combined organic layers were dried over Na₂SO₄, filtered and concentrated. The crude product was purified by flash column chromatography, eluting with hexanes/ethyl acetate (4:1) to give the desired product (1.02 g, 48%) MS (APCI) m/z 486.4.3, 488.4 (M+1).

Step B: 2-(2-(2-(dimethylamino)ethoxy)pyrimidin-5-yl)furo[2,3-c]pyridin-3-ol: To a suspension of NaH (60% suspension in mineral oil, 0.082 g, 2.06 mmol) in THF was added 2-(dimethylamino)ethanol (0.073 g, 0.823 mmol). The reaction was stirred at ambient temperature for 20 minutes before adding 3-(tert-butyldimethylsilyloxy)-2-(2-chloropyrimidin-5-yl)furo[2,3-c]pyridine (0.200 g, 0.411 mmol) was added. The reaction was stirred at 80° C. for 2 hours, then Cooled to 0° C., quenched with MeOH (1.0 mL) and concentrated. The crude product was purified by flash column chromatography, eluting with DCM/MeOH (20:1), DCM.MeOH (10:1), DCM.MeOH (5:1) to give the desired product (0.085 g, 69%). MS (APCI) m/z 300.9 (M+1).

Step C: 2-(2-(4-methylpiperazin-1-yl)pyrimidin-5-yl)furo[2,3-c]pyridin-3-yl trifluoromethanesulfonate: Prepared from the product of step B using the general procedure described in Example 55. MS (APCI) m/z 432.9 (M+1).

Step D: N-(2-(2-(2-(Dimethylamino)ethoxy)pyrimidin-5-yl)furo[2,3-c]pyridin-3-yl)quinolin-3-amine: Prepared from the product of Step C and 3-aminoquinoline using the general procedure described in Example 53. ¹H NMR (400 MHz, CDCl₃) δ 9.2 (s, 2H), 9.0 (s, 1H), 8.7 (d, J=2.7 Hz, 1H), 8.4 (d, J=5.2 Hz, 1H), 8.0 (d, J=7.7 Hz, 1H), 7.5 (m, 2H), 7.4 (m, 1H), 7.3 (d, J=5.4 Hz, 1H), 7.1 (d, J=2.5 Hz, 1H), 5.9 (bs, 1H), 4.5 (t, J=5.8 Hz, 2H), 2.8 (t, J=5.8 Hz, 2H), 2.4 (s, 6H) ppm. MS (APCI) m/z 427.0 (M+1).

### Example 163

### (E)-5-(2-(4-Morpholinopyrimidin-2-yl)furo[2,3-c]pyridin-3-ylamino)-2,3-dihydro-1H-inden-1-one oxime

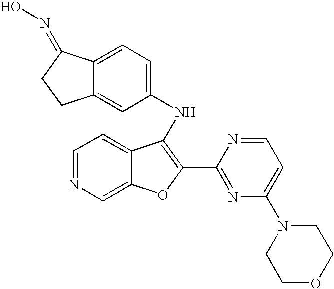

Step A: 4-(2-(Methoxymethyl)pyrimidin-4-yl)morpholine: A slurry of 4-chloro-2-(methoxymethyl)pyrimidine (0.58 g, 3.66 mmol) (J. Med. Chem. 2002, 45, 511-528) and TEA (1.53 mL, 11.0 mmol) and morpholine (0.480 mL, 5.49 mmol) in 20 mL THF was heated to 65° C. for 3 hours. The slurry was filtered through celite and concentrated to a brown oil and used without further purification. MS (APCI-pos) M+1=210.2.

Step B: (4-Morpholinolpyrimidin-2-yl)methanol: To a solution of 4-(2-(methoxymethyl)pyrimidin-4-yl)morpholine (0.766 g, 3.66 mmol) in 15 ml dichloromethane at 0° C. was added tribromoborane (0.346 mL, 3.66 mmol) dropwise. The reaction was warmed to ambient temperature over 1 hour. The reaction was quenched with saturated aqueous sodium bicarbonate. The layers were separated and the aqueous was washed with 25% MeOH/dichloromethane, dried over sodium sulfate and concentrated to an oil. The oil was filtered through a plug of SiO₂ with 10% 2M NH₃/MeOH in dichloromethane and concentrated to give the title compound (0.570 g, 79.8% for two steps) as a solid. MS (APCI-pos) M+1=196.1.

Step C: Ethyl 3-((4-morpholinopyrimidin-2-yl)methoxy)isonicotinate: PPh₃ (0.4180 g, 1.594 mmol) was dissolved in 3 mL THF and cooled to 0° C. DIAD (0.3087 mL, 1.594 mmol) was added dropwise and the reaction was stirred for 10 minutes. After 10 minutes (4-morpholinopyrimidin-2-yl)methanol (0.2852 g, 1.461 mmol) was added in 4 mL THF and stirred for 10 minutes. A slurry of ethyl 3-hydroxyisonicotinate (0.222 g, 1.328 mmol) was added and warmed to ambient temperature for 4 hours. The reaction was concentrated to a brown oil, dissolved in EtOAc and extracted with 1N HCl. The aqueous layer was neutralized with solid Na₂CO₃ and extracted with 25% IPA/dichloromethane. The combined organics were dried over sodium sulfate and concentrated to an oil. Purification by silica gel chromatography provided the title compound (0.119 g, 26% for two steps) as a white solid. MS (APCI-pos) M+1=345.2.

Step D: 2-(4-morpholinopyrimidin-2-yl)furo[2,3-c]pyridin-3-ol: To a solution ethyl 3-((4-morpholinopyrimidin-2-yl)methoxy)isonicotinate (0.119 g, 0.346 mmol) in 3 mL THF was added NaH (0.0193 g, 0.484 mmol) in one portion and the reaction was stirred at ambient temperature overnight. A white solid precipitated in solution. The solution was dissolved in 1N HCl and washed once with dichloromethane. This aqueous layer was neutralized with solid Na₂CO₃ and was extracted with 25% IPA/dichloromethane. The combined organics were dried over sodium sulfate and concentrated to an oil. Purification by silica gel chromatography provided the title compound (0.051 g, 49%) as a white solid. MS (APCI-pos) M+1=299.3.

Step E: 2-(4-morpholinopyrimidin-2-yl)furo[2,3-c]pyridin-3-yl trifluoromethanesulfonate: To a slurry of 2-(4-morpholinopyrimidin-2-yl)furo[2,3-c]pyridin-3-ol (0.0506 g, 0.170 mmol) and pyridine (0.0206 mL, 0.254 mmol) in 20 ml dichloromethane at 0° C. was added trifluoromethanesulfonic anhydride (0.0574 g, 0.204 mmol) dropwise. The reaction was warmed to ambient temperature and stirred for one hour. The reaction was quenched with water, and the aqueous was extracted with 25% IPA/dichloromethane. The combined organics were dried over sodium sulfate and concentrated to an oil, which was purified by silica gel chromatography to give the title compound (0.056 g, 76%) as a clear oil. MS (APCI-pos) M+1=430.9.

Step F: (E)-5-(2-(4-morpholinopyrimidin-2-yl)furo[2,3-c]pyridin-3-ylamino)-2,3-dihydro-1H-inden-1-one O-tert-butyldimethylsilyl oxime: The product of Step E and 5-amino-2,3-dihydro-1H-inden-1-one O-tert-butyldimethylsilyl oxime (Example 125) were reacted according to the method of Example 53 to afforded the title compound (0.0133 g, 37%) as a tan solid. MS (APCI-pos) M+1=557.2.

Step G: (E)-5-(2-(4-morpholinopyrimidin-2-yl)furo[2,3-c]pyridin-3-ylamino)-2,3-dihydro-1H-inden-1-one oxime: (E)-5-(2-(4-morpholinopyrimidin-2-yl)furo[2,3-c]pyridin-3-ylamino)-2,3-dihydroinden-1-one O-tert-butyldimethylsilyl oxime (0.0133 g, 0.0239 mmol) was dissolved in 2 ml DCM. TBAF (0.0311 ml, 0.0311 mmol) was added at ambient temperature and stirred for 1 hour. The reaction was purified by silica gel chromatography to give the title compound (0.0065 g, 62%) as an oil. MS (APCI-pos) M+1=557. ¹H NMR (400 MHz, CDCl₃) δ 9.00 (s, 1H), 8.95 (s, 1H), 8.34-8.37 (m, 2H), 7.62 (d, 1H), 7.35 (d, 1H), 6.97-6.98 (m, 2H), 6.41 (d, 1H), 3.84-3.87 (m, 4H), 3.73 (bs, 4H), 3.01 (bs, 4H).

### Example 164

### 6-(2-(4-Morpholinopyrimidin-2-yl)furo[2,3-c]pyridin-3-ylamino)naphthalen-1-ol

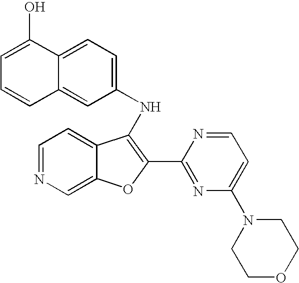

Prepared according to the procedure of Example 163, replacing (E)-5-amino-2,3-dihydroinden-1-one O-tert-butyldimethylsilyl oxime with 5-(tert-butyldimethylsilyloxy)naphthalen-2-amine to give the desired compound as a tan solid. MS (APCI-pos) M+1=440.5. ¹H NMR (400 MHz, CDCl₃) δ 9.01-9.02 (m, 2H), 8.38 (d, 1H), 8.32 (d, 1H), 8.22 (d, 1H), 7.37 (bs, 2H), 7.24-7.30 (m, 5H), 6.74 (d, 1H), 6.41 (d, 1H), 3.85-3.87 (m, 4H), 3.74-3.75 (m, 4H).

### Example 165

### (E)-6-(2-(1-Hydrazono-4-methoxybutyl)furo[2,3-c]pyridin-3-ylamino)naphthalen-1-ol

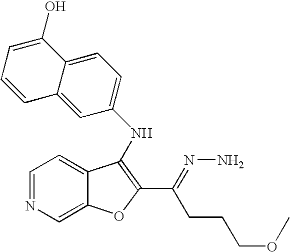

Step A: A solution of methyl 4-methoxybutanoate (1.48 g, 1.0 equiv) in methanol was treated with LiOH.H₂O (3.1 equiv), and the mixture was heated to 50° C. for 3 hours. The mixture was cooled to ambient temperature and 5.0 N HCl (7.0 mL) was added. The mixture was concentrated to dryness under reduced pressure, CH₂Cl₂ was added and the mixture sonicated for 30 minutes. The insoluble salts were removed by vacuum filtration and the filtrate concentrated to afford 4-methoxybutanoic acid as a colorless oil (1.023 g, 77%).

Step B: N,4-dimethoxy-N-methylbutanamide: The product of Step A and N,O-dimethylhydroxylamine hydrochloride were reacted according to the method of Example 56 to provide the desired compound in 29% yield.

Step C: 3-(benzyloxymethoxy)furo[2,3-c]pyridine: Prepared from the product of step B according to Example 49.

Step D: 1-(3-(benzyloxymethoxy)furo[2,3-c]pyridin-2-yl)-4-methoxybutan-1-one: 3-(Benzyloxymethoxy)furo[2,3-c]pyridine (152 mg, 1.0 equiv) was dissolved in THF (3.0 mL) under Ar and cooled to −78° C. n-BuLi was added dropwise over 1-2 minutes at −78° C. After 20 minutes, N,4-dimethoxy-N-methylbutanamide (1.3 equiv) was added dropwise (as a solution in 1.0 mL THF). The solution was allowed to warm to ambient temperature over 20 hours, and was then quenched with aq. NH₄Cl and diluted with EtOAc. The layers were separated, the aqueous layer extracted once with EtOAc, and the combined organics were dried (MgSO₄). Silica gel chromatography (eluting with 40% EtOAc/hexanes) afforded the product as a yellow oil (204 mg, 96%). MS (APCI-pos) M+1=356.0.

Step E: 1-(3-hydroxyfuro[2,3-c]pyridin-2-yl)-4-methoxybutan-1-one: To a solution of 1-(3-(benzyloxymethoxy)furo[2,3-c]pyridin-2-yl)-4-methoxybutan-1-one (135 mg, 1.0 equiv) in methanol (5 mL) was added 6N HCl (0.5 mL) and stirred for 20 hours at ambient temperature. The volatiles were removed under reduced pressure, basified carefully with saturated NaHCO₃, washed with EtOAc, then adjusted to pH 3-4 by addition of AcOH. The crude mixture was extracted with EtOAc, dried (MgSO₄), filtered, and concentrated under high vacuum to afford the crude product as a yellow solid.

Step F: 2-(4-methoxybutanoyl)furo[2,3-c]pyridin-3-yl trifluoromethanesulfonate: Prepared according to the method of Example 55 and purified by silica gel chromatography (eluting with 2% methanol/CH₂Cl₂) (overall yield of 49% for steps E and F).

Step G. 1-(3-(5-(tert-butyldimethylsilyloxy)naphthalen-2-ylamino)furo[2,3-c]pyridin-2-yl)-4-methoxybutan-1-one: Prepared from the product of Step F according to the method of Example 53 in 74% yield. MS (APCI-pos) M+1=491.3.

Step H: (E)-6-(2-(1-hydrazono-4-methoxybutyl)furo[2,3-c]pyridin-3-ylamino)naphthalen-1-ol: 1-(3-(5-(tert-Butyldimethylsilyloxy)naphthalen-2-ylamino)furo[2,3-c]pyridin-2-yl)-4-methoxybutan-1-one (26 mg) was dissolved in ethanol (1.0 mL), hydrazine-H₂O (10 equiv) was added, and the solution was heated at reflux for 72 hours. The volatiles were removed under reduced pressure, and the residue purified by silica gel chromatography (eluting with 100% EtOAc) to afford the product as a dirty yellow solid (15 mg, 73%). MS (APCI-pos) M+1=391.2. ¹H NMR (400 MHz, CDCl₃) δ 8.85 (s, 1H), 8.29-8.26 (m, 1H), 8.20-8.14 (m, 2H), 7.30-7.17 (m, 6H), 6.71-6.68 (m, 1H), 6.01-5.98 (bs, 2H), 3.45-3.42 (m, 2H), 3.38 (s, 3H), 2.91-2.87 (m, 2H), 2.04-1.98 (m, 2H).

### Example 166

### (3-(1-(Hydroxyimino)-2,3-dihydro-1H-inden-5-ylamino)furo[2,3-c]pyridin-2-yl)(tetrahydro-2H-pyran-4-yl)methanone

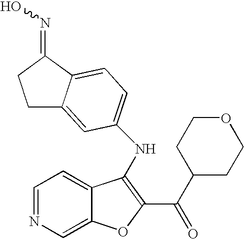

Step A: (3-(Benzyloxymethoxy)furo[2,3-c]pyridin-2-yl)(tetrahydro-2H-pyran-4-yl)methanol: 3-(Benzyloxymethoxy)furo[2,3-c]pyridine (112 mg, 1.0 equiv) was dissolved in THF (4.0 mL) and cooled to −78° C. n-BuLi (1.1 equiv) was then added dropwise, and the reaction was stirred for 1 hour at −78° C., then tetrahydro-2H-pyran-4-carbaldehyde (1.6 equiv) was added (as a solution in 1.0 mL THF) and the reaction was warmed slowly to ambient temperature over 15 hours. The reaction was treated with aqueous NH₄Cl and was extracted twice with EtOAc. After drying (MgSO₄) and filtering, the product was obtained after silica gel chromatography (eluting with ethyl acetate/hexanes) in 46% yield (75 mg). MS (APCI-pos) M+1=370.2.

Step B. (3-(Benzyloxymethoxy)furo[2,3-c]pyridin-2-yl)(tetrahydro-2H-pyran-4-yl)methanone: (3-(Benzyloxymethoxy)furo[2,3-c]pyridin-2-yl)(tetrahydro-2H-pyran-4-yl)methanol (75 mg, 1.0 equiv) was dissolved in CH₂Cl₂ and treated Dess-Martin periodinane (1.4 equiv). After stirring 30 minutes at ambient temperature, the reaction was concentrated and purified by silica gel chromatography (eluting with 60% EtOAc/hexanes) to afford the product as an oil (57 mg, 76%). MS (APCI-pos) M+1=368.0.

Step C: (3-(1-(tert-Butyldimethylsilyloxyimino)-2,3-dihydro-1H-inden-5-ylamino)furo[2,3-c]pyridin-2-yl)(tetrahydro-2H-pyran-4-yl)methanone: The compound was prepared from the product of step B following deprotection (example 49), triflation (example 55) and coupling according to Example 53.

Step D: (3-(1-(Hydroxyimino)-2,3-dihydro-1H-inden-5-ylamino)furo[2,3-c]pyridin-2-yl)(tetrahydro-2H-pyran-4-yl)methanone: (3-(1-(tert-Butyldimethylsilyloxyimino)-2,3-dihydro-1H-inden-5-ylamino)furo[2,3-c]pyridin-2-yl)(tetrahydro-2H-pyran-4-yl)methanone (19 mg) was dissolved in CH₂Cl₂ (4 mL), cooled to 0° C., and TFA (0.5 mL) was added. After 1.5 hours, the reaction mixture was concentrated, the residue basified with triethylamine, concentrated, and purified by silica gel chromatography (eluting with 100% EtOAc) to afford the product in 88% yield. MS (APCI-pos) M+1=392.3. ¹H NMR (400 MHz, CDCl₃) δ 8.98-8.95 (m, 2H), 8.45-8.33 (m, 1H), 7.69-7.64 (m, 2H), 7.27-7.25 (m, 1H), 7.13-7.07 (m, 2H), 4.15-4.09 (m, 2H), 3.66-3.59 (m, 2H), 3.55-3.46 (m, 1H), 3.10-3.00 (m, 4H), 1.98-1.90 (m, 4H).

### Example 167

### 1-(3-(5-Hydroxynaphthalen-2-ylamino)furo[2,3-c]pyridin-2-yl)-4-methoxybutan-1-one

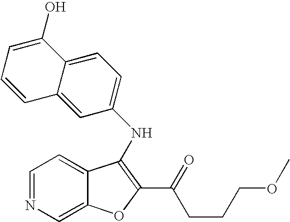

Prepared according to the method of Example 166. MS (APCI-pos) M+1=377.3. ¹H NMR (400 MHz, CDCl₃) δ 8.97-8.95 (s, 2H), 8.28-8.23 (m, 2H), 7.55-7.53 (m, 1H), 7.37-7.28 (m, 3H), 7.24-7.22 (m, 1H), 6.81-6.78 (m, 1H), 6.42 (bs, 1H), 3.57-3.53 (m, 2H), 3.38 (s, 3H), 3.13-3.08 (m, 2H), 2.14-2.05 (m, 2H).

### Example 168

### 1-(3-(1-(Hydroxyimino)-2,3-dihydro-1H-inden-5-ylamino)furo[2,3-c]pyridin-2-yl)butan-1-one

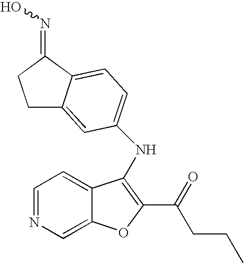

Step A: tert-Butyl 2-(methoxy(methyl)carbamoyl)furo[2,3-c]pyridin-3-ylcarbamate: Prepared from 3-(tert-butoxycarbonylamino)furo[2,3-c]pyridine-2-carboxylic acid and N,O-dimethylhydroxylamine hydrochloride according to the method of Example 56 in 52% yield.

Step B: tert-Butyl 2-butyrlfuro[2,3-c]pyridin-3-ylcarbamate: Tert-Butyl 2-(methoxy(methyl)carbamoyl)furo[2,3-c]pyridin-3-ylcarbamate (105 mg, 1.0 equiv) was dissolved in THF (2 mL) and cooled to 0° C. n-Propylmagnesium bromide (4.0 mL of a 0.91 M solution in THF, 11.2 equiv) was added in 1.0 mL portions every 30 minutes at 0° C. The reaction was then quenched with aqueous NH₄Cl and extracted with EtOAc. After drying, the product was obtained by silica gel chromatography (50% EtOAc/hexanes) as a white solid (52 mg, 52%). MS (APCI-pos) M+1=304.9.

Step C: 1-(3-Aminofuro[2,3-c]pyridin-2-yl)butan-1-one: tert-Butyl 2-butyrylfuro[2,3-c]pyridin-3-ylcarbamate (52 mg) was dissolved in CH₂Cl₂ (4 mL) and TFA (2 mL) was added. The reaction was stirred 2 hours at ambient temperature, and then concentrated. Triethylamine and CH₂Cl₂ were added, the mixture was concentrated, and the crude material purified by silica gel chromatography (50% EtOAc/hexanes) to afford the product as a solid (33 mg, 95%). MS (APCI-pos) M+1=205.3.

Step D: 1-(3-(1-(Hydroxyimino)-2,3-dihydro-1H-inden-5-ylamino)furo[2,3-c]pyridin-2-yl)butan-1-one: Following the method of Example 4 and TBAF-mediated desilylation (Example 52), the final product was obtained in 39% yield. ¹H NMR (400 MHz, CDCl₃) δ 8.95 (s, 1H), 8.87 (s, 1H), 8.34-8.32 (m, 1H), 7.66-7.64 (m, 1H), 7.28-7.26 (m, 1H), 7.14-7.06 (m, 3H), 3.68-3.64 (m, 2H), 3.09-2.94 (m, 4H), 1.87-1.80 (m, 2H), 1.09-1.04 (m, 3H).

### Example 169

### (Z)-5-(2-(Pyrimidin-2-yl)furo[2,3-c]pyridin-3-ylamino)isoindolin-1-one oxime

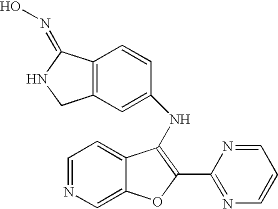

Step A: Methyl 4-bromo-2-(bromomethyl)benzoate: Methyl 4-bromo-2-methylbenzoate (10.00 g, 43.65 mmol) was dissolved in 60 mL CCl₄. N-Bromosuccinimide (7.770 g, 43.65 mmol) and benzoyl peroxide (1.057 g, 4.365 mmol) were added and the mixture was heated to reflux under argon for 24 hours. The reaction was cooled, poured into 10% HCl, washed with dichloromethane, dried over MgSO₄, filtered and concentrated to an orange wax. The crude product was purified by silica gel chromatography using 5-30% EtOAc/Hexanes.

Step B: 5-bromoisoindolin-1-one: Methyl 4-bromo-2-(bromomethyl)benzoate (5.00 g, 16.2 mmol) in NH₃ (34.8 mL, 244 mmol) was heated in a bomb-reactor at 85° C. for 36 hours, then cooled and concentrated to a solid. The solid was triturated with ethyl acetate, filtered and concentrated to a light brown solid. The mother liquor was concentrated and purified by silica gel chromatography using 1-5% MeOH/dichloromethane to provide additional product (2.5 g, 72%).

Step C: (Z)-5-Bromoisoindolin-1-one O-benzyl oxime: 5-Bromoisoindolin-1-one (3.00 g, 14.1 mmol) was suspended in 100 mL chloroform at 0° C. Triethyloxonium tetrafluoroborate (4.03 g, 21.2 mmol) was added in a single portion to the mixture and the suspension was stirred from 0° C. to ambient temperature over the 48 hours. The reaction was concentrated and the residue dissolved in 150 mL ethanol and cooled to 0° C. O-benyzyl hydroxylamine HCl (4.52 g, 28.3 mmol) and sodium carbonate (4.50 g, 42.4 mmol) were added, and the reaction was stirred from 0° C. to ambient temperature for 18 hours, then concentrated, diluted with ethyl acetate, washed with 10% citric acid, dried over sodium sulfate, filtered and concentrated to a solid. The solid was purified by silica gel chromatography with 100% dichloromethane (1.69 g, 37%).

Step D: 5-Aminoisoindolin-1-one O-benzyl oxime: A THF solution (100 mL) containing tert-butyl carbamate (2.50 g, 21.3 mmol), (Z)-5-bromoisoindolin-1-one O-benzyl oxime (1.69 g, 5.33 mmol), Pd₂ dba₃ (0.244 g, 0.266 mmol), Cs₂CO3 (2.78 g, 8.53 mmol), and XPHOS (0.254 g, 0.533 mmol) was degassed with argon. The reaction was heated at reflux for 18 hours under argon, then cooled, filtered through celite and concentrated to a solid. The compound was purified by silica gel chromatography using 100% dichloromethane to 1% MeOH/dichloromethane. The isolated solid obtained after concentration of desired fractions was dissolved in dichloromethane, and mL of TFA was added. The reaction was stirred at ambient temperature for a few hours, then concentrated. The residue was suspended in saturated bicarbonate and ethyl acetate. The organic layer was collected, washed with brine, dried over sodium sulfate, filtered and concentrated to a solid. The product was purified by silica gel chromatography using 40% EtOAc/Hex-40% EtOAc/Hex/2% MeOH (560 mg, 41%).

Step F: (Z)-5-(2-(pyrimidin-2-yl)furo[2,3-c]pyridin-3-ylamino)isoindolin-1-one O-benzyl oxime: Prepared according to the method of Example 53 and purified by column chromatography using 2% MeOH/DCM (18 mg, 31%).

Step G: (Z)-5-(2-(pyrimidin-2-yl)furo[2,3-c]pyridin-3-ylamino)isoindolin-1-one oxime: (Z)-5-(2-(pyrimidin-2-yl)furo[2,3-c]pyridin-3-ylamino)isoindolin-1-one O-1-methylphenyl oxime (0.068 g, 0.152 mmol) was suspended in 10 mL methanol. HCl (0.0417 mL, 0.167 mmol) was added and the solid went into solution. The reaction was degassed with argon, and Pd(OH)₂/C (0.00213 g, 0.0152 mmol) was added. The solution was purged with hydrogen and stirred until starting material was consumed. The reaction was filtered through Celite and the filtrated was concentrated to a yellow film. The product was purified by column chromatography using 2-8% MeOH/dichloromethane (12 mg, 22%). MS (APCI), m/z=359.2. ¹H NMR (400 MHz, d6-DMSO) δ 9.08 (1H, s), 9.02 (1H, bs), 8.96 (1H, s), 8.95 (1H, s), 8.92 (1H, s), 8.35 (1H, d, J=5.4 Hz), 7.45-7.41 (2H, m), 7.26 (1H, d, J=5.4 Hz), 7.16 (1H, s), 7.11-7.09 (1H, m), 6.74 (1H, bs), 4.36 (2H, s). MS (APCI) m/z=359.2 (M+H).

### Example 170

### (Z)-3-(1-(Hydroxyimino)isoindolin-5-ylamino)-N-isopropylfuro[2,3-c]pyridine-2-carboxamide

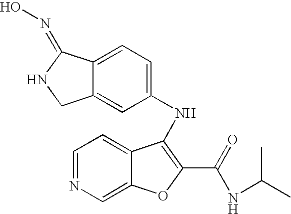

Step A: (Z)-3-(1-(Benzyloxyimino)isoindolin-5-ylamino)-N-isopropylfuro[2,3-c]pyridine-2-carboxamide: Prepared according to Example 53 using cesium carbonate as base, followed by amide bond formation as described in Example 43.

Step B: (Z)-3-(1-(Hydroxyimino)isoindolin-5-ylamino)-N-isopropylfuro[2,3-c]pyridine-2-carboxamide: The product from Step A was dissolved in 10 mL methanol. Aqueous HCl (0.0428 mL, 0.171 mmol, 4M) was added and the solution degassed with argon. Pd(OH)₂/C (0.00240 g, 0.0171 mmol) was added and the solution purged with hydrogen and stirred until reaction was complete. The reaction mixture was filtered through Celite and the filtrate was concentrated. The residue was purified by silica gel chromatography using 2-8% MeOH/dichloromethane+0.1% NH₄OH. Isolated the desired material as yellow solid (43 mg, 68%). ¹H NMR (400 mHz, MeOH-D₄) δ 8.90 (1H, s), 8.30 (1H, d, J=5.4 Hz), 7.57 (1H, d, J=7.8 Hz), 7.35 (1H, d, J=5.4 Hz), 7.1 (1H, bs), 7.07 (1H, m), 4.47 (2H, s), 4.26-4.22 (1H, m, J=6.3 Hz), 1.27 (6H, d, J=6.3 Hz). MS (APCI) m/z=366.2 (M+1).

### Example 171

### (Z)-3-(1-(Hydroxyimino)isoindolin-5-ylamino)-N-(pyridin-3-ylmethyl)furo[2,3-c]pyridine-2-carboxamide

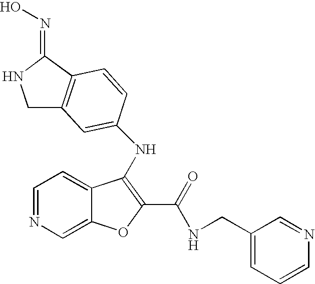

Prepared according to the method of Example 170. ¹H NMR (400 mHz, MeOH-D₄) δ 8.90 (1H, s), 8.59 (1H, m), 8.43 (1H, m), 8.30 1H, d, J=5.4 Hz), 7.88 (1H, m), 7.57 (1H, d, J=8.6 Hz), 7.43-7.39 (1H, m), 7.35 (1H, d, J=5.4 Hz), 7.12 (1H, bs), 7.10-7.04 (1H, m), 4.64 (2H, s), 4.47 (2H, s).

### Example 172

Furo[2,3-c]pyridin-3(2H)-one hydrochloride

A solution of ethyl 3-hydroxyfuro[2,3-c]pyridine-2-carboxylate was heated at reflux for 20 hours in 10% aq. HCl. The volatiles were removed under reduced pressure to afford the product. MS (APCI pos) M+1=136.1.

### Example 173

### 2-Bromo-N-(4-chlorophenyl)furo[2,3-c]pyridin-3-amine

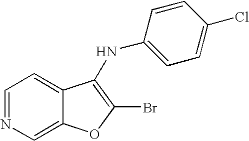

Step A: N-(4-Chlorophenyl)furo[2,3-c]pyridin-3-amine: Prepared from furo[2,3-c]pyridin-3(2H)-one hydrochloride and 4-chloroaniline according to the method of Example 54 in 78% yield. MS (APCI-pos) M+1=245.3.

Step B: 2-Bromo-N-(4-chlorophenyl)furo[2,3-c]pyridin-3-amine: N-(4-Chlorophenyl)furo[2,3-c]pyridin-3-amine (180 mg, 0.74 mmol) was dissolved in DMF (4 mL) and bromine (1.2 equiv) was added by syringe. The reaction was stirred for 20 hours, diluted with aq. NaHCO₃ and aqueous sodium thiosulfate, and extracted with ethyl acetate. Silica gel chromatography (50% EtOAc/hexanes) afforded the product as an off-white solid (63 mg). MS (APCI-pos) M+1=323.4/325.3. ¹H NMR (400 MHz, CDCl₃) δ 8.84 (s, 1H), 8.39-8.37 (m, 1H), 7.23-7.19 (m, 3H), 6.70-6.68 (m, 2H), 5.33 (bs, 1H).

### Example 174

### 6-(Furo[2,3-c]pyridin-3-ylamino)naphthalen-1-ol

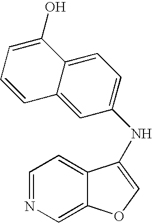

Step A: N-(5-(tert-butyldimethylsilyloxy)naphthalen-2-yl)furo[2,3-c]pyridin-3-amine: Prepared from furo[2,3-c]pyridin-3(2H)-one hydrochloride and 5-(tert-butyldimethylsilyloxy)naphthalen-2-amine according to the method of Example 54 in 84% yield. MS (APCI-pos) M+1=391.3.

Step B: 6-(furo[2,3-c]pyridin-3-ylamino)naphthalen-1-ol: Prepared form the product of Step A according to the method of Example 52 in 42% yield. MS (APCI-pos) M+1=277.3. ¹H NMR (400 MHz, d6-DMSO) δ 9.91-9.90 (m, 1H), 8.94 (br s, 1H), 8.51-8.47 (m, 2H), 8.45-8.42 (m, 1H), 8.03-8.00 (m, 1H), 7.90-7.88 (m, 1H), 7.30-7.26 (m, 1H), 7.24-7.16 (m, 3H), 6.64-6.62 (m, 1H).

### Example 175

### 6-(Furo[2,3-c]pyridin-3-yl(2-hydroxyethyl)amino)naphthalen-1-ol

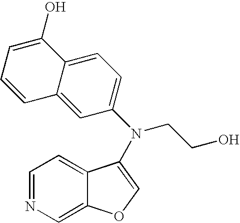

Step A: 5-Methoxynaphthalen-2-amine: Sodium hydride (1.2 equiv) was slurried in DMF (50 mL) and the reaction cooled to 0° C. 6-Aminonaphthalen-1-ol (5.3 g, 1.0 equiv) was carefully added, generating a heavy precipitate. After 30 minutes, dimethyl sulfate (1.0 equiv) was added, and the reaction was allowed to slowly warm to ambient temperature over 8 hours. The reaction was diluted with water and extracted with EtOAc. The crude extracts were dried (MgSO₄), concentrated, and purified by silica gel chromatography (eluting with 100% CH₂Cl₂) to afford the product as a solid (4.7 g, 81%).

Step B: N-(5-Methoxynaphthalen-2-yl)furo[2,3-c]pyridin-3-amine: Prepared from the product of Step A following the procedure of Example 54 in 88% yield. MS (APCI-pos) M+1=291.2.

Step C: 2-(Furo[2,3-c]pyridin-3-yl(5-methoxynaphthalen-2-yl)amino)ethanol: N-(5-Methoxynaphthalen-2-yl)furo[2,3-c]pyridin-3-amine (188 mg, 0.65 mmol) was dissolved in THF (5.0 mL) and cooled to −78° C. n-BuLi (2.2 equiv) was added dropwise via syringe and the dark solution was stirred 45 minutes, and then a freshly condensed, chilled aliquot of ethylene oxide (1.3 equiv) was added at once. The solution was allowed to warm to ambient temperature over 8 hours, and was then quenched with aq. NH₄Cl and diluted with EtOAc. The layers were separated, the organics dried (MgSO₄), and purified by silica gel chromatography (1% MeOH/CH₂Cl₂) to afford the product as a foam (105 mg, 49%). MS (APCI-pos) M+1=335.2.

Step D: 6-(Furo[2,3-c]pyridin-3-yl(2-hydroxyethyl)amino)naphthalen-1-ol: 2-(Furo[2,3-c]pyridin-3-yl(5-methoxynaphthalen-2-yl)amino)ethanol (105 mg, 0.314 mmol) was dissolved in CH₂Cl₂ (4.0 mL) and cooled in an ice bath. Boron tribromide (1.0 M in CH₂Cl₂, 3.2 equiv) was added and the reaction was allowed to warm slowly to ambient temperature over 3 hours. Cooled to 0° C. and quenched by careful addition of methanol. The mixture was concentrated under reduced pressure, and the residue was dissolved in CH₂Cl₂. Water and triethylamine were added, the layers were separated, dried over sodium sulfate, and purified by silica gel chromatography (2% methanol/CH₂Cl₂) to afford the product as an off-white foam (73 mg, 73%). MS (APCI-pos) M+1=321.2. ¹H NMR (400 MHz, d6-DMSO) δ 9.94 (s, 1H), 8.97 (s, 1H), 8.36 (s, 1H), 8.24-8.23 (m, 1H), 7.97-7.95 (m, 1H), 7.18-7.02 (m, 5H), 6.65-6.63 (m, 1H), 4.90-4.87 (m, 1H), 3.90-3.87 (m, 2H), 3.70-3.66 (m, 2H).

### Example 176

### 5-(Furo[2,3-c]pyridin-3-ylamino)-2,3-dihydro-1H-inden-1-one oxime

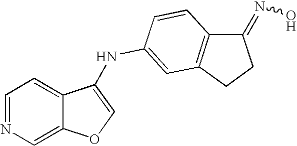

Furo[2,3-c]pyridin-3(2H)-one hydrochloride (228 mg, 1.0 equiv) and 5-amino-2,3-dihydro-1H-inden-1-one O-tert-butyldimethylsilyl oxime (1.1 equiv) were dissolved in MeOH (10 mL) and heated to 60° C. for 20 hours. The reaction was cooled, triethylamine was added, the mixture concentrated, and the residual material purified by silica gel chromatography (eluting with 4% MeOH/CHCl₃) to afford the product as a solid. ¹H NMR (400 MHz, d6-DMSO) δ 10.47-10.45 (m, 1H), 8.94-8.92 (m, 1H), 8.55-8.17 (m, 3H), 7.89-7.77 (m, 1H), 7.44-7.41 (m, 1H), 6.96-6.88 (m, 2H), 2.97-2.66 (m, 4H).

### Example 177

### N-(2-Methoxyphenyl)furo[2,3-c]pyridin-3-amine

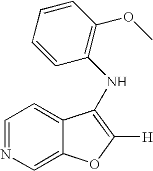

Prepared according to the method of Example 54. MS (ESI+) m/z 241.2. ¹H NMR (CDCl₃, 400 MHz) δ 8.87 (s, 1H), 8.41 (d, 1H, J=5.2 Hz), 7.81 (s, 1H), 7.46 (d, 1H, J=6.3 Hz), 6.92-6.84 (m, 4H), 6.03-5.97 (m, 1H), 3.96 (s, 3H).

### Example 178

### 5-Cloro-2-(furo[2,3-c]pyridin-3-ylamino)phenol

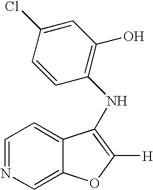

Prepared following the procedure described for Example 54, substituting 2-amino-5-chlorophenol. MS (ESI+) m/z 261.2. ¹H NMR (CDCl₃, 400 MHz) δ 8.87-8.74 (brd s, 1H), 8.42-8.30 (brd s, 1H), 7.81 (s, 1H), 7.54-7.49 (m, 1H), 6.86 (s, 1H), 6.77-6.74 (m, 2H).

### Example 179

### 3-(4-(4-Methyl-1H-pyrazol-5-yl)phenylamino)thieno[2,3-c]pyridine-2-carbohydrazide

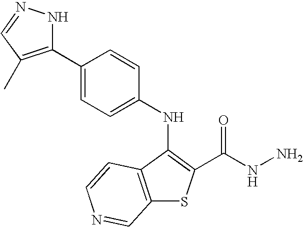

Step A: Ethyl 3-(4-propionylphenylamino)thieno[2,3-c]pyridine-2-carboxylate: Prepared according to the method of Example 2 using ethyl 3-aminothieno[2,3-c]pyridine-2-carboxylate and 1-(4-bromophenyl)propan-1-one (38% yield).

Step B: (E)-Ethyl 3-(4-(3-(dimethylamino)-2-methylacryloyl)phenylamino)thieno[2,3-c]pyridine-2-carboxylate: Ethyl 3-(4-propionylphenylamino)thieno[2,3-c]pyridine-2-carboxylate (0.3005 g, 0.8479 mmol) was dissolved in THF (4 mL) and treated with tert-butoxybis(dimethylamino)methane (0.3502 mL, 1.696 mmol). The solution was heated to reflux for 3 hours. The reaction mixture was cooled to ambient temperature and treated with additional tert-butoxybis(dimethylamino)methane (0.08754 mL, 0.4239 mmol) then reheated to reflux for 3 hours. The reaction was cooled and concentrated in vacuo, and the product used in the next step without characterization.

Step C: 3-(4-(4-methyl-1H-pyrazol-5-yl)phenylamino)thieno[2,3-c]pyridine-2-carbohydrazide: (E)-ethyl 3-(4-(3-(dimethylamino)-2-methylacryloyl)phenylamino)thieno[2,3-c]pyridine-2-carboxylate (0.347 g, 0.8474 mmol) was dissolved in ethanol (95%, 5 mL) and treated with hydrazine (0.1330 mL, 4.237 mmol). The solution was heated to reflux for 18 hours. The residue was suspended in methylene chloride (5 mL) and the solids were collected by filtration, washed with methylene chloride and air-dried to provide the product as a solid (82%). MS (ESI+) m/z 365.2. ¹H NMR (CDCl₃/CD₃OD, 400 MHz) δ 9.11-9.07 (m, 1H), 8.31-8.24 (brd s, 1H), 7.55-7.49 (m, 2H), 7.41 (s, 1H), 7.36-7.31 (m, 1H), 7.12-7.03 (m, 2H), 2.24 (s, 3H).

### Example 180

### N-(2-(Dimethylamino)ethyl)-3-(4-(4-methyl-1H-pyrazol-5-yl)phenylamino)thieno[2,3-c]pyridine-2-carboxamide

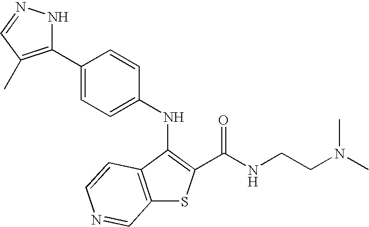

Step A: 3-(4-(4-Methyl-1H-pyrazol-5-yl)phenylamino)thieno[2,3-c]pyridine-2-carbonyl azide: A pre-cooled solution of sodium nitrite (0.0386 g, 0.560 mmol, dissolved in water (0.386 mL) was added dropwise to a cold (0° C.) solution of 3-(4-(4-methyl-1H-pyrazol-5-yl)phenylamino)thieno[2,3-c]pyridine-2-carbohydrazide (Example 179; 0.204 g, 0.560 mmol) dissolved in acetic acid (1.2 mL). The resultant solution was warmed to ambient temperature and stirred for 1 hour. The reaction mixture was diluted with ethyl acetate and water. The mixture was separated and the organic layer washed with water and saturated NaCl, dried over sodium sulfate and concentrated in vacuo to a foam (204 mg, 97%).

Step B: N-(2-(dimethylamino)ethyl)-3-(4-(4-methyl-1H-pyrazol-5-yl)phenylamino)thieno[2,3-c]pyridine-2-carboxamide: 3-(4-(4-methyl-1H-pyrazol-5-yl)phenylamino)thieno[2,3-c]pyridine-2-carbonyl azide (0.0572 g, 0.1524 mmol) was slurried in methylene chloride (1 mL) and treated with N1,N1-dimethylethane-1,2-diamine (0.06691 mL, 0.6095 mmol). The reaction immediately became homogeneous and was stirred at ambient temperature for 3 hours. The reaction mixture was applied directly to SiO₂ (Biotage 12M) and eluted with 4% MeOH/1% NH₄OH/methylene chloride. The MeOH was slowly increased to 7%. The desired product was recovered as a solid (54.6 mg, 85%). MS (ESI+) m/z 421.1. ¹H NMR (CDCl₃, 400 MHz): 9.09 (s, 1H), 8.84 (s, 1H), 8.36 (d, 1H, J=5.9 Hz), 7.52-7.46 (m, 1H), 7.44 (s, 1H), 7.04-6.98 (m, 3H), 3.51 (m, 2H), 2.53 (t, 2H), 2.28 (s, 6H), 2.23 (s, 3H).

### Example 181

### N-isopropyl-3-(4-(4-methyl-1H-pyrazol-5-yl)phenylamino)thieno[2,3-c]pyridine-2-carboxamide

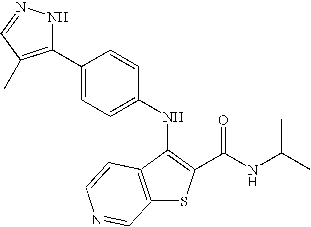

Prepared as described in Example 180 Step B, using isopropylamine, (76%). MS (ESI+) m/z 392.1. ¹H NMR (CDCl₃, 400 MHz) δ 9.10 (s, 1H), 8.87 (brd s, 1H), 8.38 (d, 1H, J=5.5 Hz), 7.54-7.48 (m, 2H), 7.44 (s, 1H), 7.34 (d, 1H, J=5.8 Hz), 7.05-7.00 (m, 2H), 5.97-5.90 (m, 1H), 4.26 (m, 1H), 2.24 (s, 3H), 1.27 (d, 6H).

### Example 182

### 3-(4-(4-Methyl-1H-pyrazol-5-yl)phenylamino)-N-(pyridin-3-ylmethyl)thieno[2,3-c]pyridine-2-carboxamide

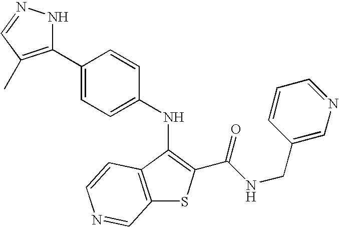

Prepared as described in Example 180, Step B, using pyridin-3-ylmethanamine, (90%). MS (ESI+) m/z 441.1. ¹H NMR (CDCl₃, 400 MHz 6: 9.09 (s, 1H), 8.62 (brd s, 1H), 8.55-8.49 (m, 2H), 8.37 (d, 1H, J=5.7 Hz), 7.70-7.65 (m, 1H), 7.52-7.47 (m, 1H), 7.44 (s, 1H), 7.36-7.32 (m, 1H), 7.29-7.24 (m, 1H), 7.02-6.97 (m, 2H), 6.94-6.89 (m, 1H), 4.63 (d, 2H, J=5.8 Hz), 2.23 (s, 3H).

### Example 183

### N-(2-(dimethylamino)ethyl)-3-(1-(hydroxyimino)-2,3-dihydro-1H-inden-5-ylamino)thieno[2,3-c]pyridine-2-carboxamide

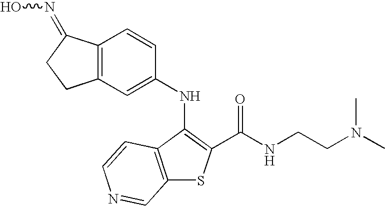

Prepared according to the method of Example 16. The title compound was isolated as a mixture of oxime isomers as a solid (89%). MS (ESI+) m/z 410.0. ¹H NMR (CDCl₃, 400 MHz): 9.06 (s, 1H), 8.66-8.64 (brd s, 2H), 8.35 (d, 1H, J=5.8 Hz), 7.41 (d, 1H, J=8.3 Hz), 7.40-7.36 (m, 1H), 6.83-6.81 (m, 1H), 6.81-6.76 (m, 1H), 6.73-6.69 (m, 1H), 3.55-3.48 (m, 4H), 2.56-2.50 (m, 4H), 2.29 (s, 6H).

### Example 184

### 3-(1-(Hydroxyimino)-2,3-dihydro-1H-inden-5-ylamino)-N-isopropylthieno[2,3-c]pyridine-2-carboxamide

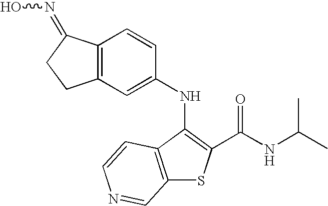

Prepared as a mixture of oxime isomers according to the method of Example 16, using isopropylamine (76%). MS (ESI+) m/z 381.2. ¹H NMR (CDCl₃, 400 MHz) δ 9.11 (s, 1H), 8.74 (brd s, 1H), 8.40 (d, 1H, J=6.3 Hz), 7.55 (d, 1H, J=8.2 Hz), 7.32 (d, 1H, J=5.6 Hz), 6.85-6.80 (m, 2H), 6.01-5.96 (m, 1H), 4.25 (m, 1H), 2.97 (m, 4H), 1.26 (d, 6H).

### Example 185

### 3-(1-(Hydroxyimino)-2,3-dihydro-1H-inden-5-ylamino)-N-(pyridin-3-ylmethyl)thieno[2,3-c]pyridine-2-carboxamide

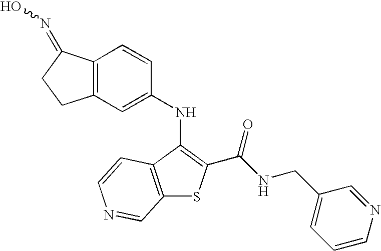

Prepared as a mixture of oxime isomers according to Example 16 using with 3-(aminomethyl)pyridine (52%). MS (ESI+) m/z 430.1. ¹H NMR (CDCl₃, 400 MHz) δ 9.11 (s, 1H), 8.66-8.59 (m, 2H), 8.59-8.54 (m, 1H), 8.39 (d, 1H, J=5.4 Hz), 7.70-7.65 (m, 1H), 7.54 (d, 1H, J=8.0 Hz), 7.31 (d, 1H, J=6.7 Hz), 6.88-6.80 (m, 2H), 4.65 (d, 2H), 2.97 (s, 4H).

### Example 186

### Ethyl 3-(4-chlorophenylamino)thieno[2,3-c]]pyridine-2-carboxylate trifluorosulfonic acid salt

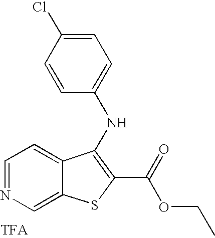

Prepared according to the method of Example 2, using ethyl 3-aminothieno[2,3-c]pyridine-2-carboxylate and 1-bromo-4-chlorobenzene. The crude product was purified by reverse phase HPLC (17%). MS (ESI+) m/z 333.2. ¹H NMR (CDCl₃, 400 MHz) δ 9.09 (s, 1H), 8.69 (brd s, 1H), 8.32 (d, 1H, J=5.3 Hz), 7.30-7.26 (m, 2H), 7.16-7.12 (m, 1H), 7.02-6.97 (m, 2H), 4.41 (q, 2H), 1.43 (t, 3H).

### Example 187

### 3-(4-Chlorophenylamino)thieno[2,3-c]pyridine-2-carboxamide

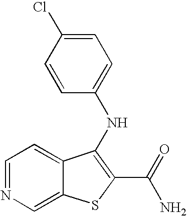

Ethyl 3-(4-chlorophenylamino)thieno[2,3-c]pyridine-2-carboxylate (0.0299 g, 0.0696 mmol) was dissolved in methanol (2 mL), treated with ammonia in MeOH (0.00994 mL, 0.0696 mmol, 7N) then the mixture was warmed to 70° C. for 8 hours. After cooling to ambient temperature, the reaction mixture developed a yellow precipitate. The solid was recovered by filtration, washed with MeOH then air-dried. The desired product was recovered as a yellow solid (14.2 mg, 67%). MS (ESI+) m/z 304.2 ¹H NMR (DMSO-d6, 400 MHz) δ 9.30 (s, 1H), 8.72 (s, 1H), 8.43 (d, 1H, J=5.5 Hz), 7.97-7.75 (brd s, 2H), 7.35 (d, 1H, J=5.4 Hz), 7.26-7.21 (m, 2H), 6.81-6.76 (m, 2H).

The compounds in Table 5 were also prepared by the methods described herein.

TABLE 5

| | |
|---|---|
| 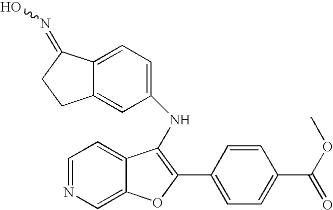 | methyl 4-(3-(1-(hydroxyimino)-2,3- dihydro-1H-inden-5-ylamino)furo[2,3- c]pyridin-2-yl)benzoate |
| 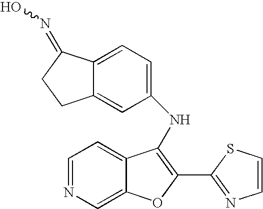 | 5-(2-(thiazol-2-yl)furo[2,3-c]pyridin-3- ylamino)-2,3-dihydro-1H-inden-1-one oxime |
| 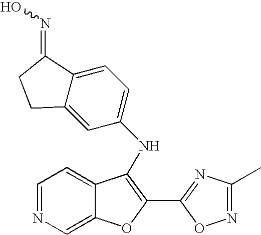 | 5-(2-(3-methyl-1,2,4-oxadiazol-5- yl)furo[2,3-c]pyridin-3-ylamino)-2,3- dihydro-1H-inden-1-one oxime |
| 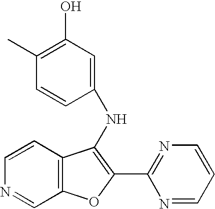 | 2-methyl-5-(2-(pyrimidin-2-yl)furo[2,3- c]pyridin-3-ylamino)phenol |
| 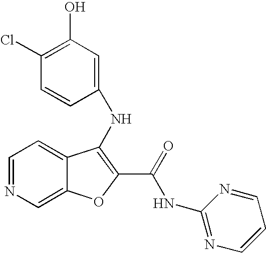 | 3-(4-chloro-3-hydroxyphenylamino)-N- (pyrimidin-2-yl)furo[2,3-c]pyridine-2- carboxamide |
| 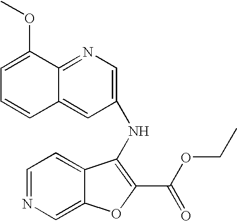 | ethyl 3-(8-methoxyquinolin-3- ylamino)furo[2,3-c]pyridine-2-carboxylate |
| 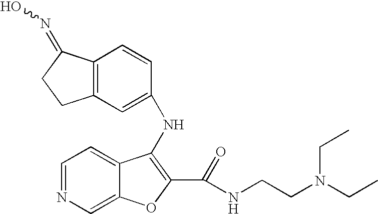 | N-(2-(diethylamino)ethyl)-3-(1- (hydroxyimino)-2,3-dihydro-1H-inden-5- ylamino)furo[2,3-c]pyridine-2- carboxamide |
| 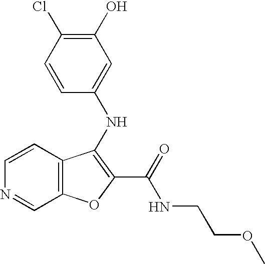 | 3-(4-chloro-3-hydroxyphenylamino)-N-(2- methoxyethyl)furo[2,3-c]pyridine-2- carboxamide |
| 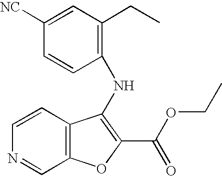 | ethyl 3-(4-cyano-2- ethylphenylamino)furo[2,3-c]pyridine-2- carboxylate |
| 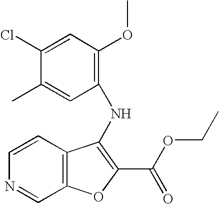 | ethyl 3-(4-chloro-2-methoxy-5- methylphenylamino)furo[2,3-c]pyridine-2- carboxylate |
| 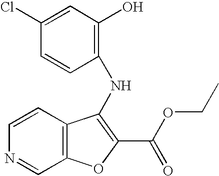 | ethyl 3-(4-chloro-2- hydroxyphenylamino)furo[2,3-c]pyridine- 2-carboxylate |
| 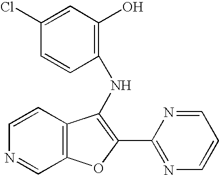 | 5-chloro-2-(2-(pyrimidin-2-yl)furo[2,3- c]pyridin-3-ylamino)phenol |
| 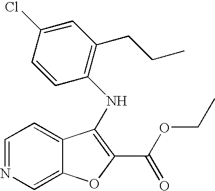 | ethyl 3-(4-chloro-2- propylphenylamino)furo[2,3-c]pyridine-2- carboxylate |
| 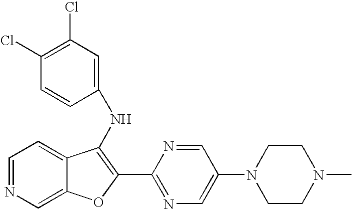 | N-(3,4-dichlorophenyl)-2-(5-(4- methylpiperazin-1-yl)pyrimidin-2- yl)furo[2,3-c]pyridin-3-amine |
| 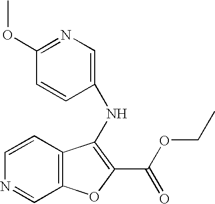 | ethyl 3-(6-methoxypyridin-3- ylamino)furo[2,3-c]pyridine-2-carboxylate |
| 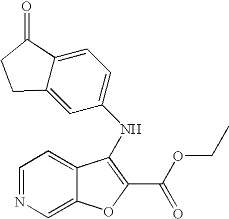 | ethyl 3-(1-oxo-2,3-dihydro-1H-inden-5- ylamino)furo[2,3-c]pyridine-2-carboxylate |
| 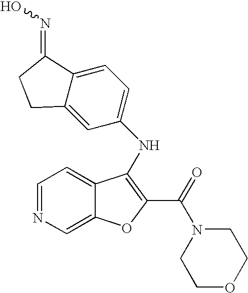 | (3-(1-(hydroxyimino)-2,3-dihydro-1H- inden-5-ylamino)furo[2,3-c]pyridin-2- yl)(morpholino)methanone |
| 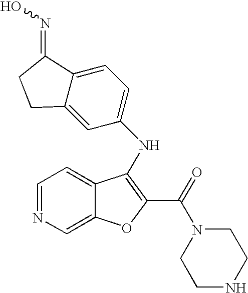 | (3-(1-(hydroxyimino)-2,3-dihydro-1H- inden-5-ylamino)furo[2,3-c]pyridin-2- yl)(piperazin-1-yl)methanone |
| 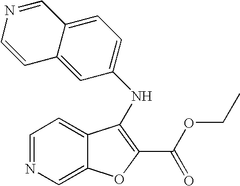 | ethyl 3-(isoquinolin-6-ylamino)furo[2,3- c]pyridine-2-carboxylate |
| 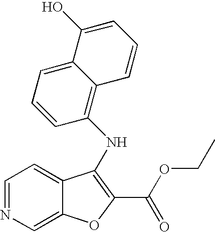 | ethyl 3-(5-hydroxynaphthalen-1- ylamino)furo[2,3-c]pyridine-2-carboxylate |
| 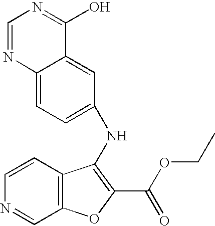 | ethyl 3-(4-hydroxyquinazolin-6- ylamino)furo[2,3-c]pyridine-2-carboxylate |
| 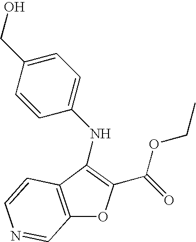 | ethyl 3-(4- (hydroxymethyl)phenylamino)furo[2,3- c]pyridine-2-carboxylate |
| 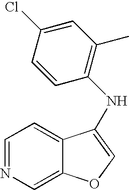 | N-(4-chloro-2-methylphenyl)furo[2,3- c]pyridin-3-amine |
| 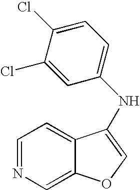 | N-(3,4-dichlorophenyl)furo[2,3-c]pyridin- 3-amine |
| 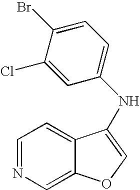 | N-(4-bromo-3-chlorophenyl)furo[2,3- c]pyridin-3-amine |
| 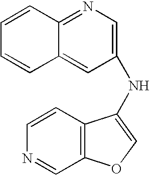 | N-(quinolin-3-yl)furo[2,3-c]pyridin-3- amine |
| 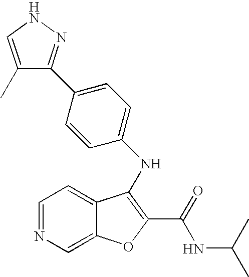 | N-isopropyl-3-(4-(4-methyl-1H-pyrazol-3- yl)phenylamino)furo[2,3-c]pyridine-2- carboxamide |
| 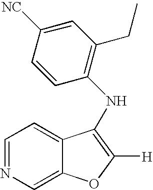 | 3-ethyl-4-(furo[2,3-c]pyridin-3- ylamino)benzonitrile |
| 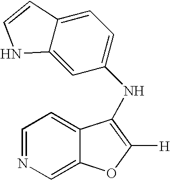 | N-(1H-indol-6-yl)furo[2,3-c]pyridin-3- amine |
| 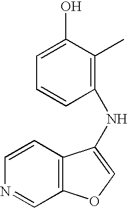 | 3-(furo[2,3-c]pyridin-3-ylamino)-2- methylphenol |
| 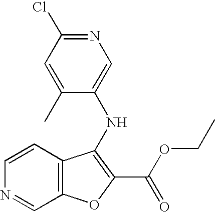 | ethyl 3-(6-chloro-4-methylpyridin-3- ylamino)furo[2,3-c]pyridine-2-carboxylate |
| 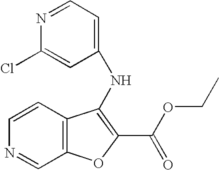 | ethyl 3-(2-chloropyridin-4- ylamino)furo[2,3-c]pyridine-2-carboxylate |
| 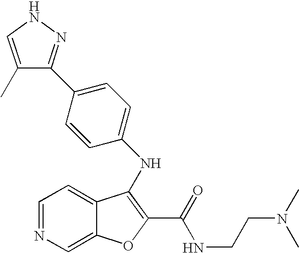 | N-(2-(dimethylamino)ethyl)-3-(4-(4- methyl-1H-pyrazol-3- yl)phenylamino)furo[2,3-c]pyridine-2- carboxamide |
| 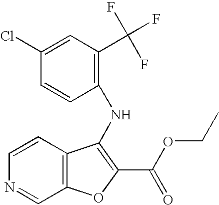 | ethyl 3-(4-chloro-2- (trifluoromethyl)phenylamino)furo[2,3- c]pyridine-2-carboxylate |
| 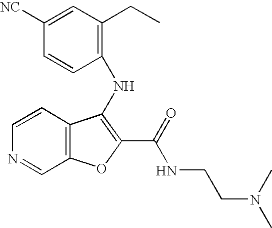 | 3-(4-cyano-2-ethylphenylamino)-N-(2- (dimethylamino)ethyl)furo[2,3-c]pyridine- 2-carboxamide |
| 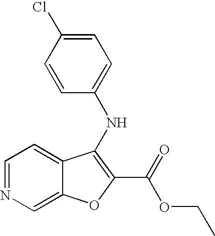 | ethyl 3-(4-chlorophenylamino)furo[2,3- c]pyridine-2-carboxylate |
| 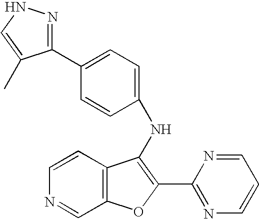 | N-(4-(4-methyl-1H-pyrazol-3-yl)phenyl)- 2-(pyrimidin-2-yl)furo[2,3-c]pyridin-3- amine |
| 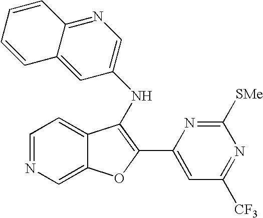 | 2-(2-(methylthio)-6- (trifluoromethyl)pyrimidin-4-yl)-N- (quinolin-3-yl)furo[2,3-c]pyridin-3-amine |
| 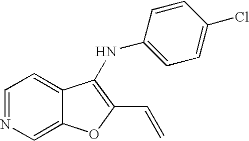 | N-(4-chlorophenyl)-2-vinylfuro[2,3- c]pyridin-3-amine |
| 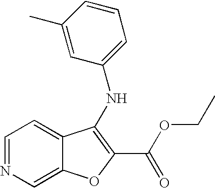 | ethyl 3-(m-tolylamino)furo[2,3-c]pyridine- 2-carboxylate |
| 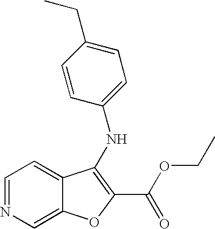 | ethyl 3-(4-ethylphenylamino)furo[2,3- c]pyridine-2-carboxylate |
| 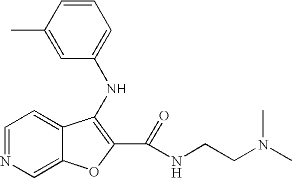 | N-(2-(dimethylamino)ethyl)-3-(m- tolylamino)furo[2,3-c]pyridine-2- carboxamide |
| 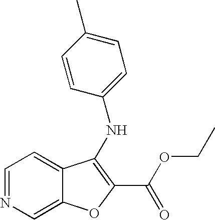 | ethyl 3-(p-tolylamino)furo[2,3-c]pyridine- 2-carboxylate |
| 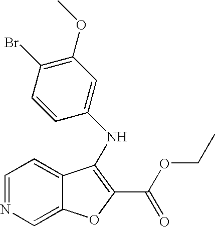 | ethyl 3-(4-bromo-3- methoxyphenylamino)furo[2,3-c]pyridine- 2-carboxylate |
| 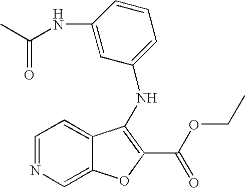 | ethyl 3-(3- acetamidophenylamino)furo[2,3- c]pyridine-2-carboxylate |
| 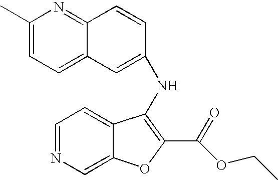 | ethyl 3-(2-methylquinolin-6- ylamino)furo[2,3-c]pyridine-2-carboxylate |
| 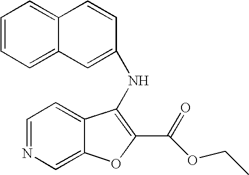 | ethyl 3-(naphthalen-2-ylamino)furo[2,3- c]pyridine-2-carboxylate |
| 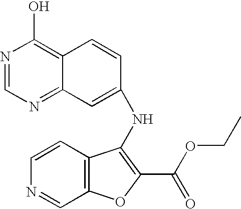 | ethyl 3-(4-hydroxyquinazolin-7- ylamino)furo[2,3-c]pyridine-2-carboxylate |
| 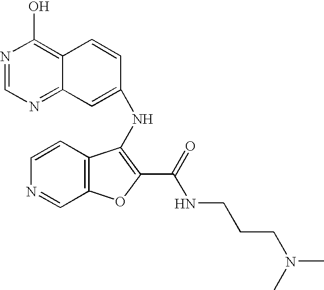 | N-(3-(dimethylamino)propyl)-3-(4- hydroxyquinazolin-7-ylamino)furo[2,3- c]pyridine-2-carboxamide |
| 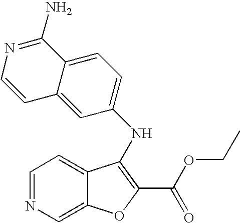 | ethyl 3-(1-aminoisoquinolin-6- ylamino)furo[2,3-c]pyridine-2-carboxylate |
| 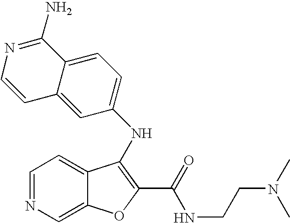 | 3-(1-aminoisoquinolin-6-ylamino)-N-(2- (dimethylamino)ethyl)furo[2,3-c]pyridine- 2-carboxamide |
| 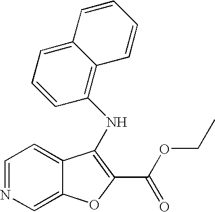 | ethyl 3-(naphthalen-1-ylamino)furo[2,3- c]pyridine-2-carboxylate |
| 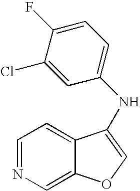 | N-(3-chloro-4-fluorophenyl)furo[2,3- c]pyridin-3-amine |
| 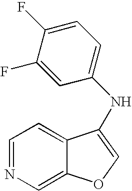 | N-(3,4-difluorophenyl)furo[2,3-c]pyridin- 3-amine |
| 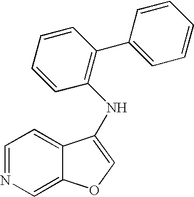 | N-(biphenyl-2-yl)furo[2,3-c]pyridin-3- amine |
| 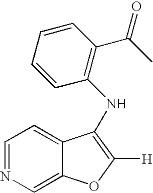 | 1-(2-(furo[2,3-c]pyridin-3- ylamino)phenyl)ethanone |
| 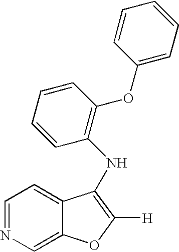 | N-(2-phenoxyphenyl)furo[2,3-c]pyridin-3- amine |
| 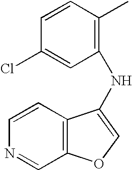 | N-(5-chloro-2-methylphenyl)furo[2,3- c]pyridin-3-amine |
| 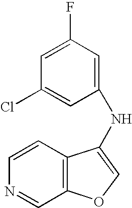 | N-(4-chloro-3-fluorophenyl)furo[2,3- c]pyridin-3-amine |
| 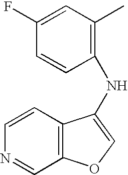 | N-(4-fluoro-2-methylphenyl)furo[2,3- c]pyridin-3-amine |
| 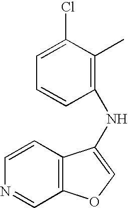 | N-(3-chloro-2-methylphenyl)furo[2,3- c]pyridin-3-amine |
| 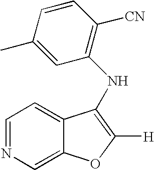 | 2-(furo[2,3-c]pyridin-3-ylamino)-4- methylbenzonitrile |
| 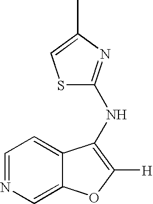 | N-(4-methylthiazol-2-yl)furo[2,3- c]pyridin-3-amine |
| 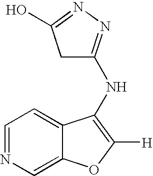 | 5-(furo[2,3-c]pyridin-3-ylamino)-4H- pyrazol-3-ol |
| 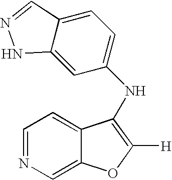 | N-(1H-indazol-6-yl)furo[2,3-c]pyridin-3- amine |
| 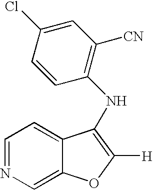 | 5-chloro-2-(furo[2,3-c]pyridin-3- ylamino)benzonitrile |
| 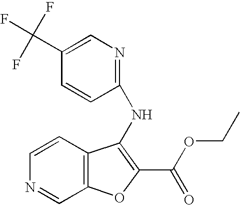 | ethyl 3-(5-(trifluoromethyl)pyridin-2- ylamino)furo[2,3-c]pyridine-2-carboxylate |
| 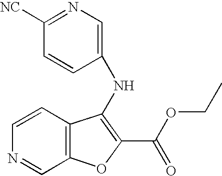 | ethyl 3-(6-cyanopyridin-3- ylamino)furo[2,3-c]pyridine-2-carboxylate |
| 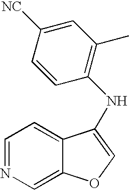 | 4-(furo[2,3-c]pyridin-3-ylamino)-3- methylbenzonitrile |
| 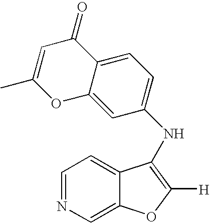 | 7-(furo[2,3-c]pyridin-3-ylamino)-2- methyl-4H-chromen-4-one |
| 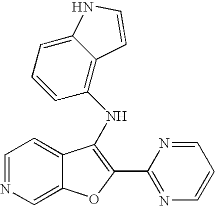 | N-(1H-indol-4-yl)-2-(pyrimidin-2- yl)furo[2,3-c]pyridin-3-amine |
| 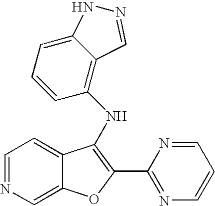 | N-(1H-indazol-4-yl)-2-(pyrimidin-2- yl)furo[2,3-c]pyridin-3-amine |
| 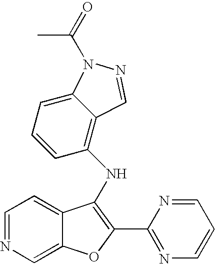 | 1-(4-(2-(pyrimidin-2-yl)furo[2,3-c]pyridin- 3-ylamino)-1H-indazol-1-yl)ethanone |

### Example A

### B-Raf IC₅₀ Assay Protocol

Activity of human recombinant B-Raf protein may be assessed in vitro by assay of the incorporation of radiolabelled phosphate to recombinant MAP kinase (MEK), a known physiologic substrate of B-Raf, according to U.S. Publication No. 2004/127496 and PCT Publication No. WO 03/022840. Catalytically active human recombinant B-Raf protein is obtained by purification from sf9 insect cells infected with a human B-Raf recombinant baculovirus expression vector. To ensure that all substrate phosphorylation resulted from B-Raf activity, a catalytically inactive form of MEK was utilized. This protein is purified from bacterial cells expression mutant inactive MEK as a fusion protein with glutathione-S-transferase (GST-kdMEK).

The activity/inhibition of V600E full-length B-Raf was estimated by measuring the incorporation of radiolabeled phosphate from [γ-³³P]ATP into FSBA-modified wild-type MEK. The 30-μL assay mixtures contained 25 mM Na Pipes, pH 7.2, 100 mM KCl, 10 mM MgCl₂, 5 mM O-glycerophosphate, 100 μM Na Vanadate, 4 μM ATP, 500 nCi [γ-³³P]ATP, 1 μM FSBA-MEK and 20 nM V600E full-length B-Raf. Incubations were carried out at 22° C. in a Costar 3365 plate (Corning). Prior to the assay, the B-Raf and FSBA-MEK were preincubated together in assay buffer at 1.5× (20 μL of 30 nM and 1.5 μM, respectively) for 15 minutes, and the assay was initiated by the addition of 10 μL of 12 μM ATP. Following the 60-minute incubation, the assay mixtures were quenched by the addition of 200 μL of 25% TCA, the plate was mixed on a rotary shaker for 10 minutes, and the product was captured on a Perkin-Elmer GF/B filter plate using a Tomtec Mach III Harvester. After sealing the bottom of the plate, 32 μL of Bio-Safe II (Research Products International) scintillation cocktail were added to each well and the plate was top-sealed and counted in a Topcount NXT (Packard).

### Example B

### In Vitro B-Raf Assay

The activity of compounds of this invention as B-Raf inhibitors may be determined by the following in vitro, fluorescence anisotropy kinase binding assay, according to U.S. Publication No. 2004/127496 and WO 03/022840.

The kinase enzyme, fluorescent ligand and a variable concentration of test compound are incubated together to reach thermodynamic equilibrium under conditions such that in the absence of test compound the fluorescent ligand is significantly (>50%) enzyme bound and in the presence of a sufficient concentration (>10×Ki) of a potent inhibitor the anisotropy of the unbound fluorescent ligand is measurably different from the bound value.

The concentration of kinase enzyme is preferably greater than or equal to 1×K_{f}. The concentration of fluorescent ligand required will depend on the instrumentation used, and the fluorescent and physicochemical properties. The concentration used must be lower than the concentration of kinase enzyme, and preferably less than half the kinase enzyme concentration.

A typical protocol is:
All compounds dissolved in buffer of comparison 50 mM HEPES, pharmaceutical 7.5, 1 mM CHAPS, 10 mM MgCl₂.
B-Raf Enzyme concentration: 1 nM
Fluorescent ligand concentration: 0.5 nM
Test compound concentration: 0.5 nM-100 μM
Components incubated in 10 μL final volume in LJL HE 384 type B black microtitre plate until equilibrium reached (about 3 to 30 hours)
Fluorescence anisotropy read by LJL Acquest.

Kᵢ=dissociation constant for inhibitor binding, and K_{f}=dissociation constant for fluorescent ligand binding. The fluorescent ligand may be a rhodamine- or fluorescein-type dye.

Alternative assay conditions of B-Raf catalytic activity utilize 3 μg of GST-kdMEK, 10 μM ATP and 2 μCi ³³P-ATP, 50 mM MOPS, 0.1 mM EDTA, 0.1M sucrose, 10 mM MgCl₂ plus 0.1% dimethylsulphoxide (containing compound where appropriate) in a total reaction volume of 30 μL. Reactions are incubated at 25° C. for 90 minutes and reactions terminated by addition of EDTA to a final concentration of 50 μM. 10 μL of reaction is spotted to P30 phosphocellulose paper and air dried. Following four washes in ice cold 10% trichloroacetic acid, 0.5% phosphoric acid, papers are air dried prior to addition of liquid scintillant and radioactivity is measured in a scintillation counter.

The activity of compounds of this invention may also be determined by the assays described in WO 99/10325; McDonald, O. B., et al., (1999) Anal. Biochem. 268:318-329, and AACR meeting New Orleans 1998 Poster 3793.

### Example C

### Neuroprotection In Vitro Assay

The neuroprotective properties of compounds of this invention may be determined by the following in vitro assay in Rat Hippocampal Slice Cultures, according to U.S. Publication No. 2004/127496; U.S. Publication No. 2004/082014; and WO 03/022840.

Organotypic cultures provide an intermediate between dissociated neuronal cell cultures and in-vivo models of oxygen and glucose deprivation (OGD). The majority of glial-neuronal interactions and neuronal circuitry are maintained in cultured hippocampal slices, so facilitating investigation of the patterns of death among differing cell types in a model that resembles the in vivo situation. These cultures allow the study of delayed cellular damage and death 24 hours, or more, post-insult and permit assessment of the consequences of long-term alterations in culture conditions. A number of laboratories have reported delayed neuronal damage in response to OGD in organotypic cultures of the hippocampus (Vornov et al., Stroke, (1994) 25:57465; Newell et al., (1995) Brain Res. 676:38-44). Several classes of compounds have been shown to protect in this model, including EAA antagonists (Strasser et al., Brain Res., (1995) 687:167-174), Na channel blockers (Tasker et al., J. Neurosci., (1992) 12:98-4308) and Ca channel blockers (Pringle et al., (1996) Stroke 7:2124-2130).

Organotypic hippocampal slice cultures were prepared using the method of Stoppini et al (1995) J. Neurosci. Methods 37:173-182. Briefly, 400 micron sections prepared from hippocampi of 7-8 day postnatal Sprague Dawley rats are cultured on semiporous membranes for 9-12 days. OGD is then induced by incubation in serum and glucose-free medium in an anaerobic chamber for 45 minutes. Cultures are then returned to the air/CO₂ incubator for 23 hours before analysis. Propidium iodide (PI) is used as an indicator of cell death. PI is non-toxic to neurones and has been used in many studies to ascertain cell viability. In damaged neurons PI enters and binds to nucleic acids. Bound PI shows increased emission at 635 nm when excited at 540 nm. One PI fluorescence image and one white light image are taken and the proportion of cell death analyzed. The area of region CA1 is defined from the white light image and superimposed over the PI image. The PI signal is thresholded and area of PI damage expressed as a percentage of the CA1 area. Correlation between PI fluorescence and histologically confirmed cell death has been validated previously by Niss1-staining using cresyl fast violet (Newell et al., (1995) J. Neurosci. 15:7702-7711).

### Example D

### Spectrophotometric ERK Inhibition Assay

The ERK inhibition properties of compounds of this invention may also be determined by the following spectrophotometric coupled-enzyme assay (Fox et al., (1998) Protein Sci 7:2249). In this assay, a fixed concentration of activated ERK2 (10 nM) is incubated with various concentrations of the compound in DMSO (2.5%) for 10 min. at 30° C. in 0.1 M HEPES buffer, pH 7.5, containing 10 mM MgCl₂, 2.5 mM phosphoenolpyruvate, 200 μM NADH, 150 μg/mL pyruvate kinase, 50 μg/mL lactate dehydrogenase, and 200 μM erktide peptide. The reaction is initiated by the addition of 65 μM ATP. The rate of decrease of absorbance at 340 nm is monitored, which indicates the extent of uninhibited enzyme present in the assay. The IC₅₀ is evaluated from the rate data as a function of inhibitor concentration.

### Example E

### Cellular ERK 1/2 Phosphorylation Assay

Inhibition of basal ERK1/2 phosphorylation was determined by the following in vitro cellular proliferation assay, which comprises incubating cells with a compound of Formula I-II for 1 hour and quantifying the fluorescent pERK signal on fixed cells and normalizing to total ERK signal.

Materials and Methods: Malme-3M cells were obtained from ATCC and grown in RPMI-1640 supplemented with 10% fetal bovine serum. Cells were plated in 96-well plates at 15,000 cells/well and allowed to attach for 1-2 hours. Diluted compounds were then added at a final concentration of 1% DMSO. After 1 hour, cells were washed with PBS and fixed in 3.7% formaldehyde in PBS for 15 minutes. This was followed by washing in PBS/0.2% Triton X-100 and permeabilizing in 100% MeOH for 15 minutes. Cells were blocked in Odyssey blocking buffer (LI-COR Biosciences) for at least 1 hour. Antibodies to phosphorylated ERK (Cell Signaling #9106, monoclonal) and total ERK (Santa Cruz Biotechnology #sc-94, polyclonal) were added to the cells and incubated for at least 1 hour. After washing with PBS/0.2% TritonX-100, the cells were incubated with fluorescently-labeled secondary antibodies (goat anti-rabbit IgG-IRDye800, Rockland and goat anti-mouse IgG-Alexa Fluor 680, Molecular Probes) for an additional hour. Cells were then washed and analyzed for fluorescence at both wavelengths using the Odyssey Infrared Imaging System (LI-COR Biosciences). Phosphorylated ERK signal was normalized to total ERK signal.

### Example F

### Cell Viability Assay

Viable cells were quantified after a 3 day incubation with compounds of this invention using the MTS/PMS calorimetric assay from Promega.

Materials and Methods: Malme-3M cells were plated in 96 well plates at a density of 20,000 cells/well. The cells were allowed to attach for 1-2 hours. Diluted compounds were then added to the cells at a final concentration of 0.5% DMSO. After 3 days, the number of viable cells was determined using the MTS assay (Promega, CellTiter 96 Aqueous Non-radioactive Cell Proliferation Assay). Briefly, MTS reagents were added to the cells and incubated for 1 hour. Absorbance at 490 nm was then read using a microplate reader. Background from medium only wells was subtracted.

While the invention has been described in conjunction with the enumerated embodiments, it will be understood that they are not intended to limit the invention to those embodiments. On the contrary, the invention is intended to cover all alternatives, modifications and equivalents, which may be included within the scope of the present invention as defined by the claims. Thus, the foregoing description is considered as illustrative only of the principles of the invention.

The words “comprise,” “comprising,” “include,” “including,” and “includes” when used in this specification and in the following claims are intended to specify the presence of stated features, integers, components, or steps, but they do not preclude the presence or addition of one or more other features, integers, components, steps, or groups thereof.

What is claimed is:

1. A compound selected from Formula I: 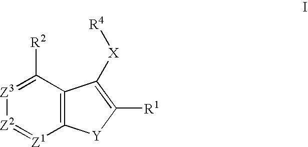
and stereoisomers, geometric isomers, tautomers, solvates, metabolites and pharmaceutically acceptable salts thereof, wherein:
X is selected from NR³, O, C(═O), and S;
Y is O or S;
Z¹, Z², and Z³ are independently selected from CR⁵ and N, and one or two of Z¹, Z², and Z³ is N;
R¹, R² and R⁵ are independently selected from H, F, Cl, Br, I, —C(═Y¹)R, —C(═Y¹)OR, —C(═Y¹)NR₂, —NR₂, —N⁺R³, —N(R)C(═Y¹)R, —N(R)C(═Y¹)OR, —N(R)C(═Y¹)NR₂, —NR-alkylaryl, —NRSO₂NRR, —OR, —OC(═Y¹)R, —OC(═Y¹)OR, —OC(═Y¹)NR₂, —OS(O)₂(OR), —OP(═Y¹)(OR)₂, —OP(OR)₂, —P(═Y¹)(OR)₂, —P(═Y¹)(OR)NR₂, —SR, —S(O)R, —S(O)₂R, —S(O)₂NRR, —S(O)(OR), —S(O)₂(OR), —SC(═Y¹)R, —SC(═Y¹)OR, —SC(═Y¹)NR₂, C₁-C₈ alkylhalide, C₁-C₈ alkylsulfonate, C₁-C₈ alkylamino, C₁-C₈ alkylhydroxyl, C₁-C₈ alkylthiol, 5-7 membered ring lactam, 5-7 membered ring lactone, 5-7 membered ring sultam, C₁-C₈ alkyl, C₂-C₈ alkenyl, C₂-C₈ alkynyl, C₆-C₂₀ aryl, C₃-C₁₂ carbocycle, and C₁-C₂₀ heterocyclyl;
R³ is selected from H, C₁-C₈ alkyl, C₂-C₈ alkenyl, C₂-C₈ alkynyl, C₆-C₂₀ aryl, C₃-C₁₂ carbocycle, C₁-C₂₀ heterocyclyl, and a protecting group;
R⁴ is selected from phenyl, 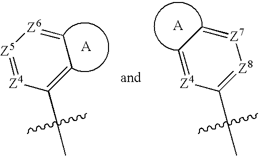
wherein the wavy line indicates the attachment to X;
Z⁴, Z⁵, Z⁶, Z⁷, and Z⁸ are independently selected from CR⁵ and N;
A is (i) an optionally substituted 5 or 6 membered fused heterocyclic ring having one or two heteroatoms independently selected from O, N, and S, (ii) an optionally substituted 5 or 6 membered fused carbocyclic ring, or (iii) an optionally substituted fused phenyl ring;
each R is independently H, C₁-C₈ alkyl, C₂-C₈ alkenyl, C₂-C₈ alkynyl, C₆-C₂₀ aryl, C₁-C₂₀ heterocyclyl, or a protecting group;
Y¹ is independently selected from O, S, NR, N⁺(O)(R), N(OR), N⁺(O)(OR), and N—NRR; and
each alkyl, alkenyl, alkynyl, aryl, phenyl, carbocyclyl, and heterocyclyl is optionally substituted with one or more substituents independently selected from F, Cl, Br, I, CN, CF₃, OR, SR, R, ═O, ═S, ═NR, ═N⁺(O)(R), ═N(OR), —N⁺(O)(OR), ═N—NR₂, —C(═Y¹)R, —C(═Y¹)OR, —C(═Y¹)NR₂, —NR₂, —N⁺R₃, —N(R)C(═Y¹)R, —N(R)C(═Y¹)OR, —N(R)C(═Y¹)NR₂, —SR, —OC(═Y¹)R, —OC(═Y¹)OR, —OC(═Y¹)NR₂, —OS(O)₂(OR), —OP(═Y¹)(OR)₂, —OP(OR)₂, —P(═Y¹)(OR)₂, —P(═Y¹)(OR)NR₂, —S(O)R, —S(O)₂R, —S(O)₂NR, —S(O)(OR), —S(O)₂(OR), —SC(═Y¹)R, —SC(═Y¹)OR, and —SC(═Y¹)NR₂.

2. The compound of claim 1 wherein R⁴ is selected from the structures:

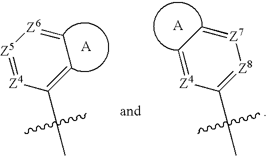

3. The compound of claim 1 having the structure: 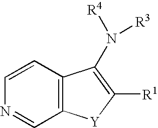
wherein:
Y is S or O;
R¹ is H, I, Br, CH═CH₂, C(═O)OR^{a}, C(═O)R^{b}, CH(OH)Ar, (C₁-C₆ alkyl)OH, C(═NNH₂)(C₁-C₃ alkyl)-O(C₁-C₃ alkyl), C(═O)NR^{c}R^{d}, NHR^{e}, NHC(═O)(C₁-C₆ alkyl), Ar, hetAr, or a saturated or partially unsaturated heterocyclyl;
R³ is H, C₁-C₆ alkyl, or CH₂CH₂OH;
R⁴ is 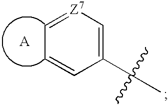
Z⁷ is N or CR⁵;
R⁵ is H or OH;
A is:
(i) a fused 6 membered heteroaryl ring having one or two ring nitrogen atoms and optionally substituted with one to three groups independently selected from C₁-C₃ alkyl, OH, OCH₃, NH₂, F, Cl, Br, I, oxo, and ═NOR^{f};
(ii) a fused 5 membered heteroaryl ring having a ring nitrogen atom and optionally having a second ring heteroatom selected from N and O, wherein said ring is optionally substituted with one or two groups independently selected from NH₂, OR^{f}, F, Cl, Br, I, C₁-C₃ alkyl, oxo and ═NOR^{f};
(iii) a fused 5-6 membered saturated or partially unsaturated heterocyclic ring having one or two ring heteroatoms independently selected from N and O and optionally substituted with one or two groups independently selected from C₁-C₆ alkyl, oxo, and ═NOR^{f};
(iv) a fused 5-6 membered carbocyclic ring optionally substituted with oxo, NH₂, and ═NOR^{f}; or
(v) a fused phenyl ring optionally substituted with one to three groups independently selected from F, Cl, Br, I, OR^{f}, and NH₂;
Ar is phenyl optionally substituted with one to three groups independently selected from OCH₃, CN, C(═O)NR^{f}R^{g}, CF₃, F, Cl, Br, I, NR^{f}R^{g}, C(═O)OR^{f}, and C₁-C₆ alkyl;
hetAr is a 5-6 membered heteroaryl having a ring nitrogen atom and optionally having one or two additional ring heteroatoms independently selected from N, O and S, wherein said heteroaryl is optionally substituted with one to three groups independently selected from (i) C₁-C₆ alkyl, (ii) (C₁-C₆ alkyl)OH, (iii) NR^{f}R^{g}, (iv) (CH₂)₀₋₁-heterocycle or C(═O)heterocycle, wherein said heterocycle is a 6 membered ring having 1 or 2 ring atoms independently selected from N and O and optionally substituted with C₁-C₆ alkyl, (v) C(═O)OR^{f}, (vi) (C₁-C₆ alkyl)NR^{f}R^{g}, (vii) C(═O)NH(C₁-C₆ alkyl)NR^{f}R^{g}, (viii) O—(C₁-C₆)NR^{f}R^{g}, (ix) SMe and (x) CF₃;
R^{a} is H, C₁-C₆ alkyl, or (C₁-C₆ alkyl)-NR^{f}R^{g};
R^{b} is H, Ar, C₁-C₆ alkyl, (C₁-C₆ alkyl)-O(C₁-C₆ alkyl), or a 6 membered heterocycle having 1-2 ring heteroatoms independently selected from N and O;
R^{c} is H or (C₁-C₆ alkyl);
R^{d} is H, C₁-C₆ alkyl, (C₁-C₆ alkyl)NR^{f}R^{g}, NH₂, Ar, (CH₂)₀₋₂-hetAr, (C₁-C₆ alkyl)-OR^{f}, (C₁-C₆ alkyl)-SO₂CH₃, (C₁-C₆ alkyl)CH(OH)(C₁-C₆ alkyl), (C₁-C₆ alkyl)CH(OH)(C₁-C₆ alkyl)OH, (C₁-C₆ alkyl)C(═O)NR^{f}R^{g}, or (CH₂)₀₋₂-heterocycle wherein said heterocycle is a 5-6 membered ring having 1-2 ring atoms independently selected from N and O and optionally substituted with C₁-C₆ alkyl,
or R^{c} and R^{d} together with the nitrogen atom to which they are attached form a 5-6 membered heterocyclic ring having a ring nitrogen atom and optionally having a second ring heteroatom selected from N and O, said ring being optionally substituted with one to three groups independently selected from C₁-C₆ alkyl;
R^{e} is H, C₁-C₆ alkyl, (C₁-C₆ alkyl)O(C₁-C₆ alkyl), or (C₁-C₆ alkyl)NR^{f}R^{g}; and
R^{f} and R^{g} are independently H or C₁-C₆ alkyl, or R^{g} is CH₂Ph.

4. The compound of claim 1 wherein R³ is H.

5. The compound of claim 1 wherein Z⁷ is CR⁵.

6. The compound of claim 1 wherein R⁴ is selected from the structures:

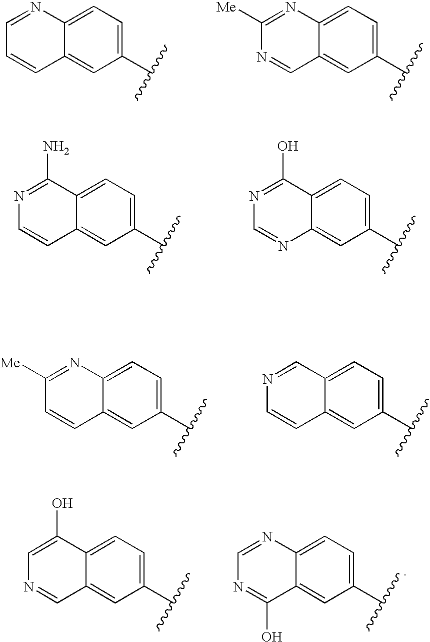

7. The compound of claim 1 wherein R⁴ is selected from the structures:

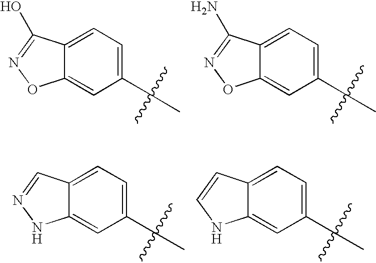

8. The compound of claim 2 wherein A is a fused 5-6 membered saturated or partially unsaturated heterocyclic ring substituted with oxo or ═NOR^{f}.

9. The compound of claim 1, wherein R⁴ is selected from the structures:

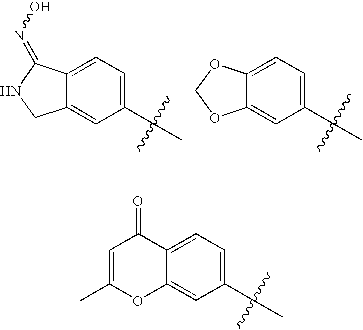

10. The compound of claim 2, wherein A is a fused 5 membered carbocyclic ring substituted with oxo or ═NOR^{f}.

11. The compound of claim 2 wherein R⁴ is selected from the structures:

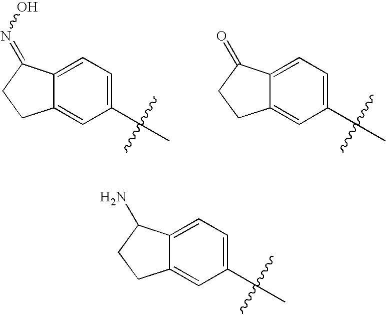

12. The compound of claim 1, wherein R⁴ is selected from the structures:

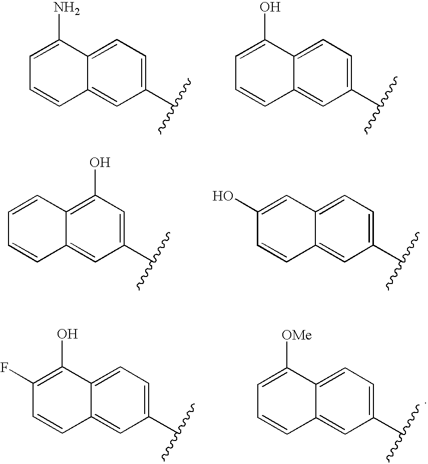

13. The compound of claim 3 wherein R¹ is C(═O)OR^{a}.

14. The compound of claim 13, wherein R¹ is CO₂H, CO₂CH₃, CO₂CH₂CH₃, CO₂CH₂CH₂CH₃, CO₂CH(CH₃)₂ or CO₂CH₂CH₂N(CH₃)₂.

15. The compound of claim 3 wherein R¹ is C(═O)R^{b}.

16. The compound of claim 15, wherein R¹ is C(═O)(4-methoxyphenyl), C(═O)(tetrahydro-2H-pyran-4-yl)C(═O)CH₂CH₂CH₃, C(═O)CH(CH₃)₂, or C(═O)CH₂CH₂CH₂OCH₃.

17. The compound of claim 3 wherein R¹ is CH(OH)R^{b}.

18. The compound of claim 17, wherein R¹ is CH(OH)(4-methoxyphenyl).

19. The compound of claim 3 wherein R¹ is (C₁-C₆ alkyl)OH.

20. The compound of claim 19, wherein R¹ is CH₂OH or CH₂CH₂OH.

21. The compound of claim 3 wherein R¹ is C(═O)NR^{c}R^{d}.

22. The compound of claim 21, wherein R^{d} is (C₁-C₆ alkyl)NH₂, (C₁-C₆ alkyl)NH(C₁-C₆ alkyl), (C₁-C₆ alkyl)N(C₁-C₆ alkyl)₂, (C₁-C₆ alkyl)-heteorcyclyl, (C₁-C₆ alkyl)SO₂CH₃, or (C₁-C₆ alkyl)C(═O)NR^{f}R^{g} and A is other than a cycloalkyl or heterocyclic ring substituted with oxo or ═NOR^{f}.

23. The compound according to claim 21, wherein R¹ is selected from the structures:

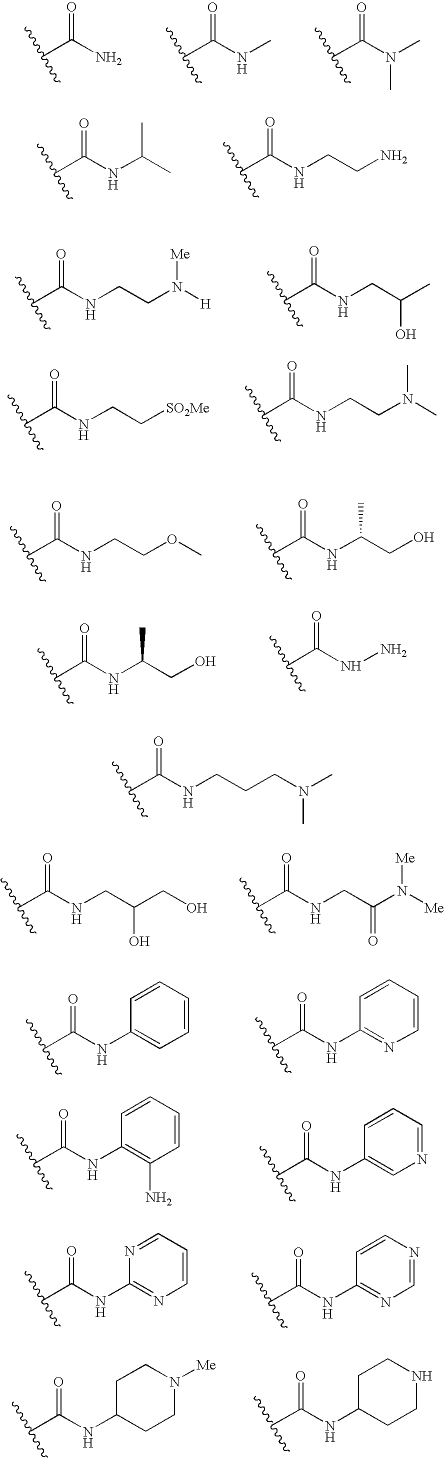

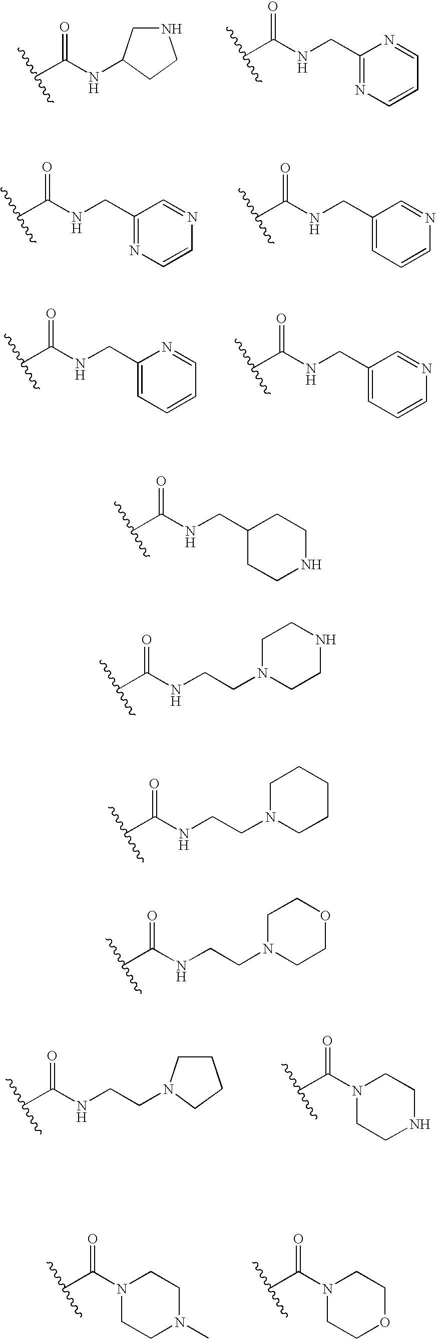

24. The compound of claim 3 wherein R¹ is NHR^{e}.

25. The compound of claim 24, wherein R¹ is NHCH₂CH₃, NHCH₂CH₂CH₃, NHCH₂CH₂OCH₃, NHCH₂CH₂CH₂N(CH₂CH₃)₂, or NH(4-methoxyphenyl).

26. The compound of claim 3 wherein R¹ is NHC(═O)(C₁-C₆ alkyl).

27. The compound of claim 26, wherein R¹ is NHC(═O)CH₂CH₃.

28. The compound of claim 3 wherein R¹ is Ar.

29. The compound of claim 28, wherein R¹ is selected from the structures:
phenyl, 4-methoxyphenyl, 4-cyanophenyl, 4-methylphenyl, 4-trifluoromethylphenyl, 2-trifluoromethylphenyl, 4-carbamoylphenyl, and 4-acetoxyphenyl.

30. The compound of claim 3 wherein R¹ is hetAr.

31. The compound of claim 30 wherein R¹ is selected from the structures:

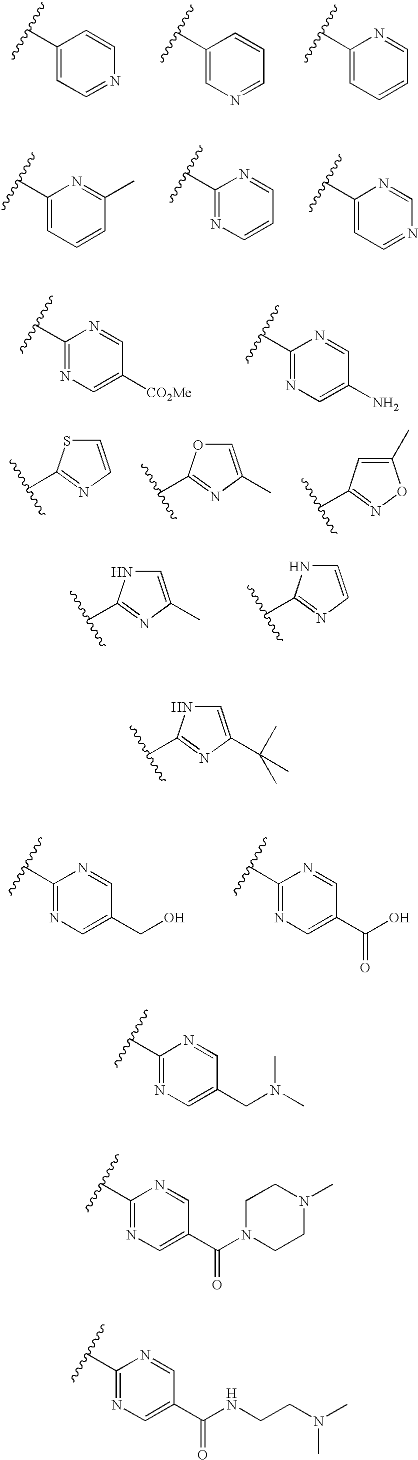

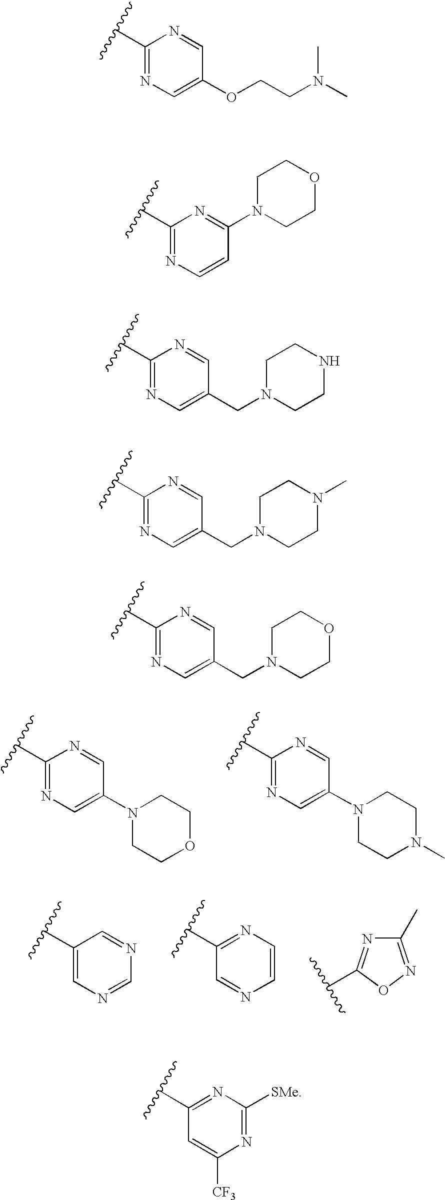

32. The compound of claim 1, selected from:
(E)-ethyl 3-(1-(hydroxyimino)-2,3-dihydro-1H-inden-5-ylamino)thieno[2,3-c]pyridine-2-carboxylate;
(Z)-ethyl 3-(1-(hydroxyimino)-2,3-dihydro-1H-inden-5-ylamino)thieno[2,3-c]pyridine-2-carboxylate;
5-(2-phenylthieno[2,3-c]pyridin-3-ylamino)-2,3-dihydroinden-1-one oxime;
3-(1-(hydroxyimino)-2,3-dihydro-1H-inden-5-ylamino)-N-methylthieno[2,3-c]pyridine-2-carboxamide;
E-(3-(1-(hydroxyimino)-2,3-dihydro-1H-inden-5-ylamino)thieno[2,3-c]pyridin-2-yl)(4-methoxyphenyl)methanone oxime;
Z-(3-(1-(hydroxyimino)-2,3-dihydro-1H-inden-5-ylamino)thieno[2,3-c]pyridin-2-yl)(4-methoxyphenyl)methanone oxime;
(E)-5-(2-(hydroxy(4-methoxyphenyl)methyl)thieno[2,3-c]pyridin-3-ylamino)-2,3-dihydroinden-1-one oxime;
(E)-3-(1-(hydroxyimino)-2,3-dihydro-1H-inden-5-ylamino)thieno[2,3-c]pyridine-2-carboxylic acid;
(E)-1-(3-(1-(hydroxyimino)-2,3-dihydro-1H-inden-5-ylamino)thieno[2,3-c]pyridin-2-yl)butan-1-one;
(E)-isopropyl 3-(1-(hydroxyimino)-2,3-dihydro-1H-inden-5-ylamino)thieno[2,3-c]pyridine-2-carboxylate;
5-(2-(pyridin-3-yl)thieno[2,3-c]pyridin-3-ylamino)-2,3-dihydroinden-1-one oxime;
3-(1-(hydroxyimino)-2,3-dihydro-1H-inden-5-ylamino)-N,N-dimethylthieno[2,3-c]pyridine-2-carboxamide;
3-(1-(hydroxyimino)-2,3-dihydro-1H-inden-5-ylamino)-N-phenylthieno[2,3-c]pyridine-2-carboxamide;
tert-butyl 2-iodothieno[2,3-c]pyridin-3-ylcarbamate;
5-(2-(4-methoxyphenyl)thieno[2,3-c]pyridin-3-ylamino)-2,3-dihydroinden-1-one oxime;
5-(2-iodothieno[2,3-c]pyridin-3-ylamino)-2,3-dihydroinden-1-one oxime;
(E,Z)-(3-(1-(hydroxyimino)-2,3-dihydro-1H-inden-5-ylamino)thieno[2,3-c]pyridin-2-yl)(morpholino)methanone;
5-(2-(hydroxymethyl)thieno[2,3-c]pyridin-3-ylamino)-2,3-dihydroinden-1-one oxime;
ethyl 3-(1-(hydroxyimino)-2,3-dihydro-1H-inden-5-ylamino)thieno[3,2-c]pyridine-2-carboxylate;
2-(dimethylamino)ethyl-3-(1-(hydroxyimino)-2,3-dihydro-1H-inden-5-ylamino)thieno[2,3-c]pyridine-2-carboxylate;
ethyl 3-(1-(hydroxyimino)-2,3-dihydro-1H-inden-5-ylamino)furo[2,3-c]pyridine-2-carboxylate;
methyl 3-(1-(hydroxyimino)-2,3-dihydro-1H-inden-5-ylamino)furo[2,3-c]pyridine-2-carboxylate;
isopropyl 3-(1-(hydroxyimino)-2,3-dihydro-1H-inden-5-ylamino)furo[2,3-c]pyridine-2-carboxylate;
5-(2-(pyridin-4-yl)furo[2,3-c]pyridin-3-ylamino)-2,3-dihydroinden-1-one oxime;
5-(2-(pyridin-2-yl)furo[2,3-c]pyridin-3-ylamino)-2,3-dihydroinden-1-one oxime;
4-(3-(1-(hydroxyimino)-2,3-dihydro-1H-inden-5-ylamino)furo[2,3-c]pyridin-2-yl)benzonitrile;
4-(3-(1-(hydroxyimino)-2,3-dihydro-1H-inden-5-ylamino)furo[2,3-c]pyridin-2-yl)benzamide;
(1-(hydroxyimino)-2,3-dihydro-1H-inden-5-yl)(2-(4-methoxybenzylamino)-thieno[2,3-c]pyridin-3-yl)methanone;
(2-(ethylamino)thieno[2,3-c]pyridin-3-yl)(1-(hydroxyimino)-2,3-dihydro-1H-inden-5-yl)methanone;
(1-(hydroxyimino)-2,3-dihydro-1H-inden-5-yl)(2-(propylamino)thieno[2,3-c]pyridin-3-yl)methanone;
(1-(hydroxyimino)-2,3-dihydro-1H-inden-5-yl)(2-(2-methoxyethylamino)thieno[2,3-c]pyridin-3-yl)methanone;
(2-(3-(diethylamino)propylamino)thieno[2,3-c]pyridin-3-yl)(1-(hydroxyimino)-2,3-dihydro-1H-inden-5-yl)methanone;
N-(3-(1-(hydroxyimino)-2,3-dihydro-1H-indene-5-carbonyl)thieno[2,3-c]pyridin-2-yl)propionamide;
5-(2-(pyrimidin-2-yl)furo[2,3-c]pyridin-3-ylamino)-2,3-dihydroinden-1-one oxime; 5-(2-(5-methylisoxazole-3-yl)furo[2,3-c]pyridin-3-ylamino)-2,3-dihydroinden-1-one oxime;
5-(2-(2-(trifluoromethyl)phenyl)furo[2,3-c]pyridin-3-ylamino)-2,3-dihydroinden-1-one oxime;
5-(2-(6-methylpyridin-2-yl)furo[2,3-c]pyridin-3-ylamino)-2,3-dihydroinden-1-one oxime;
N-(2-(dimethylamino)ethyl)-3-(1-(hydroxyimino)-2,3-dihydro-1H-inden-5-ylamino)furo[2,3-c]pyridine-2-carboxamide;
3-(1-(hydroxyimino)-2,3-dihydro-1H-inden-5-ylamino)-N-isopropylfuro[2,3-c]pyridine-2-carboxamide;
3-(1-(hydroxyimino)-2,3-dihydro-1H-inden-5-ylamino)-N-(pyridin-2-yl)furo[2,3-c]pyridine-2-carboxamide;
3-(1-(hydroxyimino)-2,3-dihydro-1H-inden-5-ylamino)-N-(pyridin-2-ylmethyl)furo[2,3-c]pyridine-2-carboxamide;
3-(1-(hydroxyimino)-2,3-dihydro-1H-inden-5-ylamino)-N-(pyridin-3-ylmethyl)furo[2,3-c]pyridine-2-carboxamide;
5-(2-(4-(trifluoromethyl)phenyl)furo[2,3-c]pyridin-3-ylamino)-2,3-dihydro-1H-inden-1-one oxime;
5-(2-(Pyridin-2-yl)furo[2,3-c]pyridin-3-ylamino)-2,3-dihydro-1H-inden-1-one;
5-(2-p-Tolylfuro[2,3-c]pyridin-3-ylamino)-2,3-dihydro-1H-inden-1-one oxime;
6-(2-(4-ethyl-1H-imidazol-2-yl)furo[2,3-c]pyridin-3-ylamino)naphthalen-1-ol;
6-(2-(4-tert-butyl-1H-imidazol-2-yl)furo[2,3-c]pyridin-3-ylamino)naphthalen-1-ol;
Ethyl 3-(5-aminonaphthalen-2-ylamino)furo[2,3-c]pyridine-2-carboxylate;
Ethyl 3-(2-methylquinazolin-6-ylamino)furo[2,3-c]pyridine-2-carboxylate;
Ethyl 3-(6-fluoro-5-hydroxynaphthalen-2-ylamino)furo[2,3-c]pyridine-2-carboxylate;
Ethyl 3-((5-hydroxynaphthalen-2-yl)(methyl)amino)furo[2,3-c]pyridine-2-carboxylate;
Ethyl 3-(3-aminobenzo[d]isoxazol-6-ylamino)furo[2,3-c]pyridine-2-carboxylate;
3-(3-Hydroxybenzo[d]isoxazol-6-ylamino)furo[2,3-c]pyridine-2-carboxylate;
Ethyl 3-(4-hydroxyisoquinolin-7-ylamino)furo[2,3-c]pyridine-2-carboxylate;
ethyl 3-(isoquinolin-3-ylamino)furo[2,3-c]pyridine-2-carboxylate;
Ethyl 3-(6-hydroxynaphthalen-2-ylamino)furo[2,3-c]pyridine-2-carboxylate;
Ethyl 3-(5-hydroxynaphthalen-2-ylamino)furo[2,3-c]pyridine-2-carboxylate;
Ethyl 3-(3-hydroxy-4-methoxyphenylamino)furo[2,3-c]pyridine-2-carboxylate;
Ethyl 3-(quinolin-6-ylamino)furo[2,3-c]pyridine-2-carboxylate;
Ethyl 3-(benzo[d][1,3]dioxol-5-ylamino)furo[2,3-c]pyridine-2-carboxylate;
Methyl 3-(isoquinolin-4-ylamino)furo[2,3-c]pyridine-2-carboxylate;
Methyl 3-(quinolin-3-ylamino)furo[2,3-c]pyridine-2-carboxylate;
N-(2-(Dimethylamino)ethyl)-3-(8-hydroxyquinolin-3-ylamino)furo[2,3-c]pyridine-2-carboxamide;
N-(2-(dimethylamino)ethyl)-3-(5-hydroxyquinolin-2-ylamino)furo[2,3-c]pyridine-2-carboxamide;
N-(2-(dimethylamino)ethyl)-3-(1-oxo-2,3-dihydro-1H-inden-5-ylamino)furo[2,3-c]pyridine-2-carboxamide;
N-(2,3-dihydroxypropyl)-3-(5-hydroxynaphthalen-2-ylamino)furo[2,3-c]pyridine-2-carboxamide;
N-(3-(dimethylamino)propyl)-3-(1-(hydroxyimino)-2,3-dihydro-1H-inden-5-ylamino)furo[2,3-c]pyridine-2-carboxamide;
3-(1-(hydroxyimino)-2,3-dihydro-1H-inden-5-ylamino)-N-(piperidin-4-yl)furo[2,3-c]pyridine-2-carboxamide;
3-(1-(hydroxyimino)-2,3-dihydro-1H-inden-5-ylamino)-N-(pyridin-3-yl)furo[2,3-c]pyridine-2-carboxamide;
3-(1-(hydroxyimino)-2,3-dihydro-1H-inden-5-ylamino)-N-(piperidin-4-ylmethyl)furo[2,3-c]pyridine-2-carboxamide;
3-(1-(hydroxyimino)-2,3-dihydro-1H-inden-5-ylamino)-N-(1-methylpiperidin-4-yl)furo[2,3-c]pyridine-2-carboxamide;
3-(1-(hydroxyimino)-2,3-dihydro-1H-inden-5-ylamino)-N-(2-(pyrrolidin-1-yl)ethyl)furo[2,3-c]pyridine-2-carboxamide;
3-(1-(hydroxyimino)-2,3-dihydro-1H-inden-5-ylamino)-N-(2-(piperidin-1-yl)ethyl)furo[2,3-c]pyridine-2-carboxamide;
3-(1-(hydroxyimino)-2,3-dihydro-1H-inden-5-ylamino)-N-(2-morpholinoethyl)furo[2,3-c]pyridine-2-carboxamide;
3-(5-hydroxynaphthalen-2-ylamino)-N-(2-methoxyethyl)furo[2,3-c]pyridine-2-carboxamide;
3-(5-hydroxynaphthalen-2-ylamino)-N-isopropylfuro[2,3-c]pyridine-2-carboxamide;
N-(2-(dimethylamino)ethyl)-3-(5-hydroxynaphthalen-2-ylamino)furo[2,3-c]pyridine-2-carboxamide;
3-(5-hydroxynaphthalen-2-ylamino)-N-(pyridin-3-ylmethyl)furo[2,3-c]pyridine-2-carboxamide;
N-(2-(dimethylamino)ethyl)-3-(4-hydroxynaphthalen-2-ylamino)furo[2,3-c]pyridine-2-carboxamide;
3-(5-hydroxynaphthalen-2-ylamino)-N-(pyrimidin-4-yl)furo[2,3-c]pyridine-2-carboxamide;
(R)-3-(1-(hydroxyimino)-2,3-dihydro-1H-inden-5-ylamino)-N-(1-hydroxypropan-2-yl)furo[2,3-c]pyridine-2-carboxamide;
(S)-3-(1-(hydroxyimino)-2,3-dihydro-1H-inden-5-ylamino)-N-(1-hydroxypropan-2-yl)furo[2,3-c]pyridine-2-carboxamide;
3-(1-(hydroxyimino)-2,3-dihydro-1H-inden-5-ylamino)-N-(2-(piperazin-1-yl)ethyl)furo[2,3-c]pyridine-2-carboxamide;
3-(1-(hydroxyimino)-2,3-dihydro-1H-inden-5-ylamino)-N-(2-methoxyethyl)furo[2,3-c]pyridine-2-carboxamide;
3-(1-(hydroxyimino)-2,3-dihydro-1H-inden-5-ylamino)-N-(pyrimidin-2-ylmethyl)furo[2,3-c]pyridine-2-carboxamide;
3-(1-(hydroxyimino)-2,3-dihydro-1H-inden-5-ylamino)-N-(pyrimidin-4-yl)furo[2,3-c]pyridine-2-carboxamide;
3-(1-(hydroxyimino)-2,3-dihydro-1H-inden-5-ylamino)-N-(pyrimidin-2-yl)furo[2,3-c]pyridine-2-carboxamide;
N-(2-(dimethylamino)-2-oxoethyl)-3-(1-(hydroxyimino)-2,3-dihydro-1H-inden-5-ylamino)furo[2,3-c]pyridine-2-carboxamide;
3-(1-(hydroxyimino)-2,3-dihydro-1H-inden-5-ylamino)-N-(2-(methylsulfonyl)ethyl)furo[2,3-c]pyridine-2-carboxamide;
3-(1-(hydroxyimino)-2,3-dihydro-1H-inden-5-ylamino)-N-(2-(methylamino)ethyl)furo[2,3-c]pyridine-2-carboxamide;
3-(1-(hydroxyimino)-2,3-dihydro-1H-inden-5-ylamino)-N-(pyrrolidin-3-yl)furo[2,3-c]pyridine-2-carboxamide;
N-(2-(dimethylamino)ethyl)-3-(6-fluoro-5-hydroxynaphthalen-2-ylamino)furo[2,3-c]pyridine-2-carboxamide;
3-(6-fluoro-5-hydroxynaphthalen-2-ylamino)-N-(pyrimidin-2-yl)furo[2,3-c]pyridine-2-carboxamide;
N-(2-(dimethylamino)ethyl)-3-(quinolin-3-ylamino)furo[2,3-c]pyridine-2-carboxamide;
N-(2-aminophenyl)-3-(5-hydroxynaphthalen-2-ylamino)furo[2,3-c]pyridine-2-carboxamide;
N-(2-Aminoethyl)-3-(5-hydroxynaphthalen-2-ylamino)furo[2,3-c]pyridine-2-carboxamide;
6-(2-(4,5-Dihydro-1H-imidazol-2-yl)furo[2,3-c]pyridin-3-ylamino)naphthalen-1-ol;
5-(2-(Pyrazin-2-yl)furo[2,3-c]pyridin-3-ylamino)-2,3-dihydro-1H-inden-1-one oxime;
5-(2-(Pyrimidine-4-yl)furo[2,3-c]pyridin-3-ylamino)-2,3-dihydro-1H-inden-1-one oxime;
6-(2-(pyrimidin-2-yl)furo[2,3-c]pyridin-3-ylamino)naphthalen-1-ol;
5-(2-(pyrimidin-2-yl)furo[2,3-c]pyridin-3-ylamino)-2,3-dihydro-1H-inden-1-one;
2-(pyrimidin-2-yl)-N-(quinolin-3-yl)furo[2,3-c]pyridin-3-amine;
N-(3,4-dichlorophenyl)-2-(pyrimidin-2-yl)furo[2,3-c]pyridin-3-amine;
(Z,E)-methyl 2-(3-(1-(hydroxyimino)-2,3-dihydro-1H-inden-5-ylamino)furo[2,3-c]pyridin-2-yl)pyrimidine-5-carboxylate;
(Z,E)-(2-(3-(1-(hydroxyimino)-2,3-dihydro-1H-inden-5-ylamino)furo[2,3-c]pyridin-2-yl)pyrimidin-5-yl)(4-methylpiperazin-1-yl)methanone;
(Z,E)-N-(2-(dimethylamino)ethyl)-2-(3-(1-(hydroxyimino)-2,3-dihydro-1H-inden-5-ylamino)furo[2,3-c]pyridin-2-yl)pyrimidine-5-carboxamide;
5-(2-(5-(hydroxymethyl)pyrimidin-2-yl)furo[2,3-c]pyridin-3-ylamino)-2,3-dihydroinden-1-one oxime;
(Z,E)-2-(3-(1-(hydroxyimino)-2,3-dihydro-1H-inden-5-ylamino)furo[2,3-c]pyridin-2-yl)pyrimidine-5-carboxylic acid;
5-(2-(5-((4-Methylpiperazin-1-yl)methyl)pyrimidin-2-yl)furo[2,3-c]pyridin-3-ylamino)-2,3-dihydroinden-1-one oxime;
5-(2-(5-(morpholinomethyl)pyrimidin-2-yl)furo[2,3-c]pyridin-3-ylamino)-2,3-dihydro-1H-inden-1-one oxime;
5-(2-(5-((dimethylamino)methyl)pyrimidin-2-yl)furo[2,3-c]pyridin-3-ylamino)-2,3-dihydro-1H-inden-1-one oxime;
5-(2-(5-(piperazin-1-ylmethyl)pyrimidin-2-yl)furo[2,3-c]pyridin-3-ylamino)-2,3-dihydro-1H-inden-1-one oxime;
6-(2-(5-((4-methylpiperazin-1-yl)methyl)pyrimidin-2-yl)furo[2,3-c]pyridin-3-ylamino)naphthalen-1-ol;
6-(2-(5-(piperazin-1-ylmethyl)pyrimidin-2-yl)furo[2,3-c]pyridin-3-ylamino)naphthalen-1-ol;
Methyl 2-(3-(5-hydroxynaphthalen-2-ylamino)furo[2,3-c]pyridin-2-yl)pyrimidine-5-carboxylate;
(Z,E)-5-(2-(5-(4-methylpiperazin-1-yl)pyrimidin-2-yl)furo[2,3-c]pyridin-3-ylamino)-2,3-dihydroinden-1-one oxime;
5-(2-(5-(4-methylpiperazin-1-yl)pyrimidin-2-yl)furo[2,3-c]pyridin-3-ylamino)-2,3-dihydroinden-1-one;
N5-(2-(5-(4-Methylpiperazin-1-yl)pyrimidin-2-yl)furo[2,3-c]pyridin-3-yl)-2,3-dihydro-1H-indene-1,5-diamine;
N-(2-(5-(4-Methylpiperazin-1-yl)pyrimidin-2-yl)furo[2,3-c]pyridin-3-yl)quinolin-3-amine;
N-(2-(2-(2-(Dimethylamino)ethoxy)pyrimidin-5-yl)furo[2,3-c]pyridin-3-yl)quinolin-3-amine;
(E)-5-(2-(4-Morpholinopyrimidin-2-yl)furo[2,3-c]pyridin-3-ylamino)-2,3-dihydro-1H-inden-1-one oxime;
6-(2-(4-Morpholinopyrimidin-2-yl)furo[2,3-c]pyridin-3-ylamino)naphthalen-1-ol;
(E)-6-(2-(1-Hydrazono-4-methoxybutyl)furo[2,3-c]pyridin-3-ylamino)naphthalen-1-ol;
(3-(1-(Hydroxyimino)-2,3-dihydro-1H-inden-5-ylamino)furo[2,3-c]pyridin-2-yl)(tetrahydro-2H-pyran-4-yl)methanone;
1-(3-(5-Hydroxynaphthalen-2-ylamino)furo[2,3-c]pyridin-2-yl)-4-methoxybutan-1-one;
1-(3-(1-(Hydroxyimino)-2,3-dihydro-1H-inden-5-ylamino)furo[2,3-c]pyridin-2-yl)butan-1-one;
(Z)-5-(2-(Pyrimidin-2-yl)furo[2,3-c]pyridin-3-ylamino)isoindolin-1-one oxime;
(Z)-3-(1-(Hydroxyimino)isoindolin-5-ylamino)-N-isopropylfuro[2,3-c]pyridine-2-carboxamide;
(Z)-3-(1-(Hydroxyimino)isoindolin-5-ylamino)-N-(pyridin-3-ylmethyl)furo[2,3-c]pyridine-2-carboxamide;
6-(Furo[2,3-c]pyridin-3-ylamino)naphthalen-1-ol;
6-(Furo[2,3-c]pyridin-3-yl(2-hydroxyethyl)amino)naphthalen-1-ol;
5-(Furo[2,3-c]pyridin-3-ylamino)-2,3-dihydro-1H-inden-1-one oxime;
N-(2-(dimethylamino)ethyl)-3-(1-(hydroxyimino)-2,3-dihydro-1H-inden-5-ylamino)thieno[2,3-c]pyridine-2-carboxamide;
3-(1-(Hydroxyimino)-2,3-dihydro-1H-inden-5-ylamino)-N-isopropylthieno[2,3-c]pyridine-2-carboxamide;
3-(1-(Hydroxyimino)-2,3-dihydro-1H-inden-5-ylamino)-N-(pyridin-3-ylmethyl)thieno[2,3-c]pyridine-2-carboxamide;
methyl 4-(3-(1-(hydroxyimino)-2,3-dihydro-1H-inden-5-ylamino)furo[2,3-c]pyridin-2-yl)benzoate;
5-(2-(thiazol-2-yl)furo[2,3-c]pyridin-3-ylamino)-2,3-dihydro-1H-inden-1-one oxime;
5-(2-(3-methyl-1,2,4-oxadiazol-5-yl)furo[2,3-c]pyridin-3-ylamino)-2,3-dihydro-1H-inden-1-one oxime;
ethyl 3-(8-methoxyquinolin-3-ylamino)furo[2,3-c]pyridine-2-carboxylate;
N-(2-(diethylamino)ethyl)-3-(1-(hydroxyimino)-2,3-dihydro-1H-inden-5-ylamino)furo[2,3-c]pyridine-2-carboxamide;
ethyl 3-(1-oxo-2,3-dihydro-1H-inden-5-ylamino)furo[2,3-c]pyridine-2-carboxylate;
(3-(1-(hydroxyimino)-2,3-dihydro-1H-inden-5-ylamino)furo[2,3-c]pyridin-2-yl)(morpholino)methanone;
(3-(1-(hydroxyimino)-2,3-dihydro-1H-inden-5-ylamino)furo[2,3-c]pyridin-2-yl)(piperazin-1-yl)methanone;
ethyl 3-(isoquinolin-6-ylamino)furo[2,3-c]pyridine-2-carboxylate;
ethyl 3-(5-hydroxynaphthalen-1-ylamino)furo[2,3-c]pyridine-2-carboxylate;
ethyl 3-(4-hydroxyquinazolin-6-ylamino)furo[2,3-c]pyridine-2-carboxylate;
N-(quinolin-3-yl)furo[2,3-c]pyridin-3-amine;
N-(1H-indol-6-yl)furo[2,3-c]pyridin-3-amine;
2-(2-(methylthio)-6-(trifluoromethyl)pyrimidin-4-yl)-N-(quinolin-3-yl)furo[2,3-c]pyridin-3-amine;
ethyl 3-(2-methylquinolin-6-ylamino)furo[2,3-c]pyridine-2-carboxylate;
ethyl 3-(naphthalen-2-ylamino)furo[2,3-c]pyridine-2-carboxylate;
ethyl 3-(4-hydroxyquinazolin-7-ylamino)furo[2,3-c]pyridine-2-carboxylate;
N-(3-(dimethylamino)propyl)-3-(4-hydroxyquinazolin-7-ylamino)furo[2,3-c]pyridine-2-carboxamide;
ethyl 3-(1-aminoisoquinolin-6-ylamino)furo[2,3-c]pyridine-2-carboxylate;
3-(1-aminoisoquinolin-6-ylamino)-N-(2-(dimethylamino)ethyl)furo[2,3-c]pyridine-2-carboxamide;
ethyl 3-(naphthalen-1-ylamino)furo[2,3-c]pyridine-2-carboxylate;
N-(1H-indazol-6-yl)furo[2,3-c]pyridin-3-amine;
and salts, geometric isomers and resolved enantiomers and diastereomers thereof.

33. A compound selected from Formula II: 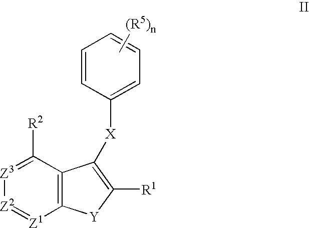
and stereoisomers, geometric isomers, tautomers, solvates, metabolites and pharmaceutically acceptable salts thereof, wherein:
X is selected from NR³, O, C(═O), and S;
Y is O or S;
Z¹, Z², and Z³ are independently selected from CR⁵ and N, and one or two of Z¹, Z², and Z³ is N;
R¹, R² and R⁵ are independently selected from H, F, Cl, Br, I, —C(═Y¹)R, —C(═Y¹)OR, —C(═Y¹)NR₂, —NR₂, —N⁺R³, —N(R)C(═Y¹)R, —N(R)C(═Y¹)OR, —N(R)C(═Y¹)NR₂, —NR-alkylaryl, —NRSO₂NRR, —OR, —OC(═Y¹)R, —OC(═Y¹)OR, —OC(═Y¹)NR₂, —OS(O)₂(OR), —OP(═Y¹)(OR)₂, —OP(OR)₂, —P(═Y¹)(OR)₂, —P(═Y¹)(OR)NR₂, —SR, —S(O)R, —S(O)₂R, —S(O)₂NRR, —S(O)(OR), —S(O)₂(OR), —SC(═Y¹)R, —SC(═Y¹)OR, —SC(═Y¹)NR₂, C₁-C₈ alkylhalide, C₁-C₈ alkylsulfonate, C₁-C₈ alkylamino, C₁-C₈ alkylhydroxyl, C₁-C₈ alkylthiol, 5-7 membered ring lactam, 5-7 membered ring lactone, 5-7 membered ring sultam, C₁-C₈ alkyl, C₂-C₈ alkenyl, C₂-C₈ alkynyl, C₆-C₂₀ aryl, C₃-C₁₂ carbocycle, and C₁-C₂₀ heterocyclyl;
R³ is selected from H, C₁-C₈ alkyl, C₂-C₈ alkenyl, C₂-C₈ alkynyl, C₆-C₂₀ aryl, C₃-C₁₂ carbocycle, C₁-C₂₀ heterocyclyl, and a protecting group;
each R is independently H, C₁-C₈ alkyl, C₂-C₈ alkenyl, C₂-C₈ alkynyl, C₆-C₂₀ aryl, C₁-C₂₀ heterocyclyl, or a protecting group;
Y¹ is independently selected from O, S, NR, N⁺(O)(R), N(OR), N⁺(O)(OR), and N—NRR;
each alkyl, alkenyl, alkynyl, aryl, phenyl, carbocyclyl, and heterocyclyl is optionally substituted with one or more substituents independently selected from F, Cl, Br, I, CN, CF₃, OR, SR, R, ═O, ═S, ═NR, ═N⁺(O)(R), ═N(OR), ═N⁺(O)(OR), ═N—NR₂, —C(═Y¹)R, —C(═Y¹)OR, —C(═Y¹)NR₂, —NR₂, —N⁺R₃, —N(R)C(═Y¹)R, —N(R)C(═Y¹)OR, —N(R)C(═Y¹)NR₂, —SR, —OC(═Y¹)R, —OC(═Y¹)OR, —OC(═Y¹)NR₂, —OS(O)₂(OR), —OP(═Y¹)(OR)₂, —OP(OR)₂, —P(═Y¹)(OR)₂, —P(═Y¹)(OR)NR₂, —S(O)R, —S(O)₂R, —S(O)₂NR, —S(O)(OR), —S(O)₂(OR), —SC(═Y¹)R, —SC(═Y¹)OR, and —SC(═Y¹)NR₂; and
n is 0, 1, 2, 3, 4 or 5.

34. The compound of claim 33 having the structure: 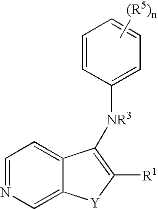
and stereoisomers, geometric isomers, tautomers, solvates, and salts thereof, wherein:
Y is O or S;
R¹ is H, I, Br, CH═CH₂, C(═O)OR^{a}, C(═O)R^{b}, CH(OH)Ar, (C₁-C₆ alkyl)OH, C(═NNH₂)(C₁-C₃ alkyl)-O(C₁-C₃ alkyl), C(═O)NR^{c}R^{d}, NHR^{e}, NHC(═O)(C₁-C₆ alkyl), Ar, hetAr or a saturated or partially unsaturated heterocyclyl;
R³ is H, C₁-C₆ alkyl or CH₂CH₂OH;
each R⁵ is independently selected from F, Cl, Br, I, CN, CF₃, C₁-C₆ alkyl, phenyl, O-phenyl, OH, OMe, CH₂OH, C(═O)(C₁-C₆ alkyl), NHC(═O)(C₁-C₄ alkyl), and 4-methylpyrazol-3-yl;
n is 0, 1, 2 or 3;
Ar is phenyl optionally substituted with one to three groups independently selected from OCH₃, CN, C(═O)NR^{f}R^{g}, CF₃, F, Cl, Br, I, NR^{f}R^{g}, C(═O)OR^{f}, and C₁-C₆ alkyl;
hetAr is a 5-6 membered heteroaryl having a ring nitrogen atom and optionally having one or two additional ring heteroatoms independently selected from N, O and S, wherein said heteroaryl is optionally substituted with one to three groups independently selected from (i) C₁-C₆ alkyl, (ii) (C₁-C₆ alkyl)OH, (iii) NR^{f}R^{g}, (iv) (CH₂)₀₋₁-heterocycle or C(═O)heterocycle, wherein said heterocycle is a 6 membered ring having 1 or 2 ring atoms independently selected from N and O and optionally substituted with C₁-C₆ alkyl, (v) C(═O)OR^{f}, (vi) (C₁-C₆ alkyl)NR^{f}R^{g}, (vii) C(═O)NH(C₁-C₆ alkyl)NR^{f}R^{g}, (viii) 0-(C₁-C₆)NR^{f}R^{g}, (ix) SMe and (x) CF₃;
R^{a} is H, C₁-C₆ alkyl, or (C₁-C₆ alkyl)-NR^{f}R^{g};
R^{b} is H, Ar, C₁-C₆ alkyl, (C₁-C₆ alkyl)-O(C₁-C₆ alkyl), or a 6 membered heterocycle having 1-2 ring heteroatoms independently selected from N and O;
R^{c} is H or (C₁-C₆ alkyl);
R^{d} is H, C₁-C₆ alkyl, (C₁-C₆ alkyl)NR^{f}R^{g}, NR^{f}R^{g}, Ar, (CH₂)₀₋₂-hetAr, (C₁-C₆ alkyl)-OR^{f}, (C₁-C₆ alkyl)-SO₂CH₃, (C₁-C₆ alkyl)CH(OH)(C₁-C₆ alkyl), (C₁-C₆ alkyl)CH(OH)(C₁-C₆ alkyl)OH, (C₁-C₆ alkyl)C(═O)NR^{f}R^{g}, or (CH₂)₀₋₁-heterocycle wherein said heterocycle is a 5-6 membered ring having 1-2 ring atoms independently selected from N and O and optionally substituted with C₁-C₆ alkyl,
or R^{c} and R^{d} together with the nitrogen atom to which they are attached form a 5-6 membered heterocyclic ring having a ring nitrogen atom and optionally having a second ring heteroatom selected from N and O, said ring being optionally substituted with C₁-C₆ alkyl;
R^{e} is H, C₁-C₆ alkyl, (C₁-C₆ alkyl)O(C₁-C₆ alkyl), or (C₁-C₆ alkyl)NR^{f}R^{g}; and
R^{f} and R^{g} are independently H or C₁-C₆ alkyl, or R^{g} is CH₂Ph.

35. A pharmaceutical composition comprised of a compound of claim 1.

36. The composition according to claim 35, additionally comprising a therapeutic agent selected from an anti-proliferative agent, an anti-inflammatory agent, an immunomodulatory agent, a neurotrophic factor, an agent for treating cardiovascular disease, an agent for treating liver disease, an anti-viral agent, an agent for treating blood disorders, an agent for treating diabetes, or an agent for treating immunodeficiency disorders.

37. A method of treating cancer in a mammal in need of such treatment, said method comprising administering to said mammal a therapeutically effective amount of a compound of claim 1.

38. A method of treating an inflammatory diseases selected from rheumatoid arthritis, psoriasis, contact dermatitis, and delayed hypersensitivity reactions in a mammal in need of such treatment, said method comprising administering to said mammal a therapeutically effective amount of a compound of claim 1.
